(19) United States
(12) Patent Application Publication
(10) Pub. No.: US 2026/0324280 A1
(43) Pub. Date: Oct. 15, 2026

(54) LIGHT-EMITTING ELEMENT, AMINE COMPOUND FOR LIGHT-EMITTING ELEMENT, AND ELECTRONIC APPARATUS INCLUDING THE LIGHT-EMITTING ELEMENT

(71) Applicant: Samsung Display Co., Ltd., Yongin-si (KR)

(72) Inventors: Ichiro IMADA, Yokohama-shi (JP); Ichinori TAKADA, Yokohama-shi (JP)

(21) Appl. No.: 19/461,968

(22) Filed: Jan. 28, 2026

(30) Foreign Application Priority Data
Mar. 24, 2025 (JP) 2025-048739

(51) Int. Cl. H10K 85/60; C07D 209/86; C07D 405/12; C09K 11/02; H10K 50/11; H10K 50/15; H10K 50/17; H10K 50/18

(52) U.S. Cl. CPC H10K 85/633; C07D 209/86; C07D 405/12; C09K 11/02; H10K 85/636; H10K 50/11; H10K 50/15; H10K 50/17; H10K 50/181; H10K 85/615; H10K 85/626; H10K 85/6572; H10K 85/6574

(57) ABSTRACT

Provided is a light-emitting element including a first electrode, a second electrode disposed on the first electrode, an emission layer disposed between the first electrode and the second electrode, and a hole transport region disposed between the first electrode and the emission layer. The hole transport region includes an amine compound according to Formula 1, resulting in the light-emitting element exhibiting a reduced driving voltage and long lifespan characteristics.

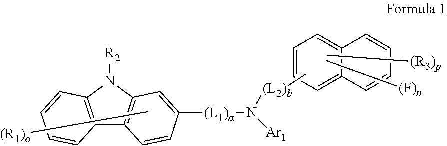

### CROSS-REFERENCE TO RELATED APPLICATIONS

This U.S. non-provisional patent application claims priority to and the benefit of Japan Patent Application No. 2025-048739, filed on Mar. 24, 2025 in the Japanese Patent Office (JPO), and all benefits accruing therefrom under 35 U.S.C. § 119, the content of which is incorporated herein its entirety by reference.

### BACKGROUND

The present disclosure herein relates to a light-emitting element, an amine compound used in the light-emitting element, and an electronic apparatus including the light-emitting element.

A display device includes an electronic apparatus, which includes an organic electroluminescence display device. An organic electroluminescence display device may include an organic light-emitting element. The organic electroluminescence display device is a display device including a so-called self-luminous-type light-emitting element, in which holes and electrons are respectively injected into an emission layer from a first electrode and a second electrode, thereby causing an emission material in the emission layer to emit light to implement a display.

For application of a light-emitting element to a display device, improvements in light efficiency and lifespan are required, and development of materials for a light-emitting element, which may stably achieve the requirements, is increasingly demanded.

### SUMMARY

For application of a light-emitting element to a display device, improvements in light efficiency and lifespan are required, and development of materials for a light-emitting element, which may stably achieve the requirements, is increasingly demanded.

The present disclosure provides a light-emitting element with a reduced driving voltage and an improved lifespan.

The present disclosure also provides an amine compound which is a material for a light-emitting element with a reduced driving voltage and long lifespan characteristics.

The present disclosure also provides a display device having excellent display quality by including a light-emitting element with a reduced driving voltage and an improved lifespan.

An embodiment of this disclosure provides a light-emitting element including a first electrode, a second electrode disposed on the first electrode, an emission layer disposed between the first electrode and the second electrode, a hole transport region disposed between the first electrode and the emission layer and including an amine compound according to Formula 1.

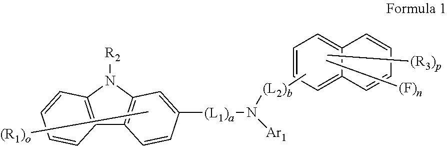

In Formula 1, L₁ and L₂ are each independently a substituted or unsubstituted arylene group having 6 to 30 carbon atoms, Ar₁ is a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms, each R₁, R₂, and each R₃ are independently a hydrogen atom, a deuterium atom, a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms, a and b are each independently an integer of 0 to 4, provided that: when a is 0, N of NAr₁ is directly bonded to the 2-position of a carbazole group, and when b is 0, N of NAr₁ is directly bonded to a naphthyl group, n is an integer of 1 to 7, o is an integer of 0 to 7, and p is an integer of 0 to (7-n).

In an embodiment, the hole transport region may include at least one among a hole injection layer, a hole transport layer, an electron-blocking layer, and an emission auxiliary layer, and the amine compound may be included in at least one among the hole injection layer, the hole transport layer, the electron-blocking layer, and the emission auxiliary layer.

In an embodiment, the hole transport region may include a hole injection layer disposed on the first electrode and a hole transport layer disposed on the hole injection layer, and the amine compound may be included in the hole transport layer.

In an embodiment, the hole transport region may include a hole injection layer disposed on the first electrode, a first hole transport layer disposed on the hole injection layer, and a second hole transport layer adjacent to a bottom of the emission layer and disposed on the first hole transport layer, and the amine compound may be included in the second hole transport layer.

In an embodiment, the amine compound may have a structure according to any one of Formula 2-1 to Formula 2-3.

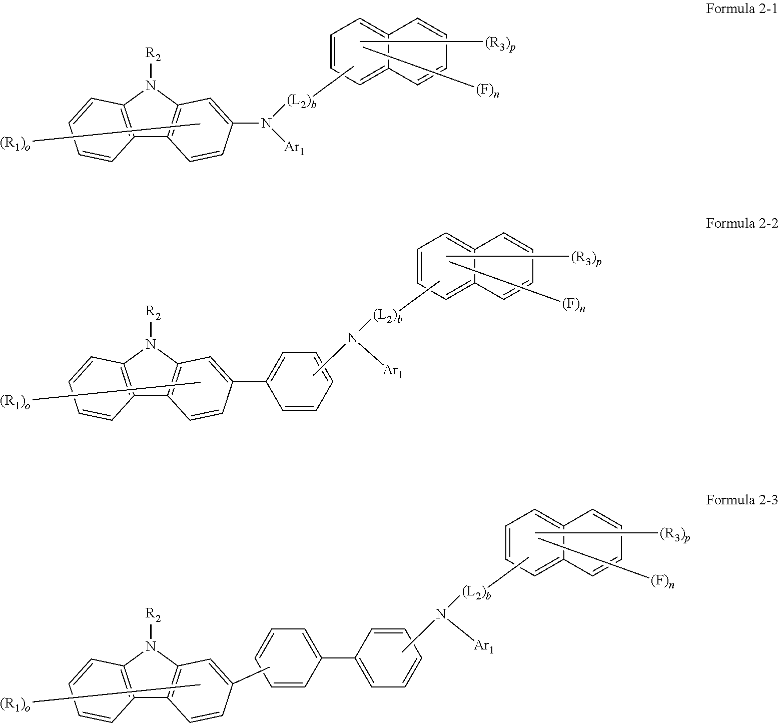

In Formula 2-1 to Formula 2-3, L₂, Ar₁, each R₁, R₂, and each R₃, b, n, o, and p are the same as defined in Formula 1.

In an embodiment, the amine compound may have a structure according to any one of Formula 3-1 to Formula 3-3:

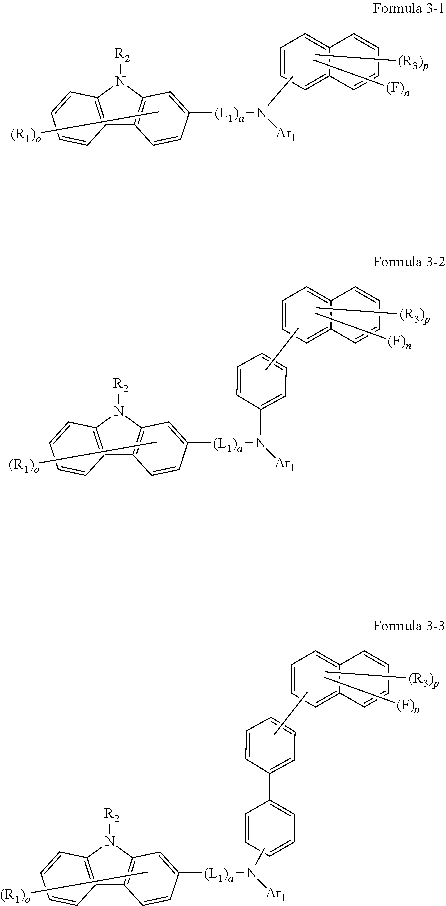

In Formula 3-1 to Formula 3-3, L₁, Ar₁, each R₁, R₂, and each, a, n, o, and p are the same as defined in Formula 1.

In an embodiment, Ar₁ may be a substituted or unsubstituted phenyl group, a substituted or unsubstituted dibenzofuran group, a substituted or unsubstituted dibenzothiophene group, a substituted or unsubstituted naphthyl group, or a substituted or unsubstituted naphthobenzofuran group.

In an embodiment, the emission layer may include a compound according to Formula E-1:

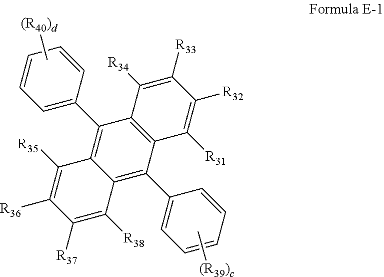

In Formula E-1, c and d are each independently an integer of 0 to 5, and R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring.

In an embodiment, the amine compound may have a structure according to any one of Compound Group 1.

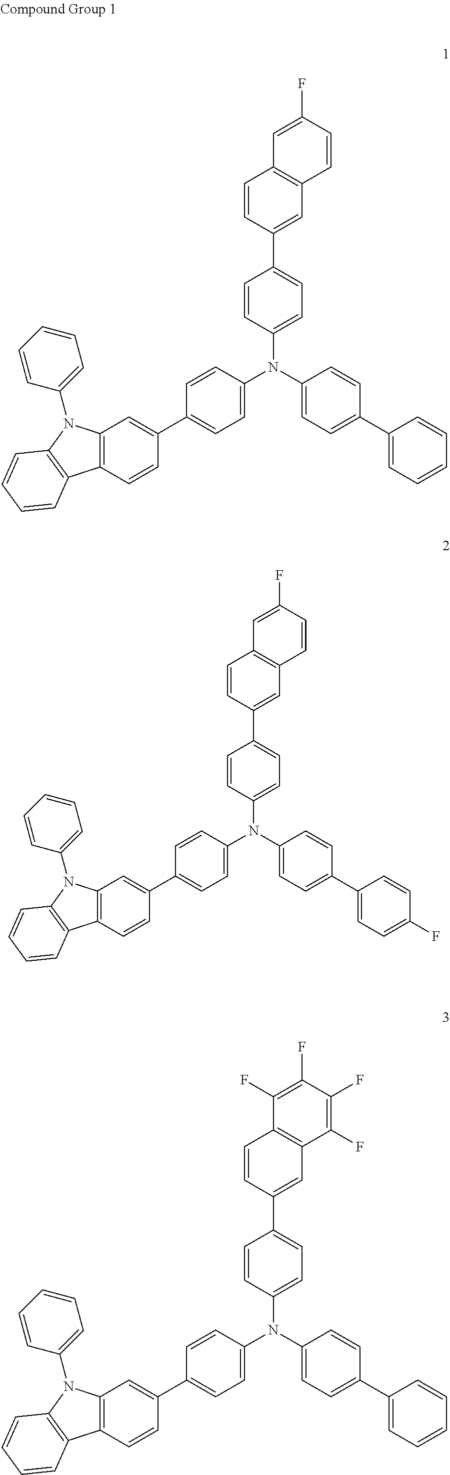 [SMILES: Fc1ccc2cc(-c3ccc(N(c4ccc(-c5ccccc5)cc4)c4ccc(-c5ccc6c7ccccc7n(-c7ccccc7)c6c5)cc4)cc3)ccc2c1 ; Fc1ccc(-c2ccc(N(c3ccc(-c4ccc5cc(F)ccc5c4)cc3)c3ccc(-c4ccc5c6ccccc6n(-c6ccccc6)c5c4)cc3)cc2)cc1 ; Fc1c(F)c(F)c2cc(-c3ccc(N(c4ccc(-c5ccccc5)cc4)c4ccc(-c5ccc6c7ccccc7n(-c7ccccc7)c6c5)cc4)cc3)ccc2c1F]

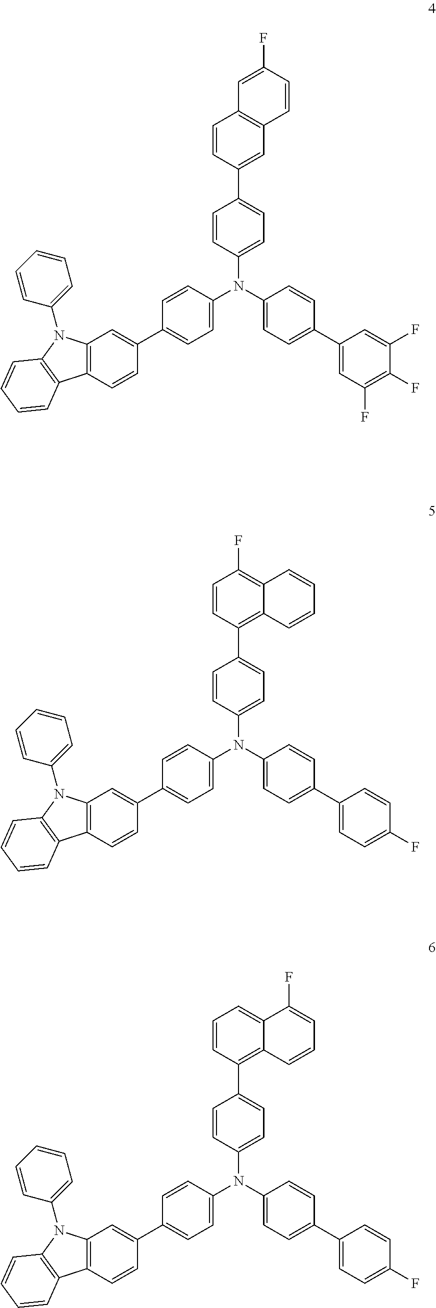 [SMILES: Fc1ccc2cc(-c3ccc(N(c4ccc(-c5cc(F)c(F)c(F)c5)cc4)c4ccc(-c5ccc6c7ccccc7n(-c7ccccc7)c6c5)cc4)cc3)ccc2c1 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)cc1 ; Fc1ccc(-c2ccc(N(c3ccc(-c4ccc5c6ccccc6n(-c6ccccc6)c5c4)cc3)c3ccc(-c4cccc5c(F)cccc45)cc3)cc2)cc1]

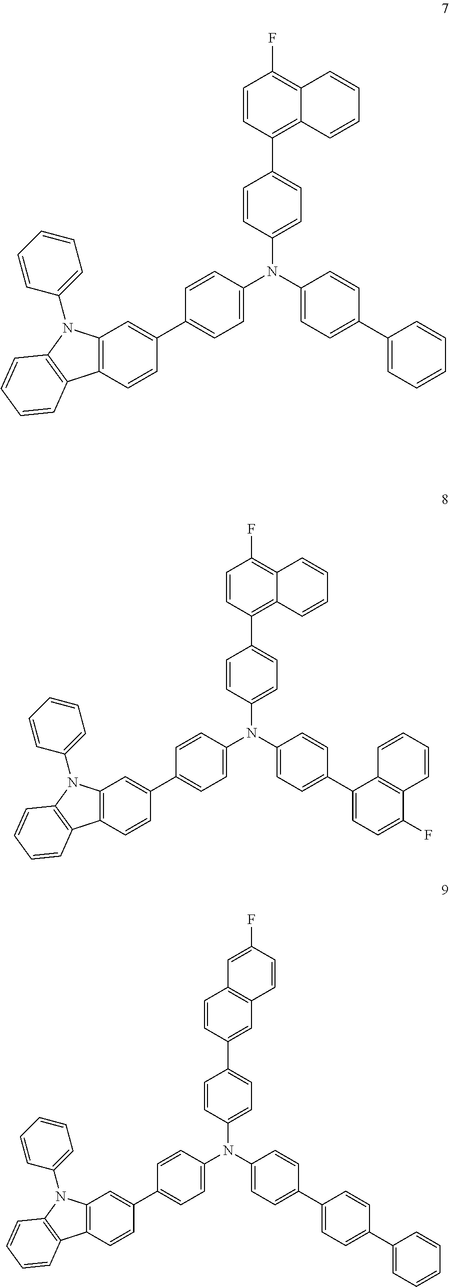 [SMILES: Fc1ccc(-c2ccc(N(c3ccc(-c4ccccc4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)c2ccccc12 ; Fc1ccc2cc(-c3ccc(N(c4ccc(-c5ccc(-c6ccccc6)cc5)cc4)c4ccc(-c5ccc6c7ccccc7n(-c7ccccc7)c6c5)cc4)cc3)ccc2c1]

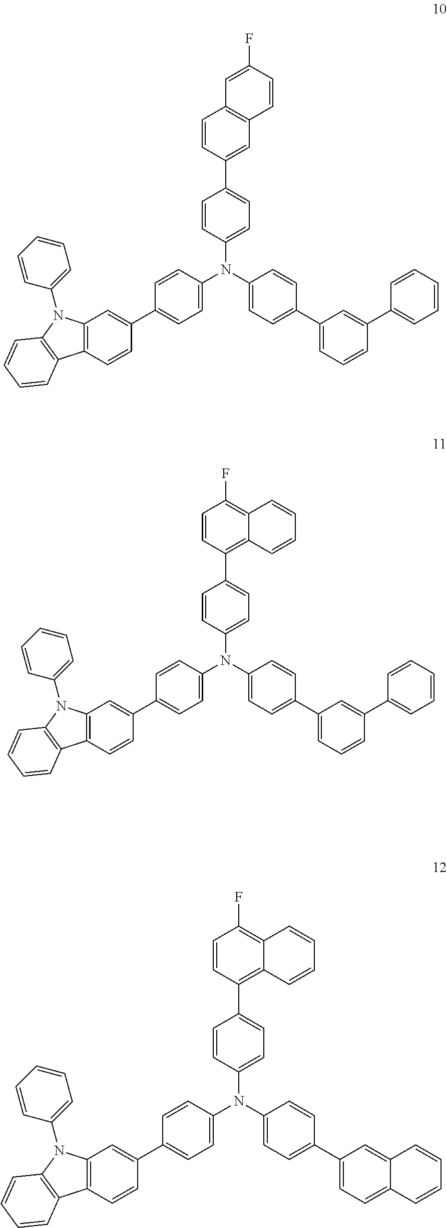 [SMILES: Fc1ccc2cc(-c3ccc(N(c4ccc(-c5cccc(-c6ccccc6)c5)cc4)c4ccc(-c5ccc6c7ccccc7n(-c7ccccc7)c6c5)cc4)cc3)ccc2c1 ; Fc1ccc(-c2ccc(N(c3ccc(-c4cccc(-c5ccccc5)c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5ccccc5c4)cc3)c3ccc(-c4ccc5c6ccccc6n(-c6ccccc6)c5c4)cc3)cc2)c2ccccc12]

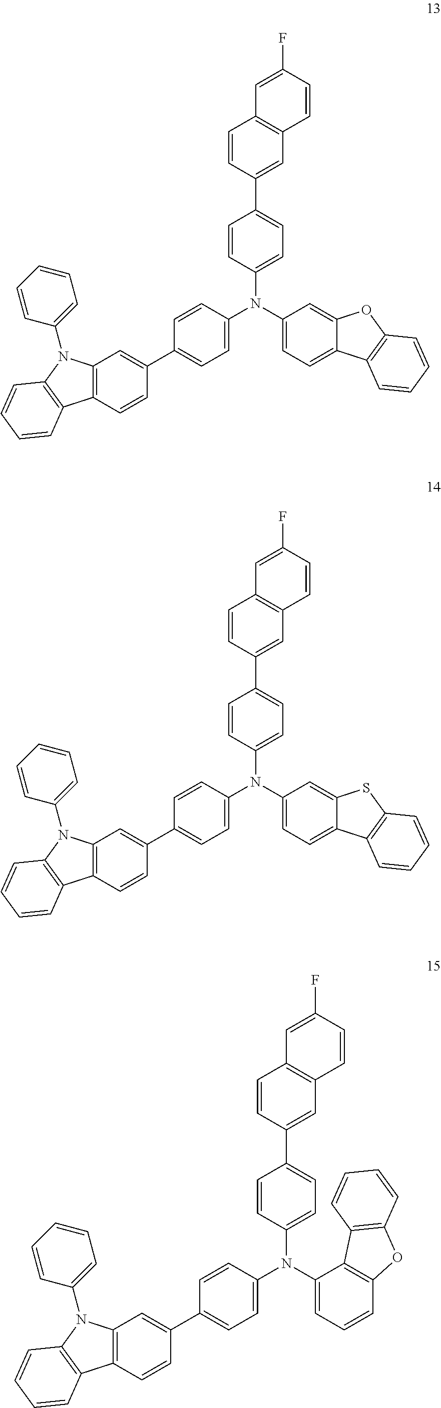 [SMILES: Fc1ccc2cc(-c3ccc(N(c4ccc(-c5ccc6c7ccccc7n(-c7ccccc7)c6c5)cc4)c4ccc5c(c4)oc4ccccc45)cc3)ccc2c1 ; Fc1ccc2cc(-c3ccc(N(c4ccc(-c5ccc6c7ccccc7n(-c7ccccc7)c6c5)cc4)c4ccc5c(c4)sc4ccccc45)cc3)ccc2c1 ; Fc1ccc2cc(-c3ccc(N(c4ccc(-c5ccc6c7ccccc7n(-c7ccccc7)c6c5)cc4)c4cccc5oc6ccccc6c45)cc3)ccc2c1]

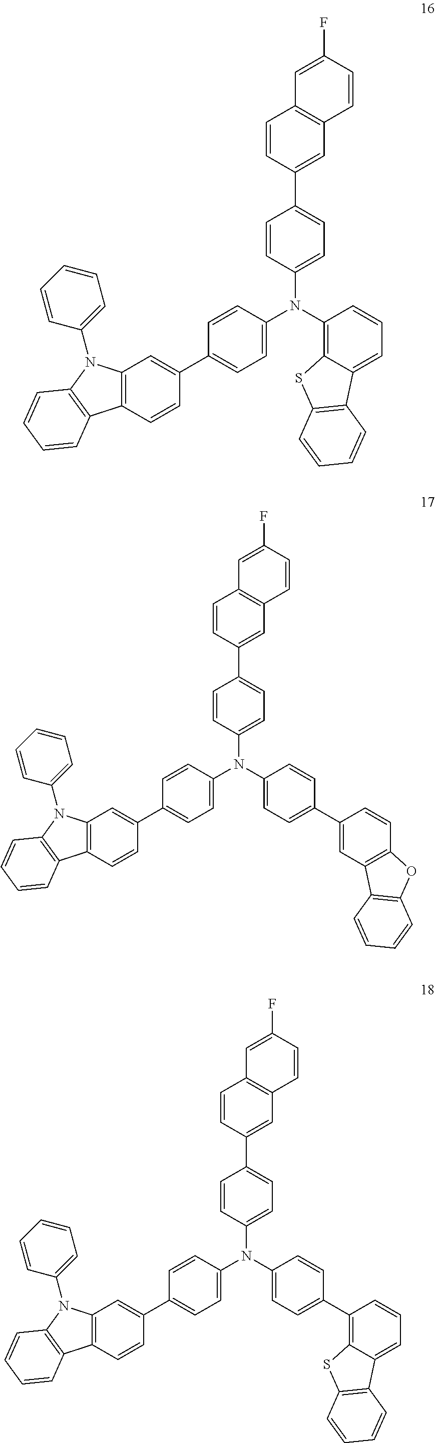 [SMILES: Fc1ccc2cc(-c3ccc(N(c4ccc(-c5ccc6c7ccccc7n(-c7ccccc7)c6c5)cc4)c4cccc5c4sc4ccccc45)cc3)ccc2c1 ; Fc1ccc2cc(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5sc5ccccc56)cc4)cc3)ccc2c1]

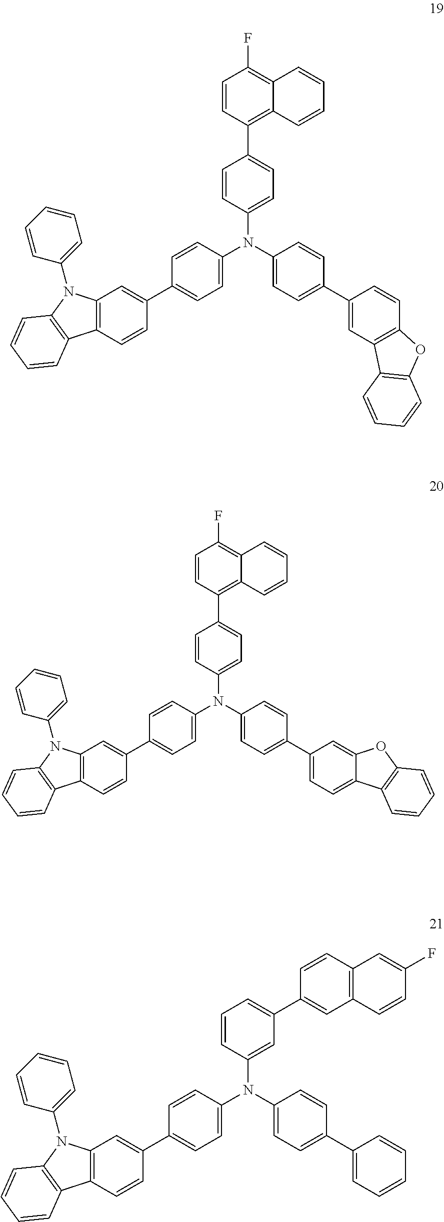 [SMILES: Fc1ccc(-c2ccc(N(c3ccc(-c4ccc5oc6ccccc6c5c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c(c4)oc4ccccc45)cc3)c3ccc(-c4ccc5c6ccccc6n(-c6ccccc6)c5c4)cc3)cc2)c2ccccc12 ; Fc1ccc2cc(-c3cccc(N(c4ccc(-c5ccccc5)cc4)c4ccc(-c5ccc6c7ccccc7n(-c7ccccc7)c6c5)cc4)c3)ccc2c1]

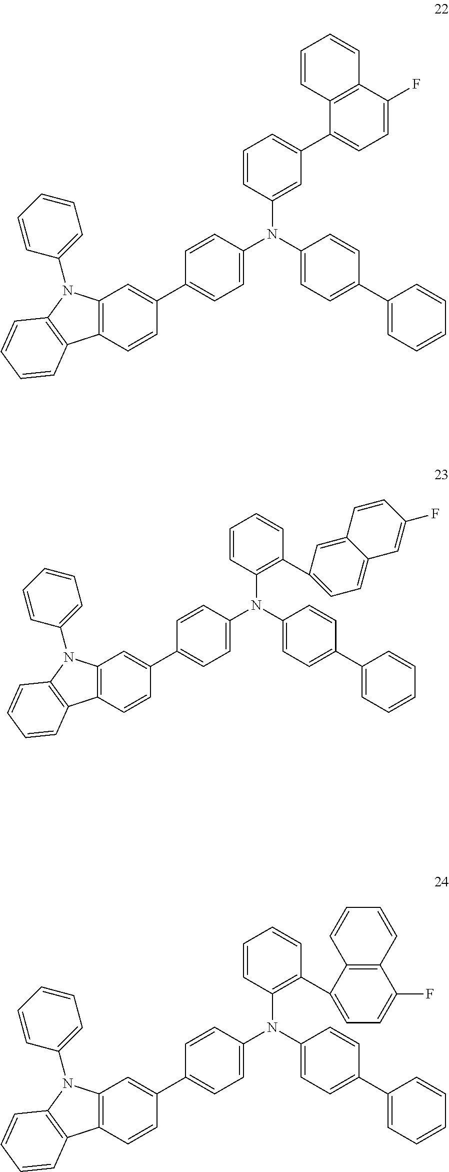 [SMILES: Fc1ccc(-c2cccc(N(c3ccc(-c4ccccc4)cc3)c3ccc(-c4ccc5c6ccccc6n(-c6ccccc6)c5c4)cc3)c2)c2ccccc12 ; Fc1ccc2cc(-c3ccccc3N(c3ccc(-c4ccccc4)cc3)c3ccc(-c4ccc5c6ccccc6n(-c6ccccc6)c5c4)cc3)ccc2c1 ; Fc1ccc(-c2ccccc2N(c2ccc(-c3ccccc3)cc2)c2ccc(-c3ccc4c5ccccc5n(-c5ccccc5)c4c3)cc2)c2ccccc12]

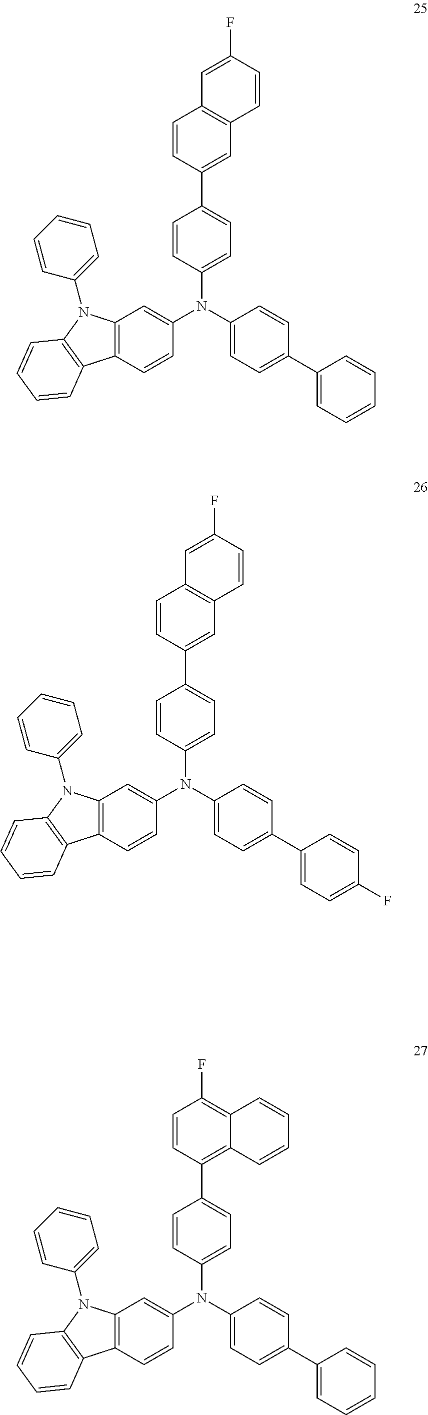 [SMILES: Fc1ccc2cc(-c3ccc(N(c4ccc(-c5ccccc5)cc4)c4ccc5c6ccccc6n(-c6ccccc6)c5c4)cc3)ccc2c1 ; Fc1ccc(-c2ccc(N(c3ccc(-c4ccc5cc(F)ccc5c4)cc3)c3ccc4c5ccccc5n(-c5ccccc5)c4c3)cc2)cc1 ; Fc1ccc(-c2ccc(N(c3ccc(-c4ccccc4)cc3)c3ccc4c5ccccc5n(-c5ccccc5)c4c3)cc2)c2ccccc12]

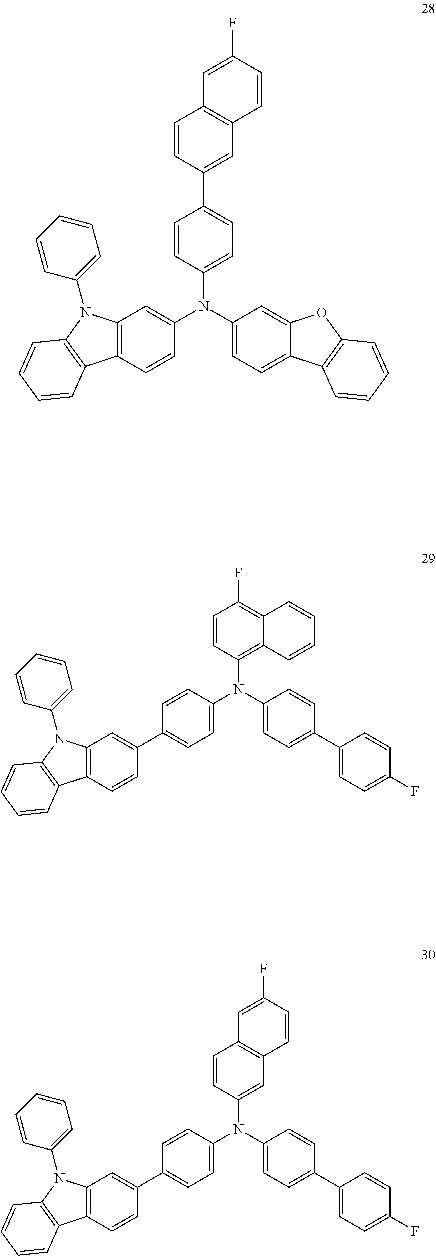 [SMILES: Fc1ccc2cc(-c3ccc(N(c4ccc5c(c4)oc4ccccc45)c4ccc5c6ccccc6n(-c6ccccc6)c5c4)cc3)ccc2c1 ; Fc1ccc(-c2ccc(N(c3ccc(-c4ccc5c6ccccc6n(-c6ccccc6)c5c4)cc3)c3ccc(F)c4ccccc34)cc2)cc1 ; Fc1ccc(-c2ccc(N(c3ccc(-c4ccc5c6ccccc6n(-c6ccccc6)c5c4)cc3)c3ccc4cc(F)ccc4c3)cc2)cc1]

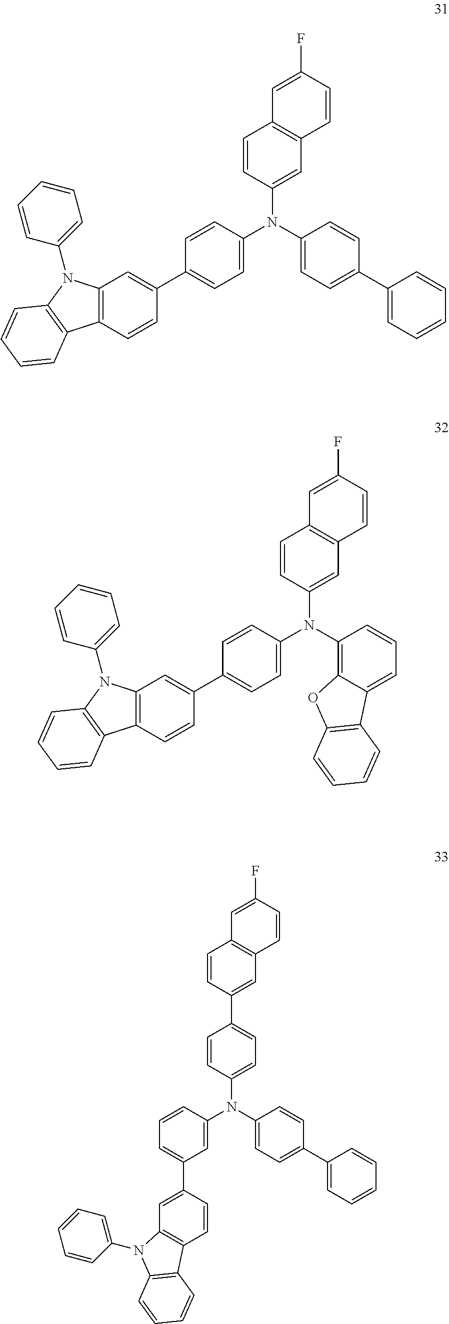 [SMILES: Fc1ccc2cc(N(c3ccc(-c4ccccc4)cc3)c3ccc(-c4ccc5c6ccccc6n(-c6ccccc6)c5c4)cc3)ccc2c1 ; Fc1ccc2cc(N(c3ccc(-c4ccc5c6ccccc6n(-c6ccccc6)c5c4)cc3)c3cccc4c3oc3ccccc34)ccc2c1 ; Fc1ccc2cc(-c3ccc(N(c4ccc(-c5ccccc5)cc4)c4cccc(-c5ccc6c7ccccc7n(-c7ccccc7)c6c5)c4)cc3)ccc2c1]

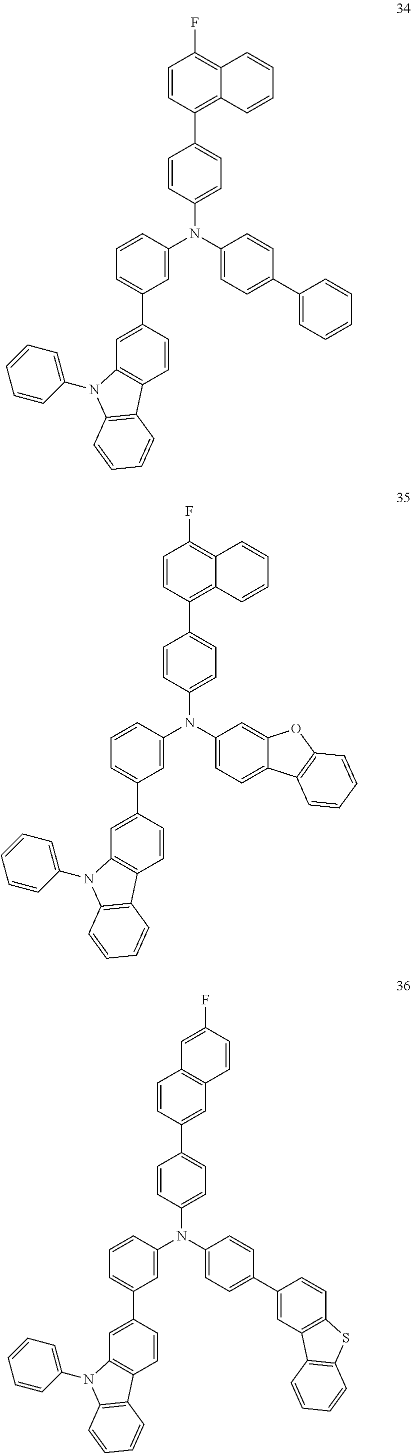 [SMILES: Fc1ccc(-c2ccc(N(c3ccc(-c4ccccc4)cc3)c3cccc(-c4ccc5c6ccccc6n(-c6ccccc6)c5c4)c3)cc2)c2ccccc12 ; Fc1ccc(-c2ccc(N(c3cccc(-c4ccc5c6ccccc6n(-c6ccccc6)c5c4)c3)c3ccc4c(c3)oc3ccccc34)cc2)c2ccccc12 ; Fc1ccc2cc(-c3ccc(N(c4ccc(-c5ccc6sc7ccccc7c6c5)cc4)c4cccc(-c5ccc6c7ccccc7n(-c7ccccc7)c6c5)c4)cc3)ccc2c1]

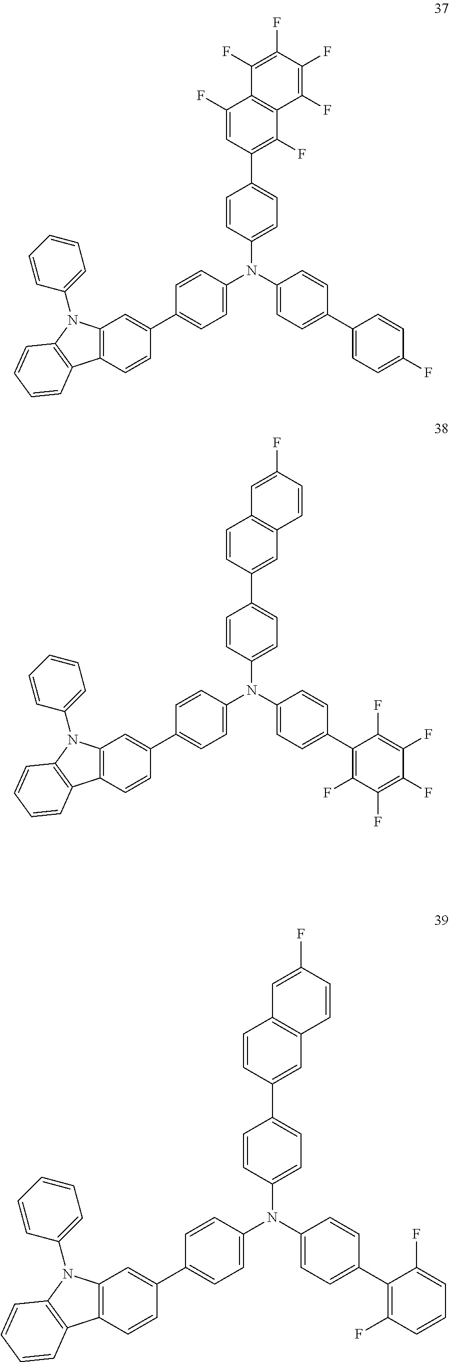 [SMILES: Fc1ccc(-c2ccc(N(c3ccc(-c4ccc5c6ccccc6n(-c6ccccc6)c5c4)cc3)c3ccc(-c4cc(F)c5c(F)c(F)c(F)c(F)c5c4F)cc3)cc2)cc1 ; Fc1ccc2cc(-c3ccc(N(c4ccc(-c5ccc6c7ccccc7n(-c7ccccc7)c6c5)cc4)c4ccc(-c5c(F)c(F)c(F)c(F)c5F)cc4)cc3)ccc2c1 ; Fc1ccc2cc(-c3ccc(N(c4ccc(-c5ccc6c7ccccc7n(-c7ccccc7)c6c5)cc4)c4ccc(-c5c(F)cccc5F)cc4)cc3)ccc2c1]

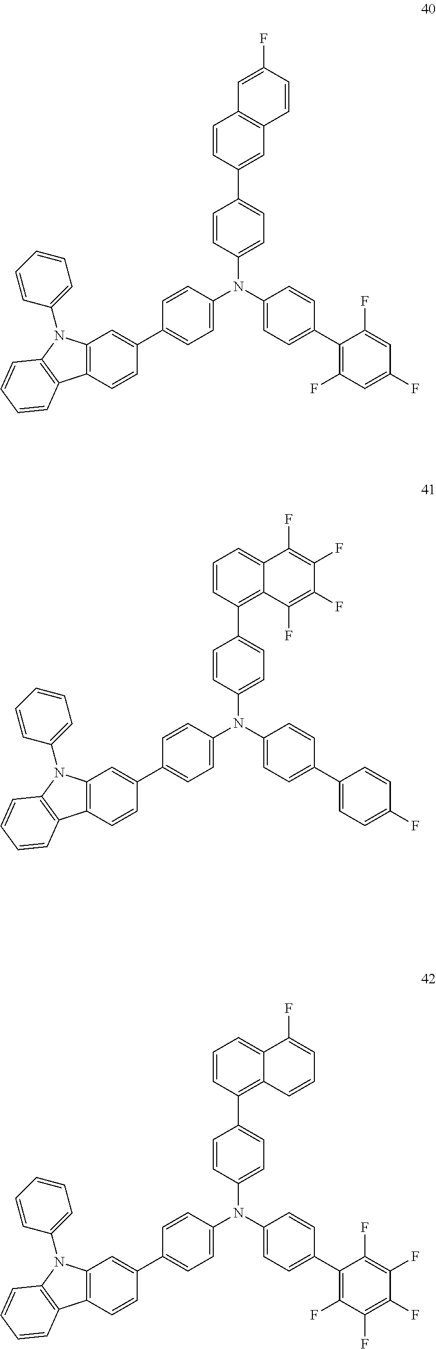 [SMILES: Fc1cc(F)c(-c2ccc(N(c3ccc(-c4ccc5cc(F)ccc5c4)cc3)c3ccc(-c4ccc5c6ccccc6n(-c6ccccc6)c5c4)cc3)cc2)c(F)c1 ; Fc1ccc(-c2ccc(N(c3ccc(-c4ccc5c6ccccc6n(-c6ccccc6)c5c4)cc3)c3ccc(-c4cccc5c(F)c(F)c(F)c(F)c45)cc3)cc2)cc1 ; Fc1c(F)c(F)c(-c2ccc(N(c3ccc(-c4ccc5c6ccccc6n(-c6ccccc6)c5c4)cc3)c3ccc(-c4cccc5c(F)cccc45)cc3)cc2)c(F)c1F]

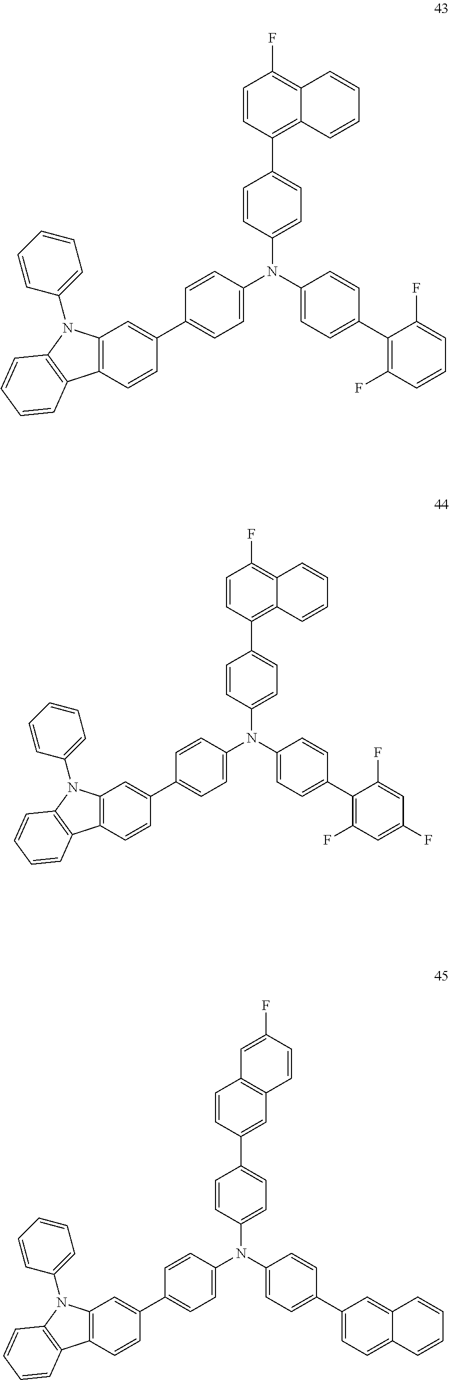 [SMILES: Fc1cccc(F)c1-c1ccc(N(c2ccc(-c3ccc4c5ccccc5n(-c5ccccc5)c4c3)cc2)c2ccc(-c3ccc(F)c4ccccc34)cc2)cc1 ; Fc1cc(F)c(-c2ccc(N(c3ccc(-c4ccc5c6ccccc6n(-c6ccccc6)c5c4)cc3)c3ccc(-c4ccc(F)c5ccccc45)cc3)cc2)c(F)c1 ; Fc1ccc2cc(-c3ccc(N(c4ccc(-c5ccc6ccccc6c5)cc4)c4ccc(-c5ccc6c7ccccc7n(-c7ccccc7)c6c5)cc4)cc3)ccc2c1]

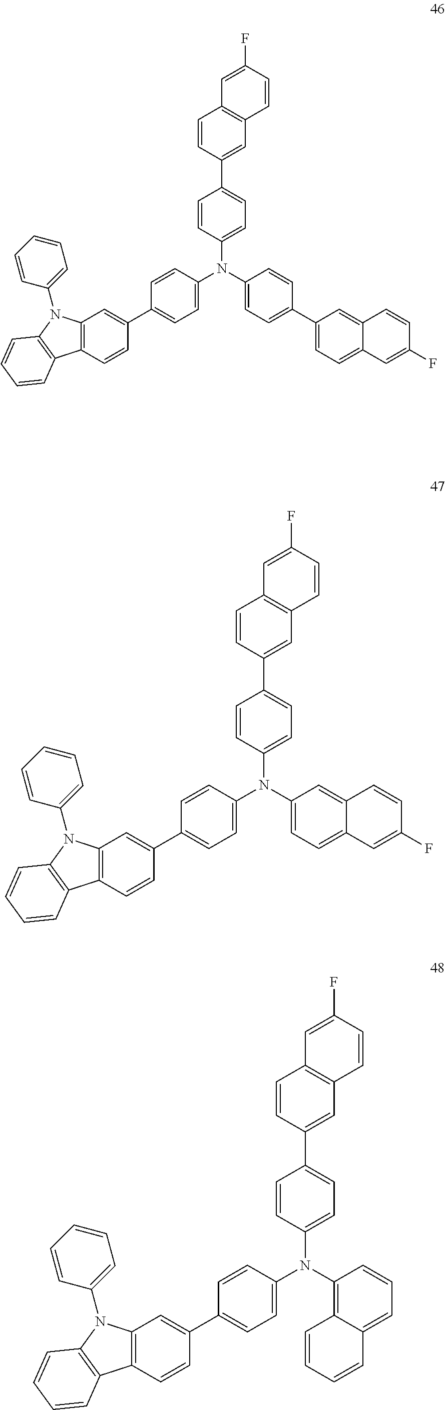 [SMILES: Fc1ccc2cc(-c3ccc(N(c4ccc(-c5ccc6cc(F)ccc6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc5cc(F)ccc5c4)cc3)ccc2c1 ; Fc1ccc2cc(-c3ccc(N(c4ccc(-c5ccc6c7ccccc7n(-c7ccccc7)c6c5)cc4)c4cccc5ccccc45)cc3)ccc2c1]

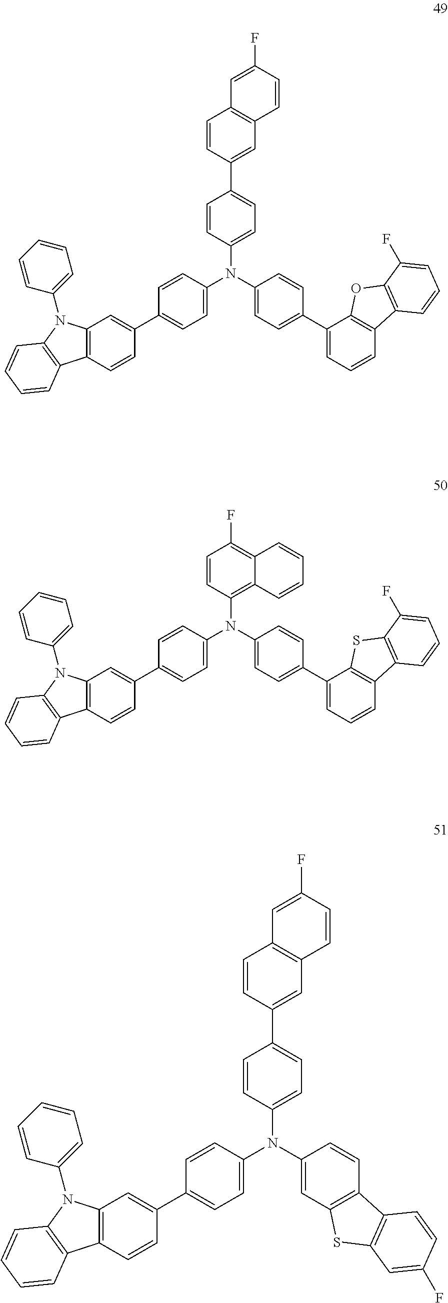 [SMILES: Fc1ccc2cc(-c3ccc(N(c4ccc(-c5ccc6c7ccccc7n(-c7ccccc7)c6c5)cc4)c4ccc(-c5cccc6c5oc5c(F)cccc56)cc4)cc3)ccc2c1 ; Fc1ccc(N(c2ccc(-c3ccc4c5ccccc5n(-c5ccccc5)c4c3)cc2)c2ccc(-c3cccc4c3sc3c(F)cccc34)cc2)c2ccccc12 ; Fc1ccc2cc(-c3ccc(N(c4ccc(-c5ccc6c7ccccc7n(-c7ccccc7)c6c5)cc4)c4ccc5c(c4)sc4cc(F)ccc45)cc3)ccc2c1]

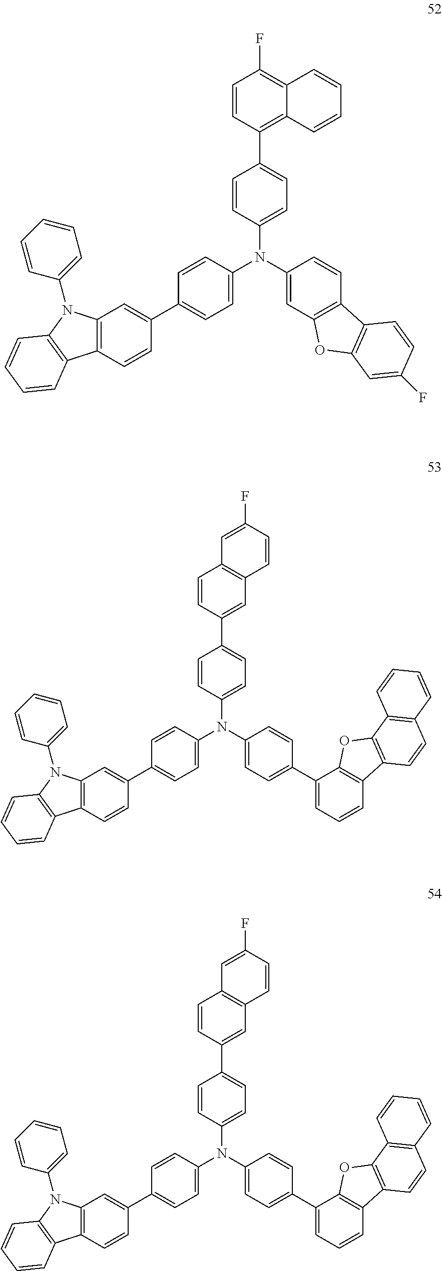 [SMILES: Fc1ccc2c(c1)oc1cc(N(c3ccc(-c4ccc5c6ccccc6n(-c6ccccc6)c5c4)cc3)c3ccc(-c4ccc(F)c5ccccc45)cc3)ccc12 ; Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1]

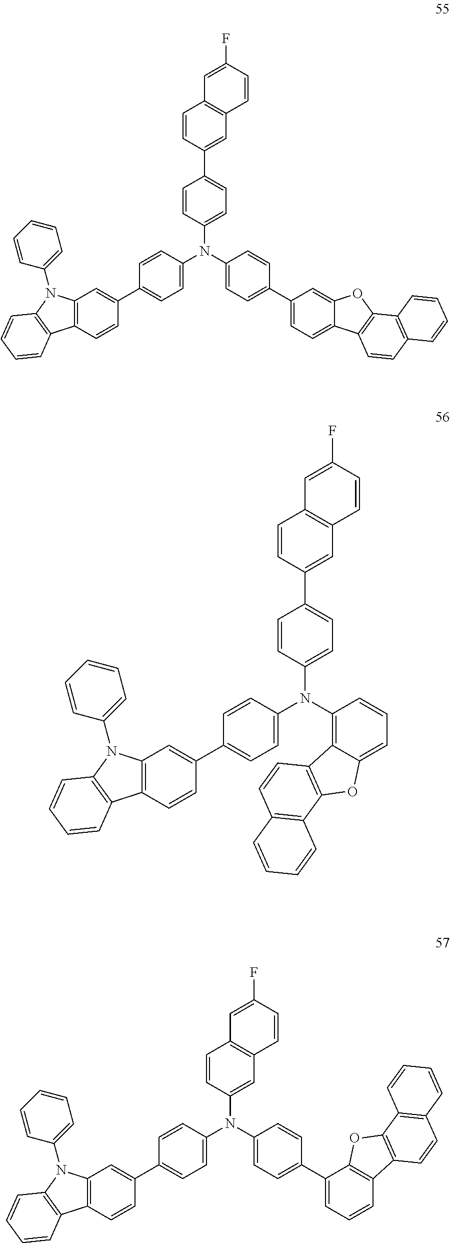 [SMILES: Fc1ccc2cc(-c3ccc(N(c4ccc(-c5ccc6c(c5)oc5c7ccccc7ccc65)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4cccc5oc6c7ccccc7ccc6c45)cc3)ccc2c1 ; Fc1ccc2cc(N(c3ccc(-c4ccc5c6ccccc6n(-c6ccccc6)c5c4)cc3)c3ccc(-c4cccc5c4oc4c6ccccc6ccc54)cc3)ccc2c1]

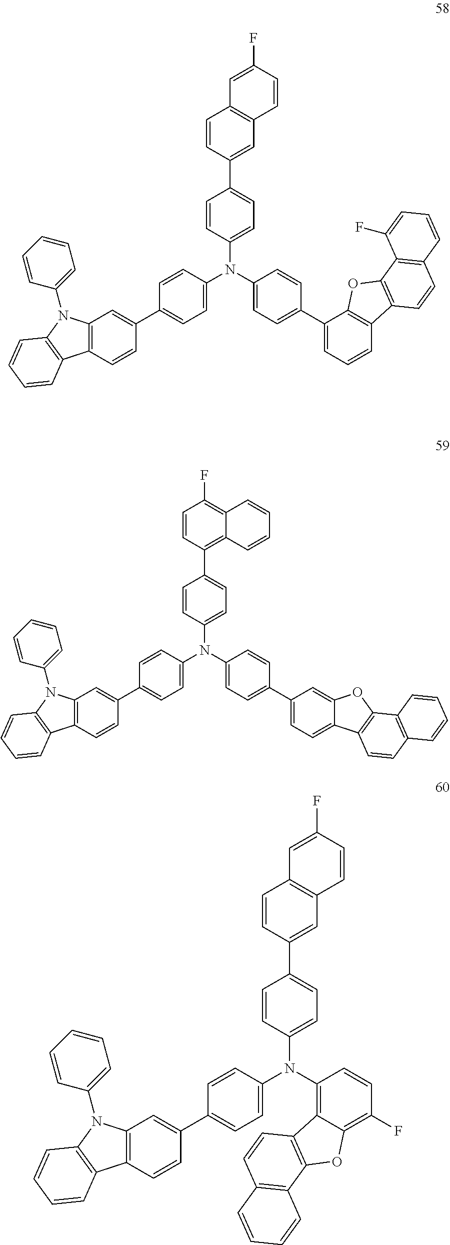 [SMILES: Fc1ccc2cc(-c3ccc(N(c4ccc(-c5ccc6c7ccccc7n(-c7ccccc7)c6c5)cc4)c4ccc(-c5cccc6c5oc5c6ccc6cccc(F)c65)cc4)cc3)ccc2c1 ; Fc1ccc(-c2ccc(N(c3ccc(-c4ccc5c(c4)oc4c6ccccc6ccc54)cc3)c3ccc(-c4ccc5c6ccccc6n(-c6ccccc6)c5c4)cc3)cc2)c2ccccc12 ; Fc1ccc2cc(-c3ccc(N(c4ccc(-c5ccc6c7ccccc7n(-c7ccccc7)c6c5)cc4)c4ccc(F)c5oc6c7ccccc7ccc6c45)cc3)ccc2c1]

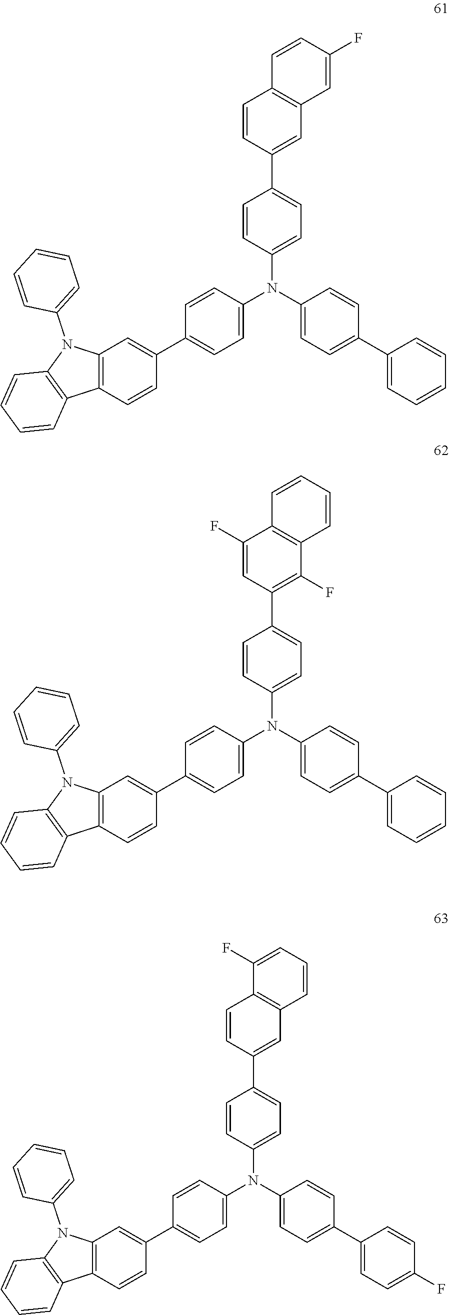 [SMILES: Fc1ccc2ccc(-c3ccc(N(c4ccc(-c5ccccc5)cc4)c4ccc(-c5ccc6c7ccccc7n(-c7ccccc7)c6c5)cc4)cc3)cc2c1 ; Fc1cc(-c2ccc(N(c3ccc(-c4ccccc4)cc3)c3ccc(-c4ccc5c6ccccc6n(-c6ccccc6)c5c4)cc3)cc2)c(F)c2ccccc12 ; Fc1ccc(-c2ccc(N(c3ccc(-c4ccc5c(F)cccc5c4)cc3)c3ccc(-c4ccc5c6ccccc6n(-c6ccccc6)c5c4)cc3)cc2)cc1]

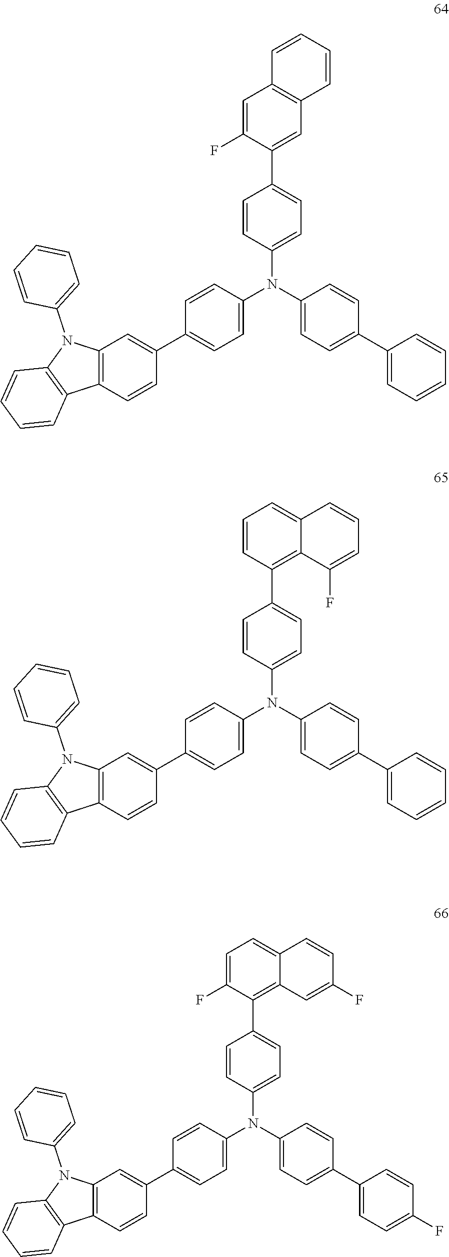 [SMILES: Fc1cc2ccccc2cc1-c1ccc(N(c2ccc(-c3ccccc3)cc2)c2ccc(-c3ccc4c5ccccc5n(-c5ccccc5)c4c3)cc2)cc1 ; Fc1cccc2cccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4c(F)ccc5ccc(F)cc45)cc3)cc2)cc1]

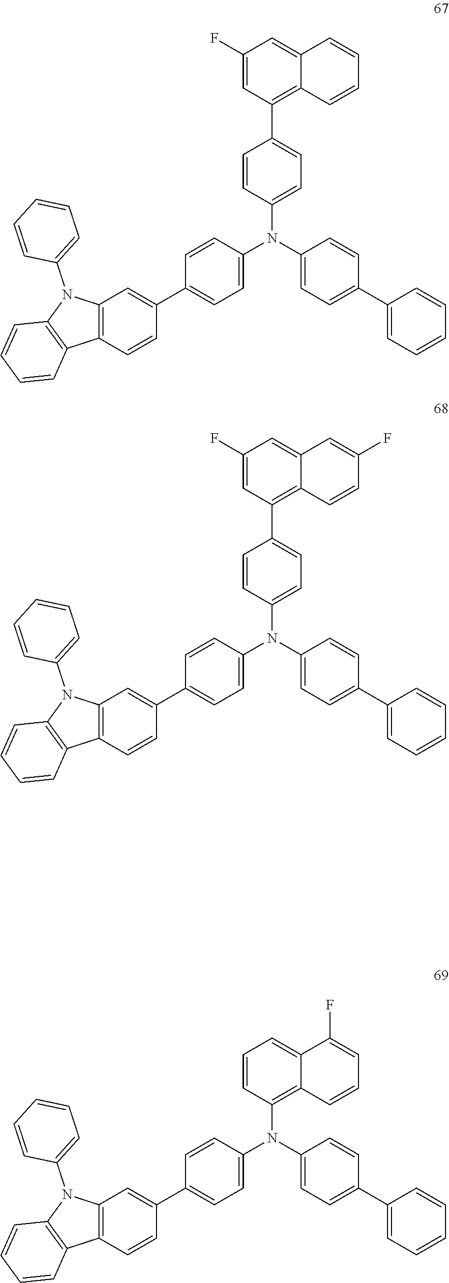 [SMILES: Fc1cc(-c2ccc(N(c3ccc(-c4ccccc4)cc3)c3ccc(-c4ccc5c6ccccc6n(-c6ccccc6)c5c4)cc3)cc2)c2ccccc2c1 ; Fc1ccc2c(-c3ccc(N(c4ccc(-c5ccccc5)cc4)c4ccc(-c5ccc6c7ccccc7n(-c7ccccc7)c6c5)cc4)cc3)cc(F)cc2c1 ; Fc1cccc2c(N(c3ccc(-c4ccccc4)cc3)c3ccc(-c4ccc5c6ccccc6n(-c6ccccc6)c5c4)cc3)cccc12]

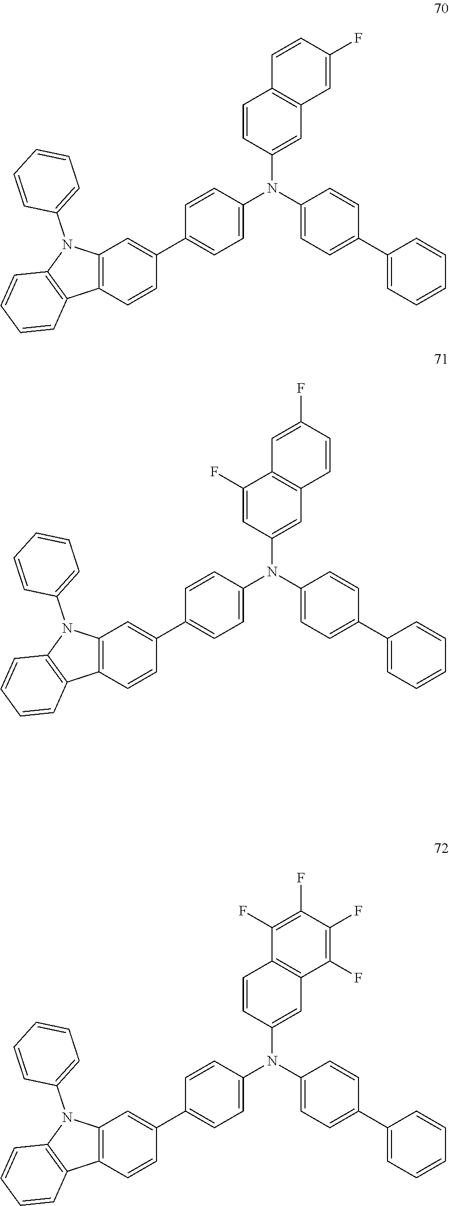 [SMILES: Fc1ccc2ccc(N(c3ccc(-c4ccccc4)cc3)c3ccc(-c4ccc5c6ccccc6n(-c6ccccc6)c5c4)cc3)cc2c1 ; Fc1ccc2cc(N(c3ccc(-c4ccccc4)cc3)c3ccc(-c4ccc5c6ccccc6n(-c6ccccc6)c5c4)cc3)cc(F)c2c1 ; Fc1c(F)c(F)c2cc(N(c3ccc(-c4ccccc4)cc3)c3ccc(-c4ccc5c6ccccc6n(-c6ccccc6)c5c4)cc3)ccc2c1F]

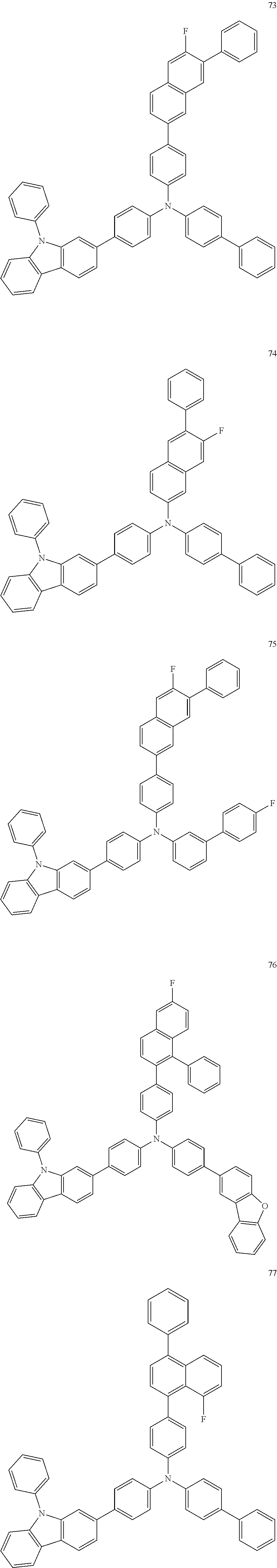

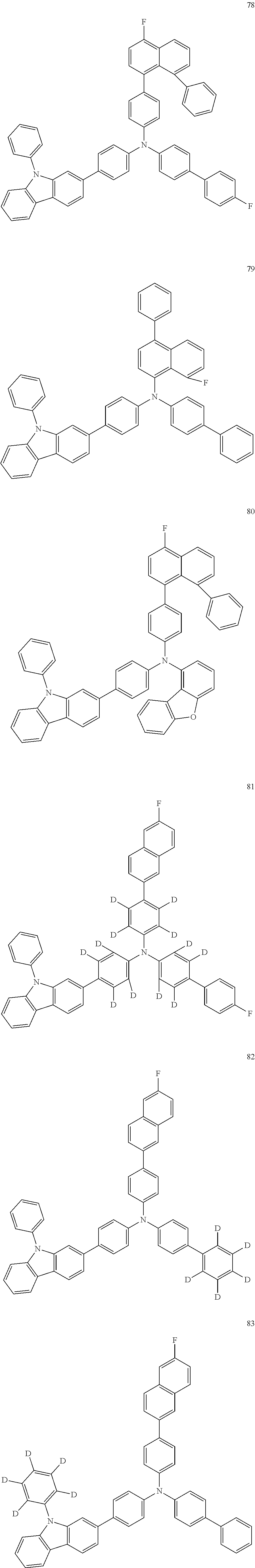

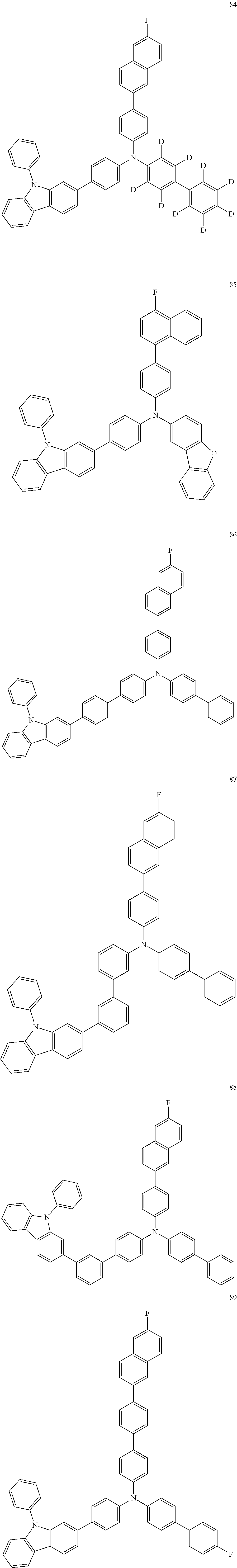

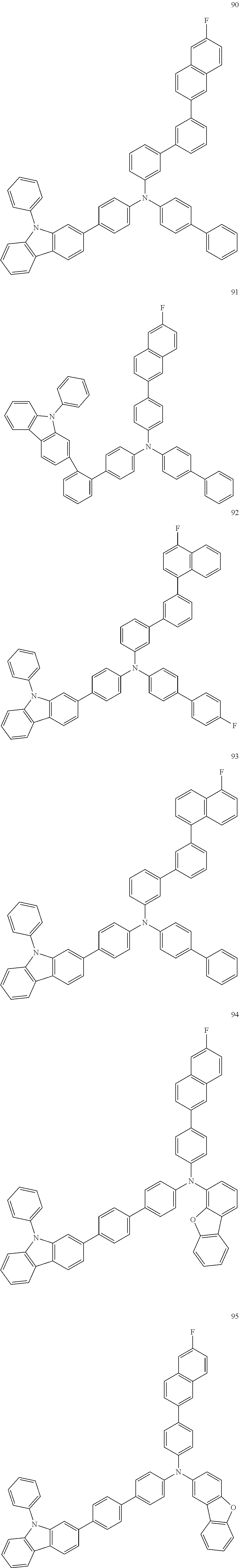

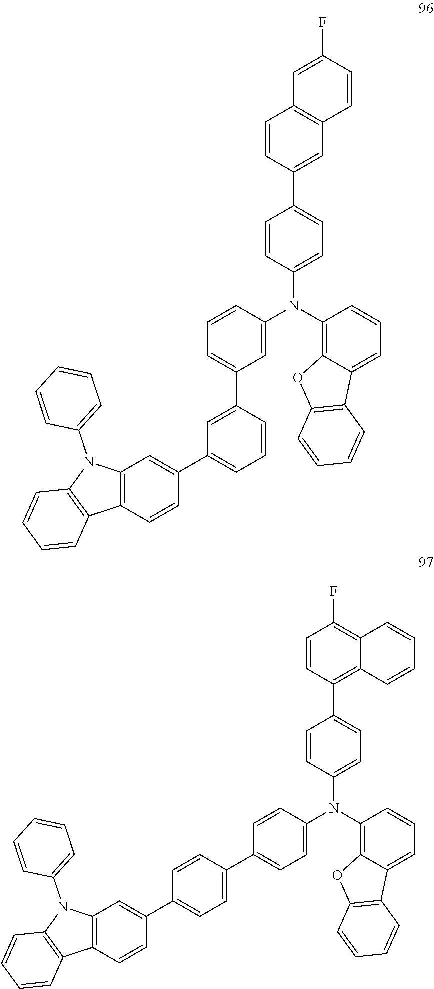 [SMILES: Fc1ccc2cc(-c3ccc(N(c4cccc(-c5cccc(-c6ccc7c8ccccc8n(-c8ccccc8)c7c6)c5)c4)c4cccc5c4oc4ccccc45)cc3)ccc2c1 ; Fc1ccc(-c2ccc(N(c3ccc(-c4ccc(-c5ccc6c7ccccc7n(-c7ccccc7)c6c5)cc4)cc3)c3cccc4c3oc3ccccc34)cc2)c2ccccc12]

In Compound Group 1, D is a deuterium atom.

In an embodiment, an amine compound may have a structure according to Formula 1.

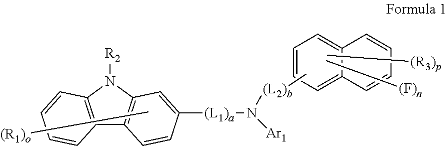

In Formula 1, L₁ and L₂ are each independently a substituted or unsubstituted arylene group having 6 to 30 ring-forming carbon atoms, Arn a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms, each R₁, R₂, and each are independently a hydrogen atom, a deuterium atom, a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms, a and b are each independently an integer of 0 to 4, provided that: when a is 0, N of NAr₁ is directly bonded to the 2-position of a carbazole group, and when b is 0, N of NAr₁ is directly bonded to a naphthyl group, n is an integer of 1 to 7, o is an integer of 0 to 7, and p is an integer of 0 to (7-n).

In an embodiment, Formula 1 may have a structure according to any one of Formula 2-1 to Formula 2-3.

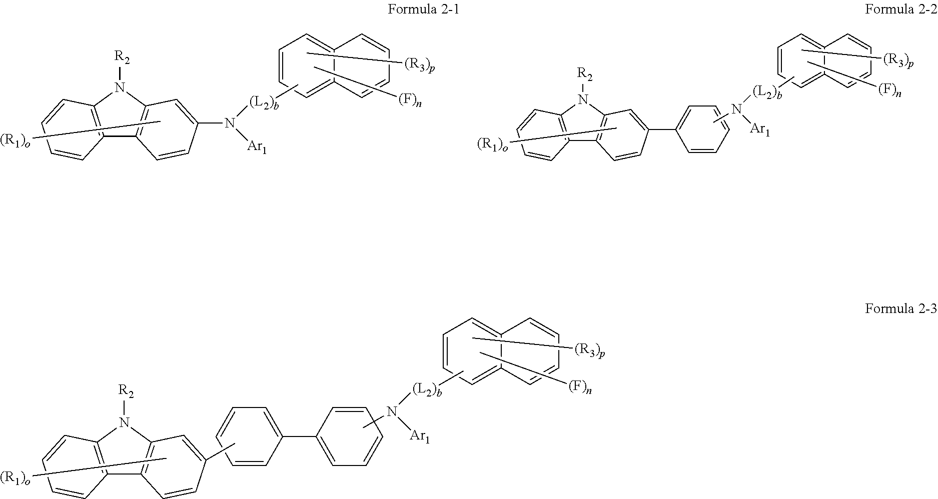

In Formula 2-1 to Formula 2-3, L₂, Ar₁, each R₁, R₂, and each, b, n, o, and p are the same as defined in Formula 1.

In an embodiment, Formula 1 may have a structure according to any one of Formula 3-1 to Formula 3-3.

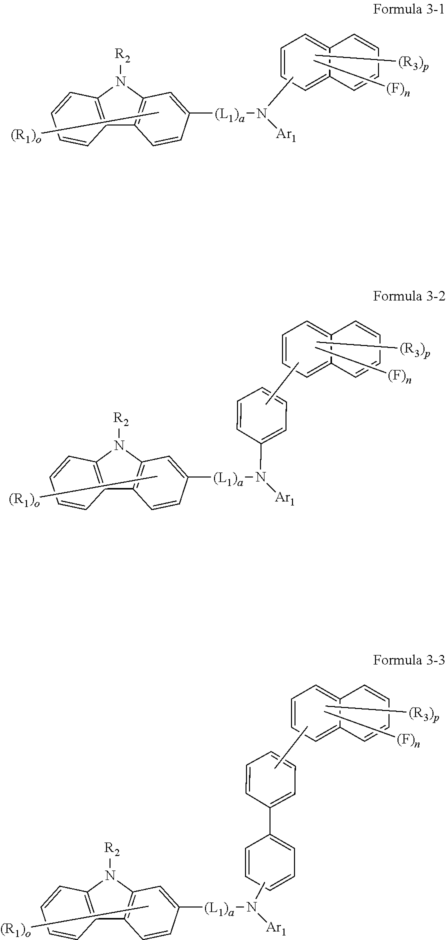

In Formula 3-1 to Formula 3-3, L₁, Ar₁, each R₁, R₂, and each, a, n, o, and p are the same as defined in Formula 1.

In an embodiment, Ar₁ may be a substituted or unsubstituted phenyl group, a substituted or unsubstituted dibenzofuran group, a substituted or unsubstituted dibenzothiophene group, a substituted or unsubstituted naphthyl group, or a substituted or unsubstituted naphthobenzofuran group.

In an embodiment, R₂ may be a substituted or unsubstituted phenyl group.

In an embodiment, Formula 1 may be a monoamine compound including no amino group as a substituent.

In an embodiment,

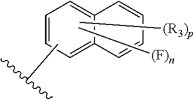

in Formula 1 may have a structure according to any one of Functional Group Set A:

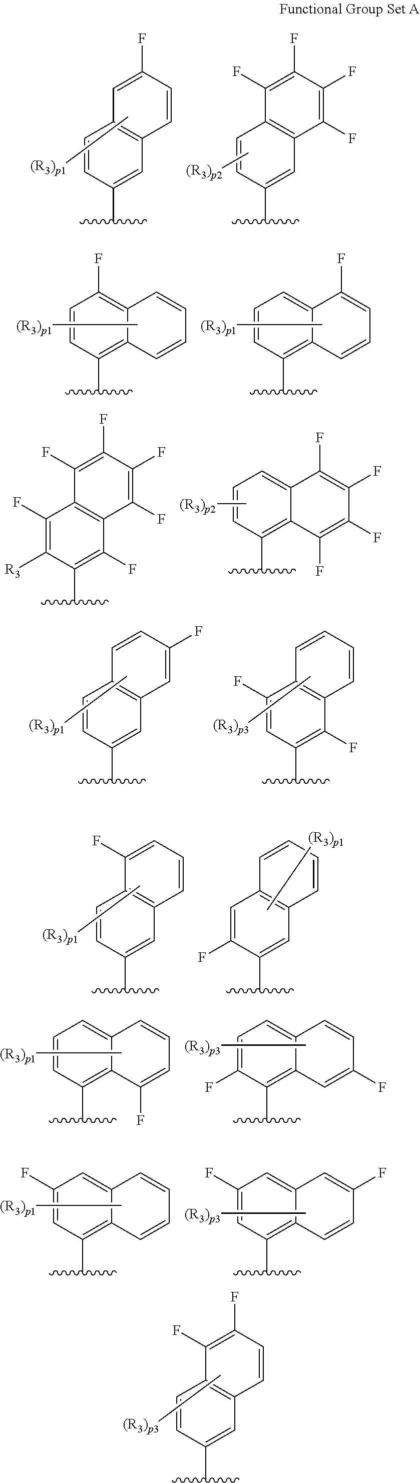

In Functional Group Set A, p1 is an integer of 0 to 6, p2 is an integer of 0 to 3, p3 is an integer of 0 to 5, and R₃ is the same as defined in Formula 1.

In an embodiment, the amine compound may have a structure according to any one of Compound Group 1:

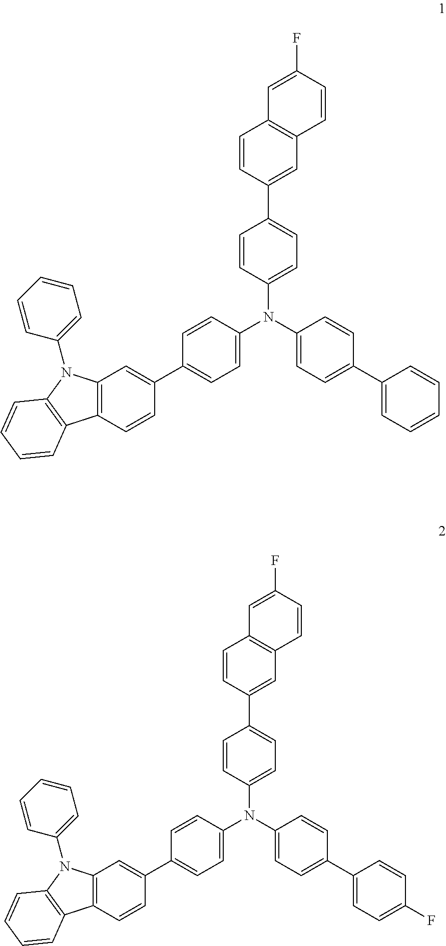 [SMILES: Fc1ccc2cc(-c3ccc(N(c4ccc(-c5ccccc5)cc4)c4ccc(-c5ccc6c7ccccc7n(-c7ccccc7)c6c5)cc4)cc3)ccc2c1 ; Fc1ccc(-c2ccc(N(c3ccc(-c4ccc5cc(F)ccc5c4)cc3)c3ccc(-c4ccc5c6ccccc6n(-c6ccccc6)c5c4)cc3)cc2)cc1]

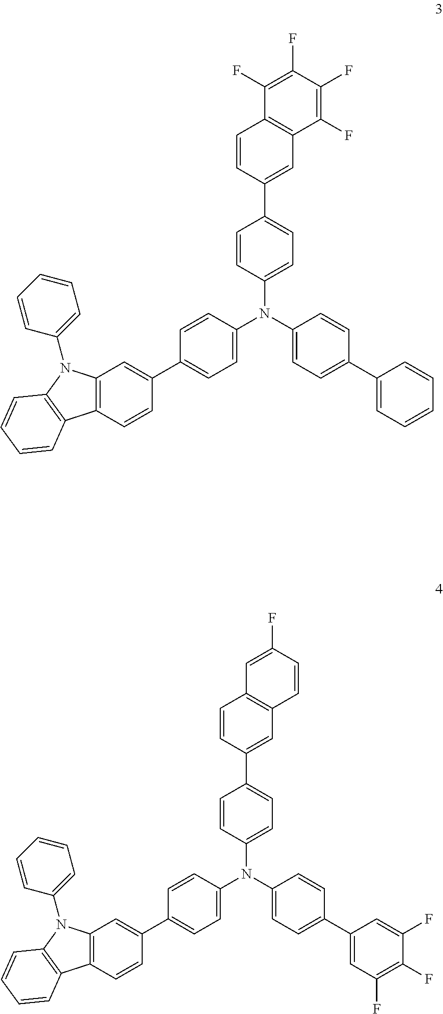 [SMILES: Fc1c(F)c(F)c2cc(-c3ccc(N(c4ccc(-c5ccccc5)cc4)c4ccc(-c5ccc6c7ccccc7n(-c7ccccc7)c6c5)cc4)cc3)ccc2c1F ; Fc1ccc2cc(-c3ccc(N(c4ccc(-c5cc(F)c(F)c(F)c5)cc4)c4ccc(-c5ccc6c7ccccc7n(-c7ccccc7)c6c5)cc4)cc3)ccc2c1]

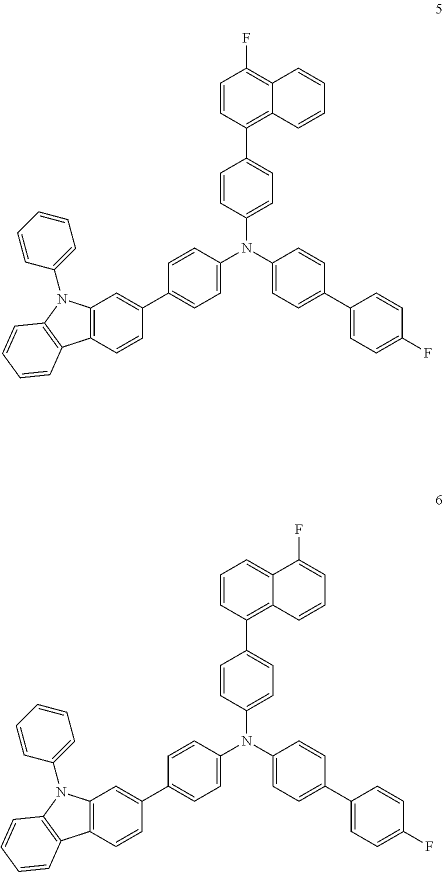 [SMILES: Fc1ccc(-c2ccc(N(c3ccc(-c4ccc5c6ccccc6n(-c6ccccc6)c5c4)cc3)c3ccc(-c4ccc(F)c5ccccc45)cc3)cc2)cc1 ; Fc1ccc(-c2ccc(N(c3ccc(-c4ccc5c6ccccc6n(-c6ccccc6)c5c4)cc3)c3ccc(-c4cccc5c(F)cccc45)cc3)cc2)cc1]

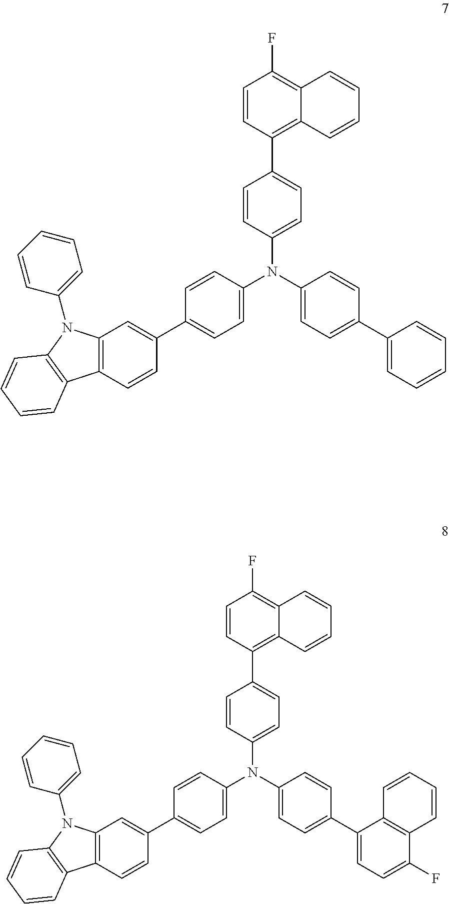 [SMILES: Fc1ccc(-c2ccc(N(c3ccc(-c4ccccc4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)c2ccccc12]

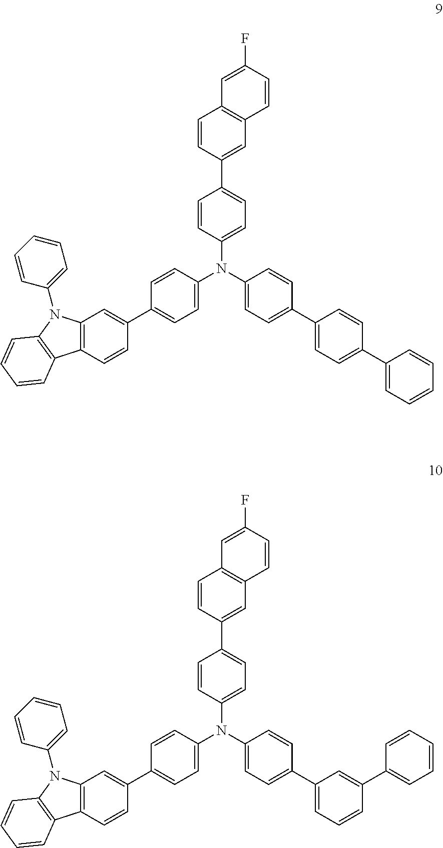 [SMILES: Fc1ccc2cc(-c3ccc(N(c4ccc(-c5ccc(-c6ccccc6)cc5)cc4)c4ccc(-c5ccc6c7ccccc7n(-c7ccccc7)c6c5)cc4)cc3)ccc2c1 ; Fc1ccc2cc(-c3ccc(N(c4ccc(-c5cccc(-c6ccccc6)c5)cc4)c4ccc(-c5ccc6c7ccccc7n(-c7ccccc7)c6c5)cc4)cc3)ccc2c1]

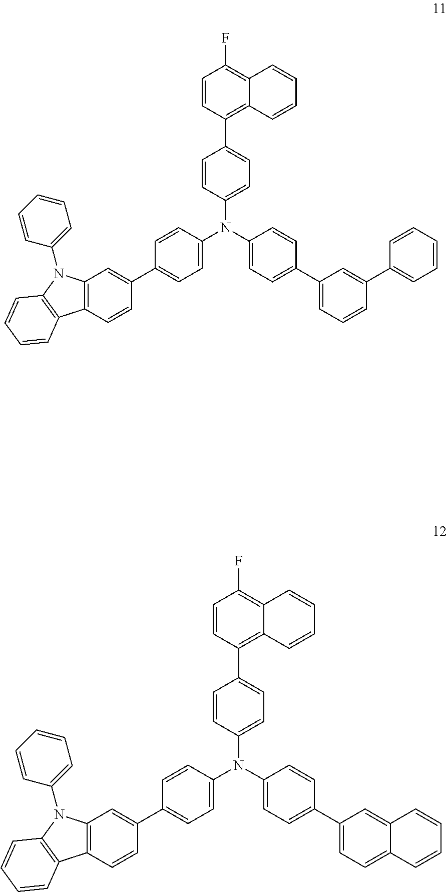 [SMILES: Fc1ccc(-c2ccc(N(c3ccc(-c4cccc(-c5ccccc5)c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5ccccc5c4)cc3)c3ccc(-c4ccc5c6ccccc6n(-c6ccccc6)c5c4)cc3)cc2)c2ccccc12]

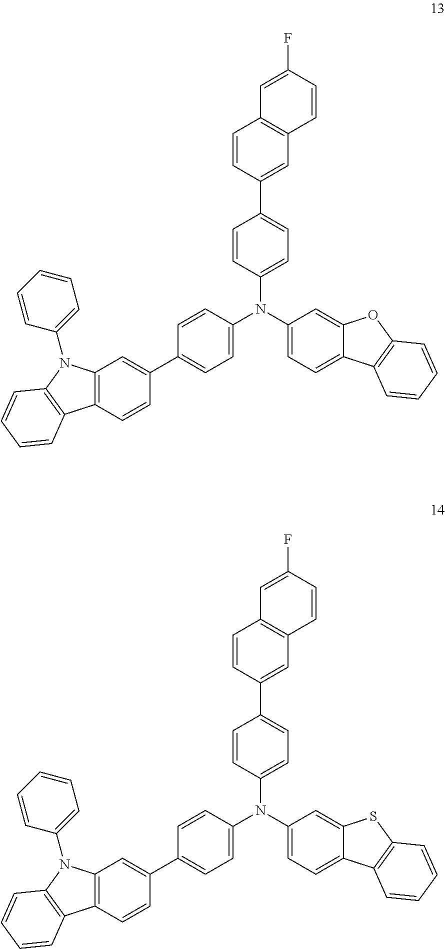 [SMILES: Fc1ccc2cc(-c3ccc(N(c4ccc(-c5ccc6c7ccccc7n(-c7ccccc7)c6c5)cc4)c4ccc5c(c4)oc4ccccc45)cc3)ccc2c1 ; Fc1ccc2cc(-c3ccc(N(c4ccc(-c5ccc6c7ccccc7n(-c7ccccc7)c6c5)cc4)c4ccc5c(c4)sc4ccccc45)cc3)ccc2c1]

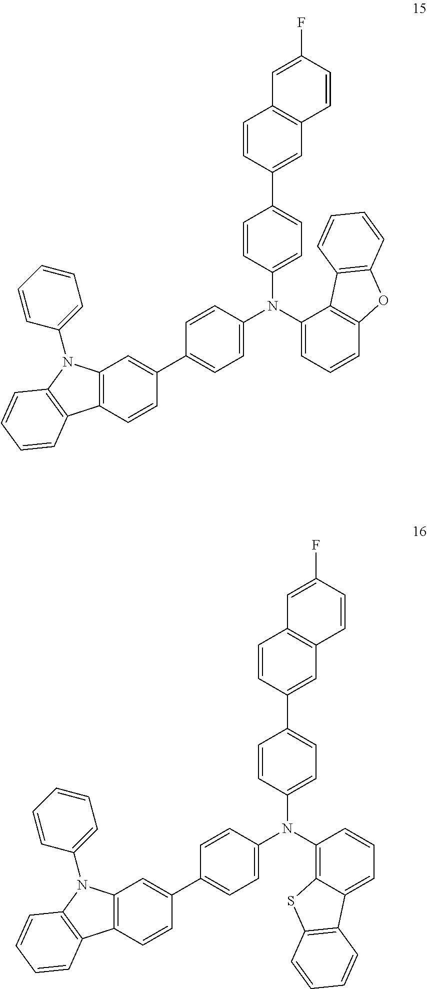 [SMILES: Fc1ccc2cc(-c3ccc(N(c4ccc(-c5ccc6c7ccccc7n(-c7ccccc7)c6c5)cc4)c4cccc5oc6ccccc6c45)cc3)ccc2c1 ; Fc1ccc2cc(-c3ccc(N(c4ccc(-c5ccc6c7ccccc7n(-c7ccccc7)c6c5)cc4)c4cccc5c4sc4ccccc45)cc3)ccc2c1]

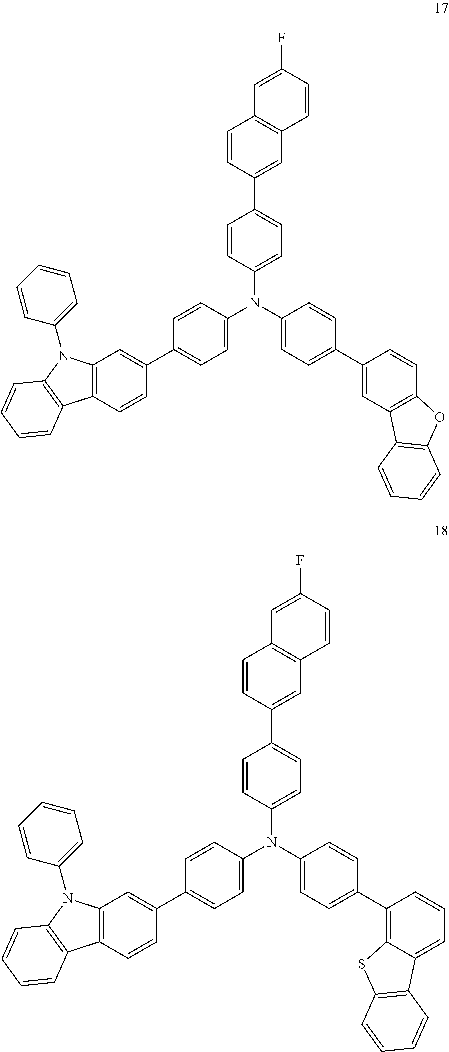 [SMILES: Fc1ccc2cc(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5sc5ccccc56)cc4)cc3)ccc2c1]

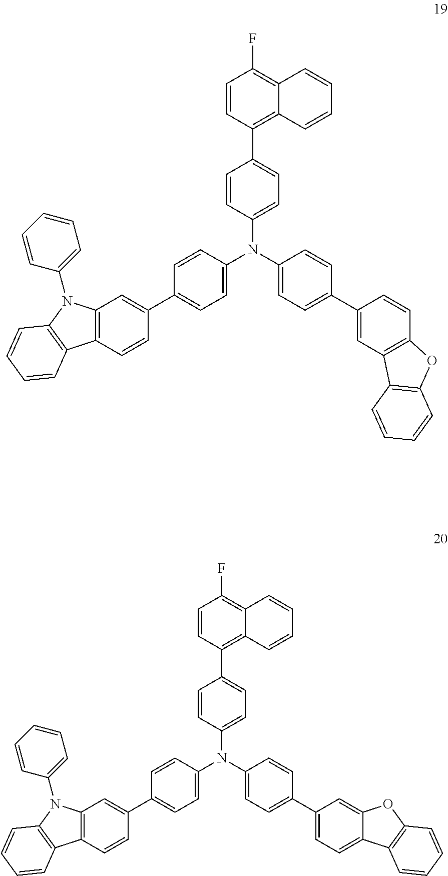 [SMILES: Fc1ccc(-c2ccc(N(c3ccc(-c4ccc5oc6ccccc6c5c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c(c4)oc4ccccc45)cc3)c3ccc(-c4ccc5c6ccccc6n(-c6ccccc6)c5c4)cc3)cc2)c2ccccc12]

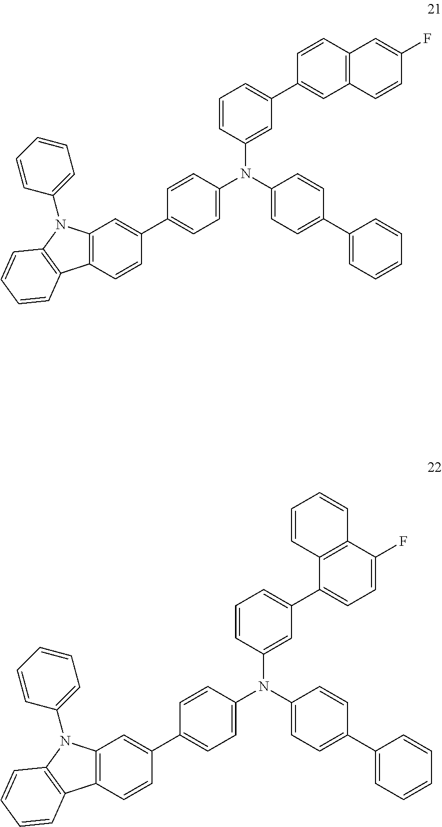 [SMILES: Fc1ccc2cc(-c3cccc(N(c4ccc(-c5ccccc5)cc4)c4ccc(-c5ccc6c7ccccc7n(-c7ccccc7)c6c5)cc4)c3)ccc2c1 ; Fc1ccc(-c2cccc(N(c3ccc(-c4ccccc4)cc3)c3ccc(-c4ccc5c6ccccc6n(-c6ccccc6)c5c4)cc3)c2)c2ccccc12]

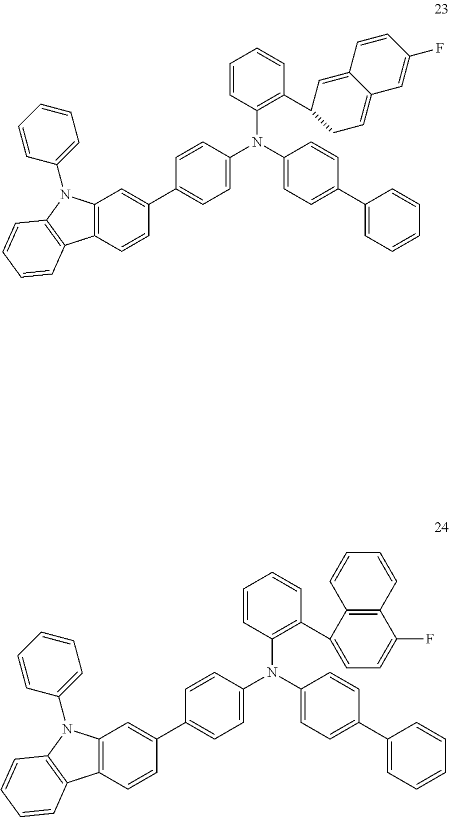 [SMILES: Fc1ccc2c(c1)=CC[C@H](c1ccccc1N(c1ccc(-c3ccccc3)cc1)c1ccc(-c3ccc4c5ccccc5n(-c5ccccc5)c4c3)cc1)C=2 ; Fc1ccc(-c2ccccc2N(c2ccc(-c3ccccc3)cc2)c2ccc(-c3ccc4c5ccccc5n(-c5ccccc5)c4c3)cc2)c2ccccc12]

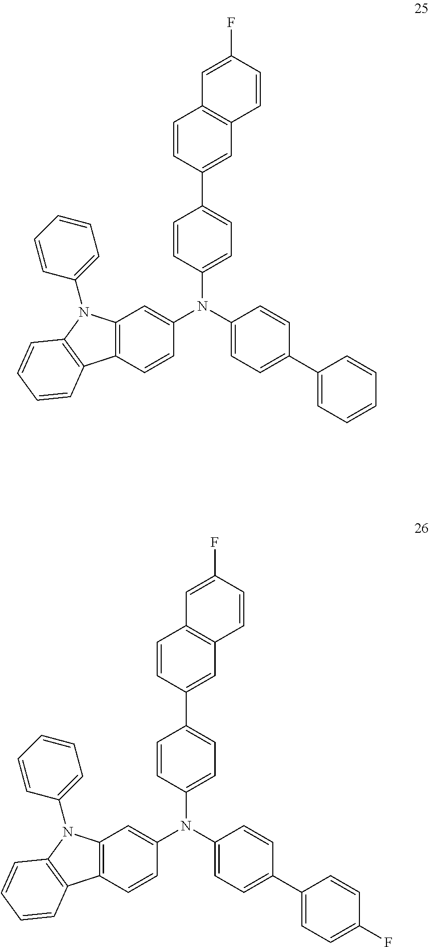 [SMILES: Fc1ccc2cc(-c3ccc(N(c4ccc(-c5ccccc5)cc4)c4ccc5c6ccccc6n(-c6ccccc6)c5c4)cc3)ccc2c1 ; Fc1ccc(-c2ccc(N(c3ccc(-c4ccc5cc(F)ccc5c4)cc3)c3ccc4c5ccccc5n(-c5ccccc5)c4c3)cc2)cc1]

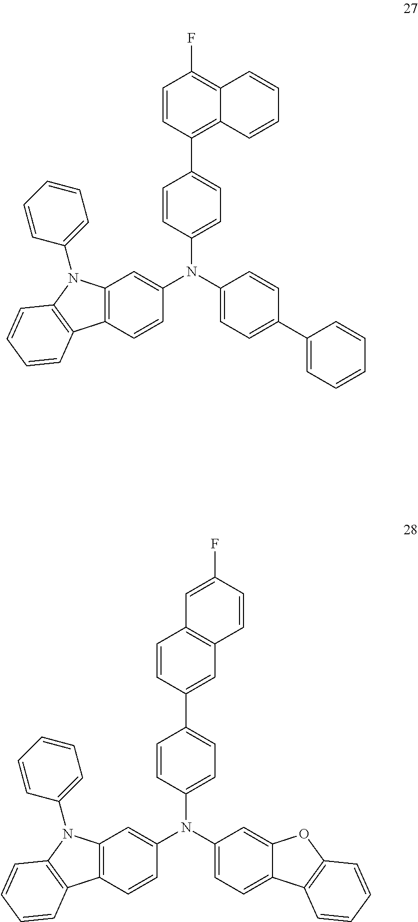 [SMILES: Fc1ccc(-c2ccc(N(c3ccc(-c4ccccc4)cc3)c3ccc4c5ccccc5n(-c5ccccc5)c4c3)cc2)c2ccccc12 ; Fc1ccc2cc(-c3ccc(N(c4ccc5c(c4)oc4ccccc45)c4ccc5c6ccccc6n(-c6ccccc6)c5c4)cc3)ccc2c1]

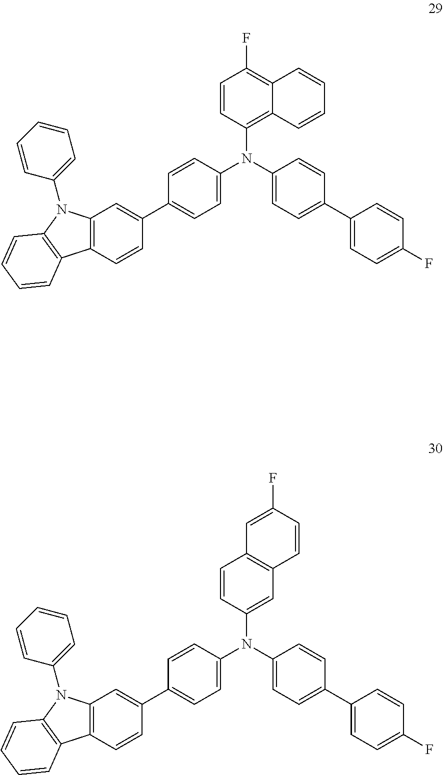 [SMILES: Fc1ccc(-c2ccc(N(c3ccc(-c4ccc5c6ccccc6n(-c6ccccc6)c5c4)cc3)c3ccc(F)c4ccccc34)cc2)cc1 ; Fc1ccc(-c2ccc(N(c3ccc(-c4ccc5c6ccccc6n(-c6ccccc6)c5c4)cc3)c3ccc4cc(F)ccc4c3)cc2)cc1]

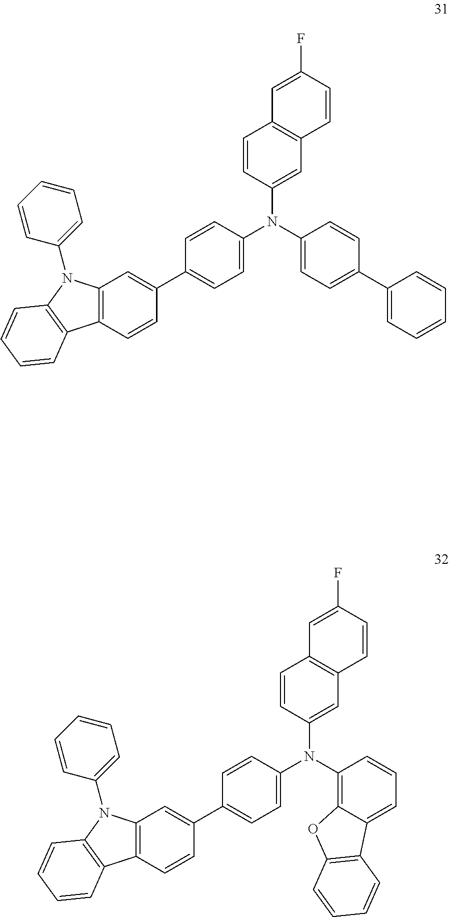 [SMILES: Fc1ccc2cc(N(c3ccc(-c4ccccc4)cc3)c3ccc(-c4ccc5c6ccccc6n(-c6ccccc6)c5c4)cc3)ccc2c1 ; Fc1ccc2cc(N(c3ccc(-c4ccc5c6ccccc6n(-c6ccccc6)c5c4)cc3)c3cccc4c3oc3ccccc34)ccc2c1]

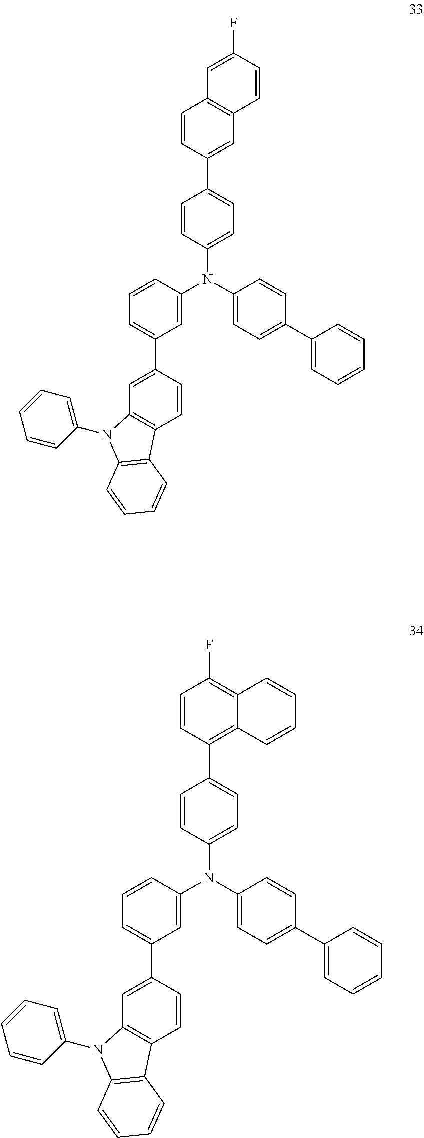 [SMILES: Fc1ccc2cc(-c3ccc(N(c4ccc(-c5ccccc5)cc4)c4cccc(-c5ccc6c7ccccc7n(-c7ccccc7)c6c5)c4)cc3)ccc2c1 ; Fc1ccc(-c2ccc(N(c3ccc(-c4ccccc4)cc3)c3cccc(-c4ccc5c6ccccc6n(-c6ccccc6)c5c4)c3)cc2)c2ccccc12]

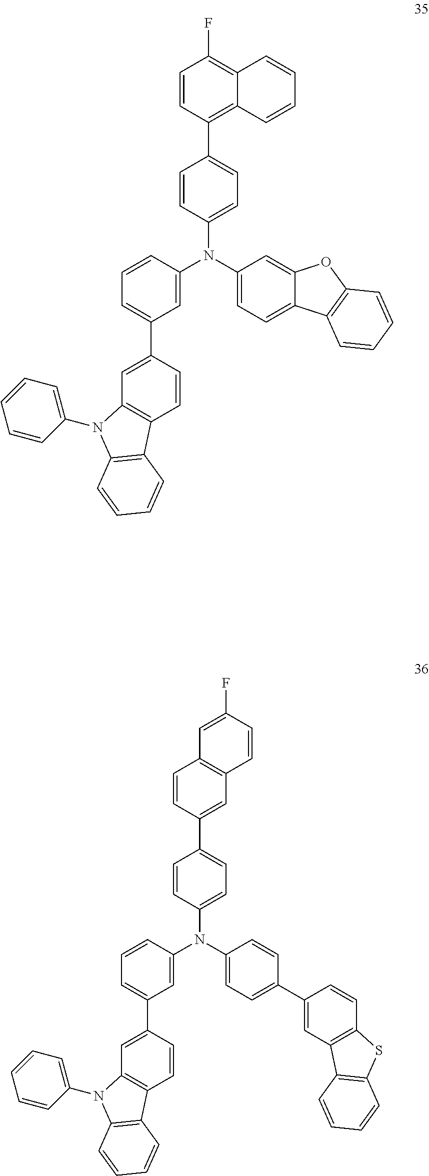 [SMILES: Fc1ccc(-c2ccc(N(c3cccc(-c4ccc5c6ccccc6n(-c6ccccc6)c5c4)c3)c3ccc4c(c3)oc3ccccc34)cc2)c2ccccc12 ; Fc1ccc2cc(-c3ccc(N(c4ccc(-c5ccc6sc7ccccc7c6c5)cc4)c4cccc(-c5ccc6c7ccccc7n(-c7ccccc7)c6c5)c4)cc3)ccc2c1]

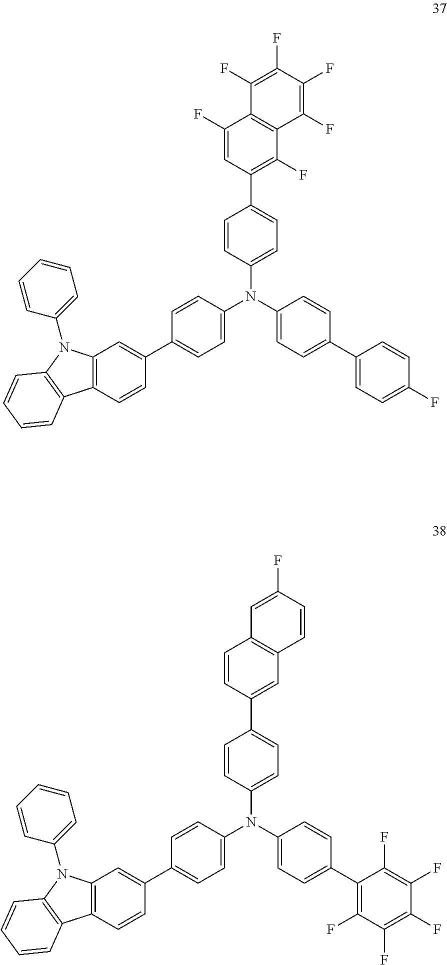 [SMILES: Fc1ccc(-c2ccc(N(c3ccc(-c4ccc5c6ccccc6n(-c6ccccc6)c5c4)cc3)c3ccc(-c4cc(F)c5c(F)c(F)c(F)c(F)c5c4F)cc3)cc2)cc1 ; Fc1ccc2cc(-c3ccc(N(c4ccc(-c5ccc6c7ccccc7n(-c7ccccc7)c6c5)cc4)c4ccc(-c5c(F)c(F)c(F)c(F)c5F)cc4)cc3)ccc2c1]

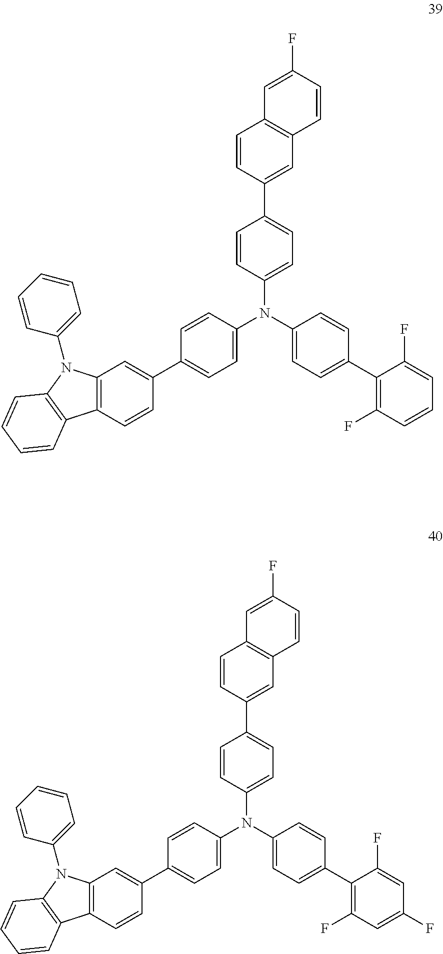 [SMILES: Fc1ccc2cc(-c3ccc(N(c4ccc(-c5ccc6c7ccccc7n(-c7ccccc7)c6c5)cc4)c4ccc(-c5c(F)cccc5F)cc4)cc3)ccc2c1 ; Fc1cc(F)c(-c2ccc(N(c3ccc(-c4ccc5cc(F)ccc5c4)cc3)c3ccc(-c4ccc5c6ccccc6n(-c6ccccc6)c5c4)cc3)cc2)c(F)c1]

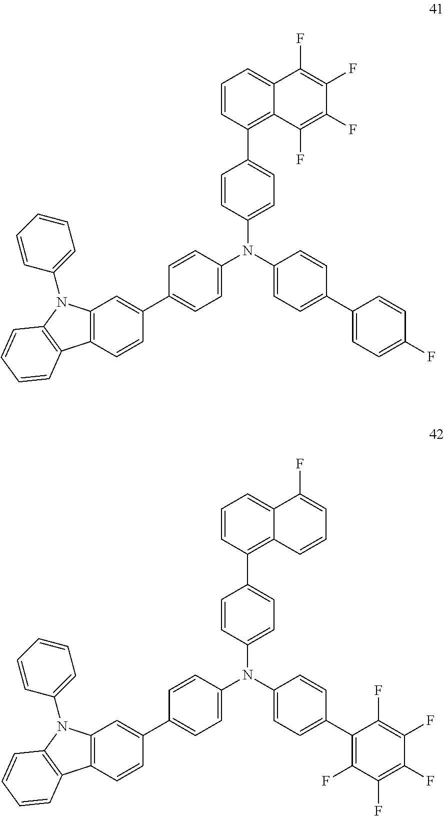 [SMILES: Fc1ccc(-c2ccc(N(c3ccc(-c4ccc5c6ccccc6n(-c6ccccc6)c5c4)cc3)c3ccc(-c4cccc5c(F)c(F)c(F)c(F)c45)cc3)cc2)cc1 ; Fc1c(F)c(F)c(-c2ccc(N(c3ccc(-c4ccc5c6ccccc6n(-c6ccccc6)c5c4)cc3)c3ccc(-c4cccc5c(F)cccc45)cc3)cc2)c(F)c1F]

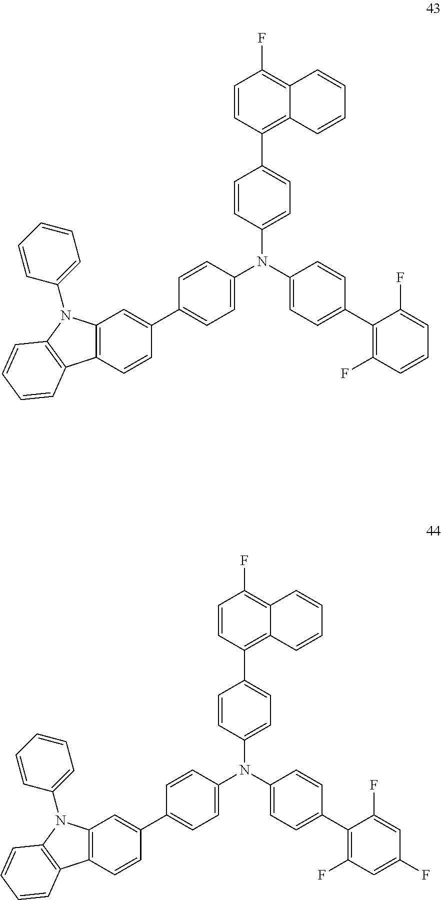 [SMILES: Fc1cccc(F)c1-c1ccc(N(c2ccc(-c3ccc4c5ccccc5n(-c5ccccc5)c4c3)cc2)c2ccc(-c3ccc(F)c4ccccc34)cc2)cc1 ; Fc1cc(F)c(-c2ccc(N(c3ccc(-c4ccc5c6ccccc6n(-c6ccccc6)c5c4)cc3)c3ccc(-c4ccc(F)c5ccccc45)cc3)cc2)c(F)c1]

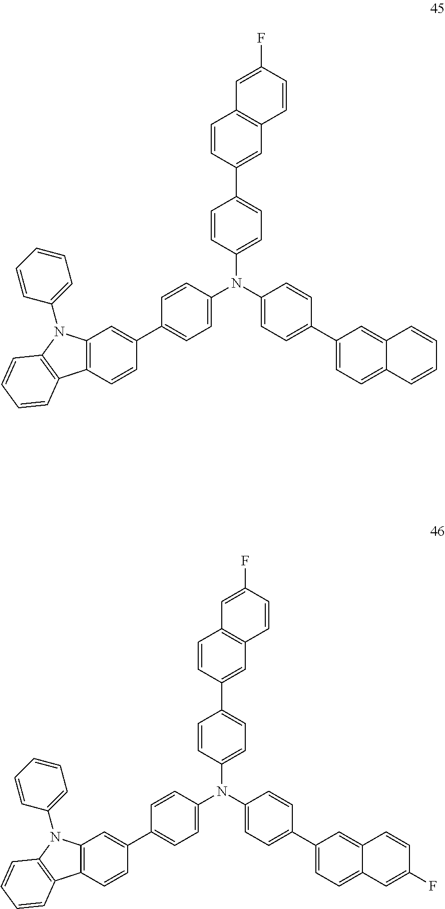 [SMILES: Fc1ccc2cc(-c3ccc(N(c4ccc(-c5ccc6ccccc6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6cc(F)ccc6c5)cc4)c4ccc(-c5ccc6c7ccccc7n(-c7ccccc7)c6c5)cc4)cc3)ccc2c1]

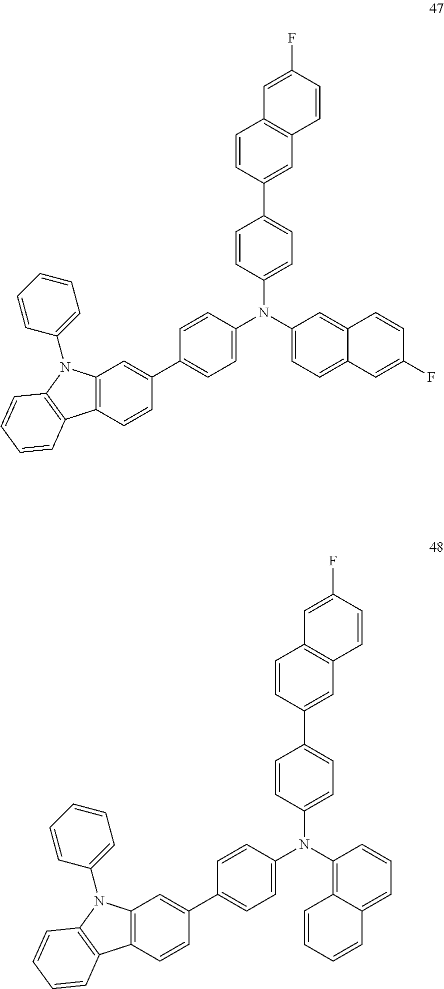 [SMILES: Fc1ccc2cc(-c3ccc(N(c4ccc(-c5ccc6c7ccccc7n(-c7ccccc7)c6c5)cc4)c4ccc5cc(F)ccc5c4)cc3)ccc2c1 ; Fc1ccc2cc(-c3ccc(N(c4ccc(-c5ccc6c7ccccc7n(-c7ccccc7)c6c5)cc4)c4cccc5ccccc45)cc3)ccc2c1]

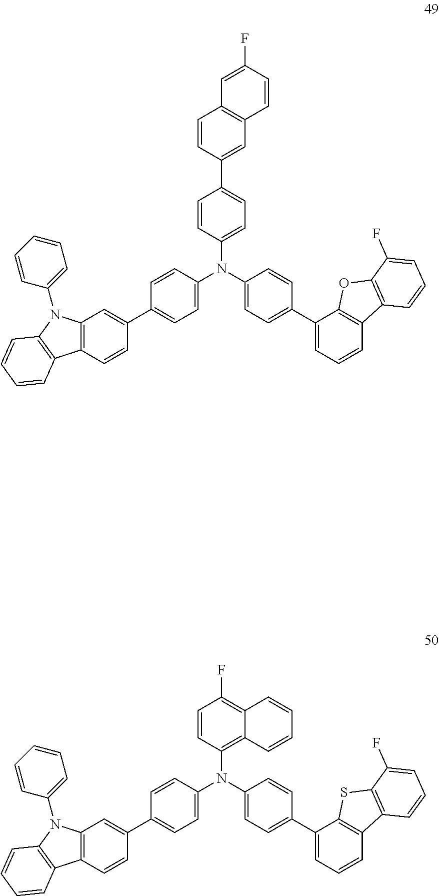 [SMILES: Fc1ccc2cc(-c3ccc(N(c4ccc(-c5ccc6c7ccccc7n(-c7ccccc7)c6c5)cc4)c4ccc(-c5cccc6c5oc5c(F)cccc56)cc4)cc3)ccc2c1 ; Fc1ccc(N(c2ccc(-c3ccc4c5ccccc5n(-c5ccccc5)c4c3)cc2)c2ccc(-c3cccc4c3sc3c(F)cccc34)cc2)c2ccccc12]

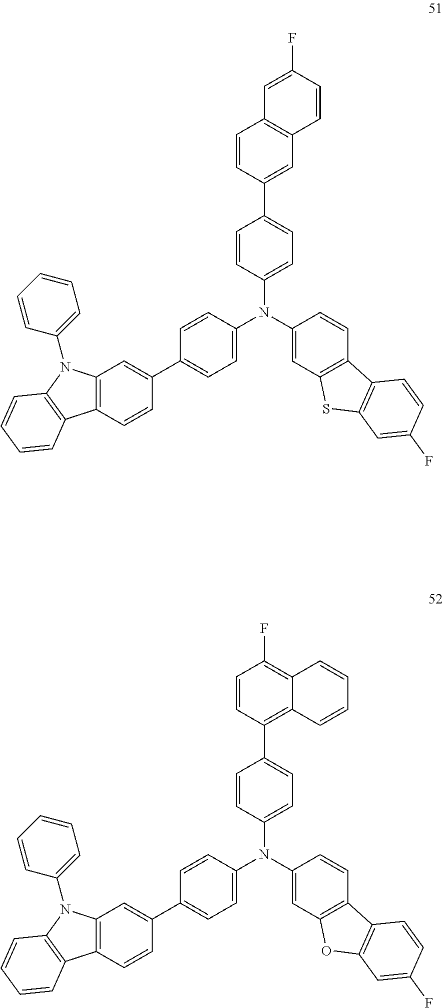 [SMILES: Fc1ccc2cc(-c3ccc(N(c4ccc(-c5ccc6c7ccccc7n(-c7ccccc7)c6c5)cc4)c4ccc5c(c4)sc4cc(F)ccc45)cc3)ccc2c1 ; Fc1ccc2c(c1)oc1cc(N(c3ccc(-c4ccc5c6ccccc6n(-c6ccccc6)c5c4)cc3)c3ccc(-c4ccc(F)c5ccccc45)cc3)ccc12]

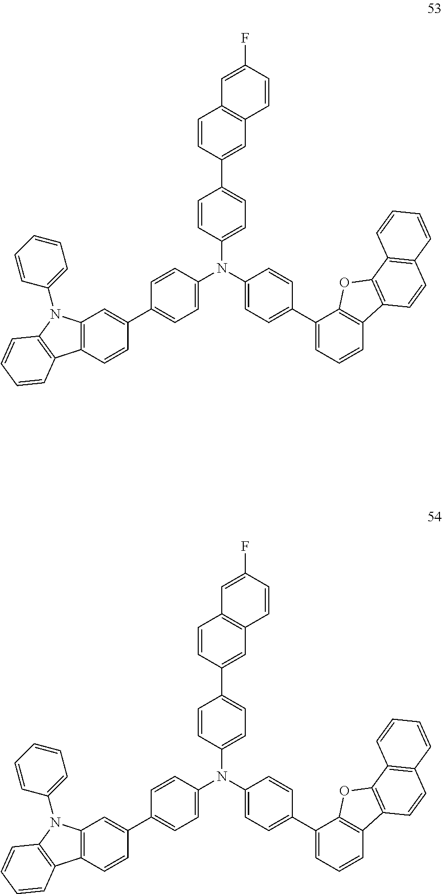 [SMILES: Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1]

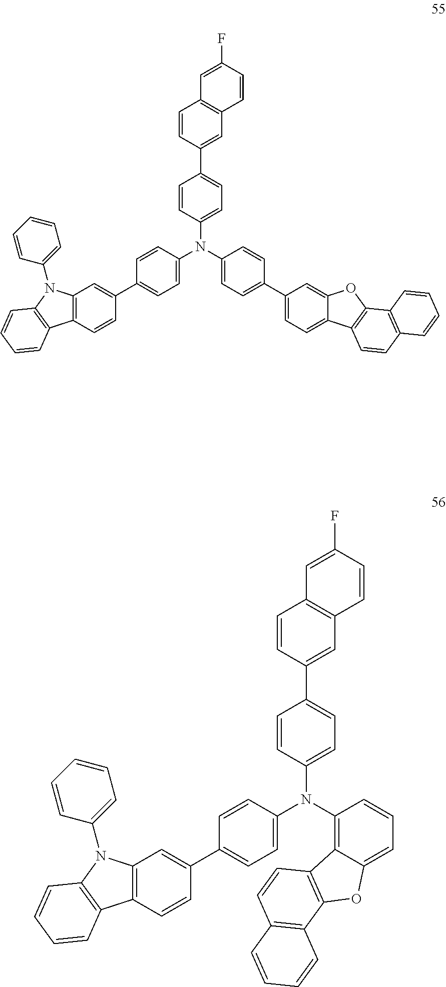 [SMILES: Fc1ccc2cc(-c3ccc(N(c4ccc(-c5ccc6c(c5)oc5c7ccccc7ccc65)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4cccc5oc6c7ccccc7ccc6c45)cc3)ccc2c1]

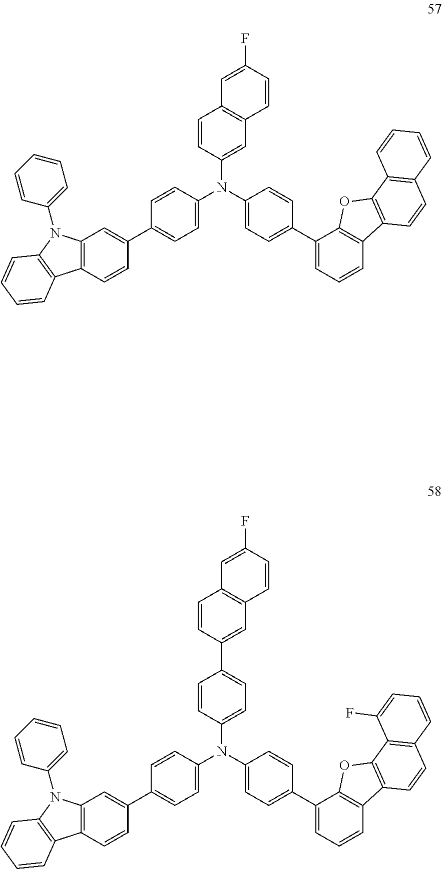 [SMILES: Fc1ccc2cc(N(c3ccc(-c4ccc5c6ccccc6n(-c6ccccc6)c5c4)cc3)c3ccc(-c4cccc5c4oc4c6ccccc6ccc54)cc3)ccc2c1 ; Fc1ccc2cc(-c3ccc(N(c4ccc(-c5ccc6c7ccccc7n(-c7ccccc7)c6c5)cc4)c4ccc(-c5cccc6c5oc5c6ccc6cccc(F)c65)cc4)cc3)ccc2c1]

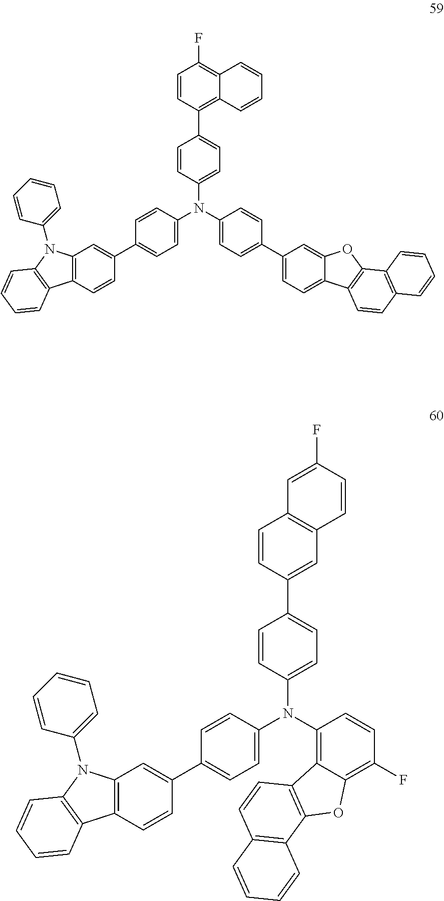 [SMILES: Fc1ccc(-c2ccc(N(c3ccc(-c4ccc5c(c4)oc4c6ccccc6ccc54)cc3)c3ccc(-c4ccc5c6ccccc6n(-c6ccccc6)c5c4)cc3)cc2)c2ccccc12 ; Fc1ccc2cc(-c3ccc(N(c4ccc(-c5ccc6c7ccccc7n(-c7ccccc7)c6c5)cc4)c4ccc(F)c5oc6c7ccccc7ccc6c45)cc3)ccc2c1]

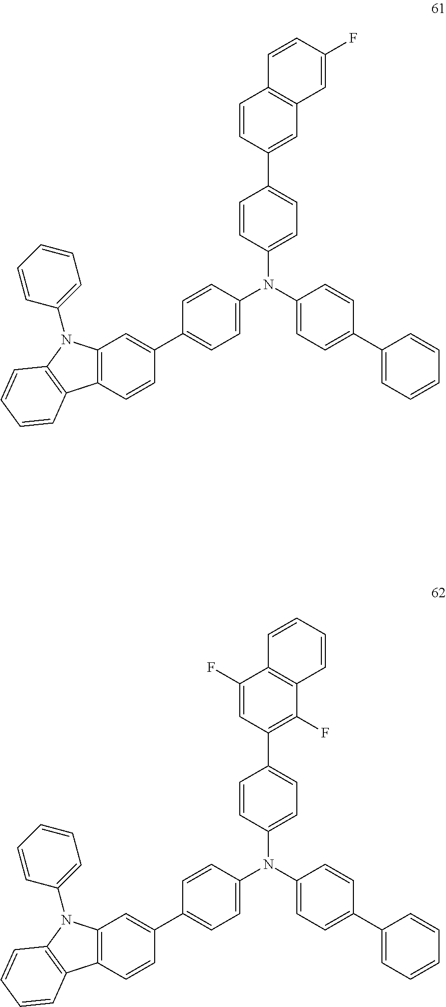 [SMILES: Fc1ccc2ccc(-c3ccc(N(c4ccc(-c5ccccc5)cc4)c4ccc(-c5ccc6c7ccccc7n(-c7ccccc7)c6c5)cc4)cc3)cc2c1 ; Fc1cc(-c2ccc(N(c3ccc(-c4ccccc4)cc3)c3ccc(-c4ccc5c6ccccc6n(-c6ccccc6)c5c4)cc3)cc2)c(F)c2ccccc12]

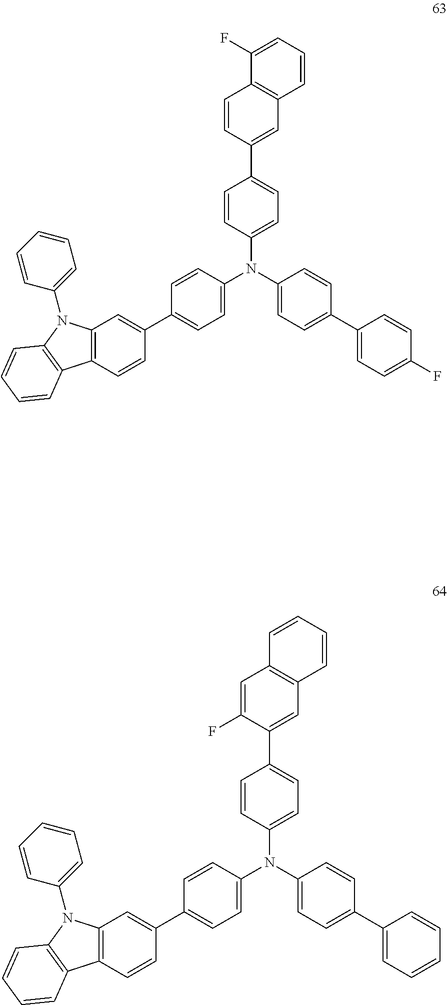 [SMILES: Fc1ccc(-c2ccc(N(c3ccc(-c4ccc5c(F)cccc5c4)cc3)c3ccc(-c4ccc5c6ccccc6n(-c6ccccc6)c5c4)cc3)cc2)cc1 ; Fc1cc2ccccc2cc1-c1ccc(N(c2ccc(-c3ccccc3)cc2)c2ccc(-c3ccc4c5ccccc5n(-c5ccccc5)c4c3)cc2)cc1]

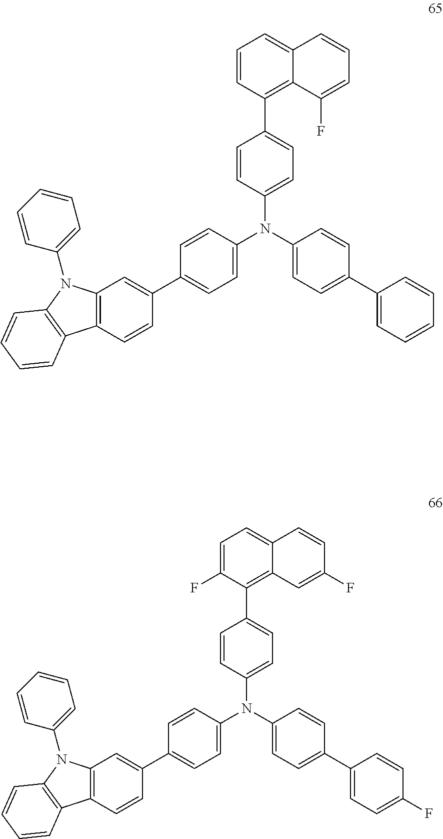 [SMILES: Fc1cccc2cccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4c(F)ccc5ccc(F)cc45)cc3)cc2)cc1]

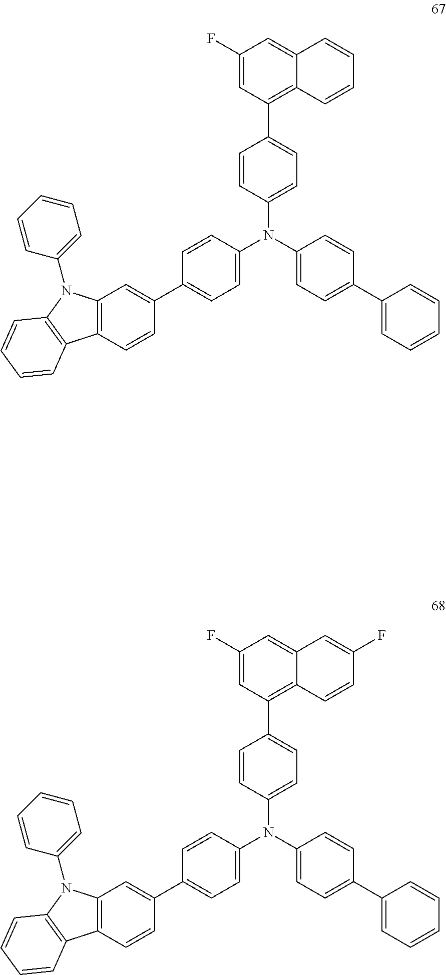 [SMILES: Fc1cc(-c2ccc(N(c3ccc(-c4ccccc4)cc3)c3ccc(-c4ccc5c6ccccc6n(-c6ccccc6)c5c4)cc3)cc2)c2ccccc2c1 ; Fc1ccc2c(-c3ccc(N(c4ccc(-c5ccccc5)cc4)c4ccc(-c5ccc6c7ccccc7n(-c7ccccc7)c6c5)cc4)cc3)cc(F)cc2c1]

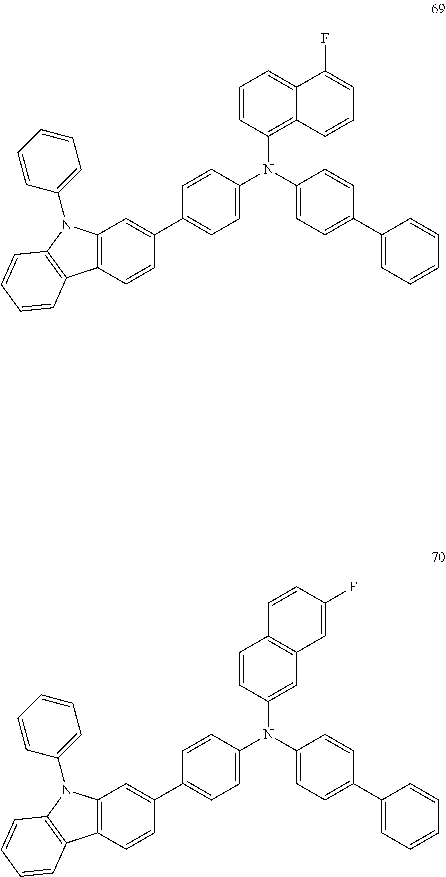 [SMILES: Fc1cccc2c(N(c3ccc(-c4ccccc4)cc3)c3ccc(-c4ccc5c6ccccc6n(-c6ccccc6)c5c4)cc3)cccc12 ; Fc1ccc2ccc(N(c3ccc(-c4ccccc4)cc3)c3ccc(-c4ccc5c6ccccc6n(-c6ccccc6)c5c4)cc3)cc2c1]

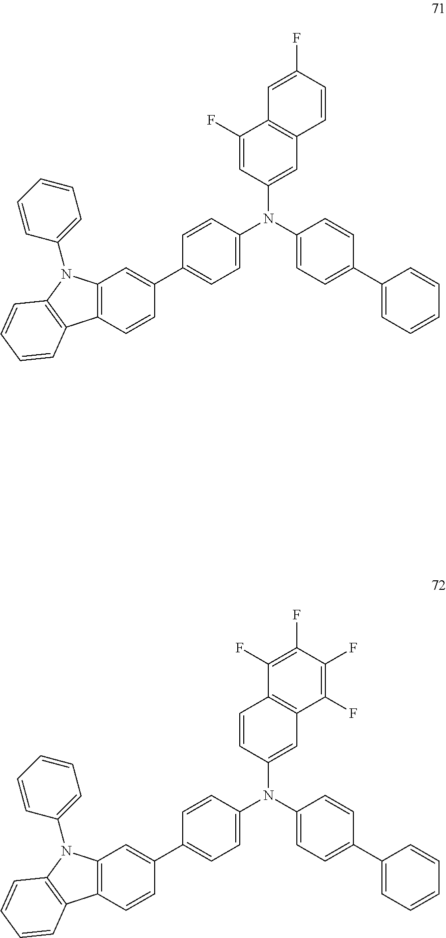 [SMILES: Fc1ccc2cc(N(c3ccc(-c4ccccc4)cc3)c3ccc(-c4ccc5c6ccccc6n(-c6ccccc6)c5c4)cc3)cc(F)c2c1 ; Fc1c(F)c(F)c2cc(N(c3ccc(-c4ccccc4)cc3)c3ccc(-c4ccc5c6ccccc6n(-c6ccccc6)c5c4)cc3)ccc2c1F]

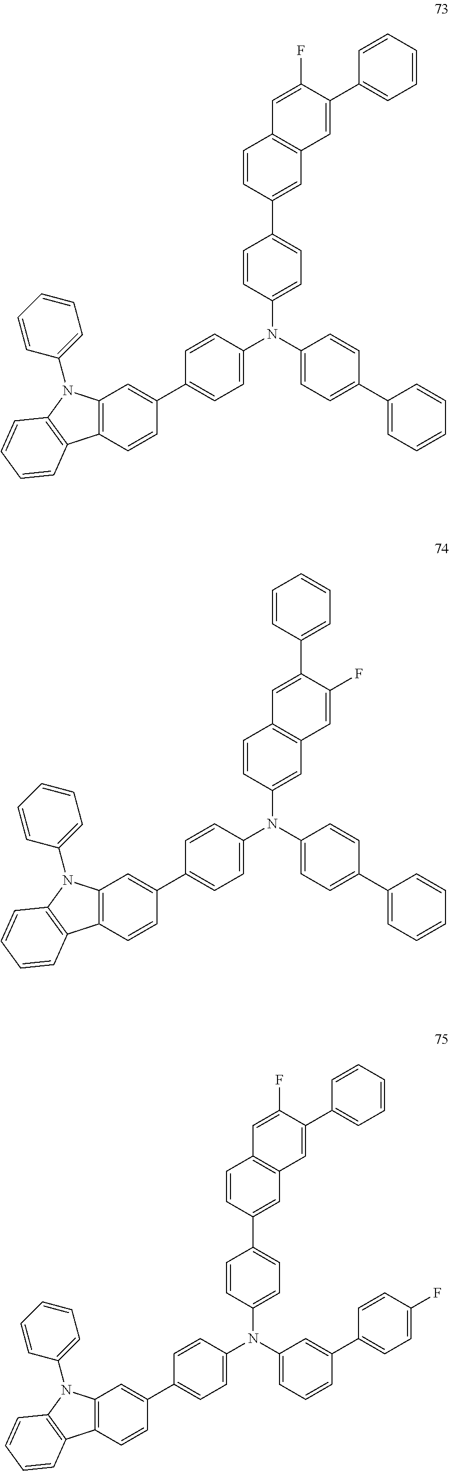 [SMILES: Fc1cc2ccc(-c3ccc(N(c4ccc(-c5ccccc5)cc4)c4ccc(-c5ccc6c7ccccc7n(-c7ccccc7)c6c5)cc4)cc3)cc2cc1-c1ccccc1 ; Fc1cc2cc(N(c3ccc(-c4ccccc4)cc3)c3ccc(-c4ccc5c6ccccc6n(-c6ccccc6)c5c4)cc3)ccc2cc1-c1ccccc1 ; Fc1ccc(-c2cccc(N(c3ccc(-c4ccc5cc(F)c(-c6ccccc6)cc5c4)cc3)c3ccc(-c4ccc5c6ccccc6n(-c6ccccc6)c5c4)cc3)c2)cc1]

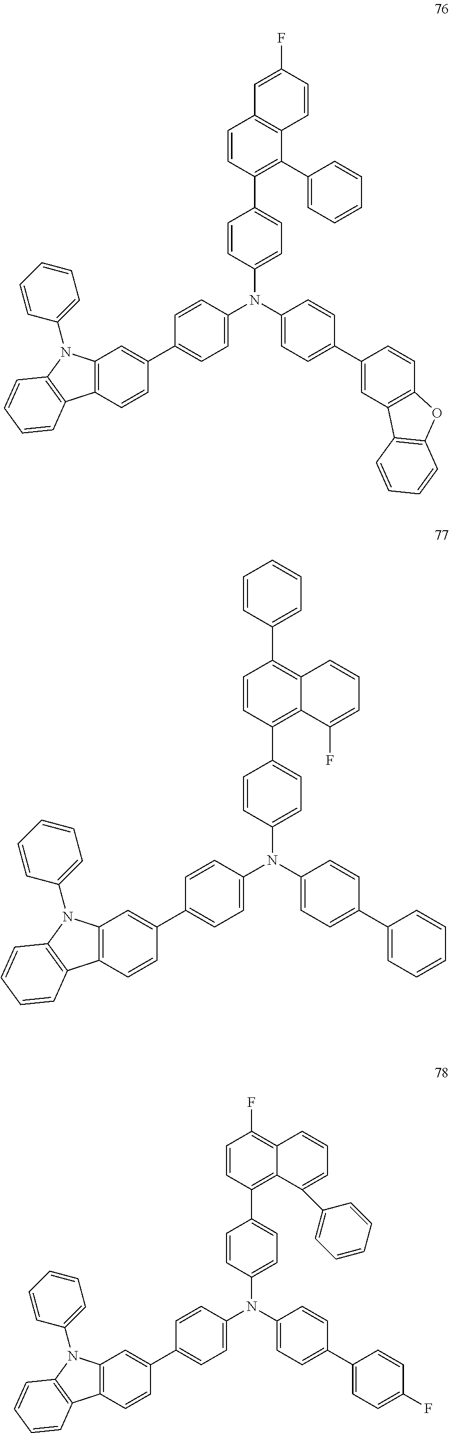 [SMILES: Fc1ccc2c(-c3ccccc3)c(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1cccc2c(-c3ccccc3)ccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4ccc(F)c5cccc(-c6ccccc6)c45)cc3)cc2)cc1]

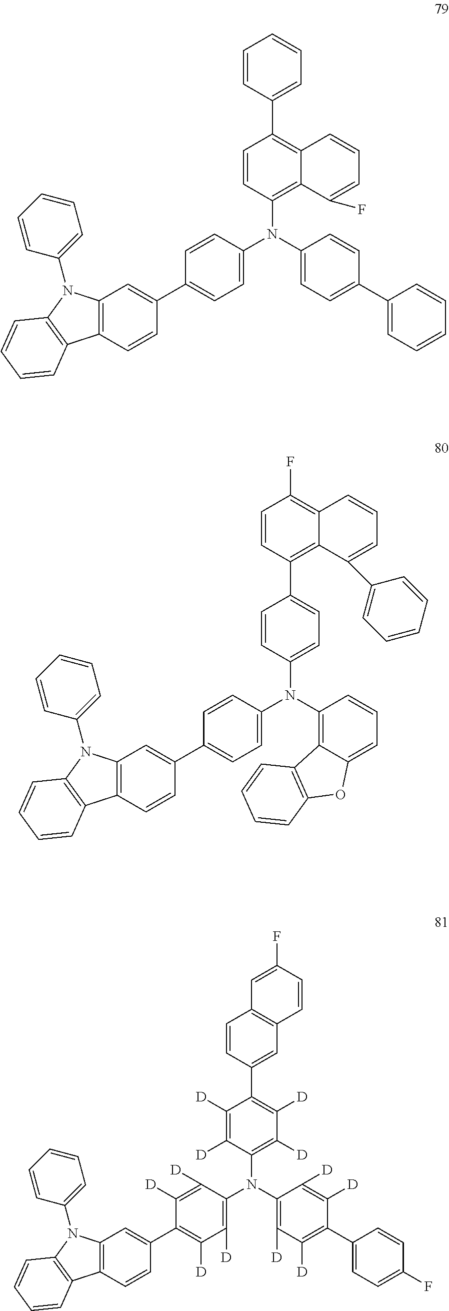 [SMILES: Fc1cccc2c(-c3ccccc3)ccc(N(c3ccc(-c4ccccc4)cc3)c3ccc(-c4ccc5c6ccccc6n(-c6ccccc6)c5c4)cc3)c12 ; Fc1ccc(-c2ccc(N(c3ccc(-c4ccc5c6ccccc6n(-c6ccccc6)c5c4)cc3)c3cccc4oc5ccccc5c34)cc2)c2c(-c3ccccc3)cccc12 ; [2H]c1c([2H])c(N(c2c([2H])c([2H])c(-c3ccc4cc(F)ccc4c3)c([2H])c2[2H])c2c([2H])c([2H])c(-c3ccc4c5ccccc5n(-c5ccccc5)c4c3)c([2H])c2[2H])c([2H])c([2H])c1-c1ccc(F)cc1]

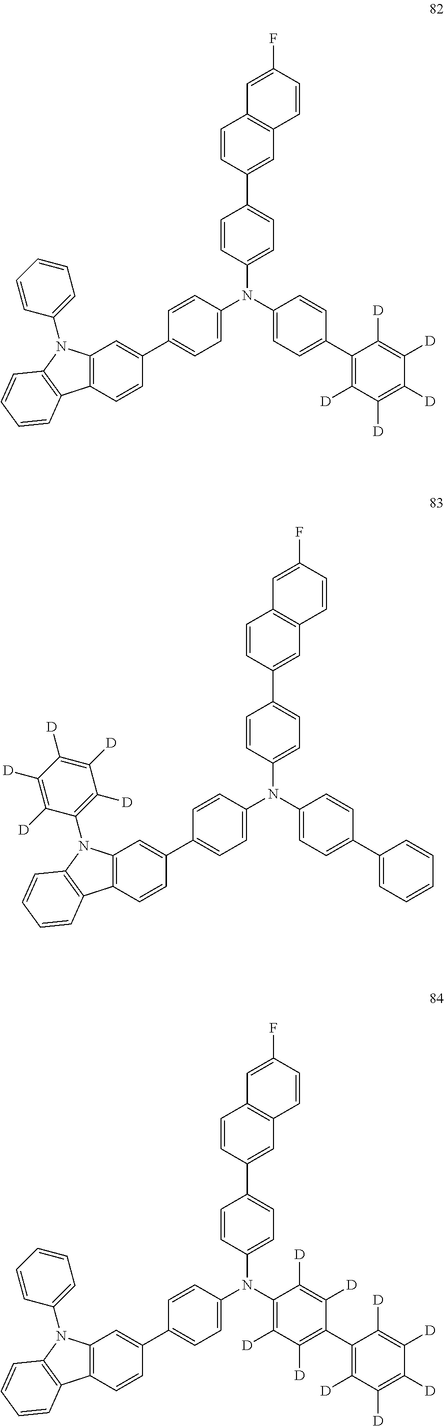 [SMILES: [2H]c1c([2H])c([2H])c(-c2ccc(N(c3ccc(-c4ccc5cc(F)ccc5c4)cc3)c3ccc(-c4ccc5c6ccccc6n(-c6ccccc6)c5c4)cc3)cc2)c([2H])c1[2H] ; [2H]c1c([2H])c([2H])c(-n2c3ccccc3c3ccc(-c4ccc(N(c5ccc(-c6ccccc6)cc5)c5ccc(-c6ccc7cc(F)ccc7c6)cc5)cc4)cc32)c([2H])c1[2H] ; [2H]c1c([2H])c([2H])c(-c2c([2H])c([2H])c(N(c3ccc(-c4ccc5cc(F)ccc5c4)cc3)c3ccc(-c4ccc5c6ccccc6n(-c6ccccc6)c5c4)cc3)c([2H])c2[2H])c([2H])c1[2H]]

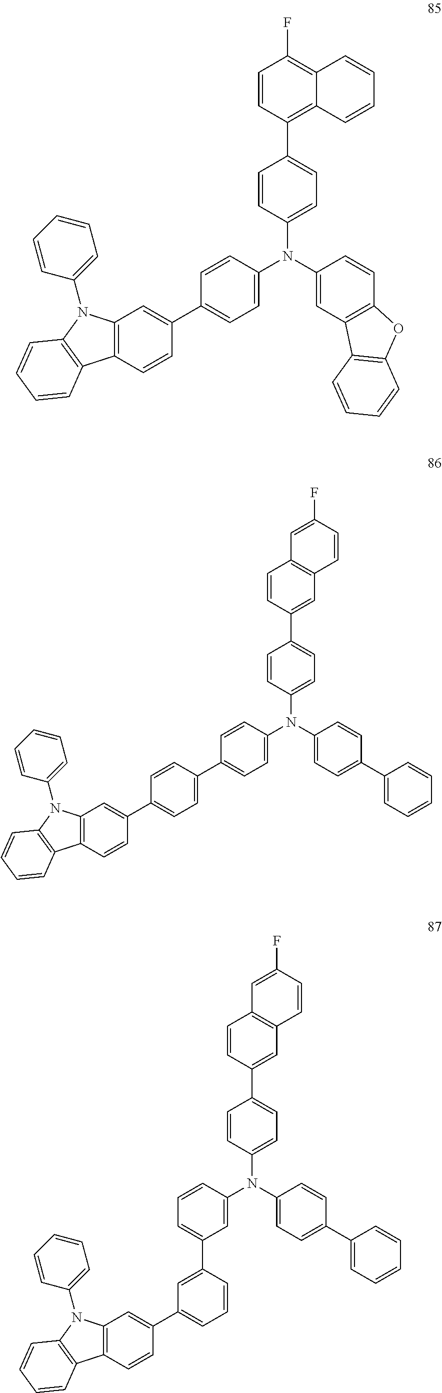 [SMILES: Fc1ccc(-c2ccc(N(c3ccc(-c4ccc5c6ccccc6n(-c6ccccc6)c5c4)cc3)c3ccc4oc5ccccc5c4c3)cc2)c2ccccc12 ; Fc1ccc2cc(-c3ccc(N(c4ccc(-c5ccccc5)cc4)c4ccc(-c5ccc(-c6ccc7c8ccccc8n(-c8ccccc8)c7c6)cc5)cc4)cc3)ccc2c1 ; Fc1ccc2cc(-c3ccc(N(c4ccc(-c5ccccc5)cc4)c4cccc(-c5cccc(-c6ccc7c8ccccc8n(-c8ccccc8)c7c6)c5)c4)cc3)ccc2c1]

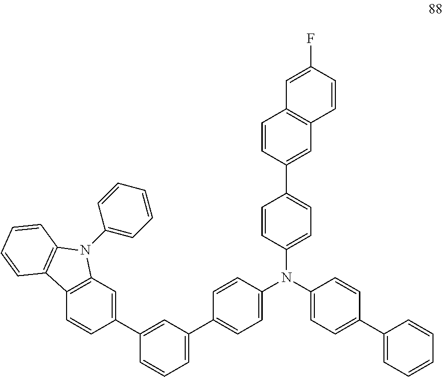 [SMILES: Fc1ccc2cc(-c3ccc(N(c4ccc(-c5ccccc5)cc4)c4ccc(-c5cccc(-c6ccc7c8ccccc8n(-c8ccccc8)c7c6)c5)cc4)cc3)ccc2c1]

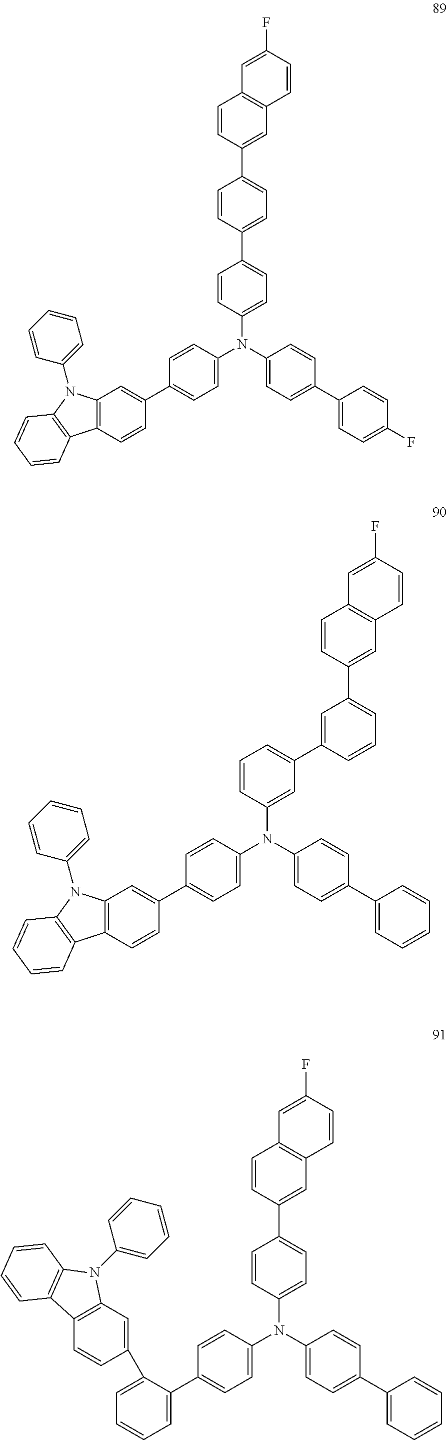 [SMILES: Fc1ccc(-c2ccc(N(c3ccc(-c4ccc(-c5ccc6cc(F)ccc6c5)cc4)cc3)c3ccc(-c4ccc5c6ccccc6n(-c6ccccc6)c5c4)cc3)cc2)cc1 ; Fc1ccc2cc(-c3cccc(-c4cccc(N(c5ccc(-c6ccccc6)cc5)c5ccc(-c6ccc7c8ccccc8n(-c8ccccc8)c7c6)cc5)c4)c3)ccc2c1 ; Fc1ccc2cc(-c3ccc(N(c4ccc(-c5ccccc5)cc4)c4ccc(-c5ccccc5-c5ccc6c7ccccc7n(-c7ccccc7)c6c5)cc4)cc3)ccc2c1]

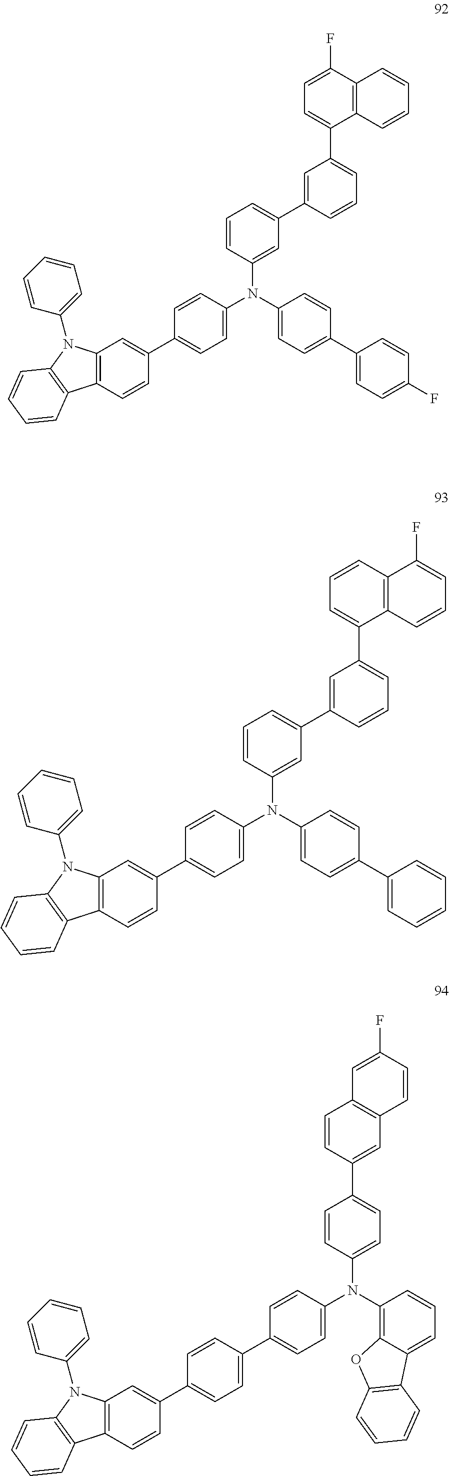 [SMILES: Fc1ccc(-c2ccc(N(c3ccc(-c4ccc5c6ccccc6n(-c6ccccc6)c5c4)cc3)c3cccc(-c4cccc(-c5ccc(F)c6ccccc56)c4)c3)cc2)cc1 ; Fc1cccc2c(-c3cccc(-c4cccc(N(c5ccc(-c6ccccc6)cc5)c5ccc(-c6ccc7c8ccccc8n(-c8ccccc8)c7c6)cc5)c4)c3)cccc12 ; Fc1ccc2cc(-c3ccc(N(c4ccc(-c5ccc(-c6ccc7c8ccccc8n(-c8ccccc8)c7c6)cc5)cc4)c4cccc5c4oc4ccccc45)cc3)ccc2c1]

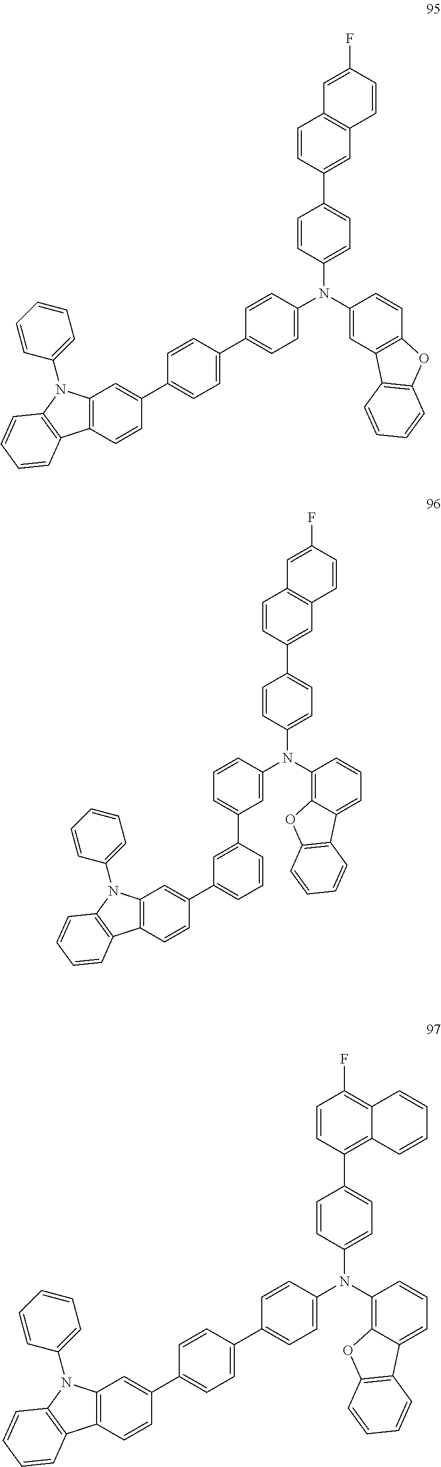 [SMILES: Fc1ccc2cc(-c3ccc(N(c4ccc(-c5ccc(-c6ccc7c8ccccc8n(-c8ccccc8)c7c6)cc5)cc4)c4ccc5oc6ccccc6c5c4)cc3)ccc2c1 ; Fc1ccc2cc(-c3ccc(N(c4cccc(-c5cccc(-c6ccc7c8ccccc8n(-c8ccccc8)c7c6)c5)c4)c4cccc5c4oc4ccccc45)cc3)ccc2c1 ; Fc1ccc(-c2ccc(N(c3ccc(-c4ccc(-c5ccc6c7ccccc7n(-c7ccccc7)c6c5)cc4)cc3)c3cccc4c3oc3ccccc34)cc2)c2ccccc12]

In Compound Group 1, D is a deuterium atom.

In an embodiment, an electronic apparatus includes a display module including a plurality of light-emitting elements, and at least one among the plurality of light-emitting elements includes a first electrode, a second electrode disposed on the first electrode, an emission layer disposed between the first electrode and the second electrode, and a hole transport region disposed between the first electrode and the emission layer and containing an amine compound according to Formula 1.

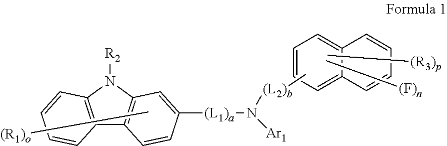

In Formula 1, L₁ and L₂ are each independently a substituted or unsubstituted arylene group having 6 to 30 carbon atoms, Ar₁ a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms, each R₁, R₂, and each are independently a hydrogen atom, a deuterium atom, a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms, a and b are each independently an integer of 0 to 4, provided that: when a is 0, N of NAr₁ is directly bonded to the 2-position of a carbazole group, and when b is 0, N of NAr₁ is directly bonded to a naphthyl group, n is an integer of 1 to 7, o is an integer of 0 to 7, and p is an integer of 0 to (7-n).

In an embodiment, the electronic apparatus may further include a plurality of display devices, controlled independently and each configured to display an image, and at least one among the display devices may include the display module.

In an embodiment, the electronic apparatus may further include at least one among a processor, a memory, and a power module.

### BRIEF DESCRIPTION OF THE FIGURES

The accompanying drawings are included to provide a further understanding and are incorporated in and constitute a part of this specification. The drawings illustrate embodiments of this disclosure and, together with the description, serve to explain certain principles. In the drawings:

FIG. 1 is a block diagram of an electronic apparatus according to an embodiment;

FIG. 2 illustrates schematic views of electronic apparatuses according to embodiments;

FIG. 3 is a plan view of a display module according to an embodiment;

FIG. 4 is a cross-sectional view illustrating a portion taken along line I-I′ in FIG. 3;

FIG. 5 is a cross-sectional view schematically illustrating a light-emitting element according to an embodiment;

FIG. 6 is a cross-sectional view schematically illustrating a light-emitting element according to an embodiment;

FIG. 7 is a cross-sectional view schematically illustrating a light-emitting element according to an embodiment;

FIG. 8 is a cross-sectional view schematically illustrating a light-emitting element according to an embodiment;

FIG. 9 is a cross-sectional view schematically illustrating a light-emitting element according to an embodiment;

FIG. 10 is a cross-sectional view schematically illustrating a light-emitting element according to an embodiment;

FIG. 11 is a cross-sectional view illustrating a display module according to an embodiment;

FIG. 12 is a cross-sectional view illustrating a display module according to an embodiment;

FIG. 13 is a cross-sectional view illustrating a display module according to an embodiment;

FIG. 14 is a cross-sectional view illustrating a display module according to an embodiment;

FIG. 15 is a perspective view of an electronic apparatus according to an embodiment;

FIG. 16 is a perspective view of an electronic apparatus according to an embodiment; and

FIG. 17 is a view illustrating an inside of a vehicle in which an electronic apparatus according to an embodiment is disposed.

### DETAILED DESCRIPTION

In this specification, it will be understood that when an element (or a region, a layer, a portion, or the like) is referred to as being “on”, “connected to” or “coupled to” another element, it may be directly disposed on, connected to, or coupled to the other element, or other elements may be disposed therebetween.

Like reference numerals or symbols refer to like elements throughout. In the drawings, the thickness, ratio, and size of the elements are exaggerated for effectively describing the technical contents. As used herein, the term “and/or” includes any and all combinations of one or more of the associated listed elements.

It will be understood that, although the terms “first”, “second”, etc. may be used herein to describe various elements, the elements are not to be limited by these terms. These terms are only used to distinguish one element, component, region, layer or section from another element, component, region, layer, or section. For instance, a first element, component, region, layer, or section discussed below could be termed a second element, component, region, layer or section without departing from the scope of the inventive concept. Similarly, a second element, component, region, layer or section could be termed a first element, component, region, layer or section. In this specification, the singular expressions “a”, “an” and “the” are intended to include the plural forms as well, unless the context clearly indicates otherwise.

In addition, the terms “below”, “under”, “on the lower side”, “above”, “over”, “on the upper side”, or the like may be used to describe the relationships between the elements illustrated in the drawings. These terms are relative concepts and are described on the basis of the directions indicated in the drawings.

It will be further understood that the terms “comprises, includes, has” and/or “comprising, including, having”, when used in this specification, specify the presence of stated features, numbers, steps, operations, elements, components or combinations thereof, but do not preclude the possibility of the presence or addition of one or more other features, numbers, steps, operations, elements, components, and/or combinations thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In this specification, the wording “substituted or unsubstituted” may refer to being substituted or unsubstituted with one or more substituents selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, the exemplified substituents may each be substituted or unsubstituted. For example, a biphenyl group may be interpreted as an aryl group and also interpreted as a phenyl group substituted with a phenyl group. The number of carbon atoms in the substituent is included in the total number of carbon atoms of the groups. For example, an unsubstituted biphenyl groups have 8 carbon atoms; a biphenyl group having a methyl substituent would have 9 carbon atoms.

In this specification, the wording “bonded to an adjacent group to form a ring” may refer to being bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, a ring formed through mutual bonding may be bonded to another ring to form a spiro structure.

In this specification, the term “adjacent group” may refer to a substituent substituted for an atom which is directly connected to an atom substituted with a corresponding substituent, another substituent substituted for the atom which is substituted with the corresponding substituent, or a substituent sterically most adjacent to the corresponding substituent. For example, in 1,2-dimethylbenzene, two methyl groups may be interpreted as mutually “adjacent groups”, and in 1,1-diethylcyclopentene, two ethyl groups may be interpreted as mutually “adjacent groups”. In addition, in 4,5-dimethylphenanthrene, two methyl groups may be interpreted as mutually “adjacent groups”.

In this specification, examples of a halogen atom include a fluorine atom, a chlorine atom, a bromine atom, or an iodine atom.

In this specification, an alkyl group may be a linear chain, or a branched chain. The carbon number of the alkyl group is 1 to 60, 1 to 30, 1 to 20, 1 to 15, 1 to 10, or 1 to 6. Examples of the alkyl group may include a methyl group, an ethyl group, a n-propyl group, an isopropyl group, a n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, a n-hexyl group, a 1-methylhexyl group, a 2-ethylhexyl group, a 2-butylhexyl group, a n-heptyl group, a 1-methylheptyl group, a 2,2-dimethylheptyl group, a 2-ethylheptyl group, a 2-butylheptyl group, a n-octyl group, a t-octyl group, a 2-ethyloctyl group, a 2-butyloctyl group, a 2-hexyloctyl group, a 3,7-dimethyloctyl group, a n-nonyl group, a n-decyl group, an adamantyl group, a 2-ethyldecyl group, a 2-butyldecyl group, a 2-hexyldecyl group, a 2-octyldecyl group, a n-undecyl group, a n-dodecyl group, a 2-ethyldodecyl group, a 2-butyldodecyl group, a 2-hexyldocecyl group, a 2-octyldodecyl group, a n-tridecyl group, a n-tetradecyl group, a n-pentadecyl group, a n-hexadecyl group, a 2-ethylhexadecyl group, a 2-butylhexadecyl group, a 2-hexylhexadecyl group, a 2-octylhexadecyl group, a n-heptadecyl group, a n-octadecyl group, a n-nonadecyl group, a n-eicosyl group, a 2-ethyleicosyl group, a 2-butyleicosyl group, a 2-hexyleicosyl group, a 2-octyleicosyl group, a n-henicosyl group, a n-docosyl group, a n-tricosyl group, a n-tetracosyl group, a n-pentacosyl group, a n-hexacosyl group, a n-heptacosyl group, a n-octacosyl group, a n-nonacosyl group, a n-triacontyl group, etc. However, an embodiment is not limited thereto.

In this specification, an alkyl group may include a cycloalkyl group (a cyclic alkyl group). The carbon number of the cycloalkyl group is 3 to 60, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group etc. However, an embodiment is not limited thereto.

In this specification, an alkenyl group refers to a hydrocarbon group including one or more carbon double bonds in the middle or at the terminal of an alkyl group having 2 or more carbon atoms. The alkenyl group may be a linear chain or a branched chain. The carbon number thereof is not particularly limited, but the alkenyl group has carbon atoms of 2 to 60, 2 to 30, 2 to 20, or 2 to 10. Examples of the alkenyl group include a vinyl group, a 1-butenyl group, a 1-pentenyl group, a 1,3-butadienyl aryl group, a styrenyl group, a styrenylvinyl group, etc. However, an embodiment is not limited thereto.

In this specification, an alkynyl group refers to a hydrocarbon group containing one or more carbon triple bonds in the middle or at the terminal of an alkyl group having 2 or more carbon atoms. The alkynyl group may be a linear chain or a branched chain. The carbon number thereof is not particularly limited, but the alkynyl group has carbon atoms of 2 to 30, 2 to 20, or 2 to 10, but the number of carbon atoms is not particularly limited. Specific examples of the alkynyl group may include an ethynyl group, a propynyl group, etc. However, an embodiment is not limited thereto.

In this specification, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

In this specification, an aryl group refers to any functional group or substituent derived from an aromatic hydrocarbon ring. The aryl group may be a monocyclic aryl group or a polycyclic aryl group. The number of ring-forming carbon atoms of the aryl group may be 6 to 60, 6 to 30, 6 to 20, or 6 to 15. Examples of the aryl group may include a phenyl group, a naphthyl group, a fluorenyl group, an anthracenyl group, a phenanthrenyl group, a biphenylyl group, a terphenylyl group, a quaterphenylyl group, a quinquephenylyl group, a sexiphenylyl group, a triphenylenyl group, a pyrenyl group, a benzofluoranthenyl group, a chrysenyl group, etc. However, an embodiment is not limited thereto.

In this specification, a fluorenyl group may be substituted, and two substituents may also be bonded to each other to form a spiro structure. Examples in which a fluorenyl group is substituted are as follows. However, an embodiment is not limited thereto.

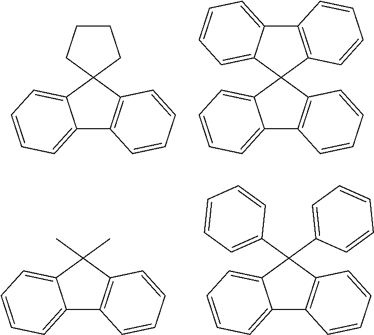

In this specification, a heterocyclic group means any optional functional group or substituent derived from a ring including one or more among B, O, N, P, S, Si, and Se as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be monocyclic or polycyclic.

In this specification, a heterocyclic group may include one or more among B, O, N, P, S, Si, and Se as heteroatoms. When the heterocyclic group includes two or more heteroatoms, two or more heteroatoms may be the same as or different from each other. The heterocyclic group may be a monocyclic heterocyclic group or a polycyclic heterocyclic group and conceptually includes a heteroaryl group. The number of ring-forming carbon atoms of the heterocyclic group may be 2 to 60, 2 to 30, 2 to 20, or 2 to 10.

In this specification, an aliphatic heterocyclic group may include one or more among B, O, N, P, S, Si, and Se, as heteroatoms. The number of ring-forming carbon atoms of the aliphatic heterocyclic group may be 2 to 30, 2 to 20, or 2 to 10. Examples of the aliphatic heterocyclic group include an oxirane group, a thiirane group, a pyrrolidine group, a piperidine group, a tetrahydrofuran group, a tetrahydrothiophene group, a thiane group, a tetrahydropyran group, an 1,4-dioxane group, etc. However, an embodiment is not limited thereto.

In this specification, a heteroaryl group may include one or more among B, O, N, P, S, Si, and Se as heteroatoms. When the heteroaryl group includes two or more heteroatoms, the two or more heteroatoms may be the same as, or different from each other. The heteroaryl group may be a monocyclic heterocyclic group, or a polycyclic heterocyclic group. The number of ring-forming carbon atoms of the heteroaryl group may be 2 to 60, 2 to 30, 2 to 20, or 2 to 10. Examples of the heteroaryl group may include a thienyl group, a furyl group, a pyrrolyl group, an imidazolyl group, a pyridyl group, a bipyridinyl group, a pyrimidinyl group, a triazinyl), a triazolyl group, an acridinyl group, a pyridazinyl group, a pyazinyl group, a quinolyl group, a quinazolinyl group, a quinoxalinyl group, a phenoxazinyl group, a phthalazinyl group, a pyridopyrimidinyl group, a pyridopyrazinyl group, a pyrazinopyrazinyl group, an isoquinolinyl group, an indolyl group, a carbazolyl group, a N-arylcarbazolyl group, a N-heteroarylcarbazolyl group, a N-alkylcarbazolyl group, a benzoxazolyl group, a benzoimidazolyl group, a benzothiazolyl group, a benzocarbazolyl group, a benzothiophenyl group, a dibenzothiophenyl group, a thienothiophenyl group, a benzofuranyl group, a phenanthrolinyl group, a thiazolyl group, an isoxazolyl group, an oxazolyl group, an oxadiazolyl group, a thiadiazolyl group, a phenothiazinyl group, a dibenzosilolyl group, or a dibenzofuranyl group, etc. However, an embodiment is not limited thereto.

In this specification, the description of the aryl group may be similarly applied to an arylene group except that the arylene group is a divalent group. The description of the heteroaryl group may be similarly applied to a heteroarylene group except that the heteroarylene group is a divalent group.

In this specification, a silyl group includes an alkyl silyl group and an aryl silyl group. Examples of the silyl group include a trimethylsilyl group, a trimethylsilyl group, a t-butyldimethylsilyl group, a vinyldimethylsilyl group, a propyldimethylsilyl group, a triphenylsilyl group, a diphenylsilyl group, a phenylsilyl group, etc. However, an embodiment is not limited thereto.

In this specification, the carbon number of a carbonyl group is not particularly limited, but the carbonyl group may have carbon atoms of 1 to 40, 1 to 30, or 1 to 20. Non-limiting examples of the carbonyl group include one or a combination of the following structures:

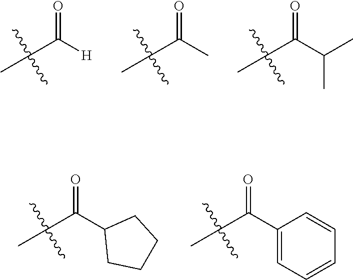

In this specification, the carbon numbers of a sulfinyl group and a sulfonyl group are not particularly limited but may be 1 to 30. The sulfinyl group may include an alkyl sulfinyl group, and an aryl sulfinyl group. The sulfonyl group may include an alkyl sulfonyl group, and an aryl sulfonyl group.

In this specification, a thio group may include an alkyl thio group and an aryl thio group. The thio group may refer to a group in which a sulfur atom is bonded to the previously defined alkyl group or aryl group. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc. However, an embodiment is not limited thereto.

In this specification, an oxy group may refer to a group in which an oxygen atom is bonded to the previously defined alkyl group or aryl group. The oxy group may include an alkoxy group and an aryl oxy group. The alkoxy group may be a linear chain, a branched chain, or a cyclic chain. The carbon number of the alkoxy group is not particularly limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, an embodiment is not limited thereto.

In this specification, a boron group may refer to a group in which a boron atom is bonded to the previously defined alkyl group or aryl group. The boron group includes an alkyl boron group and an aryl boron group. Examples of the boron group include a dimethylboron group, a diethylboron group, a t-butylmethylboron group, a diphenylboron group, a phenylboron group, etc. However, an embodiment is not limited thereto.

In this specification, the carbon number of an amine group is not particularly limited but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc. However, an embodiment is not limited thereto.

In the present specification, the alkyl group in the alkylthio group, the alkylsulfoxy group, the alkyl aryl group, the alkylamino group, the alkyl boron group, the alkyl silyl group, the alkylamine group is the same as the examples of the previously described alkyl group.

In the present specification, the aryl group in the aryloxy group, the arylthio group, the arylsulfoxy group, the aryl amino group, the aryl boron group, the aryl silyl group, the arylamine group is the same as the examples of the previously described aryl group.

In this specification, a direct linkage may indicate a single bond.

In this specification, the wording of being deuterated means that isotopic abundance of deuterium is greater than natural abundance thereof.

In this specification,

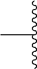

and “-*” indicate positions for linking.

Hereinafter, embodiments of this disclosure will be described with reference to drawings.

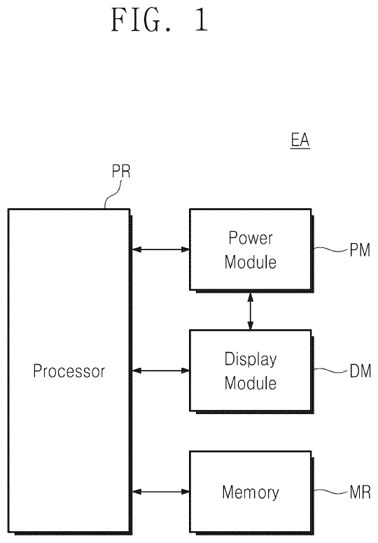

FIG. 1 is a block diagram of an electronic apparatus according to an embodiment. Referring to FIG. 1, the electronic apparatus EA according to an embodiment may include a display module DM, a processor PR, a memory MR, and a power module PM.

The processor PR may include at least one among a central processing unit (CPU), an application processor (AP), a graphic processing unit (GPU), a communication processor (CP), an image signal processor (ISP), and a controller.

The memory MR may store data information needed for the operation of the processor PR or the display module DM. When the processor PR executes an application stored in the memory MR, an image data signal and/or an input control signal are/is transmitted to the display module DM, and the display module DM may process the received signal and output image information through a display screen. The display module DM may include a display panel that displays an image.

The power module PM may include a power supply module such as a power adaptor or a battery device, and a power conversion module that converts power supplied by the power supply module to generate power needed for the operation of the electronic apparatus EA.

At least one among the previously described components of the electronic apparatus EA may be included in a display module according to embodiments, which will be described later, and a display device according to an embodiment, which includes the same. In addition, some of individual modules functionally included in one module may be included in the display device, and some others may be provided separately from the display device. For example, the display device may include the display module DM, and the processor PR, the memory MR and the power module PM may be provided in the form of other devices within the electronic apparatus EA, not the display device.

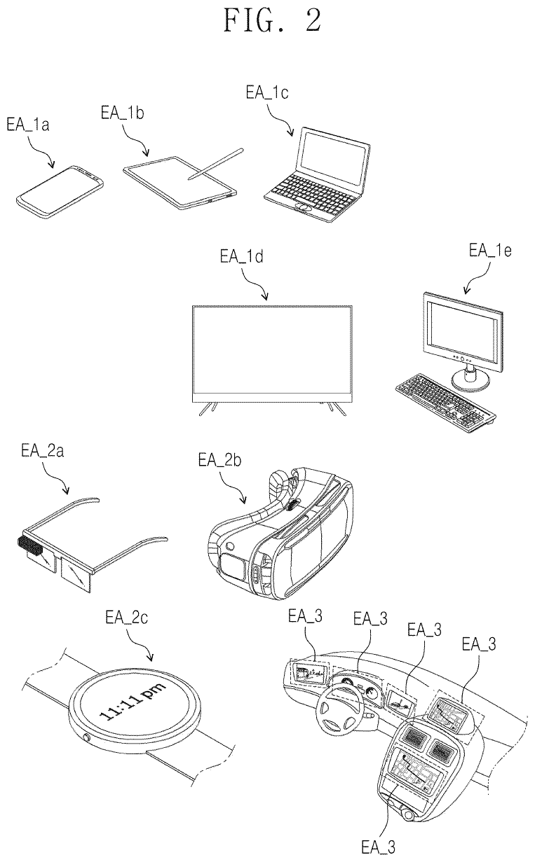

FIG. 2 shows schematic views of electronic apparatuses according to various embodiments.

Referring to FIG. 2, various electronic apparatuses including the display module according to an embodiment may include not only an image-displaying electronic apparatus such as a smart phone EA_1a, a tablet PC EA_1b, a laptop computer EA_1c, a TV EA_1d, and a desktop monitor EA_1e, but also a wearable electronic apparatus such as smart glasses EA_2a, a head mounted display EA_2b, or a smart watch EA_2c, and a vehicular electronic apparatus EA_3 such as a car dashboard, a center fascia, a center information display (CID) disposed in a dashboard, or a room mirror display, etc.

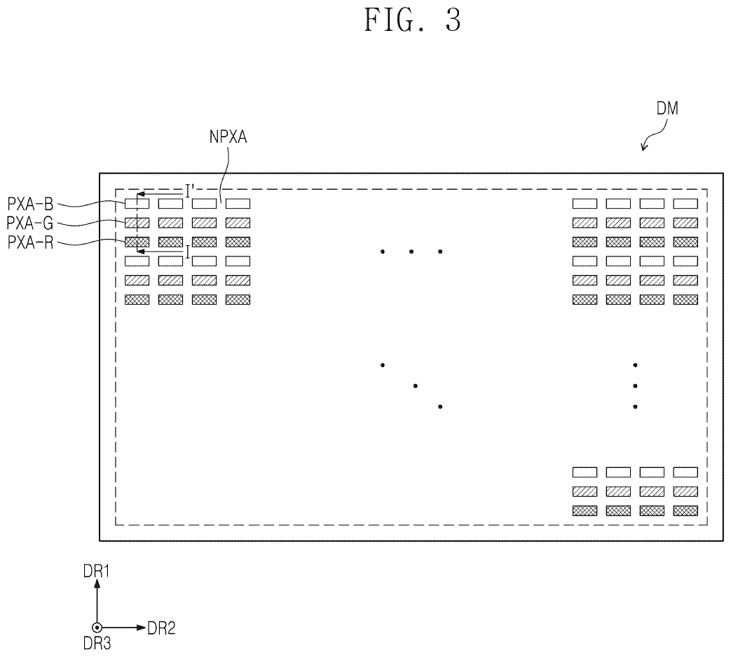

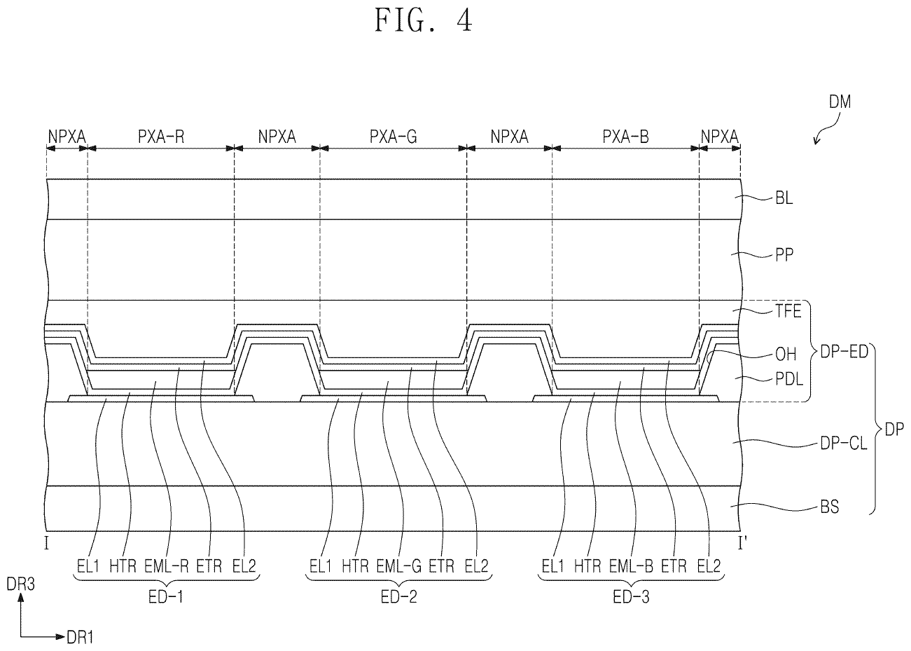

FIG. 3 is a plan view illustrating a display module DM according to an embodiment. FIG. 4 is a cross-sectional view of a display module DM according to an embodiment. FIG. 4 is a cross-sectional view illustrating a portion taken along line I-I′ in FIG. 3.

The display module DM may include a plurality of light-emitting elements ED-1, ED-2, and ED-3. In an embodiment, the display module DM may further include a display panel DP including the plurality of light-emitting elements ED-1, ED-2, and ED-3, and an optical layer PP disposed on the display panel DP.

The display panel DP may include a base layer BS, a circuit layer DP-CL provided on the base layer, and a display element layer DP-ED. The display element layer DP-ED may include a pixel-defining film PDL, light-emitting elements ED-1, ED-2, and ED-3 disposed between the pixel-defining films PDL, and an encapsulation layer TFE disposed on the light-emitting elements ED-1, ED-2, and ED-3.

The base layer BS may be a member that provides a base surface on which the circuit layer DP-CL is disposed. The base layer BS may be a glass substrate, a metal substrate, a polymer substrate, etc. However, an embodiment is not limited thereto, and the base layer BS may be an inorganic layer, an organic layer, or a composite material layer.

In an embodiment, the circuit layer DP-CL may be disposed on the base layer BS, and the circuit layer DP-CL may include a plurality of transistors (not illustrated). The transistors (not illustrated) may each include a control electrode, an input electrode, and an output electrode. For example, the circuit layer DP-CL may include a switching transistor and a driving transistor for driving the light-emitting elements ED-1, ED-2, and ED-3 in the display element layer DP-EL.

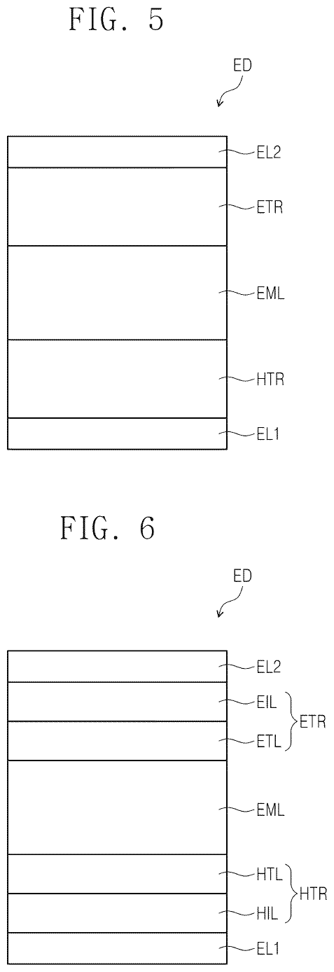

The light-emitting elements ED-1, ED-2, and ED-3 may each have a structure of a light-emitting element ED according to an embodiment, in FIG. 5 to FIG. 10, to be described later. The light-emitting elements ED-1, ED-2, and ED-3 may each include a first electrode EL1, a hole transport region HTR, an emission layer EML-R, EML-G or EML-B, an electron transport region ETR, and a second electrode EL2.

The optical layer PP may be disposed on the display panel DP and control reflected light of external light at the display panel DP. The optical layer PP may include, for example, a polarization layer or a color filter layer. Unlike what is illustrated in the drawings, the optical layer PP may be omitted in the display module DM according to an embodiment.

A base substrate BL may be disposed on the optical layer PP. The base substrate BL may be a member that provides a base surface on which the optical layer PP is disposed. The base substrate BL may be a glass substrate, a metal substrate, a plastic substrate, etc. However, an embodiment is not limited thereto, and the base substrate BL may be an inorganic layer, an organic layer, or a composite material layer. In addition, unlike what is illustrated, in an embodiment, the base substrate BL may be omitted.

The display module DM according to an embodiment may further include a filling layer (not illustrated). The filling layer (not illustrated) may be disposed between the display element layer DP-ED, and the base substrate BL. The filling layer (not illustrated) may be an organic layer. The filling layer (not illustrated) may include at least one among an acrylate-based resin, a silicon-based resin, and an epoxy-based resin.

FIG. 4 illustrates that emission layers EML-R, EML-G, and EML-B of light-emitting elements ED-1, ED-2, and ED-3 are disposed in openings OH defined in pixel-defining films PDL, and a hole transport region HTR, an electron transport region ETR, and a second electrode EL2 are provided as common layers in all of the light-emitting elements ED-1, ED-2, and ED-3. However, an embodiment is not limited thereto, and unlike what is illustrated in FIG. 4, in an embodiment, the hole transport region HTR, and the electron transport region ETR may be provided by being patterned inside the openings OH defined in the pixel-defining films PDL. For example, in an embodiment, the hole transport region HTR, the emission layers EML-R, EML-G, and EML-B, the electron transport region ETR, and the like may be provided by being patterned by an inkjet printing method.

The encapsulation layer TFE may cover the light-emitting elements ED-1, ED-2, and ED-3. The encapsulation layer TFE may seal the display element layer DP-ED. The encapsulation layer TFE may be a thin-film encapsulation layer. The encapsulation layer TFE may be one layer or a stack of multiple layers. The encapsulation layer TFE may include at least one insulation layer. The encapsulation layer TFE according to an embodiment may include at least one inorganic film (hereinafter, inorganic encapsulation film). In addition, the encapsulation layer TFE according to an embodiment may include at least one organic film (hereinafter, organic encapsulation film) and at least one inorganic encapsulation film.

The inorganic encapsulation film may protect the display element layer DP-ED from moisture/oxygen, and the organic encapsulation film may protect the display element layer DP-ED from foreign substances such as dust particles. The inorganic encapsulation film may include silicon nitride, silicon oxynitride, silicon oxide, titanium oxide, aluminum oxide, or the like, but is not particularly limited thereto. The organic encapsulation film may include an acrylate-based compound, an epoxy-based compound, etc. The organic encapsulation film may include a photopolymerizable organic material but is not particularly limited thereto.

The encapsulating layer TFE may be disposed on the second electrode EL2 and may be disposed while filling the opening OH.

Referring to FIG. 3 and FIG. 4, the display module DM may include a non-emission region NPXA and emission regions PXA-R, PXA-G and PXA-B. The emission regions PXA-R, PXA-G and PXA-B may be regions that emit light generated from the light-emitting elements ED-1, ED-2 and ED-3, respectively. The display module DM may include a first emission region PXA-R, a second emission region PXA-G and a third emission region PXA-B, which are separated from each other on a plane.

The first to third emission regions PXA-R, PXA-G and PXA-B may be respectively separated by the pixel-defining films PDL. The non-emission regions NPXA are regions between neighboring emission regions PXA-R, PXA-G and PXA-B and may respectively correspond to the pixel-defining films PDL. In this specification, the emission regions PXA-R, PXA-G and PXA-B may respectively correspond to pixels. The pixel-defining films PDL may separate the light-emitting elements ED-1, ED-2, and ED-3. Emission layers EML-R, EML-G and EML-B of the light-emitting elements ED-1, ED-2 and ED-3 may be disposed and separated in the openings OH defined in the pixel-defining films PDL.

The emission regions PXA-R, PXA-G, and PXA-B may be divided into a plurality of groups according to the color of light generated from the light-emitting elements ED-1, ED-2, and ED-3. FIG. 3 and FIG. 4 exemplarily illustrate that the display module DM according to an embodiment includes three emission regions PXA-R, PXA-G, and PXA-B respectively emitting red color light, green color light, and blue color light. For example, the first emission region PXA-R may be referred to as a red emission region, the second emission region PXA-G may be referred to as a green emission region, and the third emission region PXA-B may be referred to as a blue emission region.

In the display module DM according to an embodiment, the plurality of light-emitting elements ED-1, ED-2, and ED-3 may emit light of different wavelength regions. For example, in an embodiment, the display device DM may include a first light-emitting element ED-1 which emits red light, a second light-emitting element ED-2 which emits green light, and a third light-emitting element ED-3 which emits blue light. That is, the red emission region PXA-R, the green emission region PXA-G, and the blue emission region PXA-B of the display module DM may respectively correspond to the first light-emitting element ED-1, the second light-emitting element ED-2, and the third light-emitting element ED-3.

However, an embodiment is not limited thereto, and the first to third light-emitting elements ED-1, ED-2, and ED-3 may emit light of the same wavelength region, or at least one may emit light having a wavelength region different from the others. For example, all of the first to third light-emitting elements ED-1, ED-2, and ED-3 may emit blue light.

The emission regions PXA-R, PXA-G, and PXA-B in the display module DM according to an embodiment may be arranged in a stripe form. Referring to FIG. 3, a plurality of first emission regions PXA-R, a plurality of second emission regions PXA-G, and a plurality of third emission regions PXA-B may each be arranged along a second direction axis DR2. In addition, the first emission region PXA-R, the second emission region PXA-G, and the third emission region PXA-B may be alternately arranged in this order along a first direction axis DR1.

FIG. 3 and FIG. 4 illustrate that the emission regions PXA-R, PXA-G, and PXA-B have similar areas, but an embodiment is not limited thereto. The areas of the emission regions PXA-R, PXA-G, and PXA-B may differ from each other according to the wavelength region of light emitted. The areas of the emission regions PXA-R, PXA-G, and PXA-B may refer to planar areas when viewed on a plane defined by the first direction axis DR1 and the second direction axis DR2.

The arrangement form of the emission regions PXA-R, PXA-G, and PXA-B is not limited to what is illustrated in FIG. 3, and the order in which the first emission region PXA-R, the second emission region PXA-G, and the third emission region PXA-B are arranged may be provided in various combinations according to display quality characteristics to be required for the display module DM and a display device including the same. For example, the arrangement form of the emission regions PXA-R, PXA-G, and PXA-B may be a PENTILE™ arrangement form or a Diamond Pixel™ arrangement form.

In addition, the areas of the emission regions PXA-R, PXA-G, and PXA-B may be different from each other. For example, in an embodiment, the area of the second emission region PXA-G which is the green emission region may be smaller than the area of the third emission region PXA-B which is the blue emission region, but an embodiment is not limited thereto.

Hereinafter, FIG. 5 to FIG. 10 are each a cross-sectional view schematically illustrating a light-emitting element according to an embodiment. The light-emitting element ED according to an embodiment may include a first electrode EL1, a hole transport region HTR, an emission layer EML, an electron transport region ETR, and a second electrode EL2, stacked in sequence.

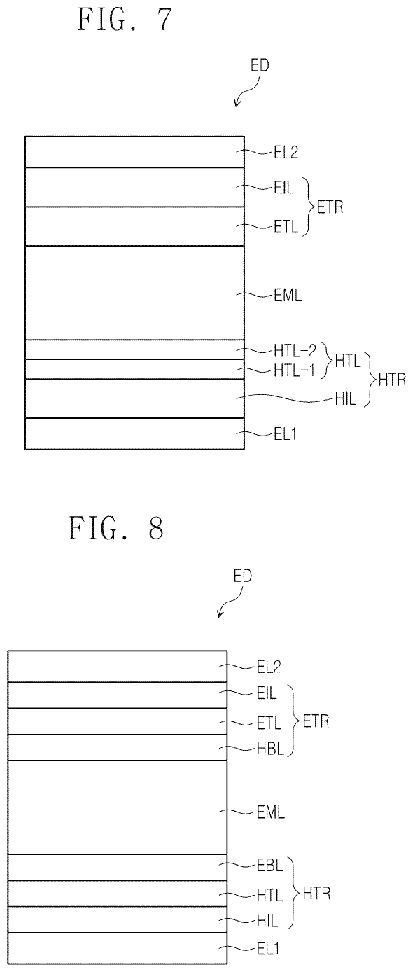

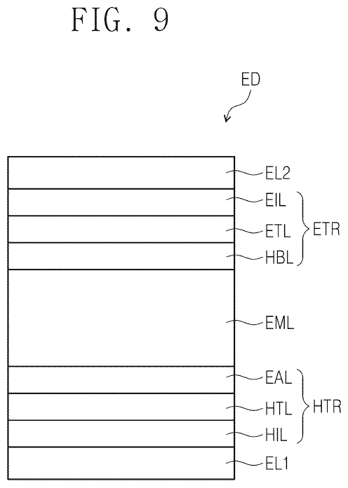

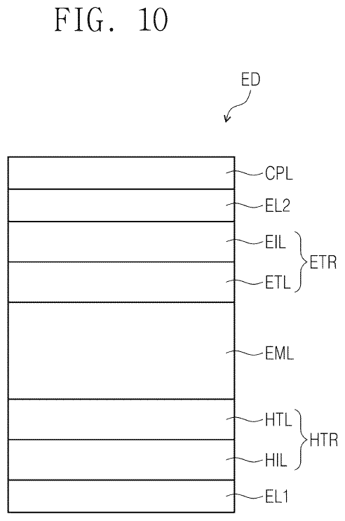

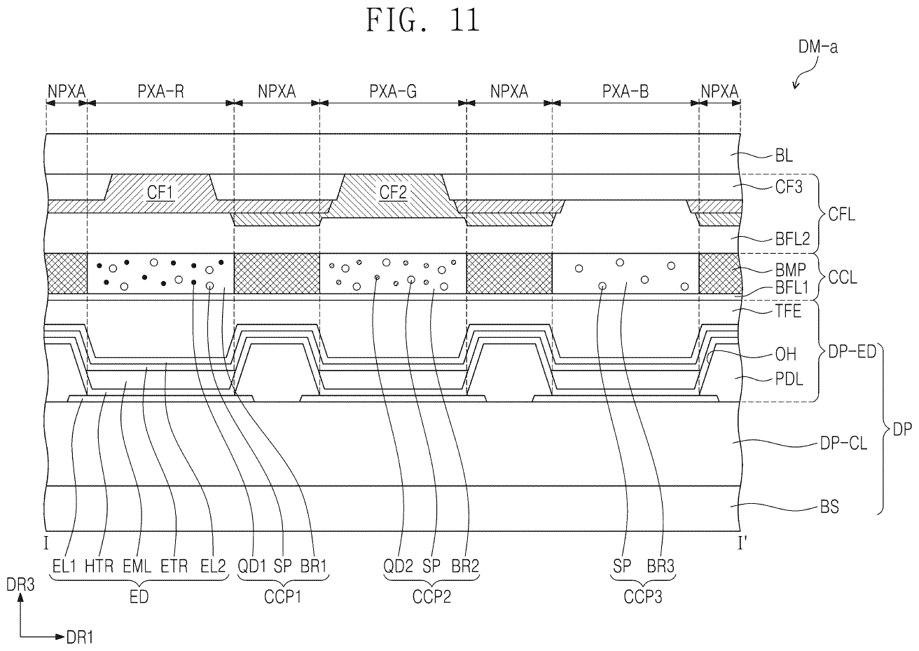

FIG. 6 illustrates, compared to FIG. 5, a cross-sectional view of a light-emitting element ED according to an embodiment in which a hole transport region HTR includes a hole injection layer HIL, and a hole transport layer HTL, and an electron transport region ETR includes an electron injection layer EIL and an electron transport layer ETL. FIG. 7 illustrates, compared to FIG. 5, a cross-sectional view of a light-emitting element ED in which a hole transport layer HTL includes a first hole transport layer HTL-1 and a second hole transport layer HTL-2. In addition, FIG. 8 illustrates, compared to FIG. 5, a cross-sectional view of a light-emitting element ED according to an embodiment in which a hole transport region HTR includes a hole injection layer HIL, a hole transport layer HTL, and an electron-blocking layer EBL, and an electron transport region ETR includes an electron injection layer EIL, an electron transport layer ETL, and a hole-blocking layer HBL. FIG. 9 illustrates, compared to FIG. 5, a cross-sectional view of a light-emitting element ED according to an embodiment in which a hole transport region HTR includes a hole injection layer HIL, a hole transport layer HTL, and an emission auxiliary layer EAL, and an electron transport region ETR includes an electron injection layer EIL, an electron transport layer ETL, and a hole-blocking layer HBL. FIG. 10 illustrates, compared to FIG. 6, a cross-sectional view of a light-emitting element ED including a capping layer CPL disposed on a second electrode EL2.

The first electrode EL1 has conductivity. The first electrode EL1 may include a metal material, a metal alloy or a conductive compound. The first electrode EL1 may be an anode or a cathode. However, an embodiment is not limited thereto. In addition, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn and Zn, a compound of two or more selected therefrom, a mixture of two or more selected therefrom, or an oxide thereof.

If the first electrode EL1 is the transmissive electrode, the first electrode EL1 may include a transparent metal oxide such as indium tin oxide (ITO), indium zinc oxide (IZO), zinc oxide (ZnO), and indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (for example, a mixture of Ag and Mg). Alternatively, the first electrode EL1 may have a multi-layer structure including a reflective layer or a transflective layer including the previously described materials, and a transparent conductive layer including indium tin oxide (ITO), indium zinc oxide (IZO), zinc oxide (ZnO), or indium tin zinc oxide (ITZO). For example, the first electrode EL1 may have a three-layer structure of ITO/Ag/ITO but is not limited thereto. In addition, an embodiment is not limited thereto. The first electrode EL1 may include the previously described metal materials, a combination of two or more metal materials selected from among the previously described metal materials, oxides of the previously described metal materials, etc. The thickness of the first electrode EL1 may be about 700 Å to about 10,000 Å. For example, the thickness of the first electrode EL1 may be about 1,000 Å to about 3,000 Å.

The hole transport region HTR is provided on the first electrode EL1. The hole transport region HTR may include at least one among a hole injection layer HIL, a hole transport layer HTL, an emission auxiliary layer EAL, and an electron-blocking layer EBL. The thickness of the hole transport region HTR may be, for example, about 50 Å to about 15,000 Å. The emission auxiliary layer EAL may also be referred to as a buffer layer.

The hole transport region HTR may have a single layer including a single material, a single layer including a plurality of different materials, or a multi-layer structure having a plurality of layers including a plurality of different materials.

For example, the hole transport region HTR may have a single-layer structure of a hole injection layer HIL or a hole transport layer HTL, and also have a single-layer structure including a hole injection material and a hole transport material. In addition, the hole transport region HTR may have a single-layer structure including a plurality of different materials, or have a structure of a hole injection layer HIL/hole transport layer HTL, a hole injection layer HIL/hole transport layer HTL/emission auxiliary layer EAL, a hole injection layer HIL/emission auxiliary layer EAL, a hole transport layer HTL/emission auxiliary layer EAL, a hole injection layer HIL/hole transport layer HTL/emission auxiliary layer EAL or a hole injection layer HIL/hole transport layer HTL/electron-blocking layer EBL, sequentially stacked from a first electrode EL1, but an embodiment is not limited thereto.

In addition, although not illustrated, the hole transport region HTR may have a structure of first hole transport layer/second hole transport layer/third hole transport layer, sequentially stacked from a first electrode EL1.

In addition, a hole transport layer HTL may have a single-layer structure, or a multi-layer structure. When the hole transport layer HTL has a multi-layer structure, at least one layer in the hole transport layer HTL may include an amine compound according to an embodiment. In an embodiment, when the hole transport layer HTL has a multi-layer structure, an uppermost layer in the hole transport layer HTL may include the amine compound according to an embodiment. In the hole transport layer HTL, the uppermost layer may be a layer adjacent to the emission layer EML. For example, the hole transport region HTR may have a two-layer structure of a hole injection layer HIL disposed on the first electrode EL1, and a first hole transport layer HTL-1 (FIG. 7) disposed on the hole injection layer HIL and a second hole transport layer HTL-2 (FIG. 7) disposed on the first hole transport layer HTL-1 (FIG. 7) adjacent to the bottom of the emission layer EML, and the second hole transport layer HTL-2 (FIG. 7) may include an amine compound according to an embodiment. However, an embodiment is not limited thereto.

The hole transport region HTR may be formed by using various methods such as a vacuum deposition method, a spin coating method, a cast method, a Langmuir-Blodgett (LB) method, an inkjet printing method, a laser printing method, and a laser induced thermal imaging (LITI) method.

The light emitting element ED according to an embodiment may include, in the hole transport region HTR, an amine compound according to an embodiment, according to Formula 1. At least one among the hole injection layer HIL, and the hole transport layer HTL, the electron-blocking layer EBL, and the emission auxiliary layer EAL may include the amine compound according to an embodiment. For example, the light-emitting element ED according to an embodiment may include the amine compound according to an embodiment in the hole transport layer HTL. In addition, when the hole transport region HTR includes a plurality of hole transport layers which are stacked in a thickness direction, at least one hole transport layer may include the amine compound according to an embodiment, according to by Formula 1.

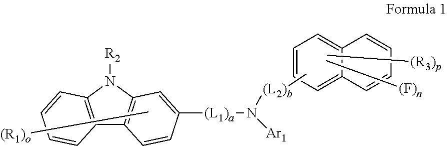

In Formula 1, L₁ and L₂ are each independently a substituted or unsubstituted arylene group having 6 to 30 carbon atoms. In Formula 1, a and b are each independently an integer of 0 to 4. When a is 0, N of NAr₁ is directly bonded to the 2-position of a carbazole group, and when b is 0, N of NAr₁ is directly bonded to a naphthyl group.

In Formula 1, Ar₁ is a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms. For example, Ar₁ is a substituted or unsubstituted phenyl group, a substituted or unsubstituted dibenzofuran group, a substituted or unsubstituted dibenzothiophene group, a substituted or unsubstituted naphthyl group, or a substituted or unsubstituted naphthobenzofuran group.

In Formula 1, n is an integer of 1 to 7. Therefore, the naphthyl group is substituted with at least one fluorine atom. For example, n may be 1, 2, 4, or 6.

In Formula 1, each R₁, R₂, and each are independently a hydrogen atom, a deuterium atom, a substituted or unsubstituted aryl group having 6 to 40 ring-forming carbon atoms, or a substituted or unsubstituted heteroaryl group having 5 to 40 ring-forming carbon atoms, or a substituted or unsubstituted alkyl group having 1 to 20 carbon atoms.

When each R₁, R₂, and each are independently a substituted or unsubstituted aryl group having 6 to 40 ring-forming carbon atoms, the aryl group may be a substituted or unsubstituted phenyl group, a substituted or unsubstituted naphthyl group, a substituted or unsubstituted fluorenyl group, a substituted or unsubstituted anthracenyl group, a substituted or unsubstituted phenanthrenyl group, a substituted or unsubstituted biphenylyl group, a substituted or unsubstituted terphenylyl group, a substituted or unsubstituted quaterphenylyl group, a substituted or unsubstituted quinquephenylyl group, a substituted or unsubstituted sexiphenylyl group, a substituted or unsubstituted triphenylenyl group, a substituted or unsubstituted pyrenyl group, a substituted or unsubstituted benzofluoranthenyl group, or a substituted or unsubstituted chrysenyl group, etc. For example, R₂ may be a substituted or unsubstituted phenyl group. For example, R₂ may be an unsubstituted phenyl group. For example, R₃ may be an unsubstituted phenyl group.

When each R₁, R₂, and each are independently a substituted or unsubstituted heteroaryl group having 2 to 40 ring-forming carbon atoms, the heteroaryl group may include one or more among B, O, N, P, S, Si, and Se as ring-forming heteroatoms. The heteroaryl group may be a thienyl group, a furyl group, a pyrrolyl group, an imidazolyl group, a pyridyl group, a bipyridinyl group, a pyrimidinyl group, a triazinyl group, a triazolyl group, an acridinyl group, a pyridazinyl group, a pyrazinyl group, a quinolyl group, a quinazolinyl group, a quinoxalinyl group, a phenoxazinyl group, a phthalazinyl group, a pyridopyrimidinyl group, a pyridopyrazinyl group, a pyrazinopyrazinyl group, an isoquinolinyl group, an indolyl group, a carbazolyl group, a N-arylcarbazolyl group, a N-heteroarylcarbazolyl group, a N-alkylcarbazolyl group, a benzoxazolyl group, a benzoimidazolyl group, a benzothiazolyl group, a benzocarbazolyl group, a benzothiophenyl group, a dibenzothiophenyl group, a thienothiophenyl group, a benzofuranyl group, a phenanthrolinyl group, a thiazolyl group, an isoxazolyl group, an oxazolyl group, a oxadiazolyl group, a thiadiazolyl group, a phenothiazinyl group, a dibenzosilolyl group, or a dibenzofuranyl group, etc.

In Formula 1, o is an integer of 0 to 7. When o is 0, the R₁ position remains unsubstituted and is occupied by the inherent hydrogen. When o is 2 or greater, a plurality of substituents of R₁ may be the same as or different from each other.

In Formula 1, p is an integer of 0 to 7-n. If p is 0, the R₃ position remains unsubstituted and is occupied by the inherent hydrogen. For example, when n is 1, p may be an integer of 0 to 6. For example, when n is 2, p may be an integer of 0 to 5. For example, when n is 4, p may be an integer of 0 to 3. If p is 2 or greater, a plurality of substituents of R₃ may be the same as or different from each other.

The amine compound according to an embodiment is an amine compound including a 2-carbazole group, and a naphthyl group substituted with at least one fluorine atom. Specifically, the amine compound according to an embodiment has a structure in which a central nitrogen atom is bonded to the 2-position of a carbazole group

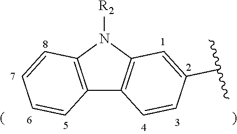

via a linker L₁ or a direct linkage Specifically, the amine compound according to an embodiment has a structure in which a central nitrogen atom is bonded via a linker L₂ or a direct linkage to a naphthyl group substituted with at least one fluorine atom. The amine compound according to an embodiment may be a monoamine compound which includes no additional amino substituent.

The amine compound according to an embodiment includes a 2-carbazole group and a naphthyl group substituted with at least one fluorine group and thus may exhibit high stability.

A reactivity may be lower at the 2-position than at the 3-position in the carbazole group. Therefore, characteristics, in which molecular stability of the amine compound increases, may be exhibited by appropriately selecting an electron-donating group and an electron-withdrawing group to be substituted for the 2-carbazole group.

A naphthyl group has a planar structure, and thus it may be easy to overlappingly stack molecules of the amine compound according to an embodiment including a naphthyl group. Therefore, in the amine compound according to an embodiment, charges may be easily transferred by hopping through which charges are transferred while jumping from one molecule to another molecule, and thus the light-emitting element may have a reduced driving voltage when the amine compound according to an embodiment is applied to the hole transport region.

A fluorine atom has the highest electronegativity among all atoms, and thus a naphthyl group, which is an aromatic compound, may be serve as an electron-withdrawing group when substituted with a fluorine atom. Therefore, when the naphthyl group is substituted with a fluorine atom, an overall electron density of an aromatic ring decreases, and thus electrophilic substitution reaction may be suppressed. Therefore, when the amine compound according to an embodiment is used in the hole transport region of the light-emitting element, deterioration of the compound due to electrons may be suppressed, and thus the lifespan of the light-emitting element may further be improved.

In the amine compound according to an embodiment, Formula 1 may have a structure according to any one of Formula 2-1 to Formula 2-3.

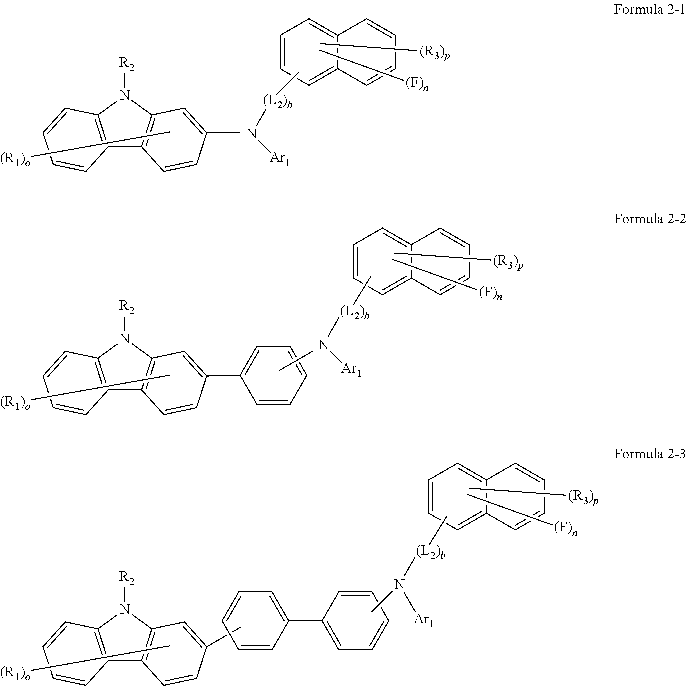

The previously presented description of the amine compound according to Formula 1 may be similarly applied to those of L₂, Ar₁, R₁ to R₃, b, n, o, and p in Formula 2-1 to Formula 2-3.

In the amine compound according to an embodiment, Formula 1 may have a structure according to any one of Formula 3-1 to Formula 3-3.

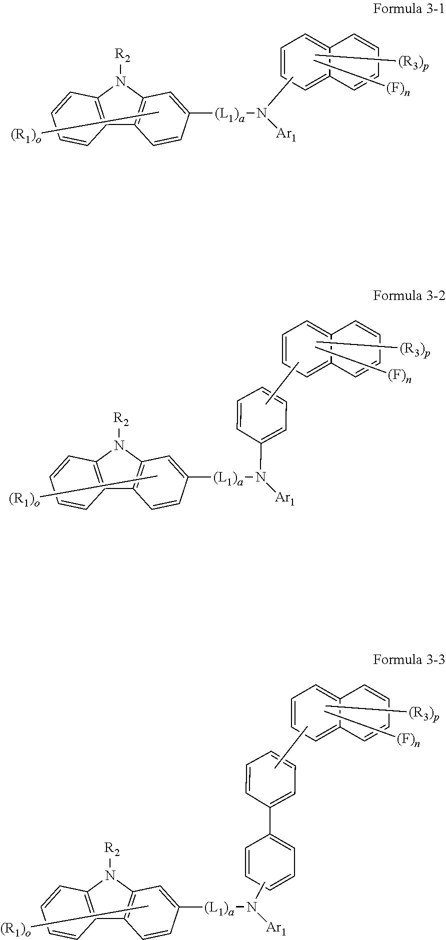

The previously presented description of the amine compound according to Formula 1 may be similarly applied to those of L₁, Ar₁, R₁ to R₃, a, n, o, and p in Formula 3-1 to Formula 3-3

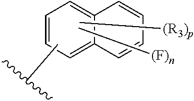

in Formula 1 of the amine compound according to an embodiment may have a structure according to any one functional group in Functional Group Set A.

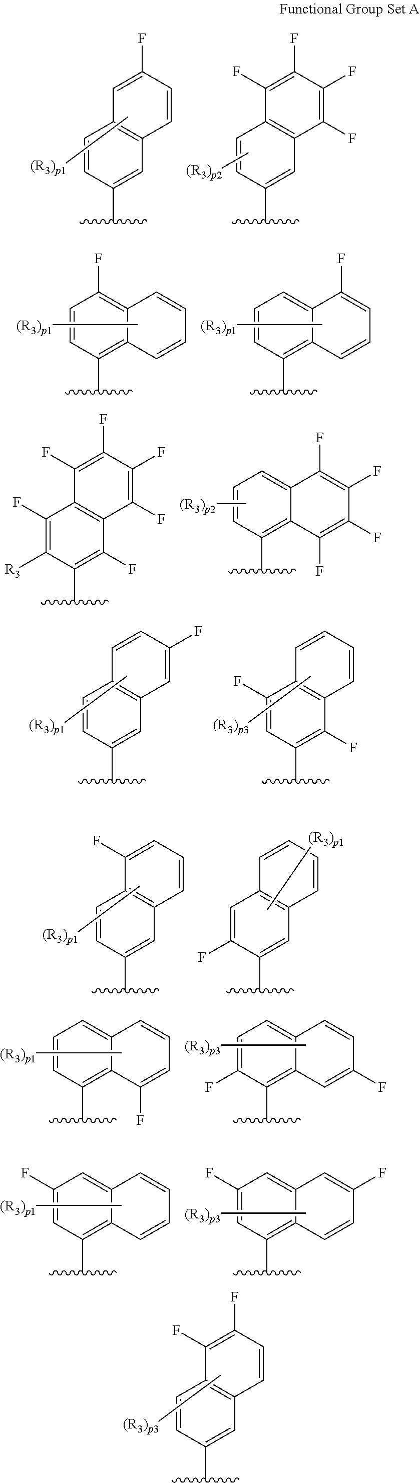

In Functional Group Set A, p1 may be an integer of 0 to 6, p2 may be an integer of 0 to 3, p3 may be an integer of 0 to 5, and the previously presented description of the amine compound according to Formula 1 may be similarly applied to R₃.

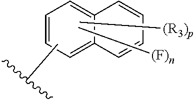

in Formula 1 of the amine compound according to an embodiment may be according to any one among functional groups in Functional Group Set B.

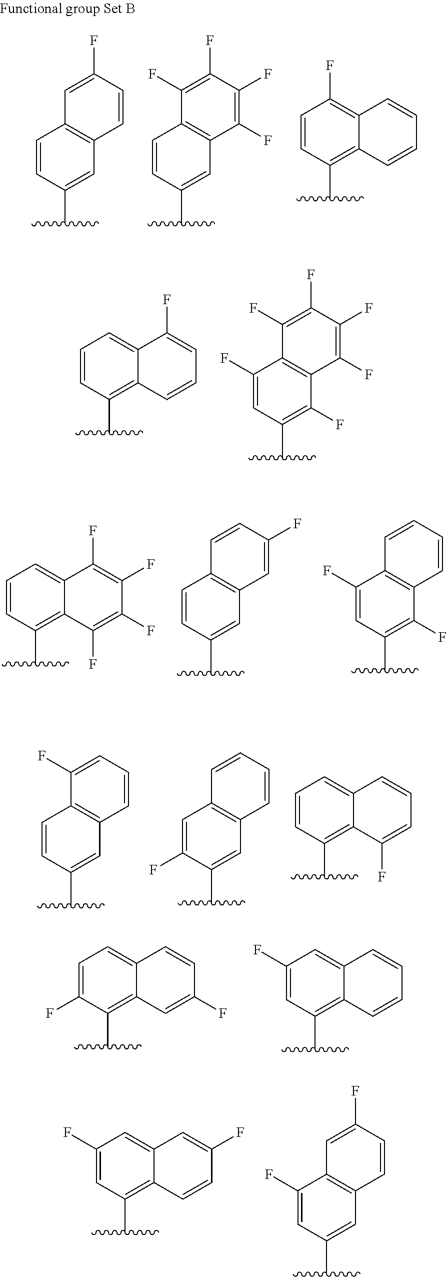

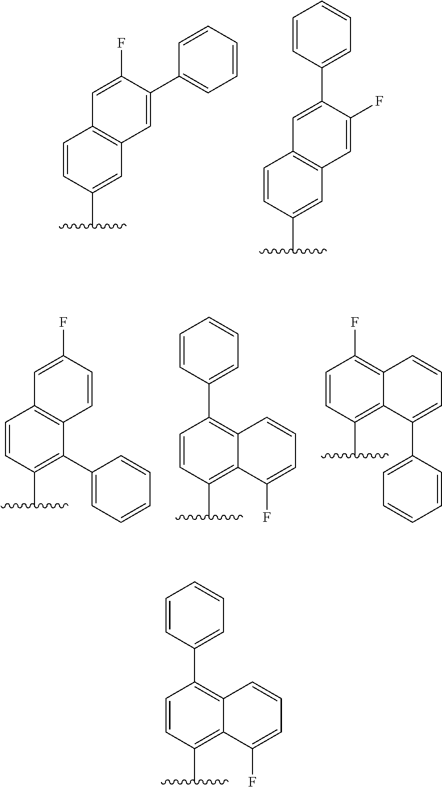

The amine compound according to an embodiment may have a structure according to any one compound in Compound Group 1. The hole transport region HTR of the light-emitting element ED according to an embodiment may include at least one of amine compounds disclosed in Compound Group 1. In Compound Group 1, D is a deuterium atom.

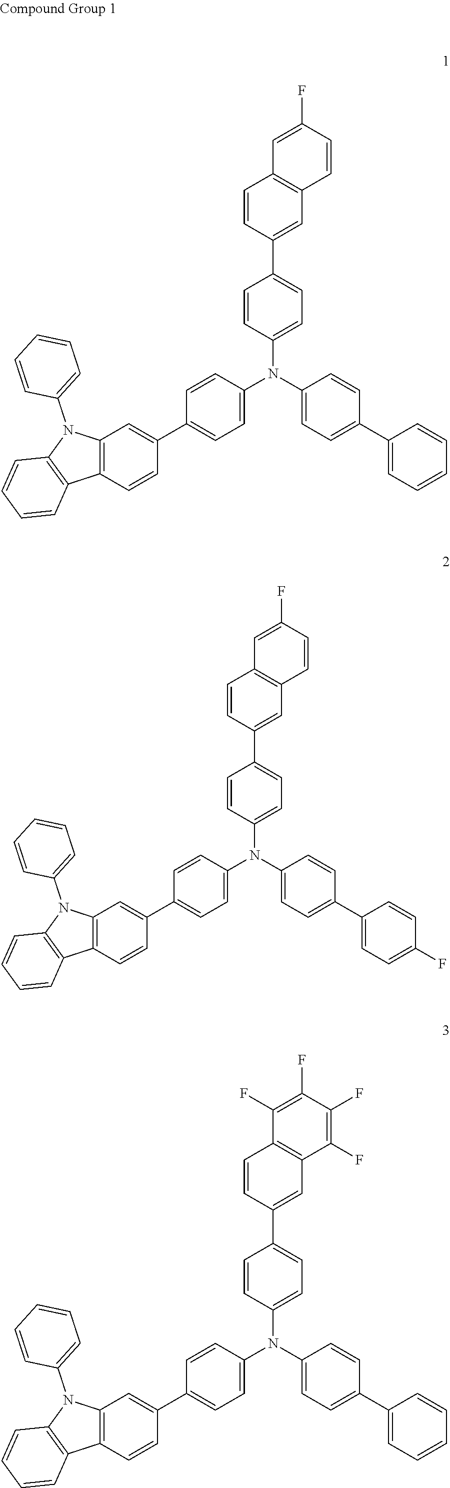 [SMILES: Fc1ccc2cc(-c3ccc(N(c4ccc(-c5ccccc5)cc4)c4ccc(-c5ccc6c7ccccc7n(-c7ccccc7)c6c5)cc4)cc3)ccc2c1 ; Fc1ccc(-c2ccc(N(c3ccc(-c4ccc5cc(F)ccc5c4)cc3)c3ccc(-c4ccc5c6ccccc6n(-c6ccccc6)c5c4)cc3)cc2)cc1 ; Fc1c(F)c(F)c2cc(-c3ccc(N(c4ccc(-c5ccccc5)cc4)c4ccc(-c5ccc6c7ccccc7n(-c7ccccc7)c6c5)cc4)cc3)ccc2c1F]

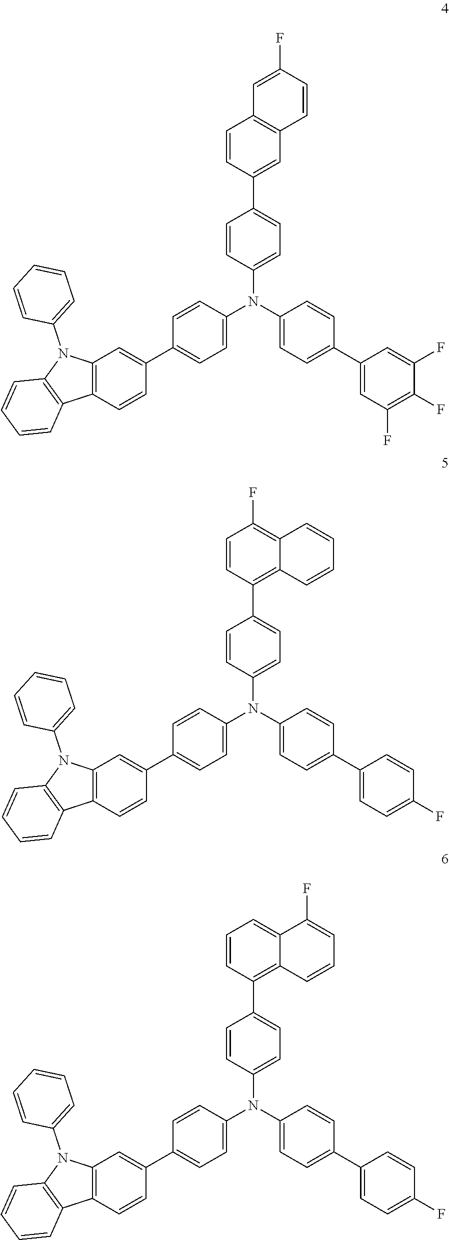 [SMILES: Fc1ccc2cc(-c3ccc(N(c4ccc(-c5cc(F)c(F)c(F)c5)cc4)c4ccc(-c5ccc6c7ccccc7n(-c7ccccc7)c6c5)cc4)cc3)ccc2c1 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)cc1 ; Fc1ccc(-c2ccc(N(c3ccc(-c4ccc5c6ccccc6n(-c6ccccc6)c5c4)cc3)c3ccc(-c4cccc5c(F)cccc45)cc3)cc2)cc1]

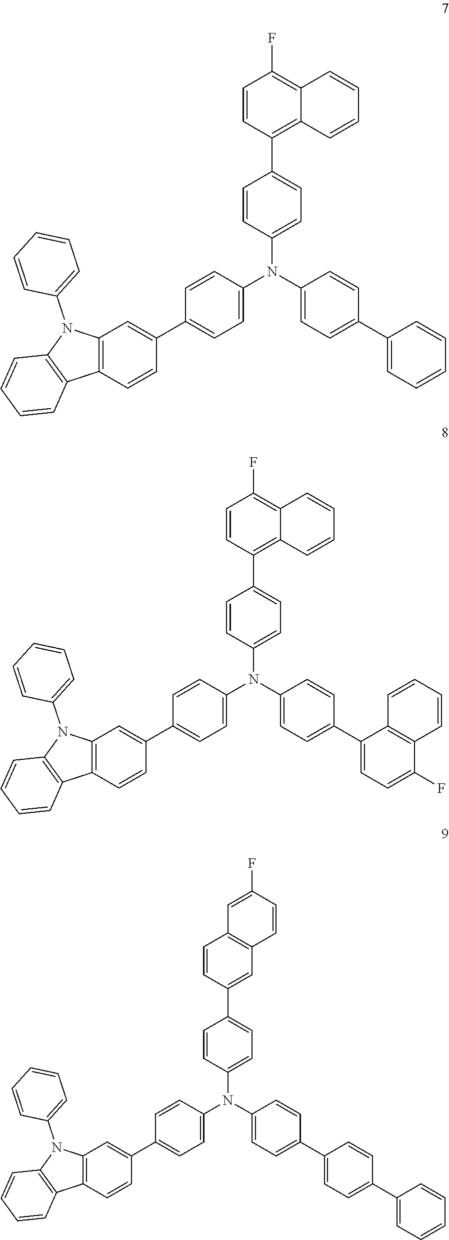 [SMILES: Fc1ccc(-c2ccc(N(c3ccc(-c4ccccc4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)c2ccccc12 ; Fc1ccc2cc(-c3ccc(N(c4ccc(-c5ccc(-c6ccccc6)cc5)cc4)c4ccc(-c5ccc6c7ccccc7n(-c7ccccc7)c6c5)cc4)cc3)ccc2c1]

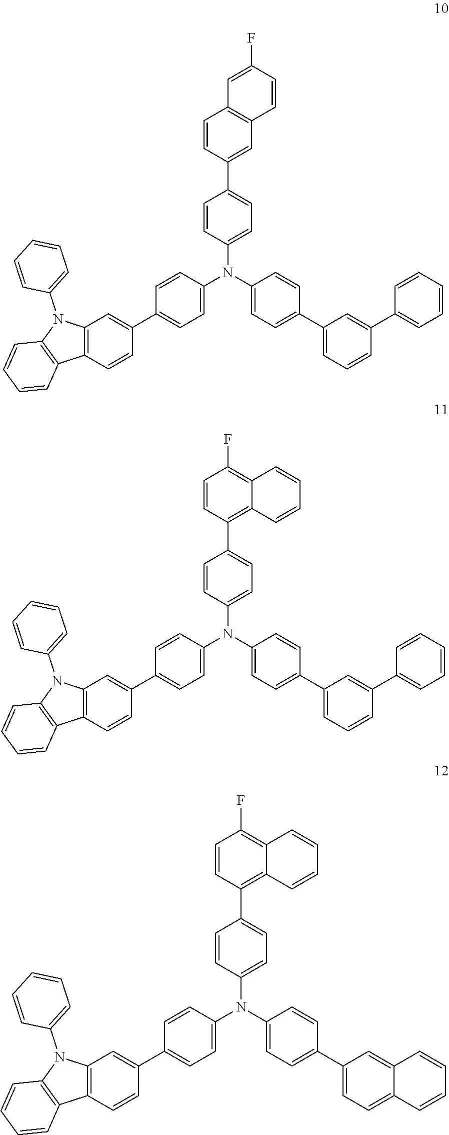 [SMILES: Fc1ccc2cc(-c3ccc(N(c4ccc(-c5cccc(-c6ccccc6)c5)cc4)c4ccc(-c5ccc6c7ccccc7n(-c7ccccc7)c6c5)cc4)cc3)ccc2c1 ; Fc1ccc(-c2ccc(N(c3ccc(-c4cccc(-c5ccccc5)c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5ccccc5c4)cc3)c3ccc(-c4ccc5c6ccccc6n(-c6ccccc6)c5c4)cc3)cc2)c2ccccc12]

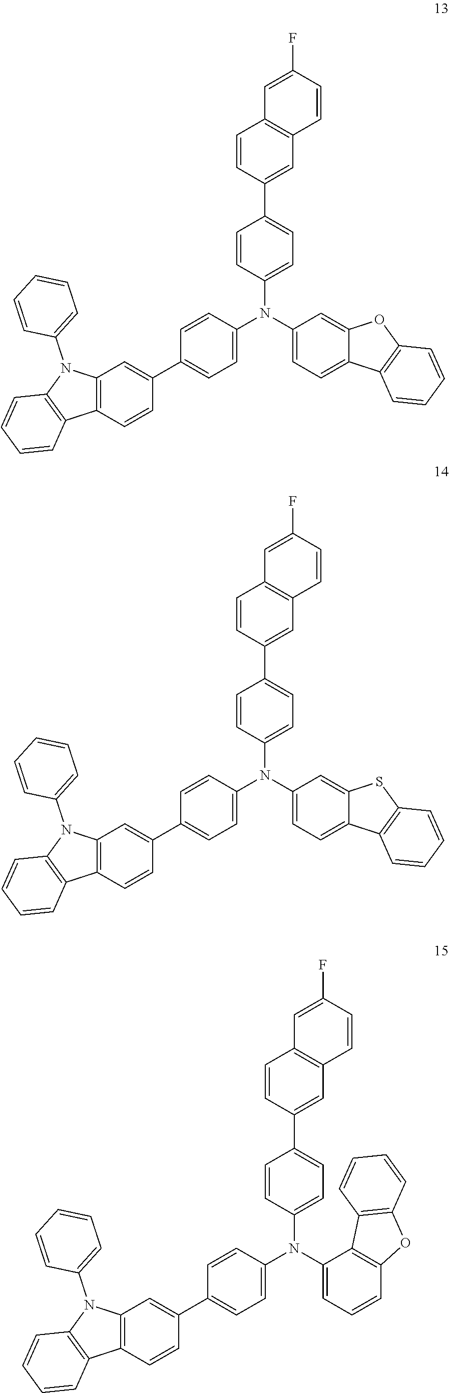 [SMILES: Fc1ccc2cc(-c3ccc(N(c4ccc(-c5ccc6c7ccccc7n(-c7ccccc7)c6c5)cc4)c4ccc5c(c4)oc4ccccc45)cc3)ccc2c1 ; Fc1ccc2cc(-c3ccc(N(c4ccc(-c5ccc6c7ccccc7n(-c7ccccc7)c6c5)cc4)c4ccc5c(c4)sc4ccccc45)cc3)ccc2c1 ; Fc1ccc2cc(-c3ccc(N(c4ccc(-c5ccc6c7ccccc7n(-c7ccccc7)c6c5)cc4)c4cccc5oc6ccccc6c45)cc3)ccc2c1]

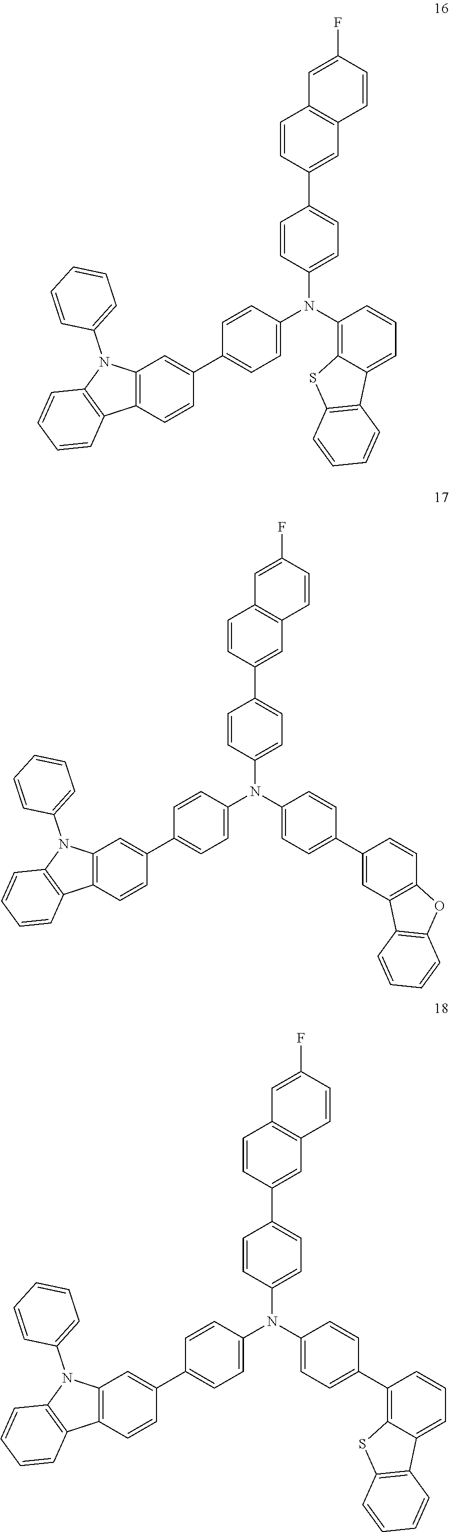 [SMILES: Fc1ccc2cc(-c3ccc(N(c4ccc(-c5ccc6c7ccccc7n(-c7ccccc7)c6c5)cc4)c4cccc5c4sc4ccccc45)cc3)ccc2c1 ; Fc1ccc2cc(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5sc5ccccc56)cc4)cc3)ccc2c1]

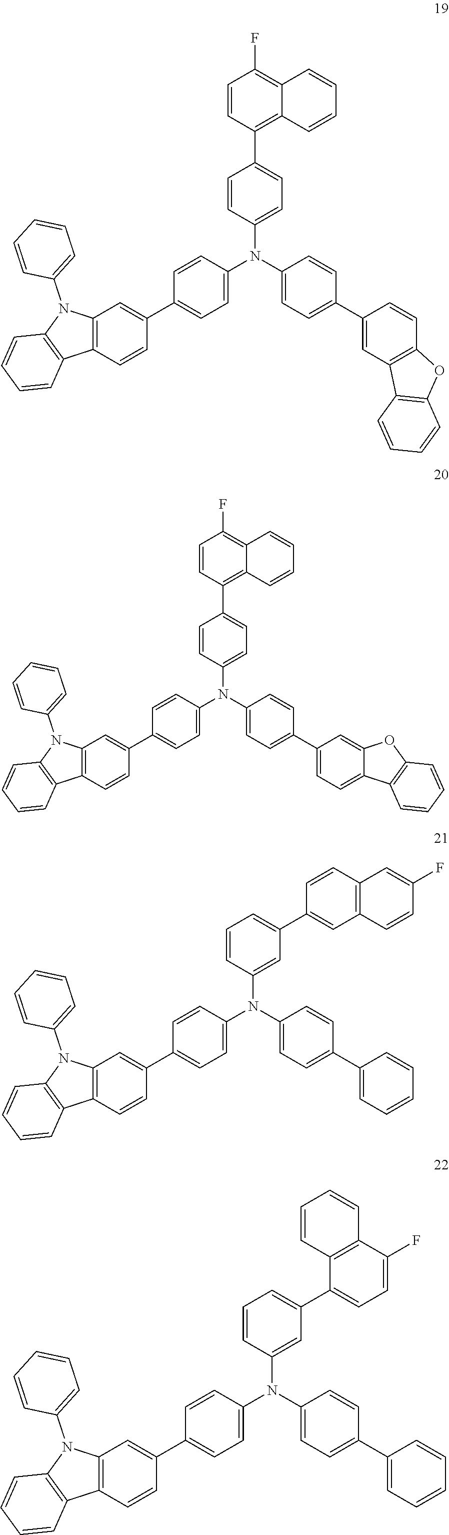 [SMILES: Fc1ccc(-c2ccc(N(c3ccc(-c4ccc5oc6ccccc6c5c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c(c4)oc4ccccc45)cc3)c3ccc(-c4ccc5c6ccccc6n(-c6ccccc6)c5c4)cc3)cc2)c2ccccc12 ; Fc1ccc2cc(-c3cccc(N(c4ccc(-c5ccccc5)cc4)c4ccc(-c5ccc6c7ccccc7n(-c7ccccc7)c6c5)cc4)c3)ccc2c1 ; Fc1ccc(-c2cccc(N(c3ccc(-c4ccccc4)cc3)c3ccc(-c4ccc5c6ccccc6n(-c6ccccc6)c5c4)cc3)c2)c2ccccc12]

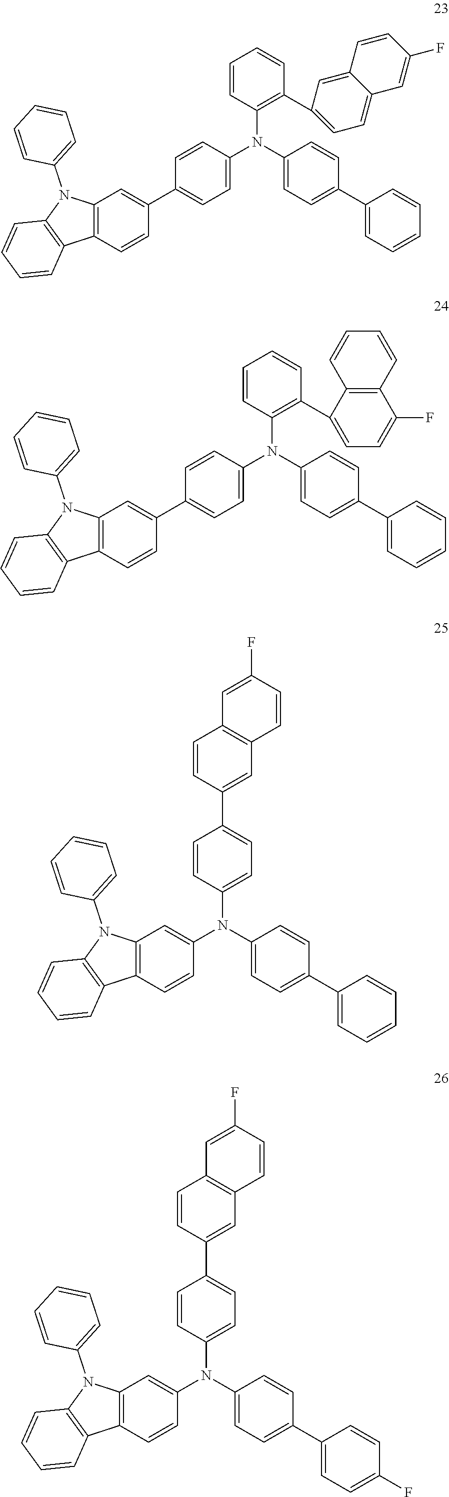 [SMILES: Fc1ccc2cc(-c3ccccc3N(c3ccc(-c4ccccc4)cc3)c3ccc(-c4ccc5c6ccccc6n(-c6ccccc6)c5c4)cc3)ccc2c1 ; Fc1ccc(-c2ccccc2N(c2ccc(-c3ccccc3)cc2)c2ccc(-c3ccc4c5ccccc5n(-c5ccccc5)c4c3)cc2)c2ccccc12 ; Fc1ccc2cc(-c3ccc(N(c4ccc(-c5ccccc5)cc4)c4ccc5c6ccccc6n(-c6ccccc6)c5c4)cc3)ccc2c1 ; Fc1ccc(-c2ccc(N(c3ccc(-c4ccc5cc(F)ccc5c4)cc3)c3ccc4c5ccccc5n(-c5ccccc5)c4c3)cc2)cc1]

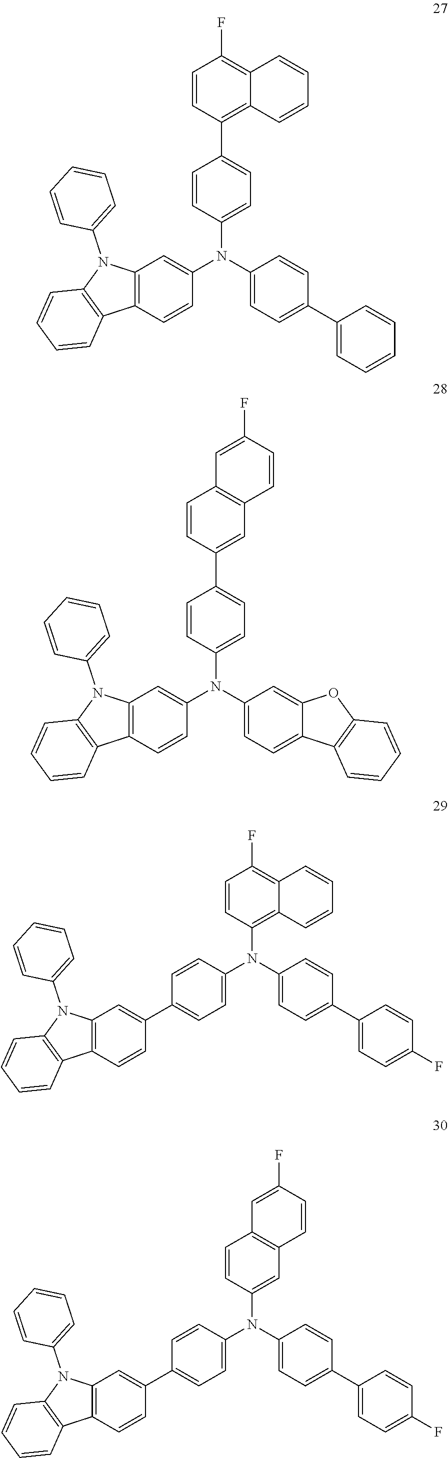 [SMILES: Fc1ccc(-c2ccc(N(c3ccc(-c4ccccc4)cc3)c3ccc4c5ccccc5n(-c5ccccc5)c4c3)cc2)c2ccccc12 ; Fc1ccc2cc(-c3ccc(N(c4ccc5c(c4)oc4ccccc45)c4ccc5c6ccccc6n(-c6ccccc6)c5c4)cc3)ccc2c1 ; Fc1ccc(-c2ccc(N(c3ccc(-c4ccc5c6ccccc6n(-c6ccccc6)c5c4)cc3)c3ccc(F)c4ccccc34)cc2)cc1 ; Fc1ccc(-c2ccc(N(c3ccc(-c4ccc5c6ccccc6n(-c6ccccc6)c5c4)cc3)c3ccc4cc(F)ccc4c3)cc2)cc1]

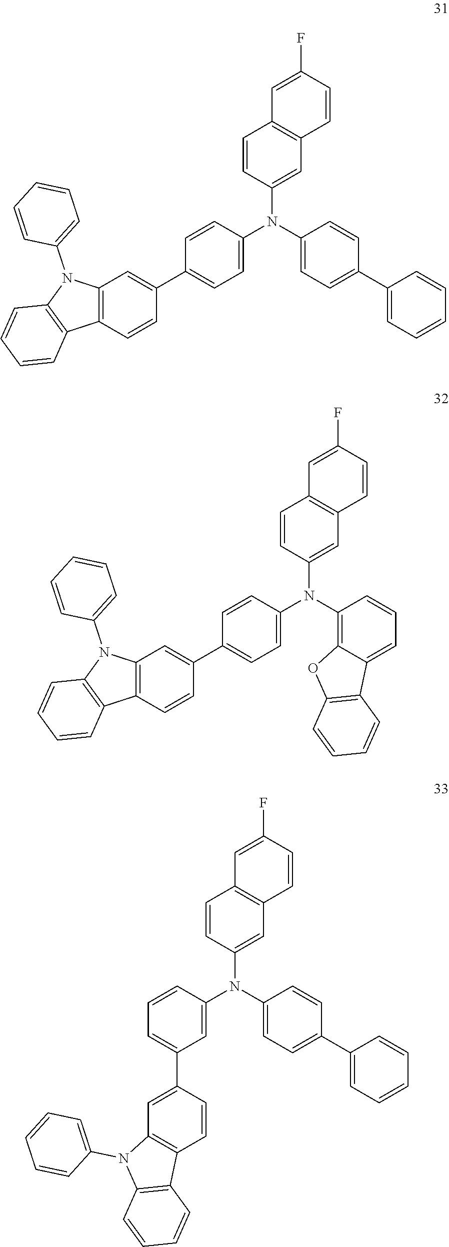 [SMILES: Fc1ccc2cc(N(c3ccc(-c4ccccc4)cc3)c3ccc(-c4ccc5c6ccccc6n(-c6ccccc6)c5c4)cc3)ccc2c1 ; Fc1ccc2cc(N(c3ccc(-c4ccc5c6ccccc6n(-c6ccccc6)c5c4)cc3)c3cccc4c3oc3ccccc34)ccc2c1 ; Fc1ccc2cc(N(c3ccc(-c4ccccc4)cc3)c3cccc(-c4ccc5c6ccccc6n(-c6ccccc6)c5c4)c3)ccc2c1]

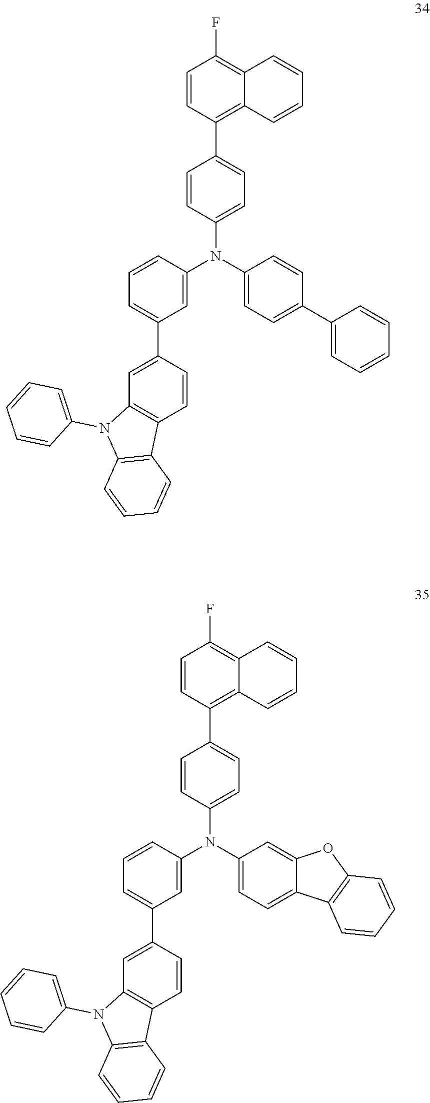 [SMILES: Fc1ccc(-c2ccc(N(c3ccc(-c4ccccc4)cc3)c3cccc(-c4ccc5c6ccccc6n(-c6ccccc6)c5c4)c3)cc2)c2ccccc12 ; Fc1ccc(-c2ccc(N(c3cccc(-c4ccc5c6ccccc6n(-c6ccccc6)c5c4)c3)c3ccc4c(c3)oc3ccccc34)cc2)c2ccccc12]

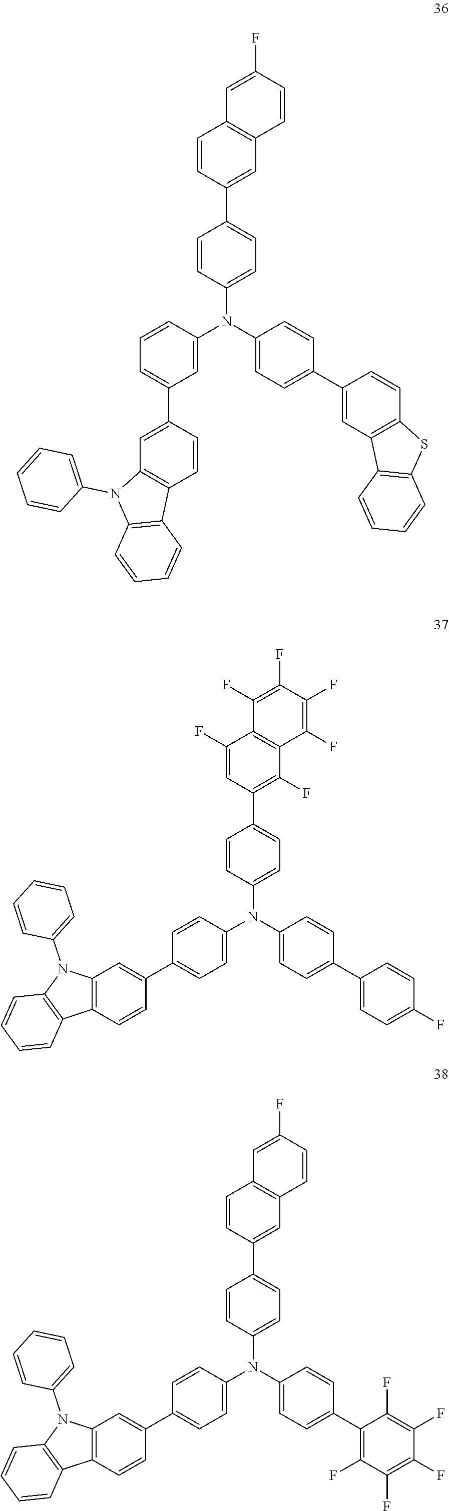 [SMILES: Fc1ccc2cc(-c3ccc(N(c4ccc(-c5ccc6sc7ccccc7c6c5)cc4)c4cccc(-c5ccc6c7ccccc7n(-c7ccccc7)c6c5)c4)cc3)ccc2c1 ; Fc1ccc(-c2ccc(N(c3ccc(-c4ccc5c6ccccc6n(-c6ccccc6)c5c4)cc3)c3ccc(-c4cc(F)c5c(F)c(F)c(F)c(F)c5c4F)cc3)cc2)cc1 ; Fc1ccc2cc(-c3ccc(N(c4ccc(-c5ccc6c7ccccc7n(-c7ccccc7)c6c5)cc4)c4ccc(-c5c(F)c(F)c(F)c(F)c5F)cc4)cc3)ccc2c1]

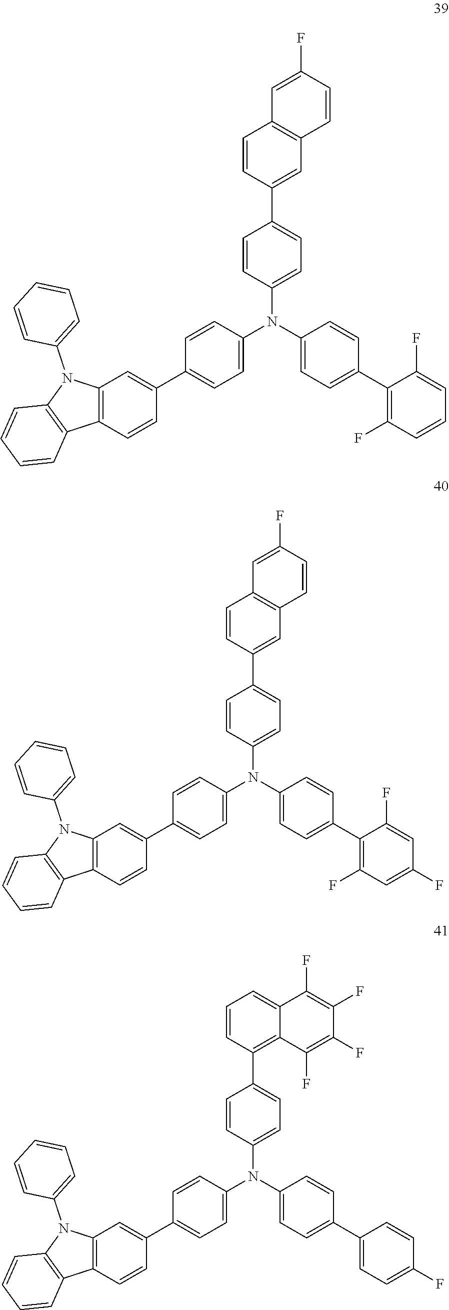 [SMILES: Fc1ccc2cc(-c3ccc(N(c4ccc(-c5ccc6c7ccccc7n(-c7ccccc7)c6c5)cc4)c4ccc(-c5c(F)cccc5F)cc4)cc3)ccc2c1 ; Fc1cc(F)c(-c2ccc(N(c3ccc(-c4ccc5cc(F)ccc5c4)cc3)c3ccc(-c4ccc5c6ccccc6n(-c6ccccc6)c5c4)cc3)cc2)c(F)c1 ; Fc1ccc(-c2ccc(N(c3ccc(-c4ccc5c6ccccc6n(-c6ccccc6)c5c4)cc3)c3ccc(-c4cccc5c(F)c(F)c(F)c(F)c45)cc3)cc2)cc1]

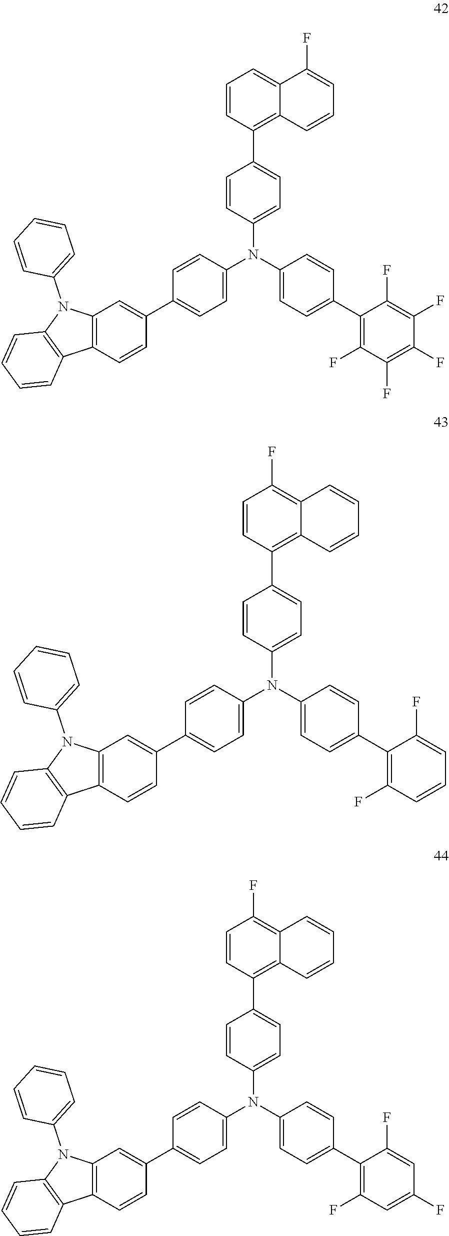 [SMILES: Fc1c(F)c(F)c(-c2ccc(N(c3ccc(-c4ccc5c6ccccc6n(-c6ccccc6)c5c4)cc3)c3ccc(-c4cccc5c(F)cccc45)cc3)cc2)c(F)c1F ; Fc1cccc(F)c1-c1ccc(N(c2ccc(-c3ccc4c5ccccc5n(-c5ccccc5)c4c3)cc2)c2ccc(-c3ccc(F)c4ccccc34)cc2)cc1 ; Fc1cc(F)c(-c2ccc(N(c3ccc(-c4ccc5c6ccccc6n(-c6ccccc6)c5c4)cc3)c3ccc(-c4ccc(F)c5ccccc45)cc3)cc2)c(F)c1]

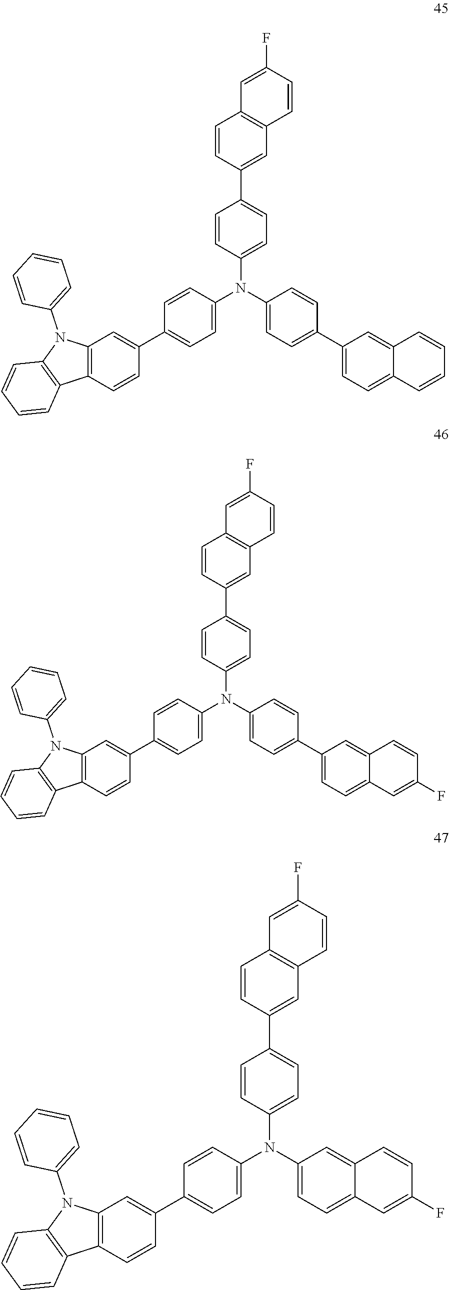 [SMILES: Fc1ccc2cc(-c3ccc(N(c4ccc(-c5ccc6ccccc6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6cc(F)ccc6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc5cc(F)ccc5c4)cc3)ccc2c1]

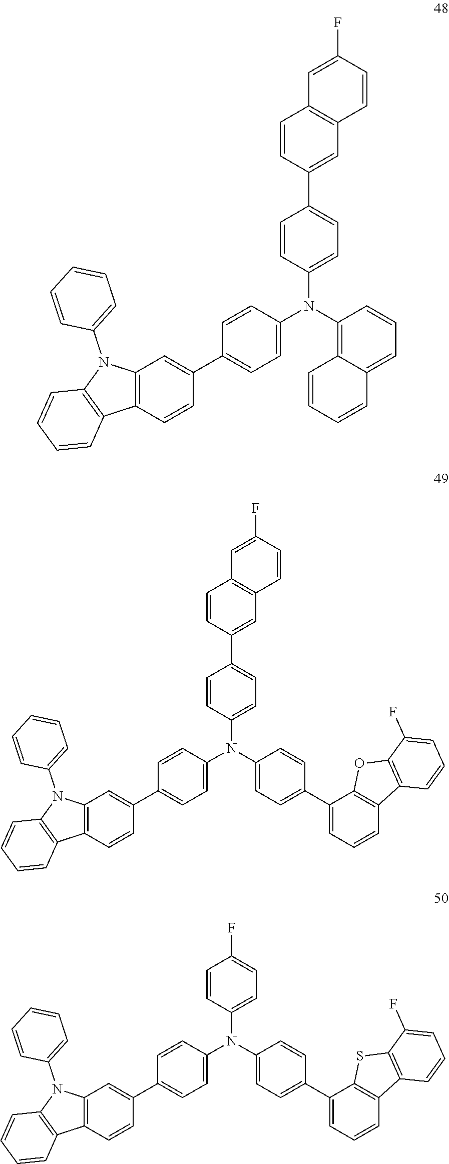 [SMILES: Fc1ccc2cc(-c3ccc(N(c4ccc(-c5ccc6c7ccccc7n(-c7ccccc7)c6c5)cc4)c4cccc5ccccc45)cc3)ccc2c1 ; Fc1ccc2cc(-c3ccc(N(c4ccc(-c5ccc6c7ccccc7n(-c7ccccc7)c6c5)cc4)c4ccc(-c5cccc6c5oc5c(F)cccc56)cc4)cc3)ccc2c1 ; Fc1ccc(N(c2ccc(-c3ccc4c5ccccc5n(-c5ccccc5)c4c3)cc2)c2ccc(-c3cccc4c3sc3c(F)cccc34)cc2)cc1]

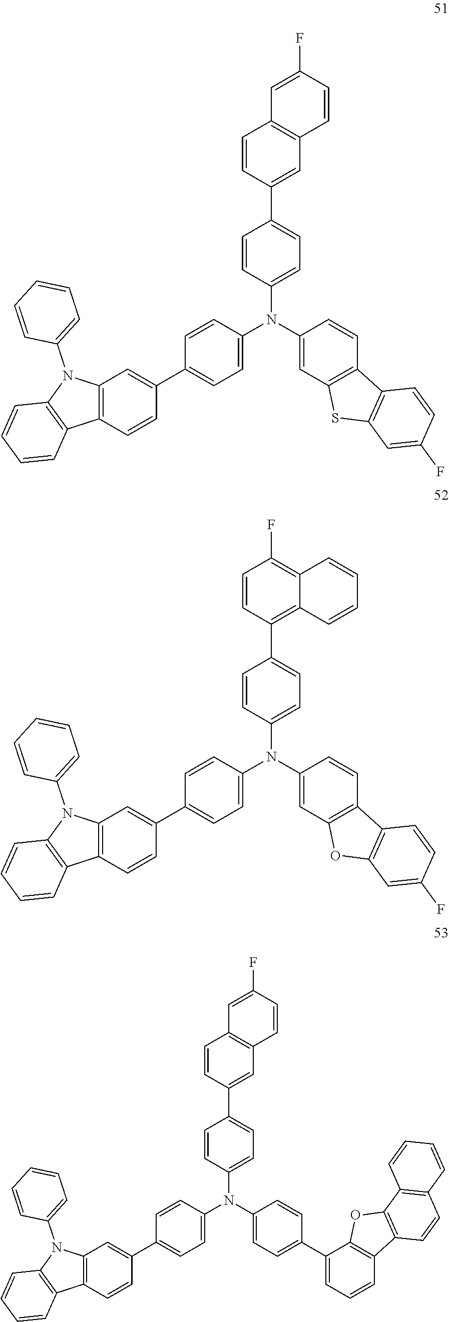 [SMILES: Fc1ccc2cc(-c3ccc(N(c4ccc(-c5ccc6c7ccccc7n(-c7ccccc7)c6c5)cc4)c4ccc5c(c4)sc4cc(F)ccc45)cc3)ccc2c1 ; Fc1ccc2c(c1)oc1cc(N(c3ccc(-c4ccc5c6ccccc6n(-c6ccccc6)c5c4)cc3)c3ccc(-c4ccc(F)c5ccccc45)cc3)ccc12 ; Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1]

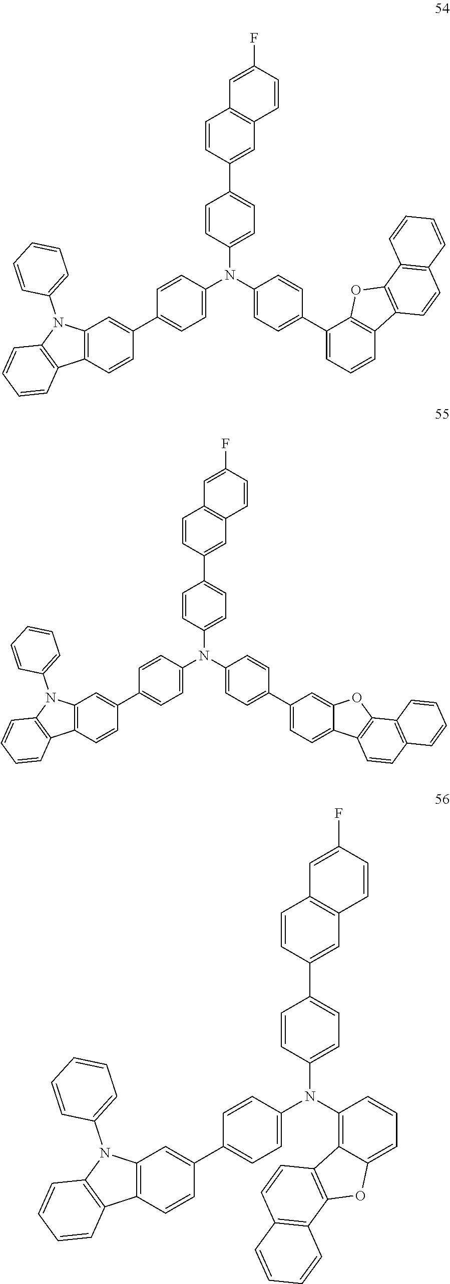 [SMILES: Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1 ; Fc1ccc2cc(-c3ccc(N(c4ccc(-c5ccc6c(c5)oc5c7ccccc7ccc65)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4cccc5oc6c7ccccc7ccc6c45)cc3)ccc2c1]

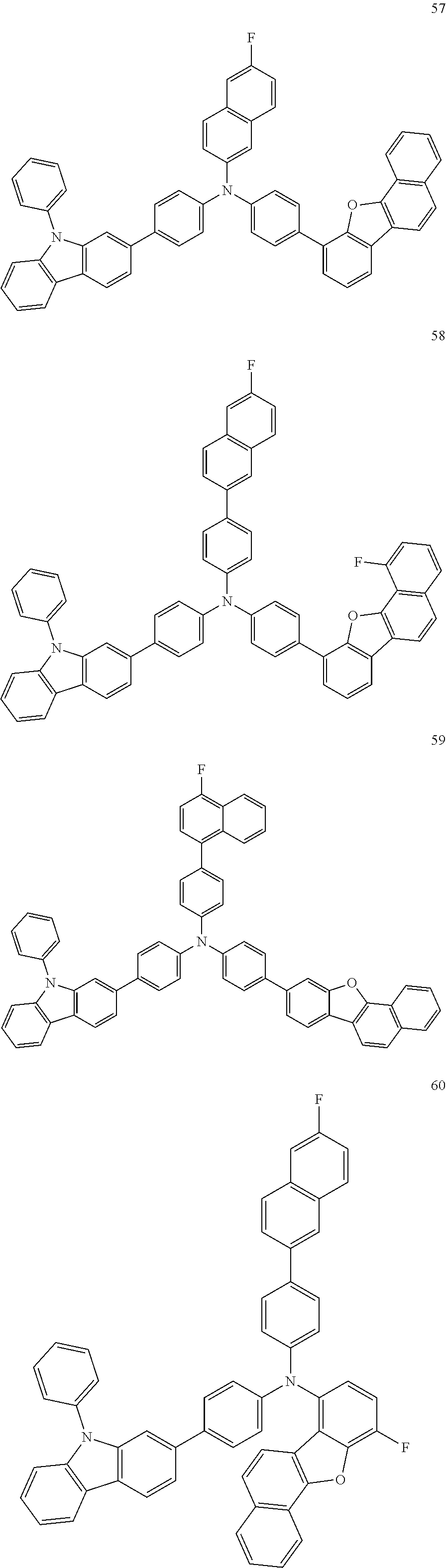 [SMILES: Fc1ccc2cc(N(c3ccc(-c4ccc5c6ccccc6n(-c6ccccc6)c5c4)cc3)c3ccc(-c4cccc5c4oc4c6ccccc6ccc54)cc3)ccc2c1 ; Fc1ccc2cc(-c3ccc(N(c4ccc(-c5ccc6c7ccccc7n(-c7ccccc7)c6c5)cc4)c4ccc(-c5cccc6c5oc5c6ccc6cccc(F)c65)cc4)cc3)ccc2c1 ; Fc1ccc(-c2ccc(N(c3ccc(-c4ccc5c(c4)oc4c6ccccc6ccc54)cc3)c3ccc(-c4ccc5c6ccccc6n(-c6ccccc6)c5c4)cc3)cc2)c2ccccc12 ; Fc1ccc2cc(-c3ccc(N(c4ccc(-c5ccc6c7ccccc7n(-c7ccccc7)c6c5)cc4)c4ccc(F)c5oc6c7ccccc7ccc6c45)cc3)ccc2c1]

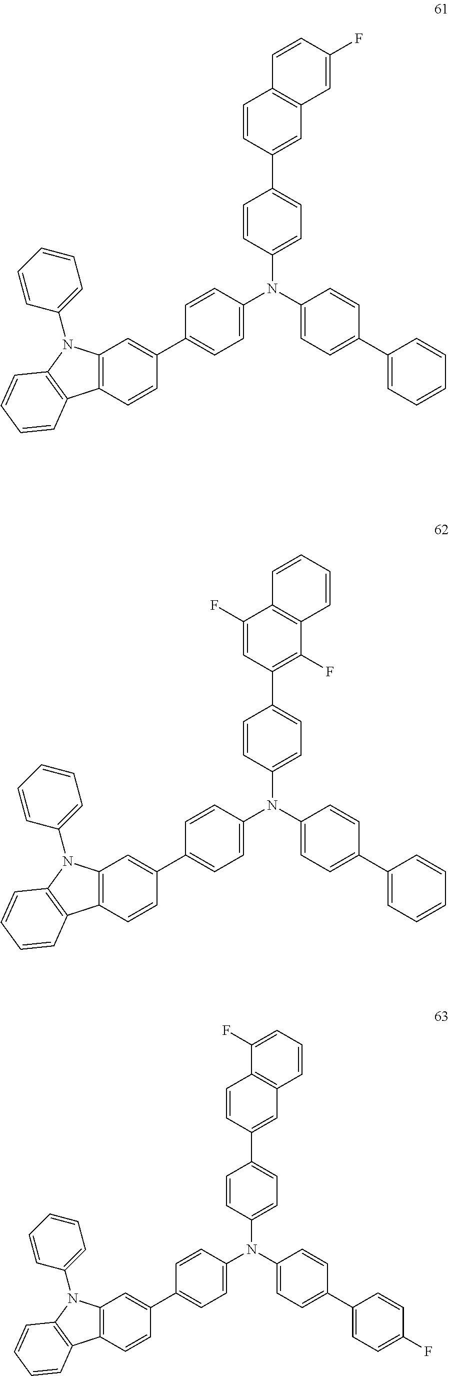 [SMILES: Fc1ccc2ccc(-c3ccc(N(c4ccc(-c5ccccc5)cc4)c4ccc(-c5ccc6c7ccccc7n(-c7ccccc7)c6c5)cc4)cc3)cc2c1 ; Fc1cc(-c2ccc(N(c3ccc(-c4ccccc4)cc3)c3ccc(-c4ccc5c6ccccc6n(-c6ccccc6)c5c4)cc3)cc2)c(F)c2ccccc12 ; Fc1ccc(-c2ccc(N(c3ccc(-c4ccc5c(F)cccc5c4)cc3)c3ccc(-c4ccc5c6ccccc6n(-c6ccccc6)c5c4)cc3)cc2)cc1]

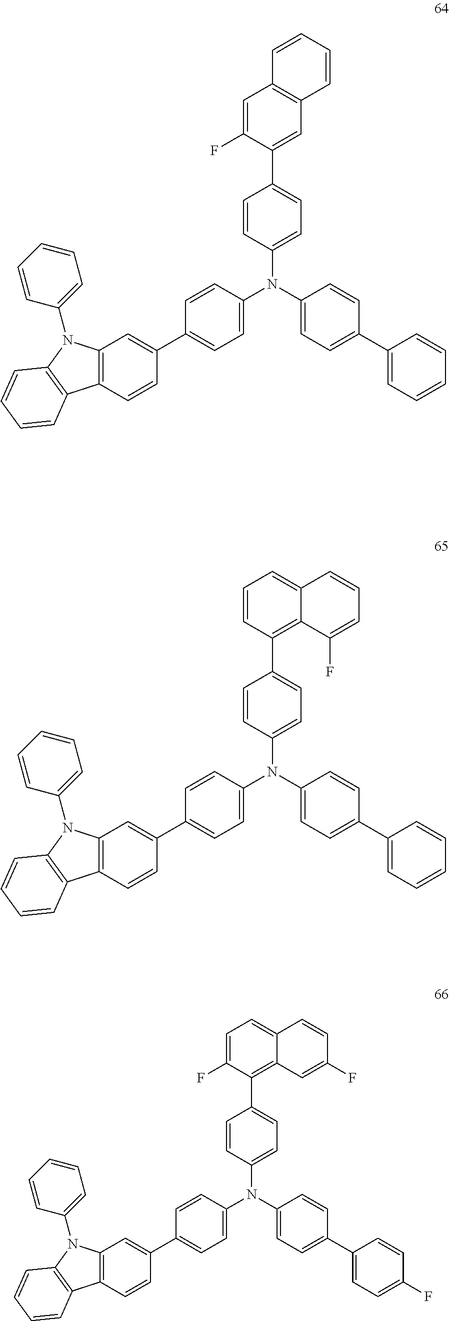 [SMILES: Fc1cc2ccccc2cc1-c1ccc(N(c2ccc(-c3ccccc3)cc2)c2ccc(-c3ccc4c5ccccc5n(-c5ccccc5)c4c3)cc2)cc1 ; Fc1cccc2cccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4c(F)ccc5ccc(F)cc45)cc3)cc2)cc1]

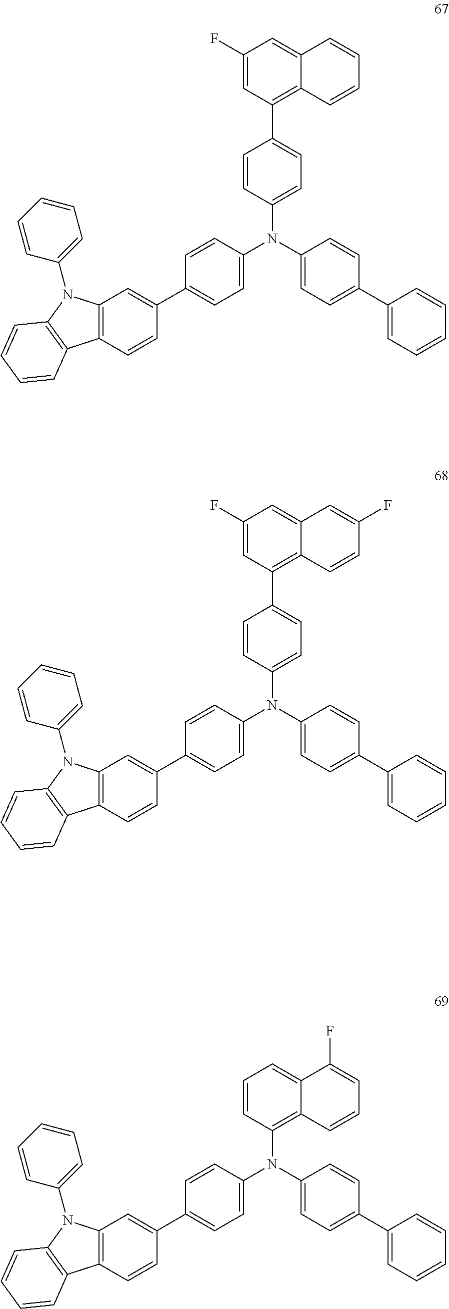 [SMILES: Fc1cc(-c2ccc(N(c3ccc(-c4ccccc4)cc3)c3ccc(-c4ccc5c6ccccc6n(-c6ccccc6)c5c4)cc3)cc2)c2ccccc2c1 ; Fc1ccc2c(-c3ccc(N(c4ccc(-c5ccccc5)cc4)c4ccc(-c5ccc6c7ccccc7n(-c7ccccc7)c6c5)cc4)cc3)cc(F)cc2c1 ; Fc1cccc2c(N(c3ccc(-c4ccccc4)cc3)c3ccc(-c4ccc5c6ccccc6n(-c6ccccc6)c5c4)cc3)cccc12]

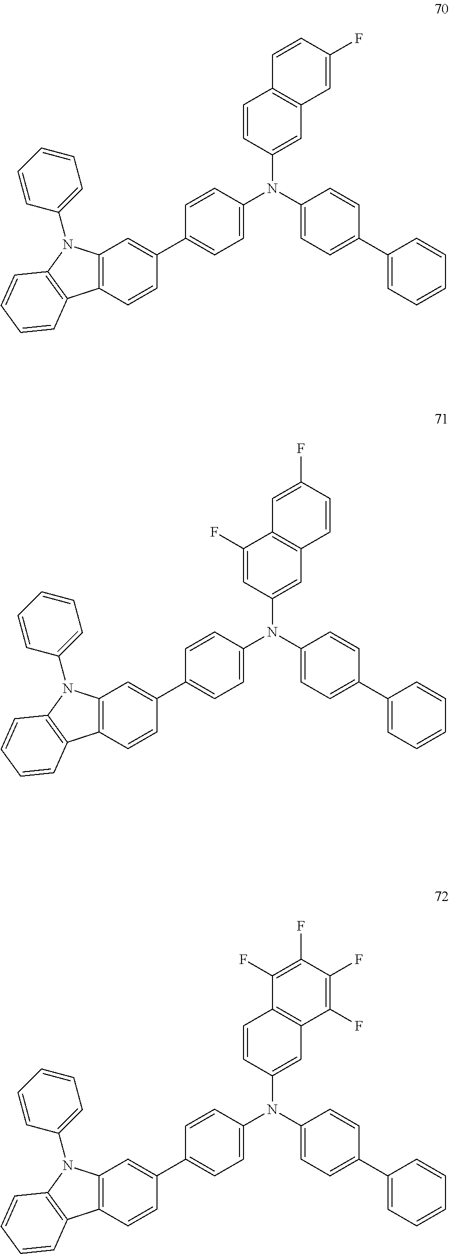 [SMILES: Fc1ccc2ccc(N(c3ccc(-c4ccccc4)cc3)c3ccc(-c4ccc5c6ccccc6n(-c6ccccc6)c5c4)cc3)cc2c1 ; Fc1ccc2cc(N(c3ccc(-c4ccccc4)cc3)c3ccc(-c4ccc5c6ccccc6n(-c6ccccc6)c5c4)cc3)cc(F)c2c1 ; Fc1c(F)c(F)c2cc(N(c3ccc(-c4ccccc4)cc3)c3ccc(-c4ccc5c6ccccc6n(-c6ccccc6)c5c4)cc3)ccc2c1F]

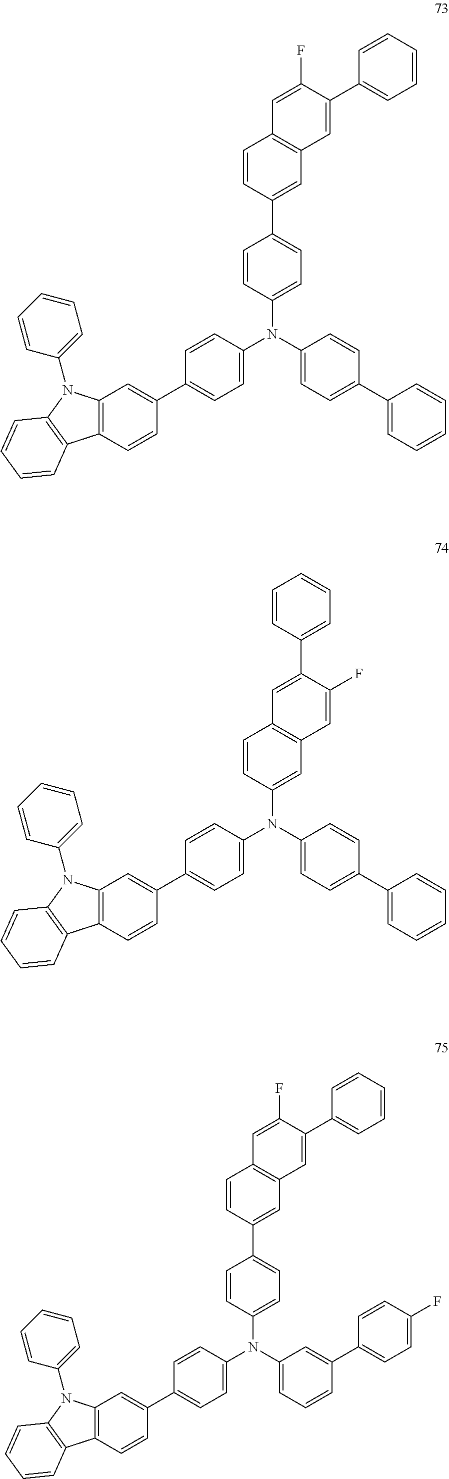 [SMILES: Fc1cc2ccc(-c3ccc(N(c4ccc(-c5ccccc5)cc4)c4ccc(-c5ccc6c7ccccc7n(-c7ccccc7)c6c5)cc4)cc3)cc2cc1-c1ccccc1 ; Fc1cc2cc(N(c3ccc(-c4ccccc4)cc3)c3ccc(-c4ccc5c6ccccc6n(-c6ccccc6)c5c4)cc3)ccc2cc1-c1ccccc1 ; Fc1ccc(-c2cccc(N(c3ccc(-c4ccc5cc(F)c(-c6ccccc6)cc5c4)cc3)c3ccc(-c4ccc5c6ccccc6n(-c6ccccc6)c5c4)cc3)c2)cc1]

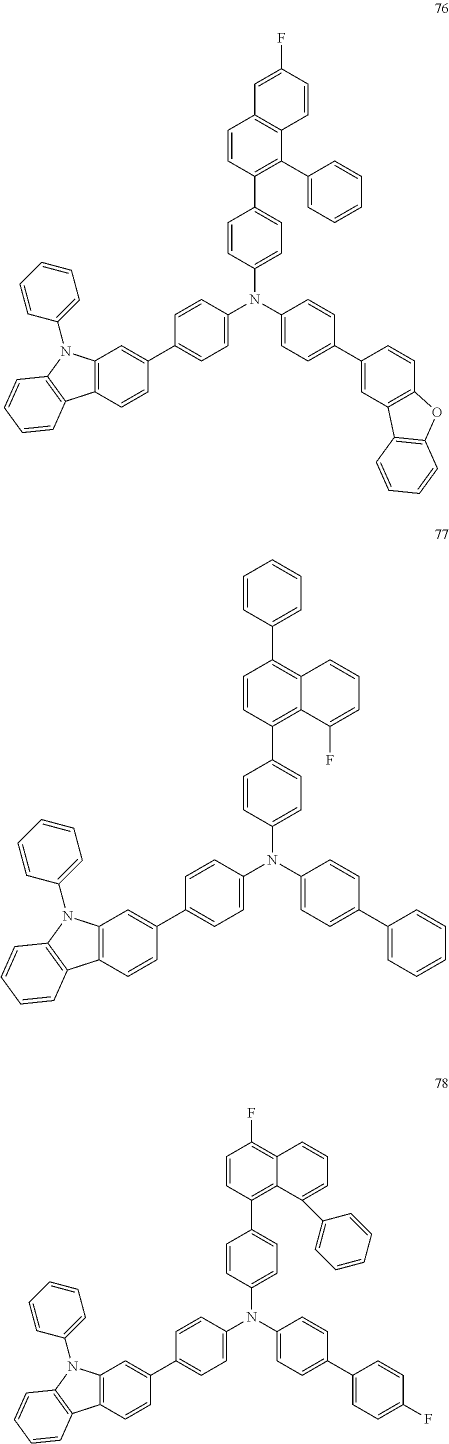 [SMILES: Fc1ccc2c(-c3ccccc3)c(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1cccc2c(-c3ccccc3)ccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4ccc(F)c5cccc(-c6ccccc6)c45)cc3)cc2)cc1]

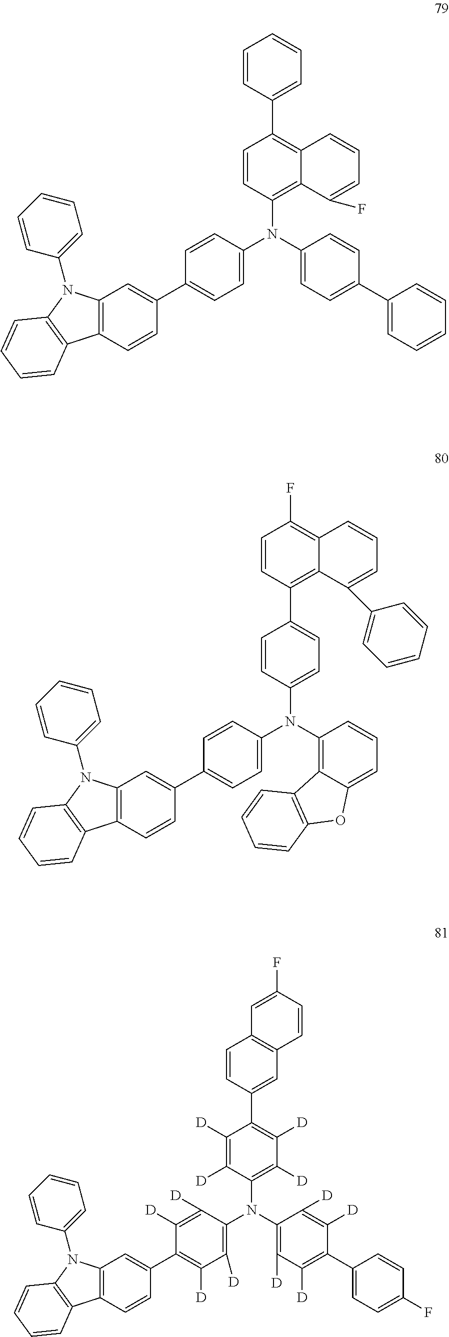 [SMILES: Fc1cccc2c(-c3ccccc3)ccc(N(c3ccc(-c4ccccc4)cc3)c3ccc(-c4ccc5c6ccccc6n(-c6ccccc6)c5c4)cc3)c12 ; Fc1ccc(-c2ccc(N(c3ccc(-c4ccc5c6ccccc6n(-c6ccccc6)c5c4)cc3)c3cccc4oc5ccccc5c34)cc2)c2c(-c3ccccc3)cccc12 ; [2H]c1c([2H])c(N(c2c([2H])c([2H])c(-c3ccc4cc(F)ccc4c3)c([2H])c2[2H])c2c([2H])c([2H])c(-c3ccc4c5ccccc5n(-c5ccccc5)c4c3)c([2H])c2[2H])c([2H])c([2H])c1-c1ccc(F)cc1]

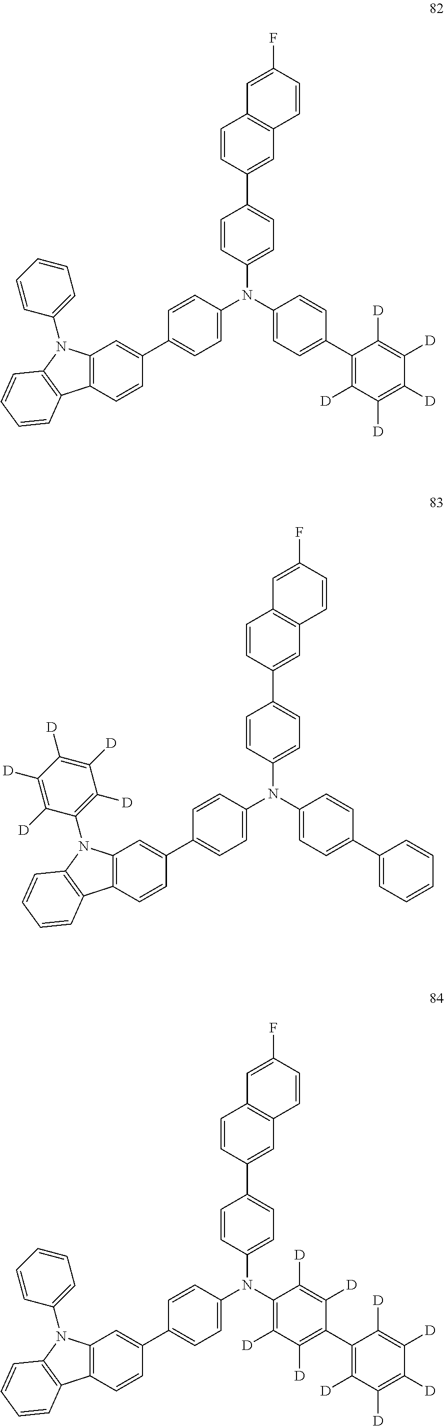 [SMILES: [2H]c1c([2H])c([2H])c(-c2ccc(N(c3ccc(-c4ccc5cc(F)ccc5c4)cc3)c3ccc(-c4ccc5c6ccccc6n(-c6ccccc6)c5c4)cc3)cc2)c([2H])c1[2H] ; [2H]c1c([2H])c([2H])c(-n2c3ccccc3c3ccc(-c4ccc(N(c5ccc(-c6ccccc6)cc5)c5ccc(-c6ccc7cc(F)ccc7c6)cc5)cc4)cc32)c([2H])c1[2H] ; [2H]c1c([2H])c([2H])c(-c2c([2H])c([2H])c(N(c3ccc(-c4ccc5cc(F)ccc5c4)cc3)c3ccc(-c4ccc5c6ccccc6n(-c6ccccc6)c5c4)cc3)c([2H])c2[2H])c([2H])c1[2H]]

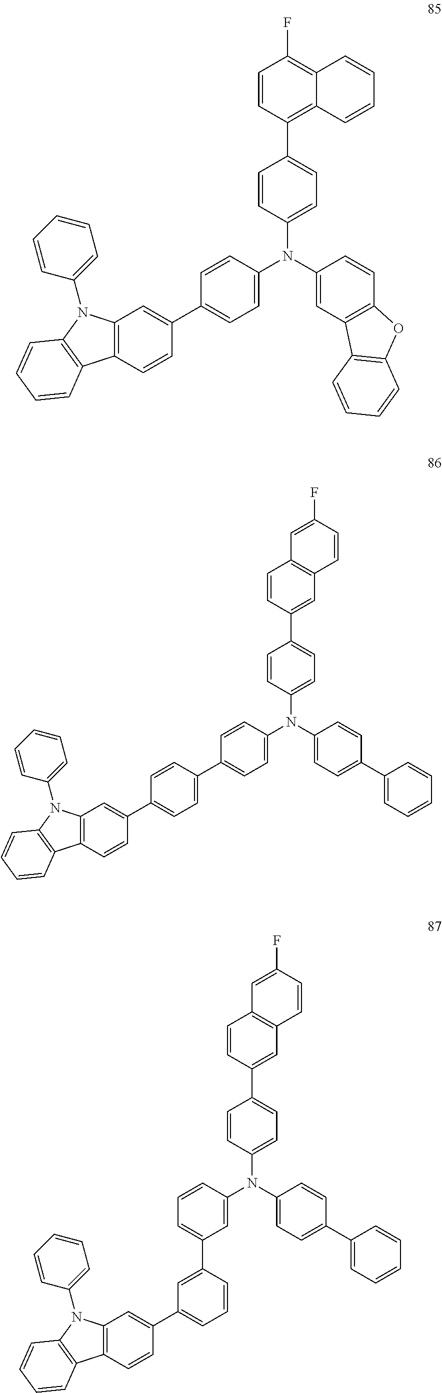 [SMILES: Fc1ccc(-c2ccc(N(c3ccc(-c4ccc5c6ccccc6n(-c6ccccc6)c5c4)cc3)c3ccc4oc5ccccc5c4c3)cc2)c2ccccc12 ; Fc1ccc2cc(-c3ccc(N(c4ccc(-c5ccccc5)cc4)c4ccc(-c5ccc(-c6ccc7c8ccccc8n(-c8ccccc8)c7c6)cc5)cc4)cc3)ccc2c1 ; Fc1ccc2cc(-c3ccc(N(c4ccc(-c5ccccc5)cc4)c4cccc(-c5cccc(-c6ccc7c8ccccc8n(-c8ccccc8)c7c6)c5)c4)cc3)ccc2c1]

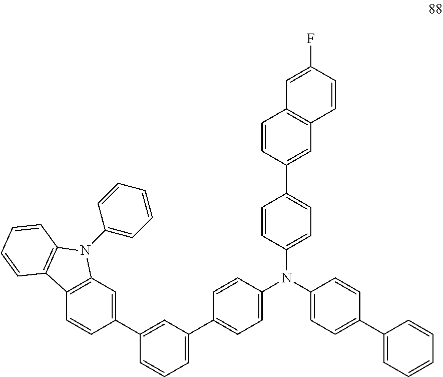 [SMILES: Fc1ccc2cc(-c3ccc(N(c4ccc(-c5ccccc5)cc4)c4ccc(-c5cccc(-c6ccc7c8ccccc8n(-c8ccccc8)c7c6)c5)cc4)cc3)ccc2c1]

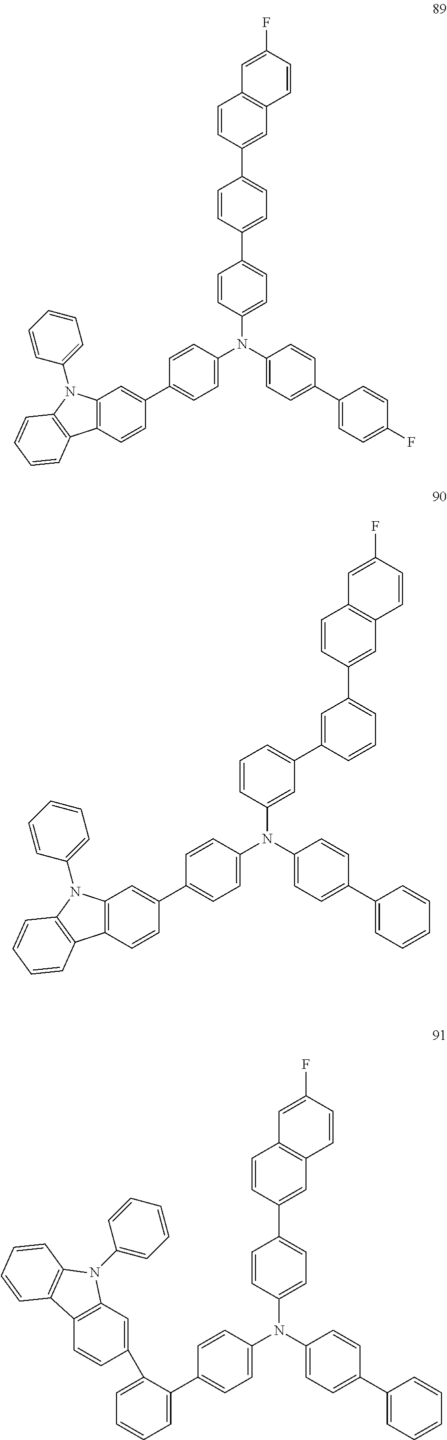 [SMILES: Fc1ccc(-c2ccc(N(c3ccc(-c4ccc(-c5ccc6cc(F)ccc6c5)cc4)cc3)c3ccc(-c4ccc5c6ccccc6n(-c6ccccc6)c5c4)cc3)cc2)cc1 ; Fc1ccc2cc(-c3cccc(-c4cccc(N(c5ccc(-c6ccccc6)cc5)c5ccc(-c6ccc7c8ccccc8n(-c8ccccc8)c7c6)cc5)c4)c3)ccc2c1 ; Fc1ccc2cc(-c3ccc(N(c4ccc(-c5ccccc5)cc4)c4ccc(-c5ccccc5-c5ccc6c7ccccc7n(-c7ccccc7)c6c5)cc4)cc3)ccc2c1]

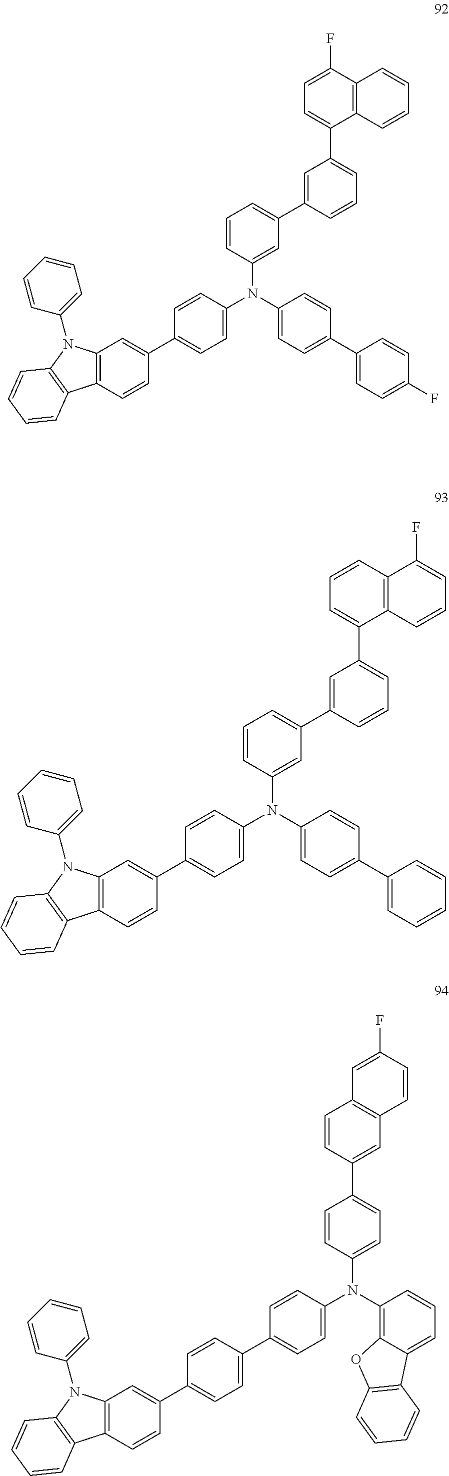 [SMILES: Fc1ccc(-c2ccc(N(c3ccc(-c4ccc5c6ccccc6n(-c6ccccc6)c5c4)cc3)c3cccc(-c4cccc(-c5ccc(F)c6ccccc56)c4)c3)cc2)cc1 ; Fc1cccc2c(-c3cccc(-c4cccc(N(c5ccc(-c6ccccc6)cc5)c5ccc(-c6ccc7c8ccccc8n(-c8ccccc8)c7c6)cc5)c4)c3)cccc12 ; Fc1ccc2cc(-c3ccc(N(c4ccc(-c5ccc(-c6ccc7c8ccccc8n(-c8ccccc8)c7c6)cc5)cc4)c4cccc5c4oc4ccccc45)cc3)ccc2c1]

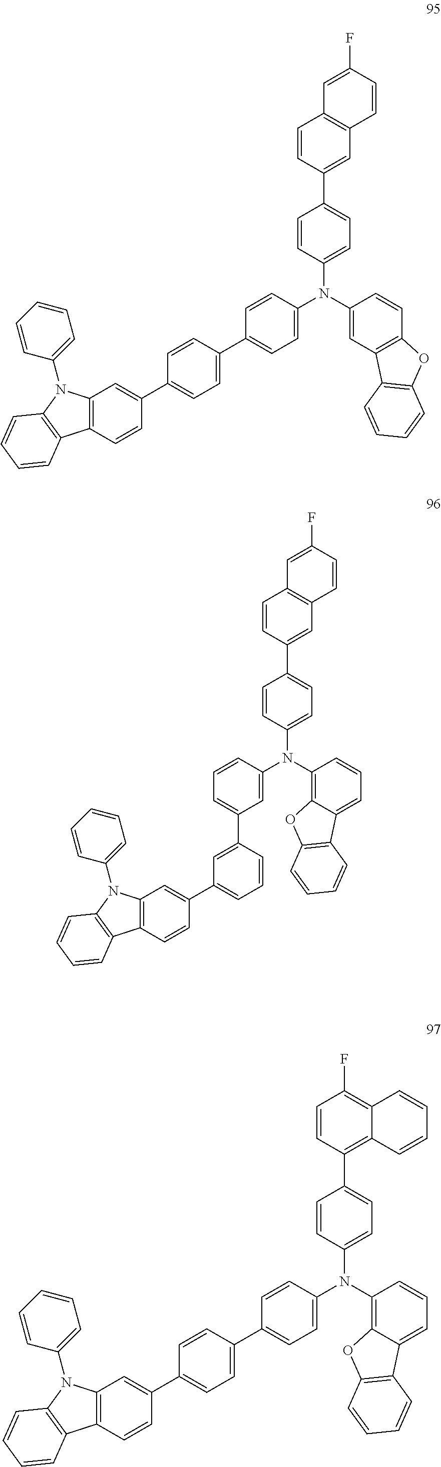 [SMILES: Fc1ccc2cc(-c3ccc(N(c4ccc(-c5ccc(-c6ccc7c8ccccc8n(-c8ccccc8)c7c6)cc5)cc4)c4ccc5oc6ccccc6c5c4)cc3)ccc2c1 ; Fc1ccc2cc(-c3ccc(N(c4cccc(-c5cccc(-c6ccc7c8ccccc8n(-c8ccccc8)c7c6)c5)c4)c4cccc5c4oc4ccccc45)cc3)ccc2c1 ; Fc1ccc(-c2ccc(N(c3ccc(-c4ccc(-c5ccc6c7ccccc7n(-c7ccccc7)c6c5)cc4)cc3)c3cccc4c3oc3ccccc34)cc2)c2ccccc12]

The amine compound according to an embodiment disclosed in Compound Group 1 and the like includes a 2-carbazole group and a naphthyl group substituted with at least one fluorene atom and thus may have high molecular stability and reliability, thereby exhibiting low driving voltage and excellent lifespan characteristics. In addition, the light-emitting element according to an embodiment including the amine compound according to an embodiment may have a reduced driving voltage and an improved lifespan.

In the light-emitting element ED according to an embodiment, the hole transport region HTR may further include a compound according to Formula H-1. For example, the light-emitting element ED according to an embodiment may include a compound according to Formula H-1 in another layer of the hole transport region HTR, which includes no amine compound according to an embodiment, according to Formula 1 described previously. However, an embodiment is not limited thereto.

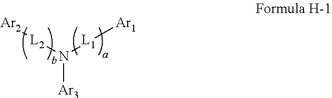

In Formula H-1, L₁ and L₂ may be each independently a direct linkage, a substituted or unsubstituted arylene group having 6 to 30 ring-forming carbon atoms, or a substituted or unsubstituted heteroarylene group having 2 to 30 ring-forming carbon atoms. Subscript a and subscript b may be each independently an integer of 0 to 10. When a or b is an integer of 2 or greater, a plurality of L₁ and a plurality of L₂ may be each independently a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. In addition, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound according to Formula H-1 may be a monoamine compound. Alternatively, the compound according to Formula H-1 may be a diamine compound, in which at least one among Ar₁ to Ar₃ includes an amine group as a substituent. In addition, the compound according to Formula H-1 may be a carbazole-based compound including a substituted or unsubstituted carbazole moiety in at least one among Ar₁ and Ar₂, or a fluorene-based compound including a substituted or unsubstituted fluorene moiety in at least one among Ar₁ and Ar₂.

The compound according to Formula H-1 may have a structure according to any one compound in Compound Group H. However, the compounds listed in Compound Group H are examples, and the compound according to Formula H-1 is not limited to the compounds listed in Compound Group H.

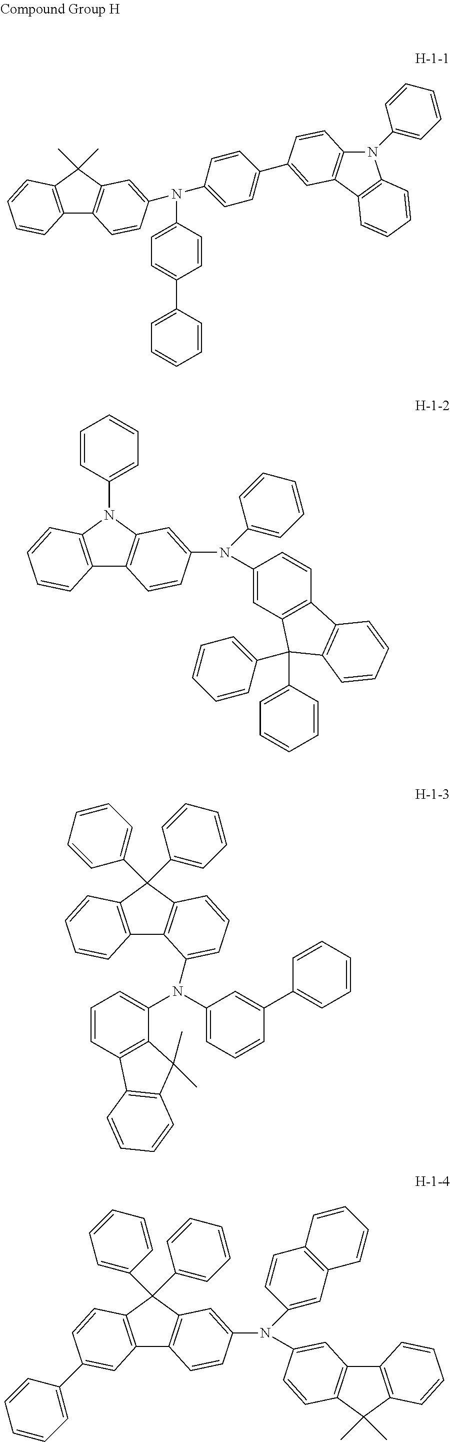 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

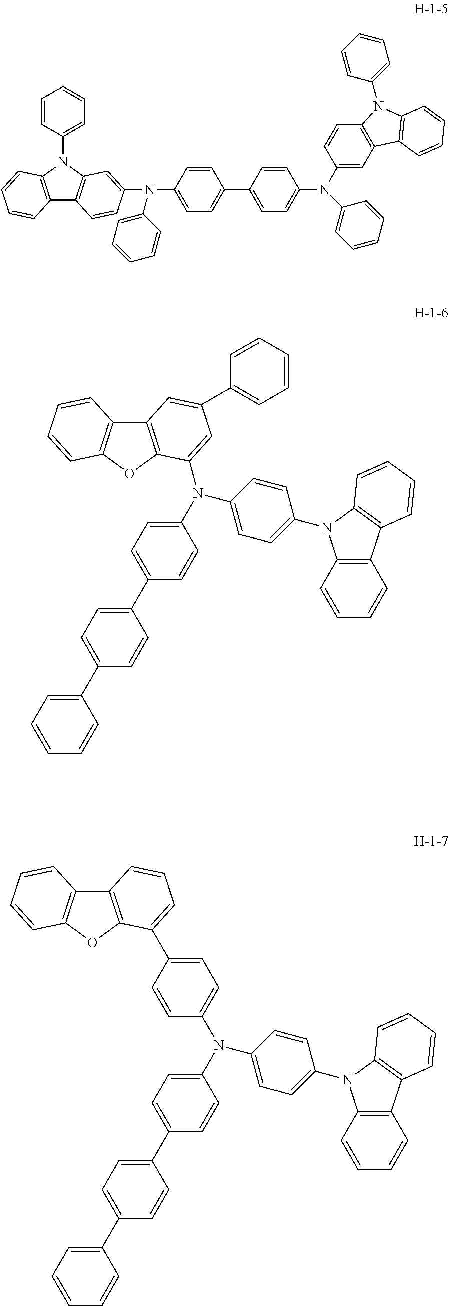 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

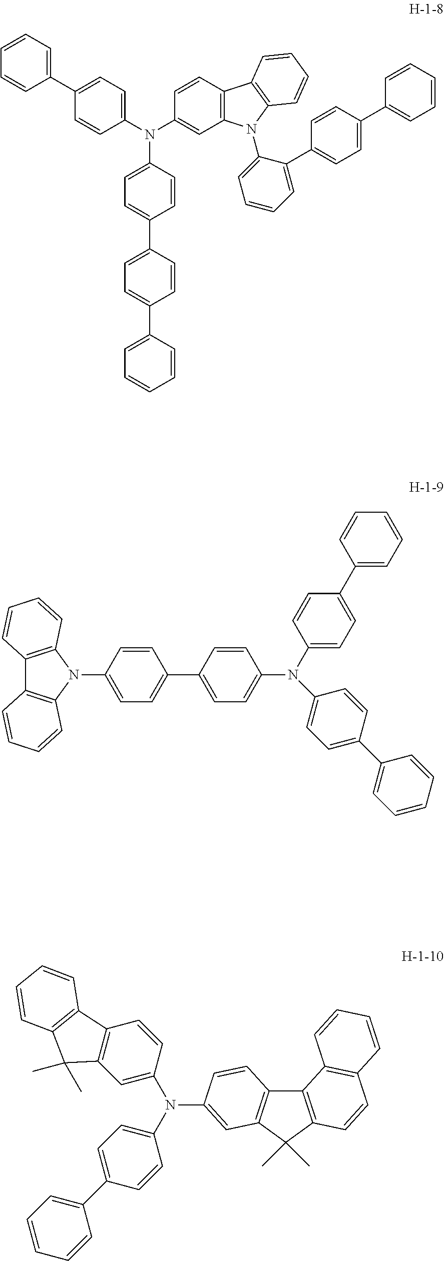 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

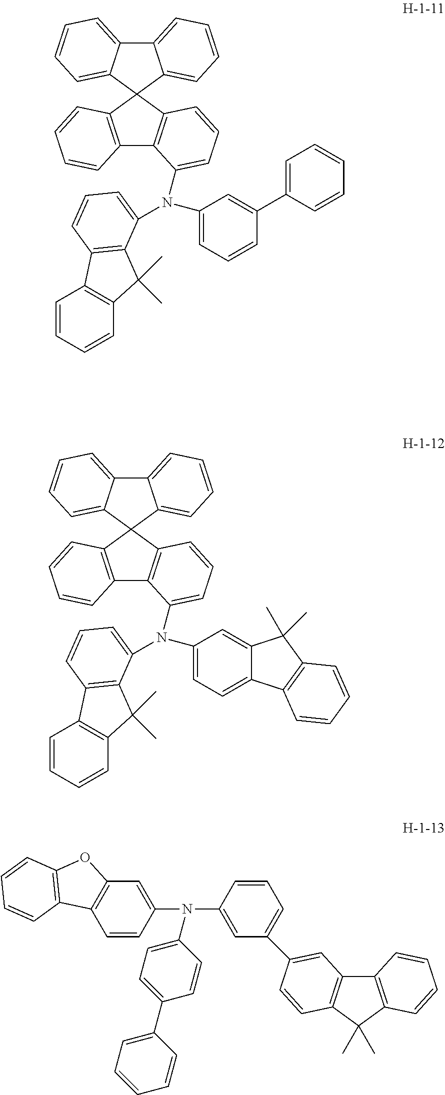 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

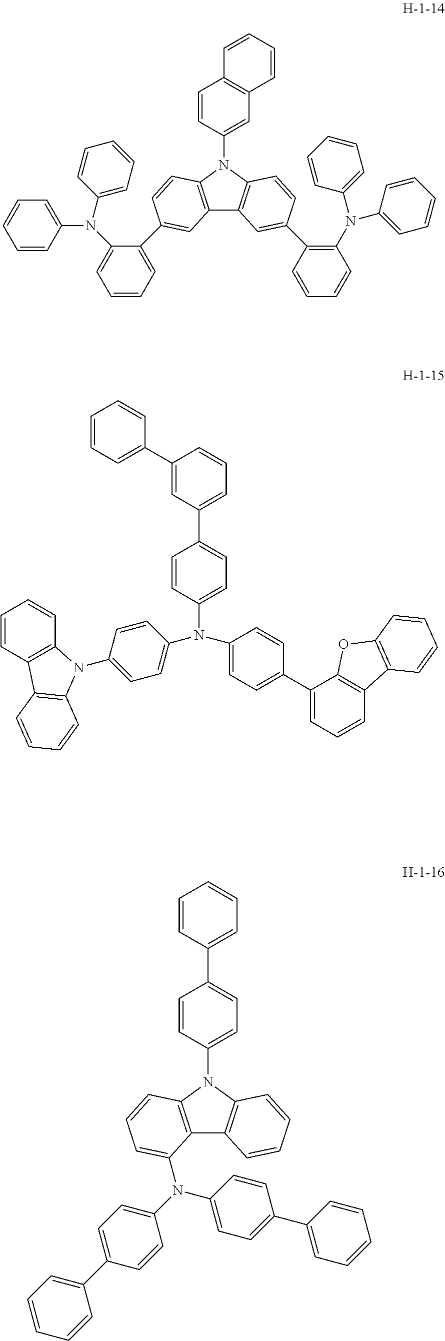 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

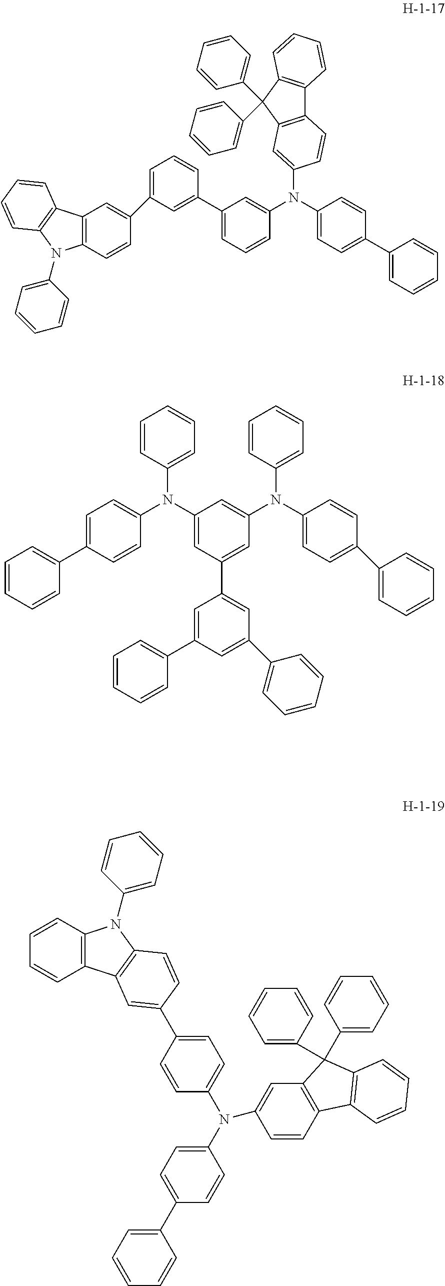 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may further include a phthalocyanine compound such as copper phthalocyanine, N¹,N^{1′}-([1,1′-biphenyl]-4,4′-diyl)bis(N¹-phenyl-N4,N4-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f. 2′,3′-h]quinoxaline-2,3,6,7,10,11-hexacarbonitrile (HATCN), etc.

The hole transport region HTR may further include a carbazole-based derivative such as N-phenyl carbazole and polyvinyl carbazole, a fluorene-based derivative, a triphenylamine-based derivative such as N,N′-bis(3-methylphenyl)-N,N′-diphenyl-[1,1′-biphenyl]-4,4′-diamine (TPD), and 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 1,3-bis(N-carbazolyl)benzene (mCP), etc.

In addition, the hole transport region HTR may further include (9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the previously presented described compounds of the hole transport region in at least one among a hole injection layer HIL, a hole transport layer HTL, an emission auxiliary layer EAL, and an electron-blocking layer EBL.

The thickness of the hole transport region HTR may be about 100 Å to about 10,000 Å, for example, about 100 Å to about 5,000 Å. When the hole transport region HTR includes a hole injection layer HIL, a thickness of the hole injection layer HIL may be, for example, about 30 Å to about 1,000 Å. When the hole transport region HTR includes a hole transport layer HTL, a thickness of the hole transport layer HTL may be about 30 Å to about 1,000 Å. For example, when the hole transport region HTR includes an electron-blocking layer EBL, a thickness of the electron-blocking layer EBL may be about 10 Å to about 1,000 Å. If the thicknesses of the hole transport region HTR, the hole injection layer HIL, the hole transport layer HTL and the electron-blocking layer EBL fall within the previously presented described ranges, satisfactory hole transport properties may be achieved without a substantial increase in a driving voltage.

The hole transport region HTR may further include a charge generating material to increase conductivity in addition to the previously described materials. The charge generating material may be dispersed uniformly or non-uniformly in the hole transport region HTR. The charge generating material may be, for example, a p-dopant. The p-dopant may include at least one among a metal halide compound, a quinone derivative, a metal oxide, and a cyano group-containing compound, but is not limited thereto. For example, the p-dopant may include a metal halide compound such as CuI and RbI, a quinone derivative such as tetracyanoquinodimethane (TCNQ) and 2,3,5,6-tetrafluoro-7,7′8,8-tetracyanoquinodimethane (F4-TCNQ), a metal oxide such as a tungsten oxide and a molybdenum oxide, a cyano group-containing compound such as dipyrazino[2,3-f. 2′,3′-h]quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but an embodiment is not limited thereto.

As previously described, the hole transport region HTR may further include at least one among an emission auxiliary layer EAL and an electron-blocking layer EBL in addition to the hole injection layer HIL, and the hole transport layer HTL. The emission auxiliary layer EAL may compensate for a resonance distance according to the wavelength of light emitted from an emission layer EML and may increase light emission efficiency by controlling a hole charge balance. In addition, the emission auxiliary layer EAL may serve to prevent electrons from being injected into the hole transport region HTR. The material that may be included in the hole transport region HTR may be included in the emission auxiliary layer EAL. The electron-blocking layer EBL is a layer that serves to prevent electrons from being injected into the hole transport region HTR from the electron transport region ETR.

Referring to FIG. 7, in the light-emitting element ED according to an embodiment, the hole transport layer HTL may have a multi-layer structure. When the hole transport layer HTL has a multi-layer structure, at least one layer of the hole transport layer HTL may include the amine compound according to an embodiment. In an embodiment, when the hole transport layer HTL has a multi-layer structure, an uppermost layer of the hole transport layer HTL may include the amine compound according to an embodiment. The uppermost layer of the hole transport layer HTL may be an adjacent layer to the emission layer EML. In an embodiment, when the hole transport layer HTL has a multi-layer structure, a material of the uppermost layer of the hole transport layer HTL may be different from those of the other layers.

In the light-emitting element ED according to an embodiment, the hole transport region HTR may have a two-layer structure of: a hole injection layer HIL disposed on the first electrode EL1; and a first hole transport layer HTL-1 disposed on the hole injection layer HIL and a second hole transport layer HTL-2 adjacent to the bottom of the emission layer EML and disposed on the first hole transport layer HTL-1. The second hole transport layer HTL-2 may include the amine compound according to an embodiment.

Materials for the hole injection layer HIL may include, for example,

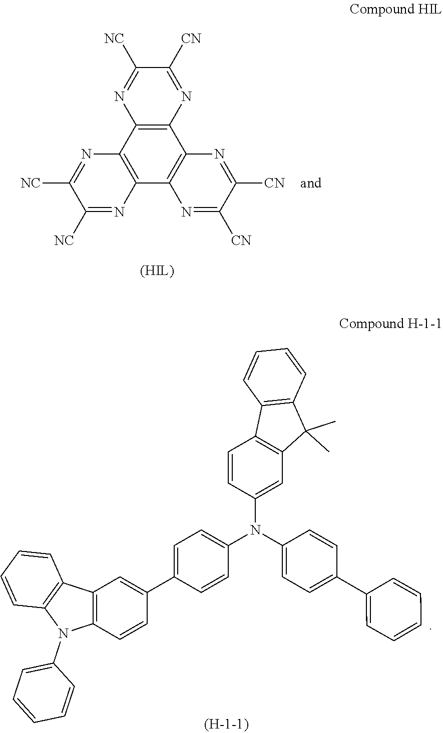 [SMILES: N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21]

Compound HIL and Compound H-1-1 may be deposited at a weight ratio of, for example, about 2:98. The first hole transport layer HTL-1 may include, for example Compound H-1-1. The second hole transport layer HTL-2 may include, for example, the amine compound according to an embodiment.

In the light-emitting element ED according to an embodiment, the emission layer EML is provided on the hole transport region HTR. The emission layer EML may have a thickness of, for example, about 100 Å to about 1,000 Å, or about 100 Å to about 300 Å. The emission layer EML may have a single layer including a single material, a single layer including a plurality of different materials, or a multi-layer structure having a plurality of layers including a plurality of different materials.

In the light-emitting element ED according to an embodiment, the emission layer EML may emit blue light. The light-emitting element ED according to an embodiment includes the previously described amine compound according to an embodiment in the hole transport region HTR and thus may exhibit high efficiency and long lifespan characteristics in a blue light emission region. The light-emitting element ED according to an embodiment includes the previously described amine compound according to an embodiment in the hole transport region HTR, and the emission layer EML may emit blue fluorescence.

In addition, in the light-emitting element ED according to an embodiment, the emission layer EML may emit light of a different wavelength region, other than blue light. The light-emitting element ED according to an embodiment includes the previously described amine compound according to an embodiment in the hole transport region HTR, and thus may exhibit high efficiency and long lifespan characteristics even in emission regions of different wavelengths other than blue light.

The light-emitting element ED according to an embodiment includes the previously described amine compound according to an embodiment in the hole transport region HTR, and the emission layer EML may emit fluorescence. However, an embodiment is not limited thereto.

In the light-emitting element ED according to an embodiment, the emission layer EML may include an anthracene derivative, a pyrene derivative, a fluoranthene derivative, a chrysene derivative, a dihydrobenzanthracene derivative, or a triphenylene derivative. Specifically, the emission layer EML may include an anthracene derivative, or a pyrene derivative.

In the light-emitting element ED according to an embodiment, illustrated in FIG. 5 to FIG. 10, the emission layer EML may include a host and a dopant, and the emission layer EML may include a compound according to Formula E-1. The compound according to Formula E-1 may be used as a fluorescent host material.

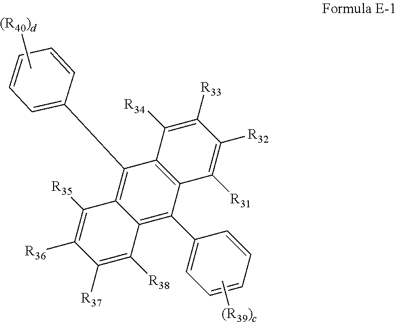

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may be each independently an integer of 0 to 5.

Formula E-1 may have a structure according to any one of Compound E1 to Compound E19.

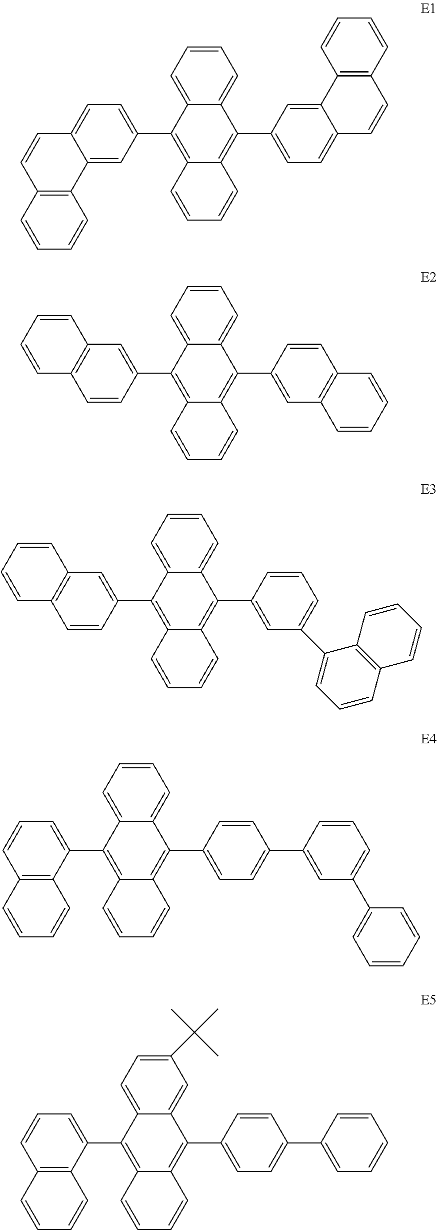

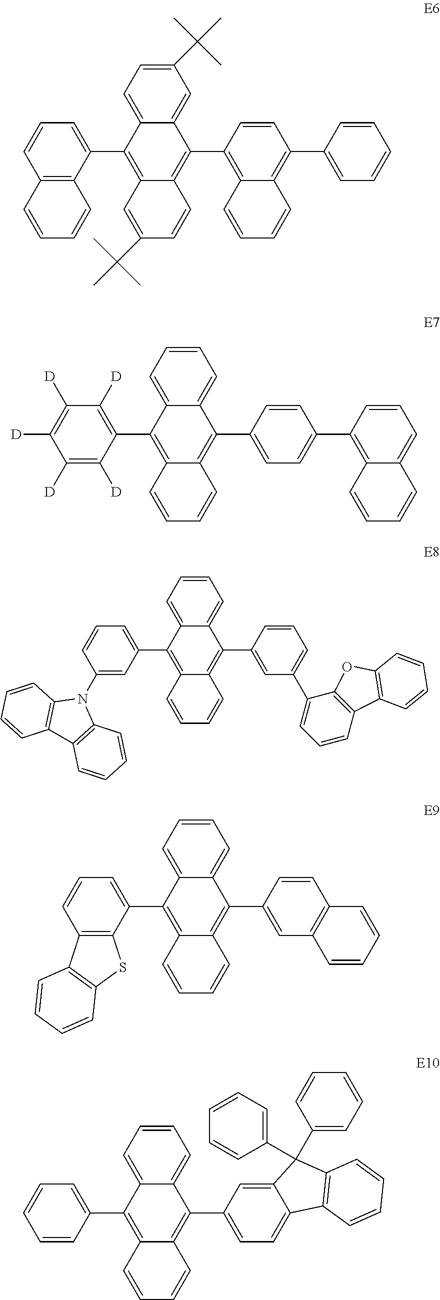

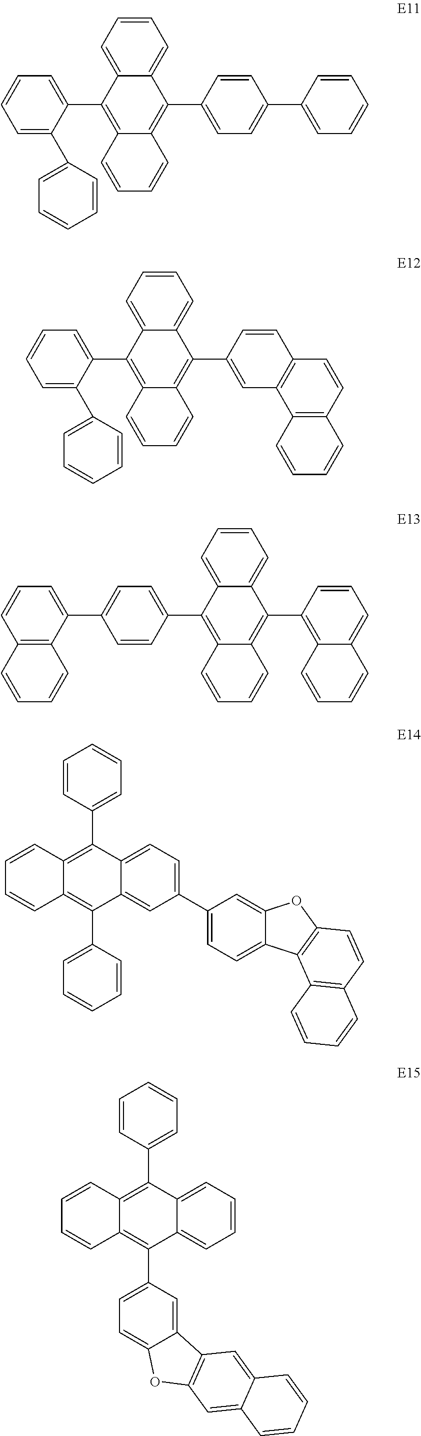

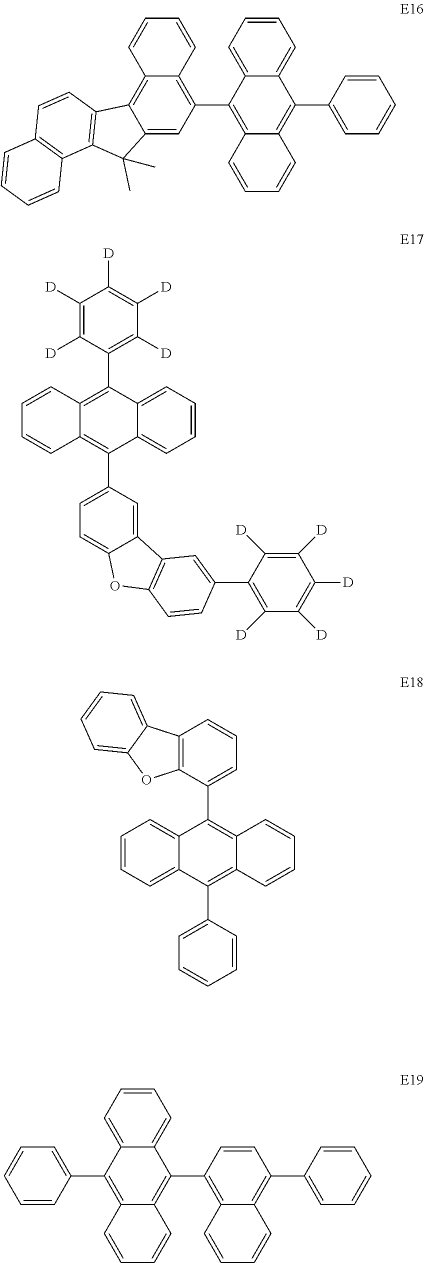

In an embodiment, the emission layer EML may further include a compound according to Formula E-2a or Formula E-2b. The compound according to Formula E-2a or Formula E-2b may be used as a host material for a phosphorescent emission layer.

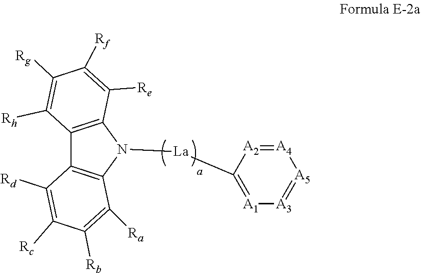

In Formula E-2a, a may be an integer of 0 to 10, Lₐ may be a direct linkage, a substituted or unsubstituted arylene group having 6 to 30 ring-forming carbon atoms, or a substituted or unsubstituted heteroarylene group having 2 to 30 ring-forming carbon atoms. If a is an integer of 2 or greater, a plurality of Lₐ may be each independently a substituted or unsubstituted arylene group having 6 to 30 ring-forming carbon atoms, or a substituted or unsubstituted heteroarylene group having 2 to 30 ring-forming carbon atoms.

In addition, in Formula E-2a, A₁ to A₅ may be each independently N, or CRᵢ. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. Rₐ to Rᵢ may be bonded to an adjacent group to form a hydrocarbon ring or a heterocycle containing N, O, S, or the like as a ring-forming atom.

In Formula E-2a, two or three selected from among A₁ to A₅ may be N, and the rest may be CRᵢ.

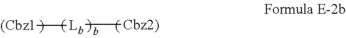

In Formula E-2b, Cbz1 and Cbz2 may be each independently an unsubstituted carbazole group, or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. b is an integer of 0 to 10, and when b is an integer of 2 or greater, a plurality of L_{b} may be each independently a substituted or unsubstituted arylene group having 6 to 30 ring-forming carbon atoms, or a substituted or unsubstituted heteroarylene group having 2 to 30 ring-forming carbon atoms.

The compound according to Formula E-2a, or Formula E-2b may have a structure according to any one compound in Compound Group E-2. However, the compounds listed in Compound Group E-2 are examples, and the compound according to Formula E-2a, or Formula E-2b is not limited to the compounds listed in Compound Group E-2.

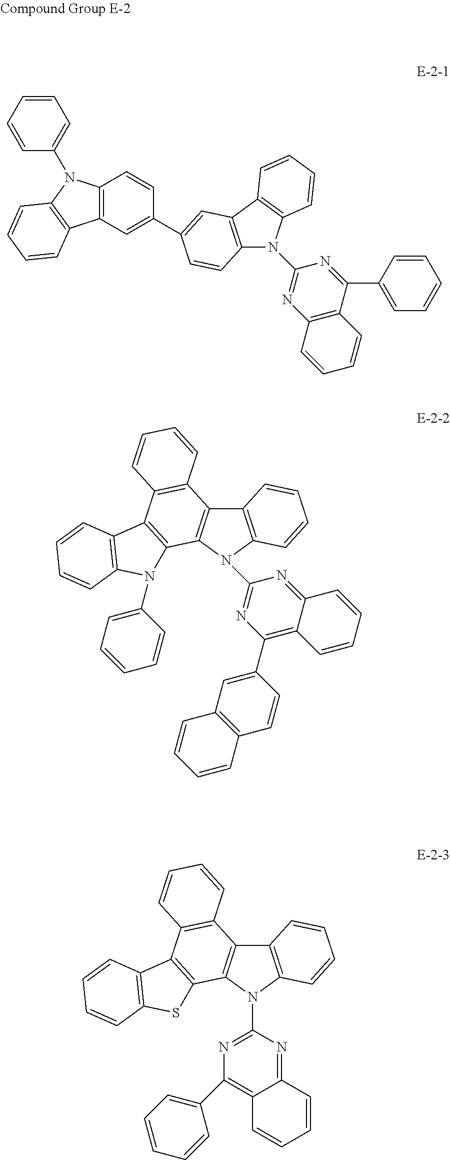 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

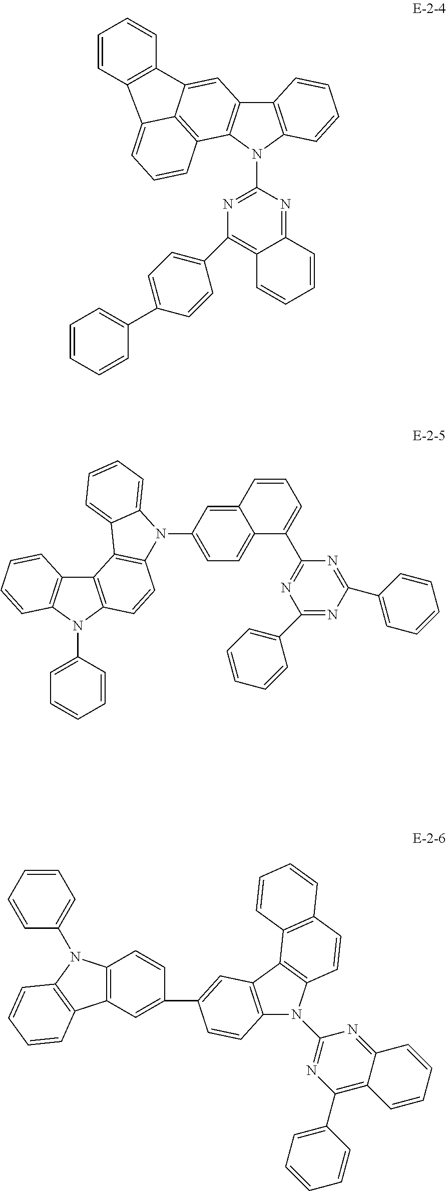 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

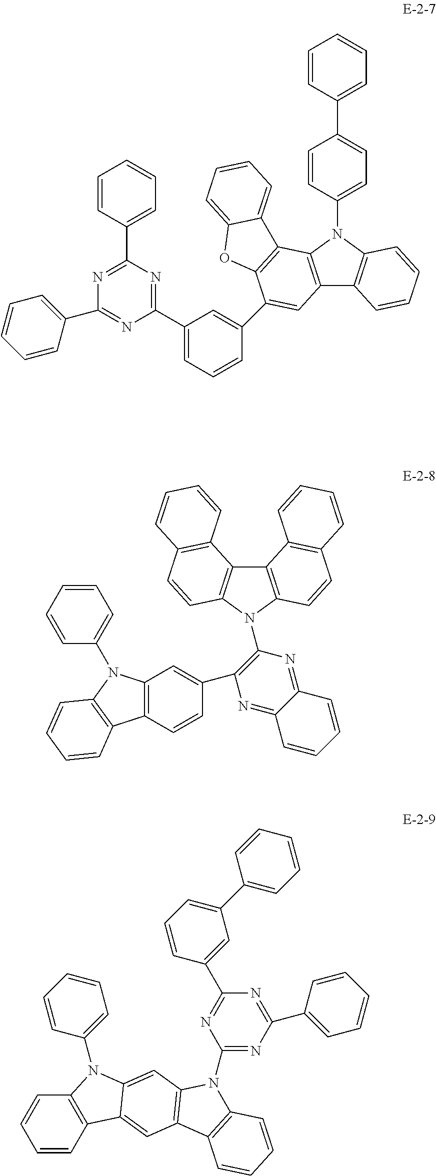 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

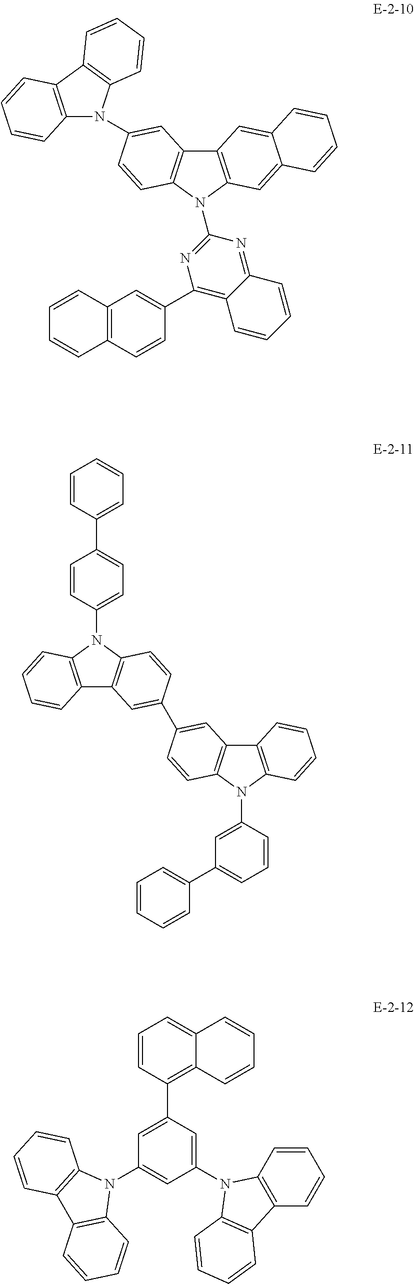 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

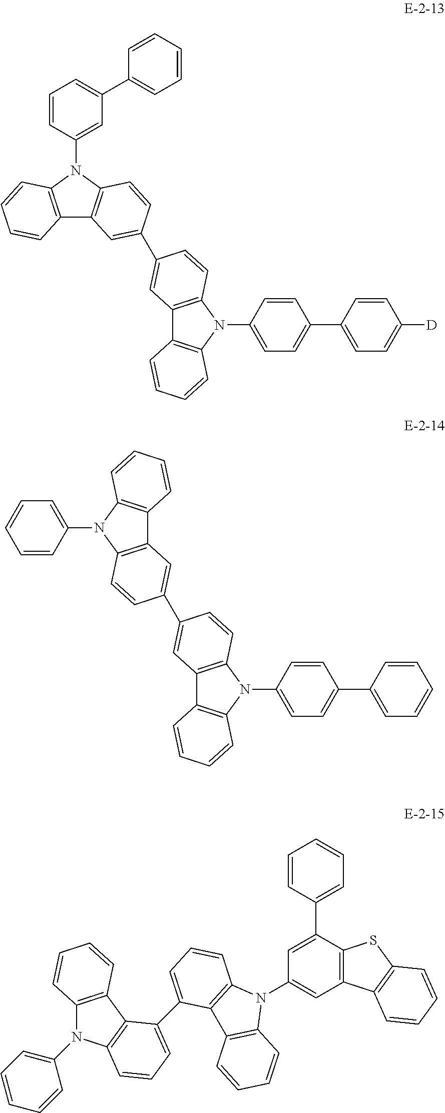 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

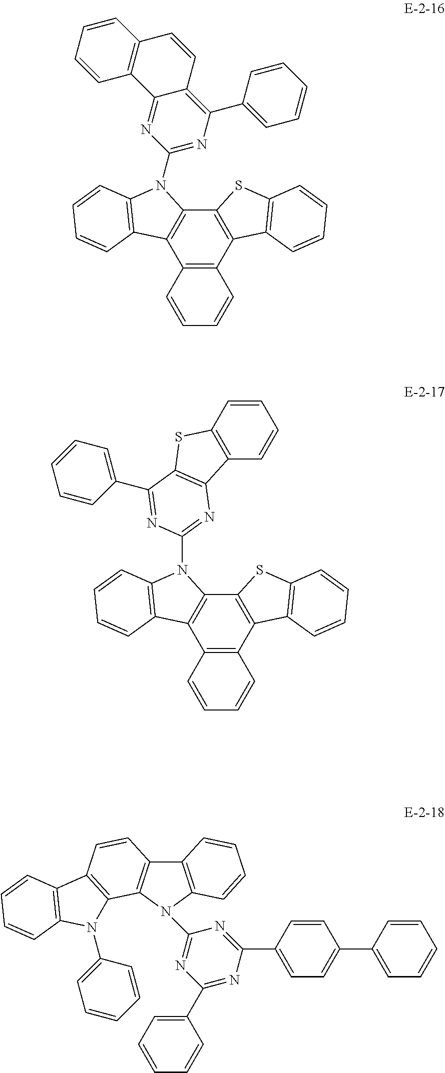 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

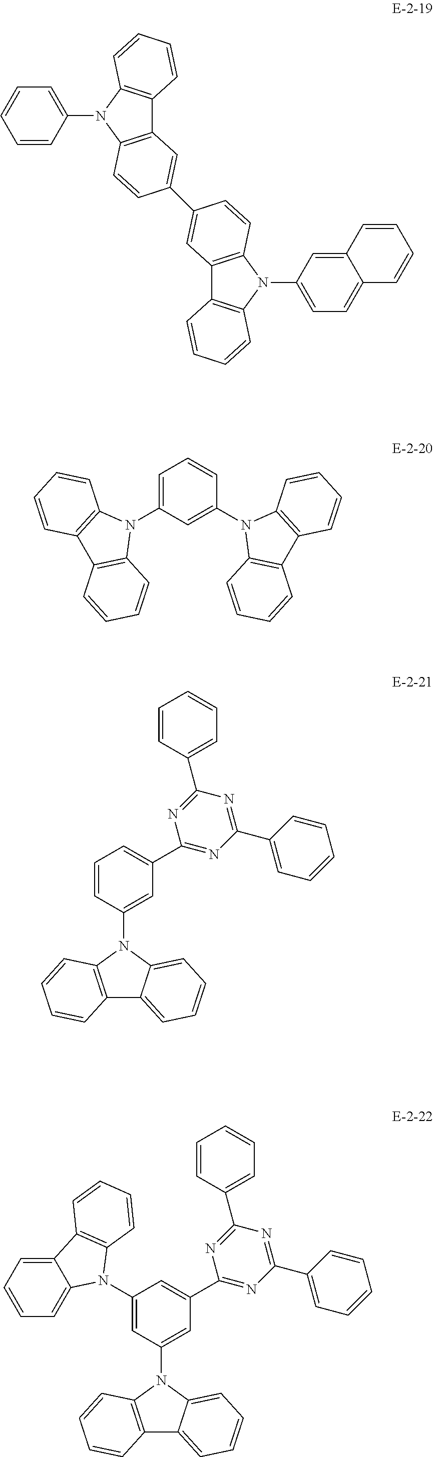 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

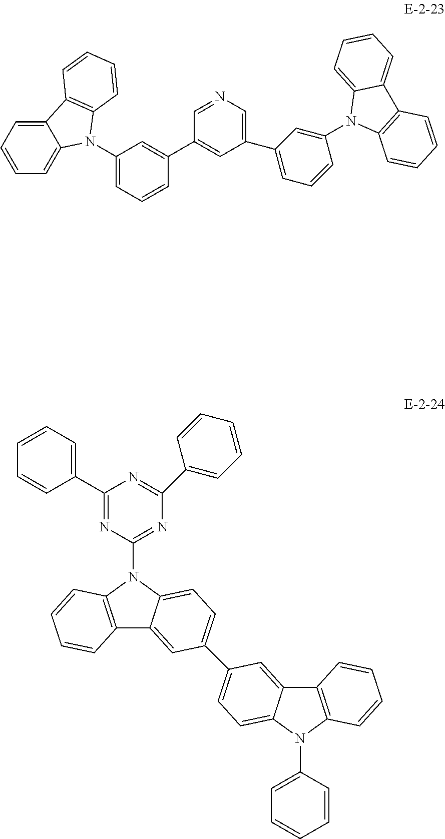 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a typical material that is known in the related art as a host material. For example, the emission layer EML may include, as a host material, at least one among bis(4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl]ether oxide (DPEPO), 4,4′-bis(N-carbazolyl)-1,1′-biphenyl) (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA) and 1,3,5-tris(1-phenyl-1H-benzo[d]imidazole-2-yl)benzene (TPBi). However, an embodiment is not limited thereto, and, for example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), and the like may be used as the host material.

The emission layer EML may include a compound according to Formula M-a, or Formula M-b. The compound according to Formula M-a, or Formula M-b may be used as a phosphorescent dopant material.

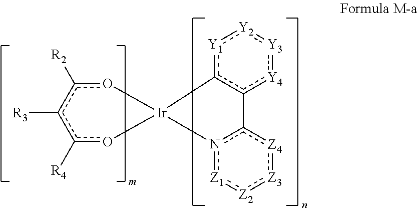

In Formula M-a, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. In Formula M-a, m is 0 or 1, and n is 2 or 3. If m is 0, n is 3, and when m is 1, n is 2 in Formula M-a.

The compound according to Formula M-a may be according to any one compound of Compounds M-a1 to M-a25. However, Compounds M-a1 to M-a25 are examples, and the compound according to Formula M-a is not limited to the compounds designated as Compounds M-a1 to M-a25.

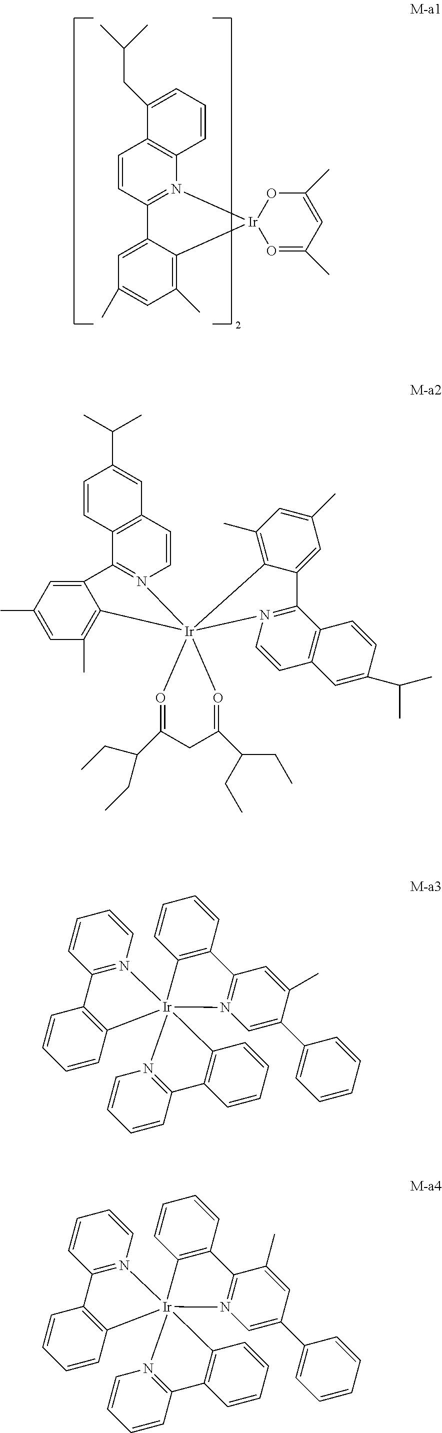

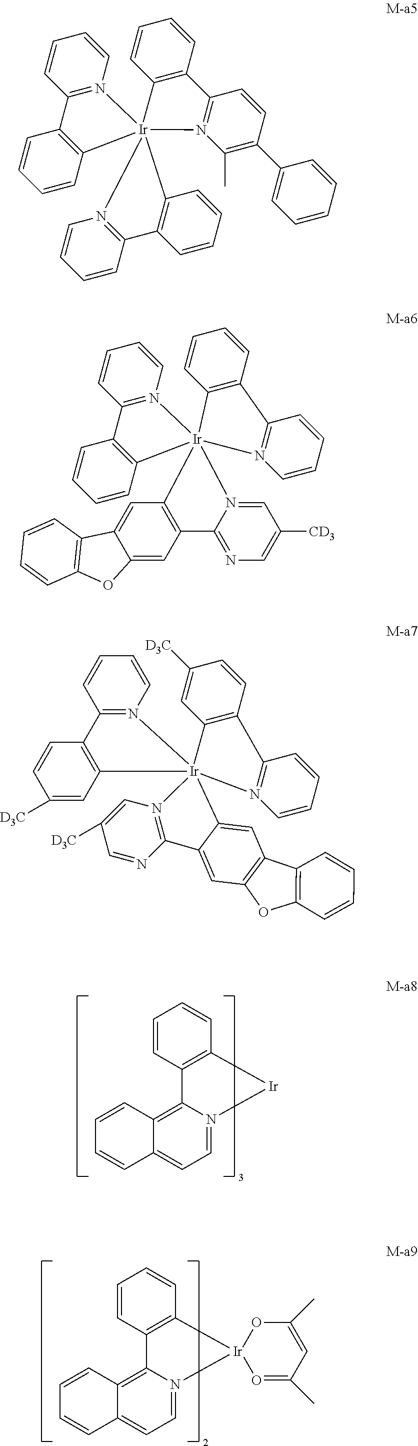

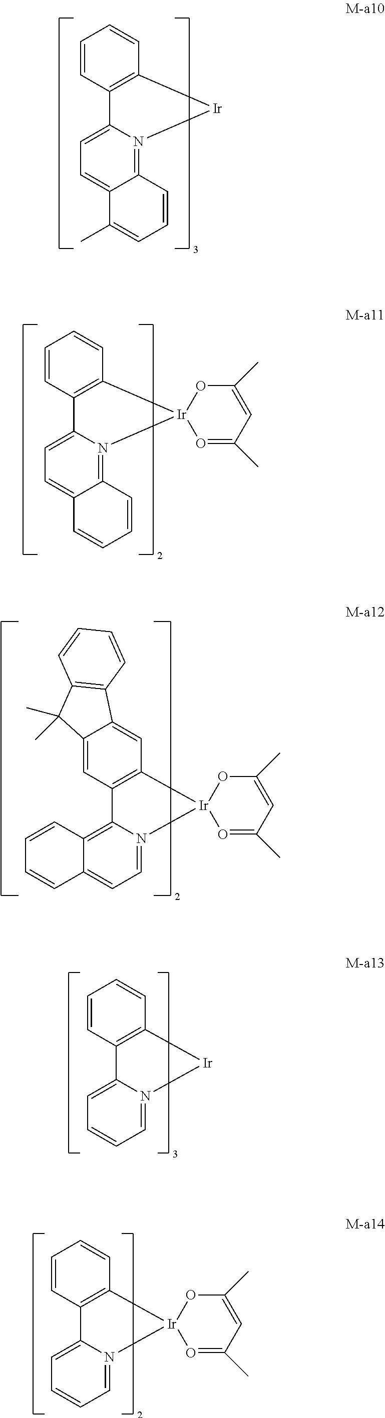

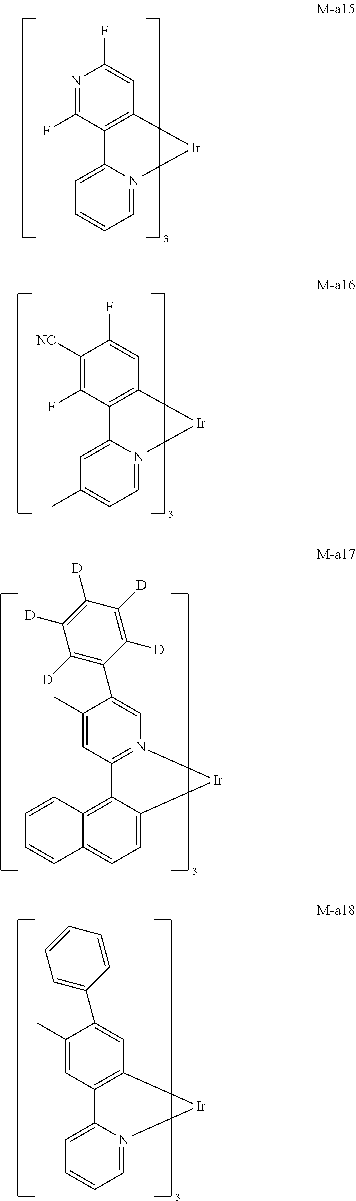

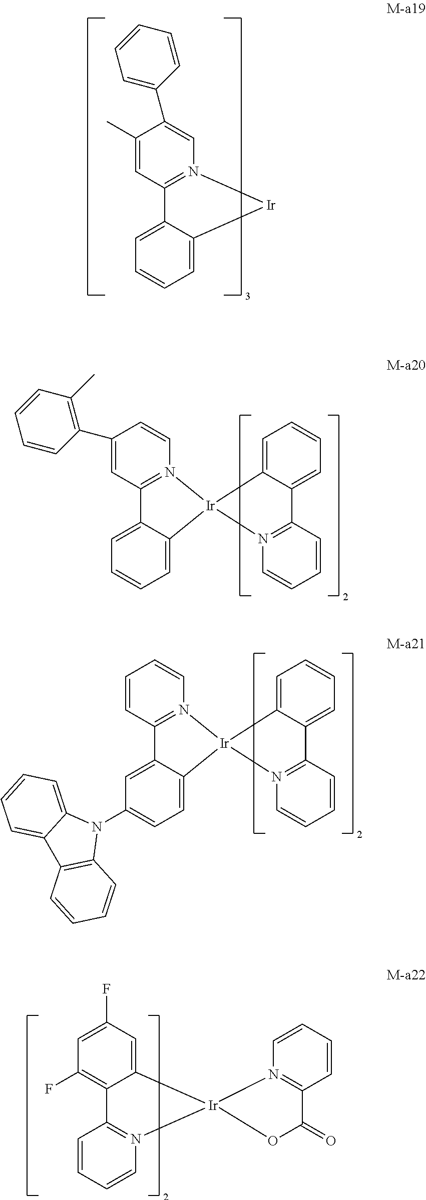

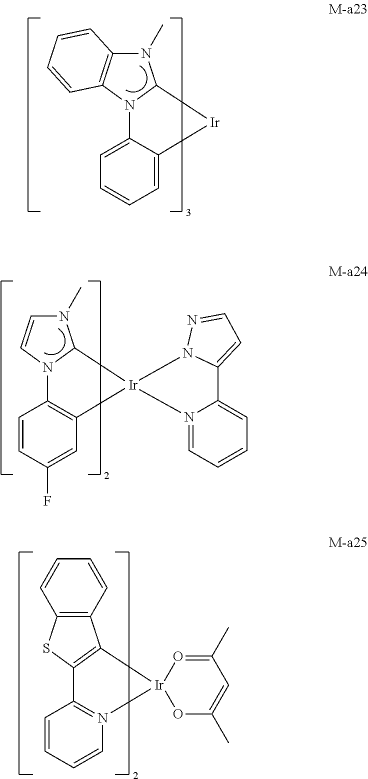

Compound M-a1 and Compound M-a2 may be used as red dopant materials, and Compound M-a3 to Compound M-a5 may be used as green dopant materials.

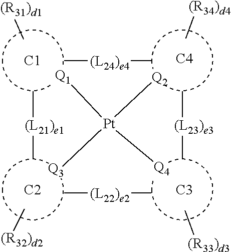

In Formula M-b, Q₁ to Q₄ are each independently C or N, and C₁ to C₄ are each independently a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ are each independently a direct linkage,

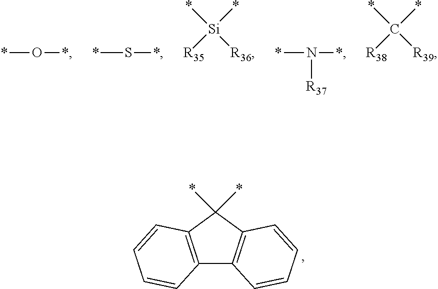

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring, and d1 to d4 are each independently an integer of 0 to 4.

The compound according to Formula M-b may be used as a blue phosphorescent dopant or a green phosphorescent dopant.

The compound according to Formula M-b may have a structure according to any one of the following compounds. However, the following compounds are examples, and the compound according to Formula M-b is not limited to the following compounds.

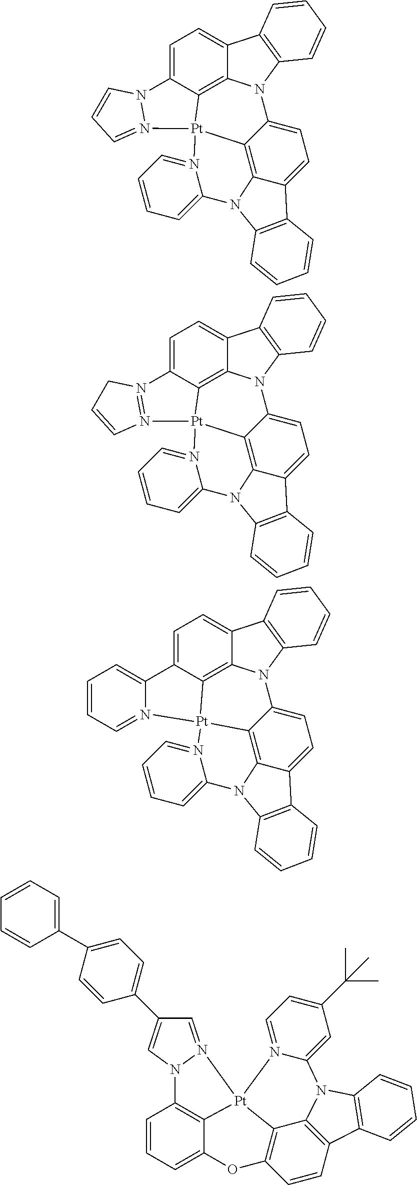

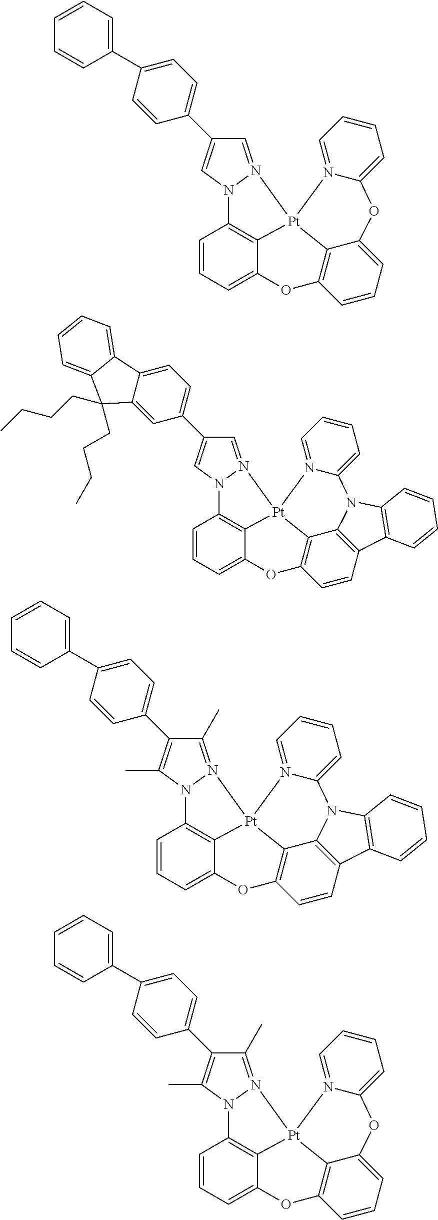

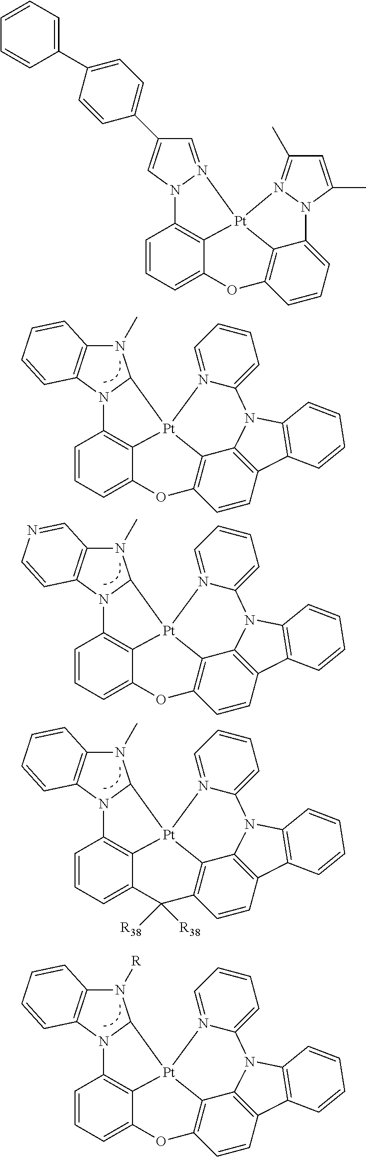

In the compounds, R, R₃₈, and R₃₉ may each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound according to any one of Formula F-a to Formula F-c. The compound according to any one of Formula F-a to Formula F-c may be used as a fluorescent dopant material.

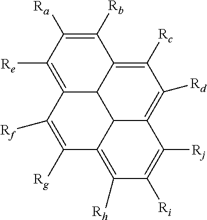

In Formula F-a, two selected from among Rₐ to Rⱼ may be each independently substituted with *—NAr₁Ar₂. The others among Rₐ to Rⱼ, which are not substituted with *—NAr₁Ar₂ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *—NAr₁Ar₂, Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. For example, at least one among Ar₁ and Ar₂ may be a heteroaryl group containing O or S as a ring-forming atom.

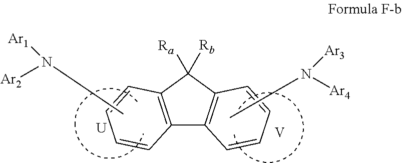

In Formula F-b, Rₐ and R_{b} may be each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. Ar₁ to Ar₄ may be each independently a substituted or unsubstituted aryl group having 6 to 30 ring-forming carbon atoms, or a substituted or unsubstituted heteroaryl group having 2 to 30 ring-forming carbon atoms.

In Formula F-b, U and V may be each independently a substituted or unsubstituted hydrocarbon ring having 5 to 30 ring-forming carbon atoms, or a substituted or unsubstituted heterocycle having 2 to 30 ring-forming carbon atoms. At least one among Ar₁ and Ar₄ may be a heteroaryl group containing O or S as a ring-forming atom.

The number of rings designated by U and V in Formula F-b may be each independently 0 or 1. For example, in Formula F-b, when the number of U or V is 1, one ring forms a fused ring at a portion designated by U or V, and when the number of U or V is 0, it means that there is no ring designated by U or V. Specifically, when the number of U is 0 and the number of V is 1, or when the number of U is 1 and the number of V is 0, a fused ring having a fluorene core in Formula F-b may be a cyclic compound having four rings. In addition, when both the numbers of U and V are 0, the fused ring in Formula F-b may be a cyclic compound having three rings. In addition, when both the numbers of U and V are 1, a fused ring having a fluorene core in Formula F-b may be a cyclic compound having five rings.

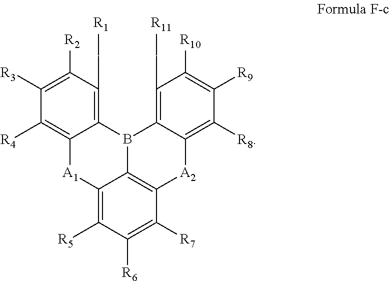

In Formula F-c, A₁ and A₂ may be each independently O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or bonded to an adjacent group to form a ring.

In Formula F-c, A₁ and A₂ may be each independently bonded to substituents of an adjacent ring to form a fused ring. For example, when A₁ and A₂ are each independently NRₘ, A₁ may be bonded to R₄ or R₅ to form a ring. In addition, A₂ may be bonded to R₇ or R₈ to form a ring.

In an embodiment, the emission layer EML may further include, as a known dopant material, a styryl derivative (e.g., 1, 4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi)), 4,4′-bis[2-(4-(N,N-diphenylamino)phenyl)vinyl]biphenyl (DPAVBi), perylene and a derivative thereof (e.g., 2, 5, 8, 11-tetra-t-butylperylene (TBP)), pyrene and a derivative thereof (e.g., 1, 1-dipyrene, 1, 4-dipyrenylbenzene, 1, 4-bis(N, N-diphenylamino)pyrene), etc.

When the light-emitting element is a phosphorescent light-emitting element, the emission layer EML may further include a known dopant material. For example, a metal complex containing iridium (Ir), platinum (Pt), osmium (Os), gold (Au), titanium (Ti), zirconium (Zr), hafnium (Hf), europium (Eu), terbium (Tb), or thulium (Tm) may be used. Specifically, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as a phosphorescent dopant. However, an embodiment is not limited thereto.

In the light-emitting element ED according to an embodiment, the emission layer EML may be a delayed fluorescent emission layer including a host and a dopant. More specifically, the emission layer EML may emit thermally activated delayed fluorescence (TADF). In the light-emitting element ED according to an embodiment, the emission layer EML may include a known thermally activated delayed fluorescent dopant.

In addition, in an embodiment, the emission layer EML of the light-emitting element ED may include a plurality of host materials, a thermally activated delayed fluorescent dopant, a phosphorescent sensitizer, etc.

The emission layer EML may include a quantum dot material. A core of the quantum dot may be selected from a group II-VI compound, a group I-II-VI compound, a group II-IV-VI compound, a group I-II-IV-VI compound, a group II-IV-V compound, a group III-VI compound, a group I-III-VI compound, a group III-V compound, a group III-II-V compound, a group IV-VI compound, a group IV element, a group IV compound, and a combination thereof.

The group II-VI compound may be selected from the group consisting of: a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof.

The group III-VI compound may include: a binary compound such as In₂S₃, and In₂Se₃; a quaternary compound such as InGaS₃, and InGaSe₃; or any combination thereof.

The group I-III-VI compound may be selected from a ternary compound selected from the group consisting of AgInS, AgInS₂, CuInS, CuInS₂, AgGaS₂, CuGaS₂ CuGaO₂, AgGaO₂, AgAlO₂, and a mixture thereof, or a quaternary compound such as AgInGaS₂, and CuInGaS₂.

The group III-V compound may be selected from the group consisting of: a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. The group III-V compound may further include a group II metal. For example, InZnP and the like may be selected as a group III-II-V compound.

The group IV-VI compound may be selected from the group consisting of: a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The group IV element may be selected from the group consisting of Si, Ge, and a mixture thereof.

The group IV compound may be a binary compound selected from the group consisting of SiC, SiGe, and a mixture thereof.

Each element included in a multi-element compound such as the binary compound, the ternary compound, and the quaternary compound may be present in a particle at a uniform concentration or non-uniform concentration. That is, the chemical formulas (e.g., SnS, SnSe, PbTe, AgInGaS₂, etc.) indicate the types of elements included the compound, and element ratios in the compound may vary. For example, AgInGaS₂ may indicate AgInₓGa₁₋ₓS₂ (x is a real number of 0 to 1).

The quantum dot may have a core-shell dual structure or a single structure in which a concentration of each element included in the corresponding quantum dot is uniform. For example, a material included in the core may be different from a material included in the shell.

The shell of the quantum dot may serve as a protection layer for preventing the core from being chemically modified to maintain semiconductor properties and/or as a charging layer for imparting electrophoretic properties to the quantum dot. The shell may have a single layer or multilayer. An interface between the core and the shell may have a concentration gradient in which a concentration of an element present in the shell decreases toward the core.

Examples of the shell of the quantum dot may include an oxide of metal or non-metal, a semiconductor compound, a combination thereof, etc. For example, the oxide of metal or non-metal may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and NiO, or a ternary compound such as MgAl₂O₄, CoFe₂O₄, NiFe₂O₄, and CoMn₂O₄, but the inventive concept is not limited thereto.

In addition, examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the inventive concept is not limited thereto.

Each element included in a multi-element compound such as the binary compound and the ternary compound may be present in particle at a uniform concentration or non-uniform concentration. That is, the chemical formulas (e.g., CdSe, ZnTeS, GaAs, CoFe₂O₄, etc.) indicate types of elements included in the compound, and element ratios in the compound may vary.

The quantum dot may have a full width of half maximum (FWHM) of about 45 nm or less, preferably about 40 nm or less, and more preferably about 30 nm or less in an emission wavelength spectrum, and color purity and color reproducibility may be improved in this range. In addition, light emitted through the quantum dot is emitted in all directions, and thus an optical viewing angle may be improved.

In addition, the shape of the quantum dot is commonly available in the art and is not particularly limited. However, more specifically, the quantum dot may adopt a spherical, pyramidal, multi-arm-shaped or cubic nanoparticle, a nanotube, a nanowire, a nanofiber, a nanoplate particle, etc.

An energy band gap may be controlled by controlling the size of the quantum dot or by controlling the element ratio in the quantum dot compound, and thus light with various wavelength bands may be emitted from the quantum dot emission layer. Therefore, by using the previously described quantum dot (using quantum dots in different sizes or with different element ratios in the quantum dot compound), a light-emitting element that emits light with various wavelengths may be achieved. Specifically, the size of the quantum dot and the element ratio in the quantum dot compound may be selected such that red, green, and/or blue light is emitted. In addition, the quantum dots may be configured to emit white light by combining various colors of light.

In the light-emitting element ED according to the embodiment illustrated in FIG. 5 to FIG. 10, an electron transport region ETR is provided on the emission layer EML. The electron transport region ETR may include at least one among a hole-blocking layer HBL, an electron transport layer ETL, and an electron injection layer EIL, but an embodiment is not limited thereto.

The electron transport region ETR may have a single layer including a single material, a single layer including a plurality of different materials, or a multi-layer structure having a plurality of layers including a plurality of different materials.

For example, the electron transport region ETR may have a single-layer structure of an electron injection layer EIL or an electron transport layer ETL, or a single-layer structure including an electron injection material and an electron transport material. In addition, the electron transport region ETR may have a single-layer structure including a plurality of different materials, or a structure of electron transport layer ETL/electron injection layer EIL, or hole-blocking layer HBL/electron transport layer ETL/electron injection layer EIL which are stacked from the emission layer EML, but an embodiment is not limited thereto. A thickness of the electron transport region ETR may be, for example, about 1,000 Å to about 1,500 Å.

The electron transport region ETR may be formed by using various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound according to Formula ET-2.

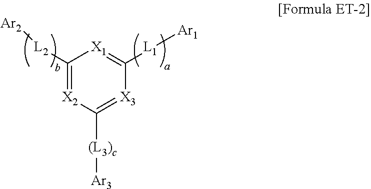

In Formula ET-2, at least one among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may be each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may be each independently an integer of 0 to 10. In Formula ET-2, L₁ to L₃ may be each independently a direct linkage, a substituted or unsubstituted arylene group having 6 to 30 ring-forming carbon atoms, or a substituted or unsubstituted heteroarylene group having 2 to 30 ring-forming carbon atoms. If a to c are each independently an integer of 2 or greater, L₁ to L₃ may be each independently a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, an embodiment is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), berylliumbis(benzoquinolin-10-olate) (Bebg₂), 9,10-di(naphthalene-2-yl)anthracene (ADN), 1,3-bis[3,5-di(pyridin-3-yl)phenyl]benzene (BmPyPhB), and a mixture thereof.

The electron transport region ETR may include at least one among Compounds ET1 to ET36.

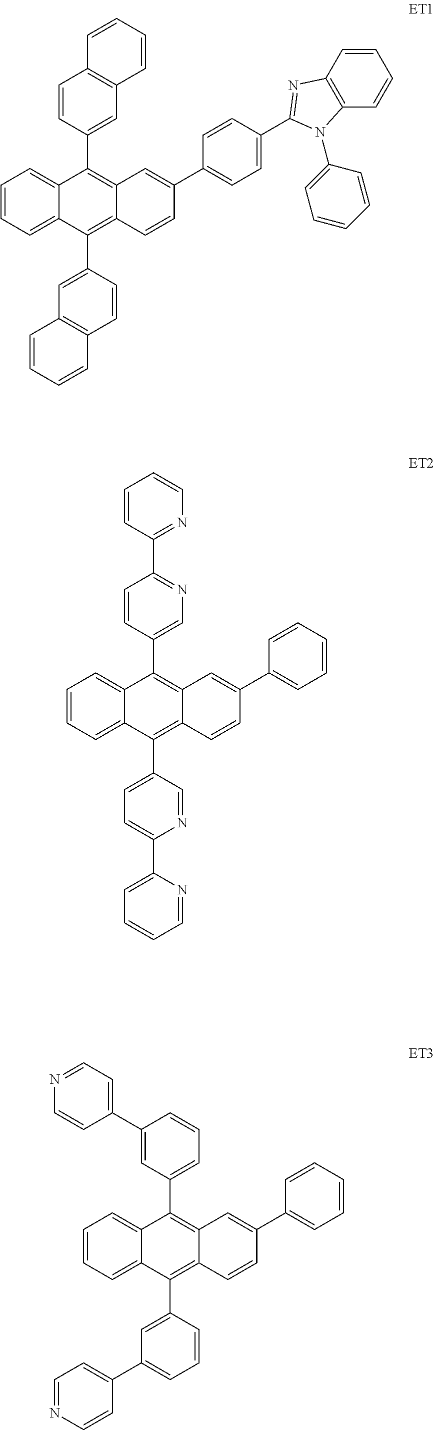 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

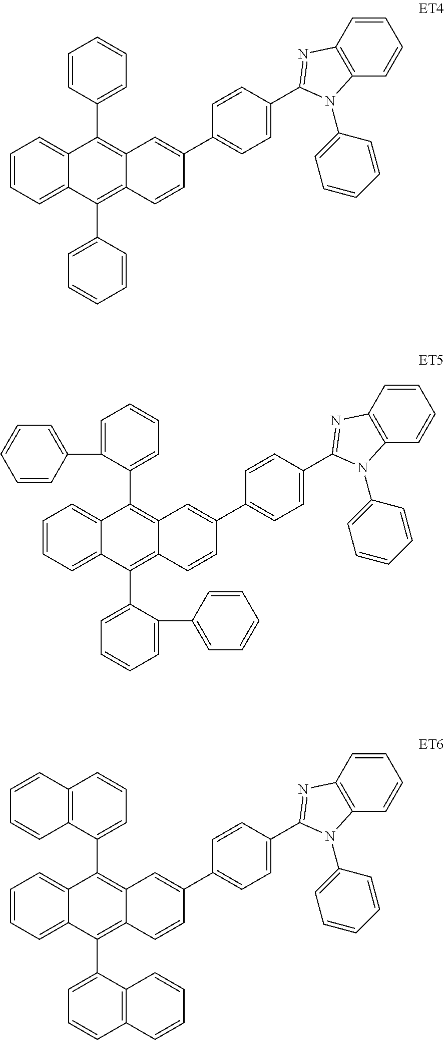 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

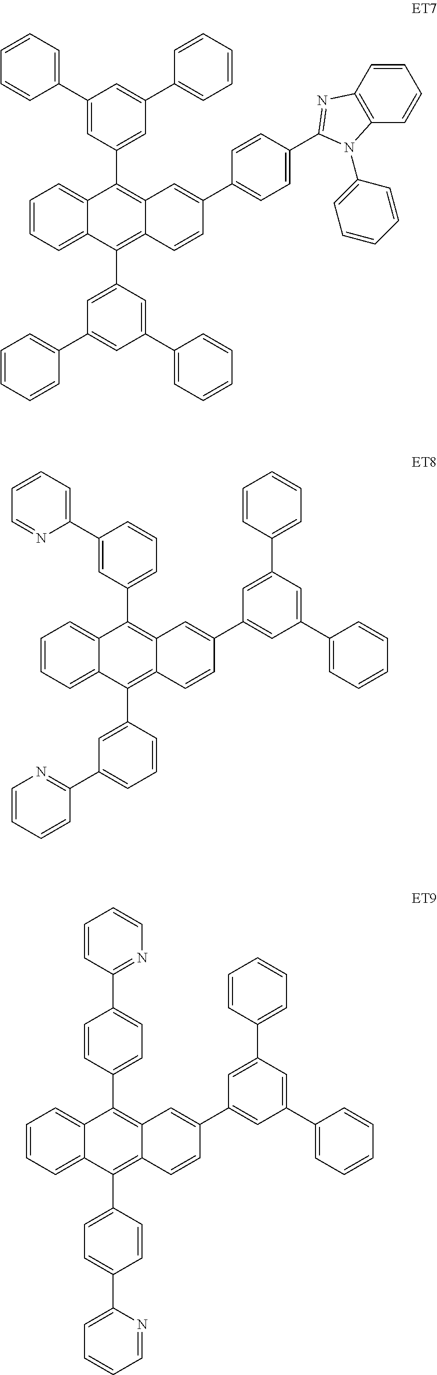 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

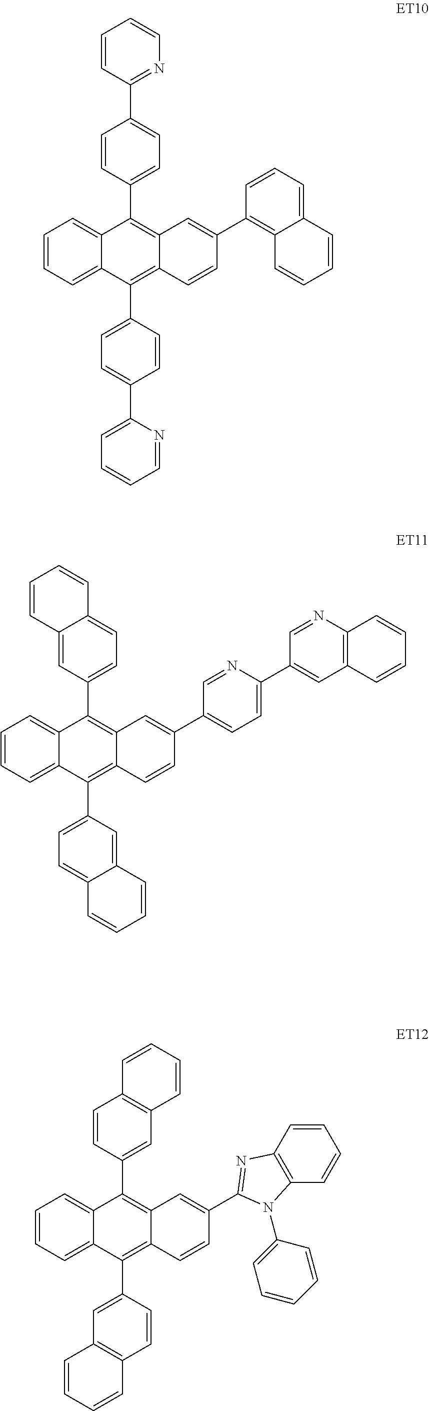 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

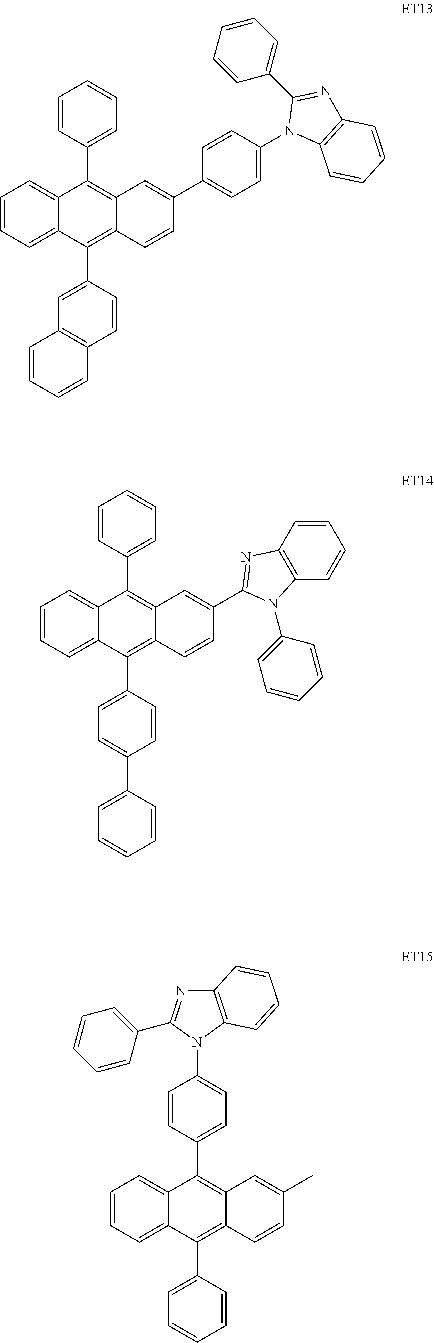 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

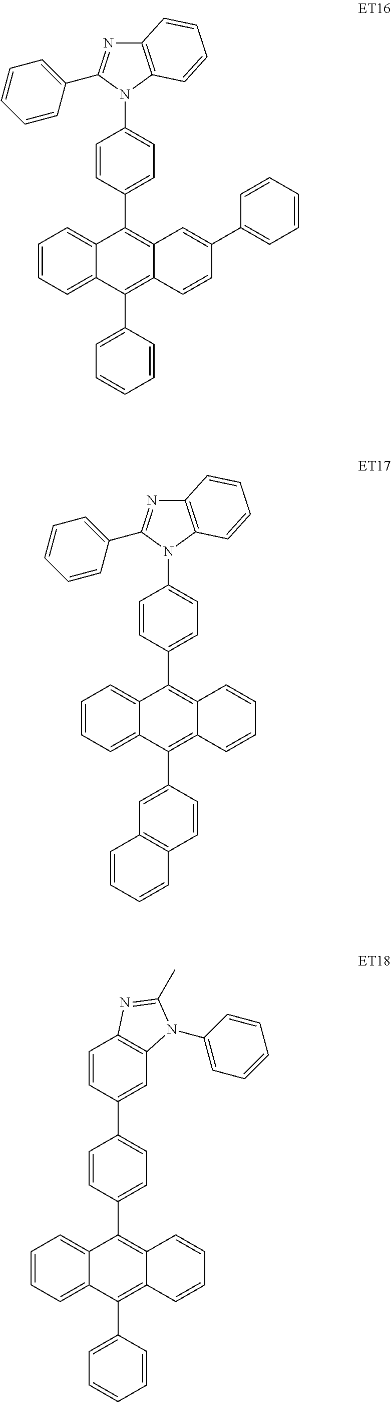 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

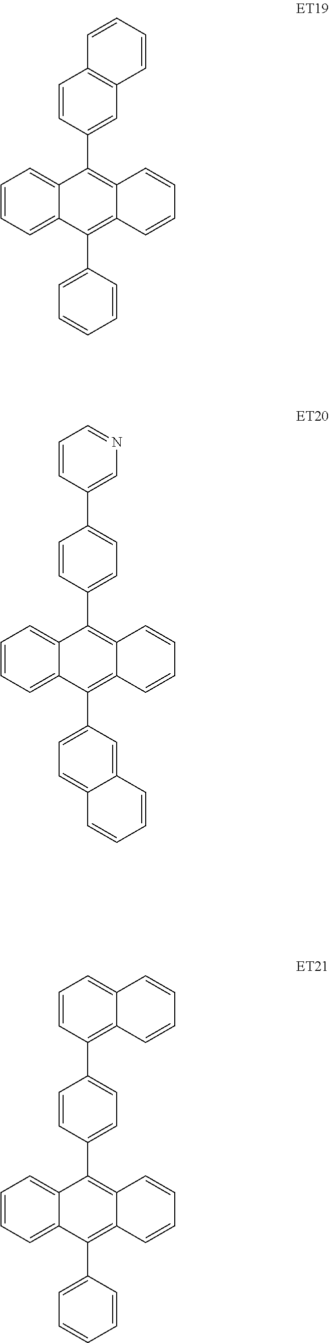

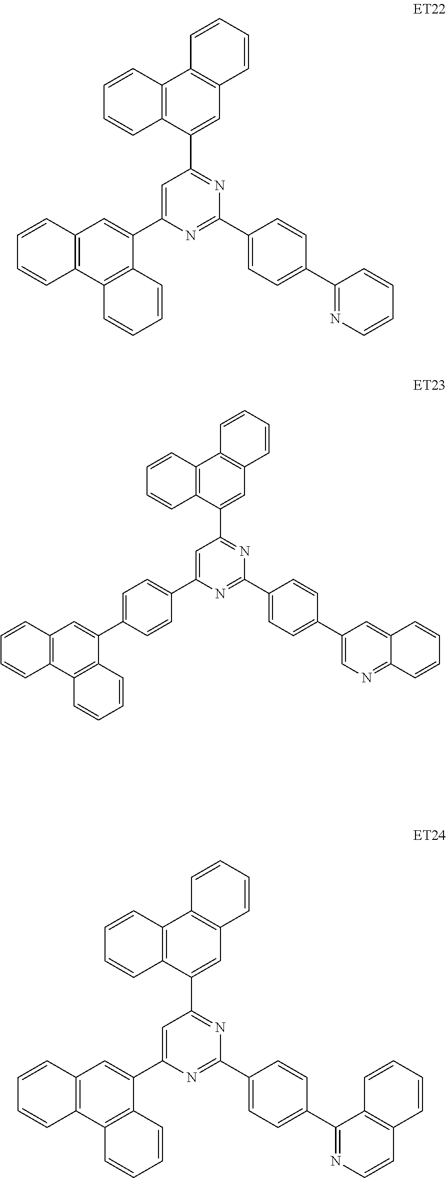 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

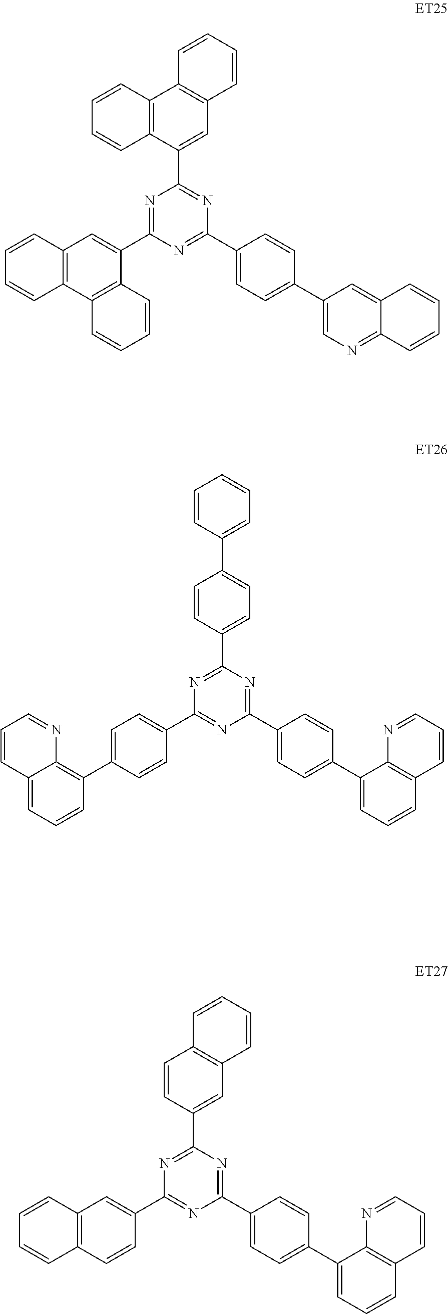 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

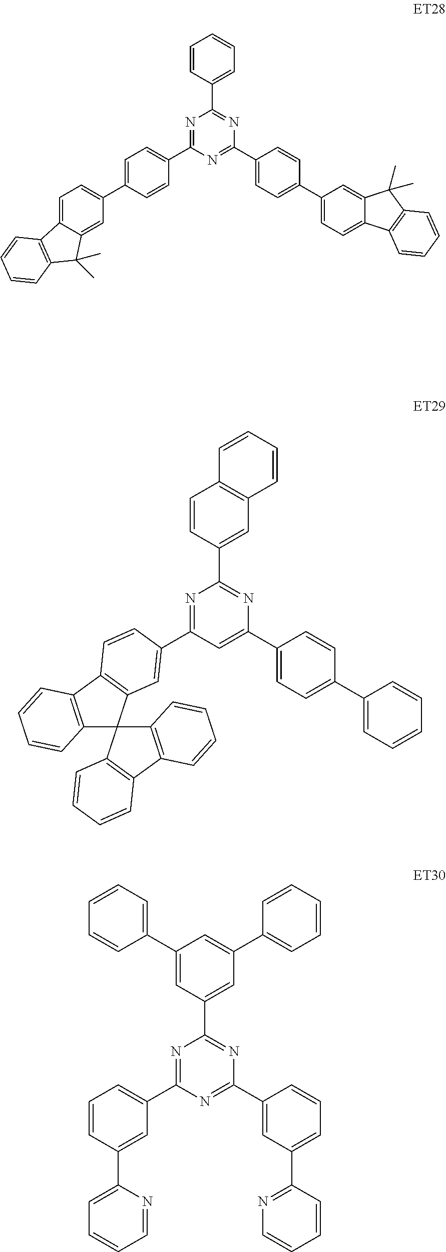 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

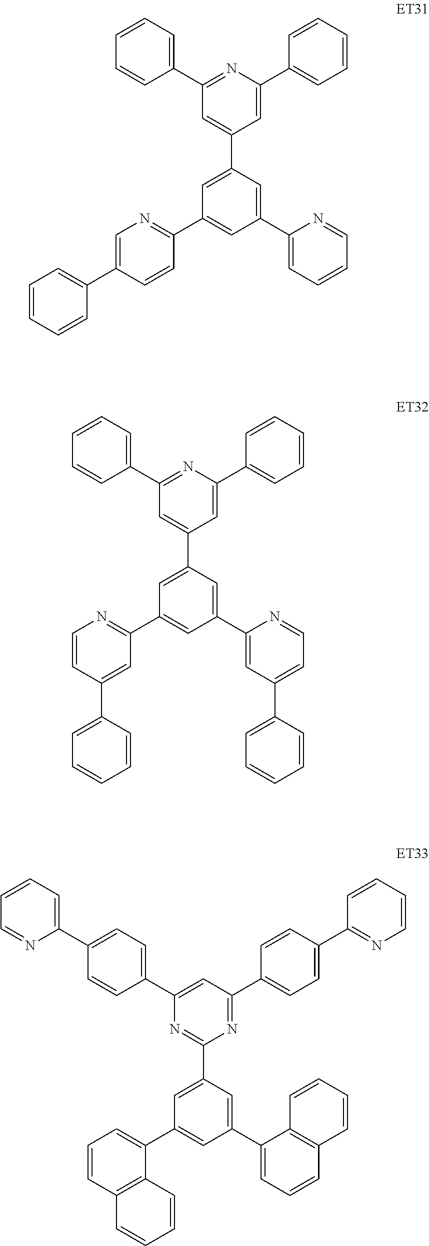 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

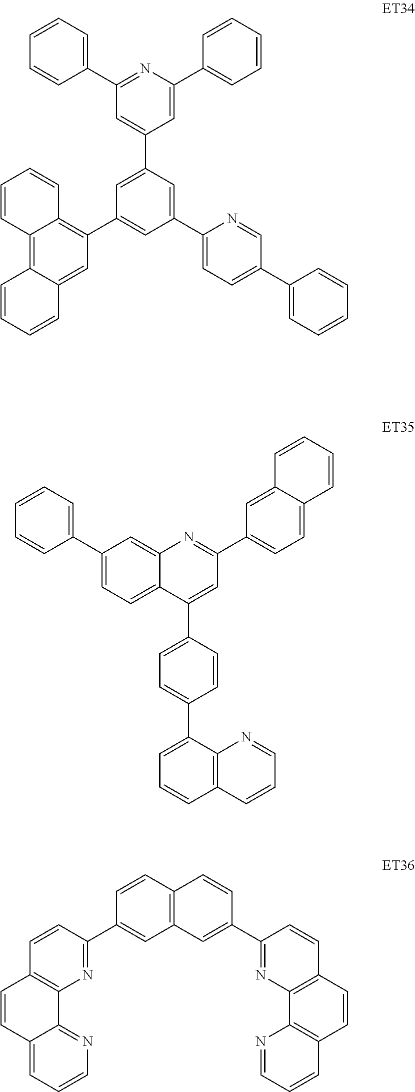 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In addition, the electron transport region ETR may include a metal halide such as LiF, NaCl, CsF, RbCl, RbI, CuI and KI, a lanthanide metal such as Yb, or a co-depositing material of the metal halide and the lanthanide metal. For example, the electron transport region ETR may include KI:Yb, RbI:Yb, LiF:Yb, etc., as the co-depositing material. A metal oxide such as Li₂O and BaO, 8-hydroxy-lithium quinolate (Liq), or the like may be used for the electron transport region ETR. However, an embodiment is not limited thereto. The electron transport region ETR also may include a mixture material of an electron transport material and an organo metal salt having insulating properties. The organo metal salt may have an energy band gap of about 4 eV or more. Specifically, the organo metal salt may include, for example, a metal acetate, a metal benzoate, a metal acetoacetate, a metal acetylacetonate, or a metal stearate.

The electron transport region ETR may further include at least one among 2,9-dimethyl-4,7-diphenyl-1,10-phenanthroline (BCP), diphenyl(4-(triphenylsilyl)phenyl)phosphine oxide (TSPO1) and 4,7-diphenyl-1,10-phenanthroline (Bphen) in addition to the above-described materials. However, an embodiment is not limited thereto.

The electron transport region ETR may include the compounds of the electron transport region in at least one among the electron injection layer EIL, the electron transport layer ETL, and the hole-blocking layer HBL.

When the electron transport region ETR includes the electron transport layer ETL, the thickness of the electron transport layer ETL may be about 100 Å to about 1,000 Å, for example, about 150 Å to about 500 Å. If the thickness of the electron transport layer ETL falls within the above-described range, satisfactory electron transport properties may be obtained without a substantial increase in a driving voltage.

When the electron transport region ETR includes the electron injection layer EIL, the thickness of the electron injection layer EIL may be about 1 Å to about 100 Å, and about 3 Å to about 90 Å. If the thickness of the electron injection layer EIL falls within the above-described range, satisfactory electron injection properties may be obtained without a substantial increase in a driving voltage.

A second electrode EL2 is provided on the electron transport region ETR. The second electrode EL2 may be a common electrode. The second electrode EL2 may be a cathode or an anode, but an embodiment is not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode or a reflective electrode. When the second electrode EL2 is the transmissive electrode, the second electrode EL2 may include a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound including the same, or a mixture thereof (for example, AgMg, AgYb, or MgAg). Alternatively, the second electrode EL2 may have a multi-layer structure including a reflective layer or a transflective layer, each including the above-described materials and a transparent conductive layer including indium tin oxide (ITO), indium zinc oxide (IZO), zinc oxide (ZnO), indium tin zinc oxide (ITZO), etc. For example, the second electrode EL2 may include the above-described metal material, a combination of two or more metal materials selected from the above-described metal materials, or oxides of the above-described metal materials.

Although not illustrated, the second electrode EL2 may be connected with an auxiliary electrode. If the second electrode EL2 is connected with the auxiliary electrode, the resistance of the second electrode EL2 may decrease.

A capping layer CPL may be further disposed on the second electrode EL2 in the light-emitting element ED according to an embodiment. The capping layer CPL may include a multilayer or a single layer.

In an embodiment, the capping layer CPL may be an organic layer or an inorganic layer. For example, when the capping layer CPL includes an inorganic material, the inorganic material may include: an alkali metal compound such as LiF; an alkali earth metal compound such as MgF₂; SiON; SiN_{X}; SiOy; etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol sol-9-yl) triphenylamine (TCTA), etc., or includes an epoxy resin, or acrylate such as methacrylate. However, an embodiment is not limited thereto, and the capping layer CPL may include at least one among Compounds P1 to P5.

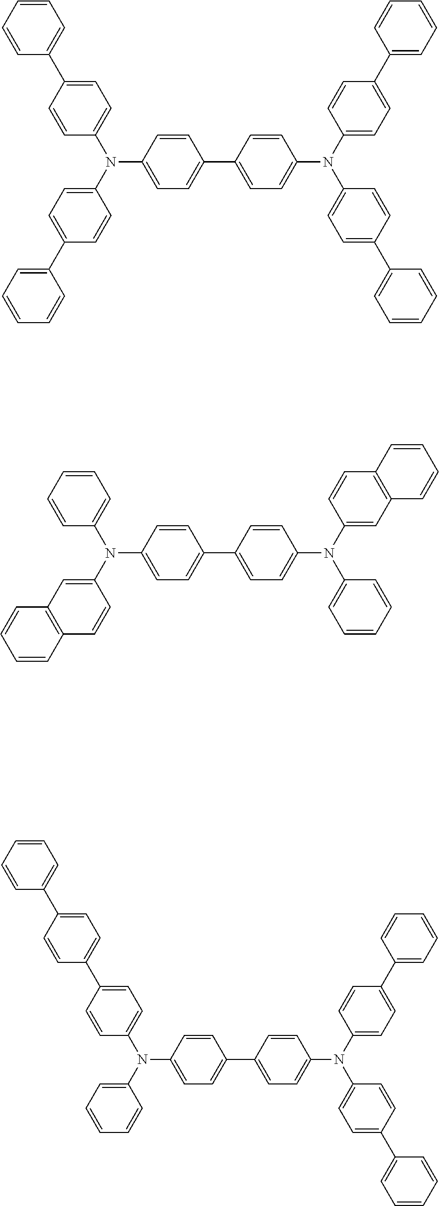 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

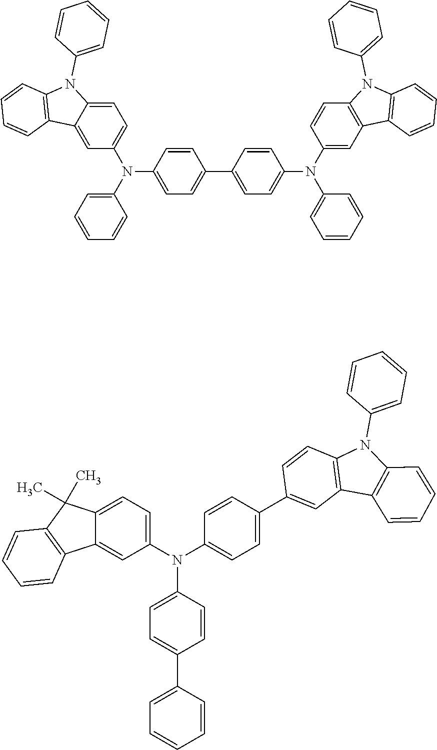 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be about 1.6 or more. Specifically, the refractive index of the capping layer CPL with respect to light with a wavelength range of about 550 nm to about 660 nm may be about 1.6 or more.

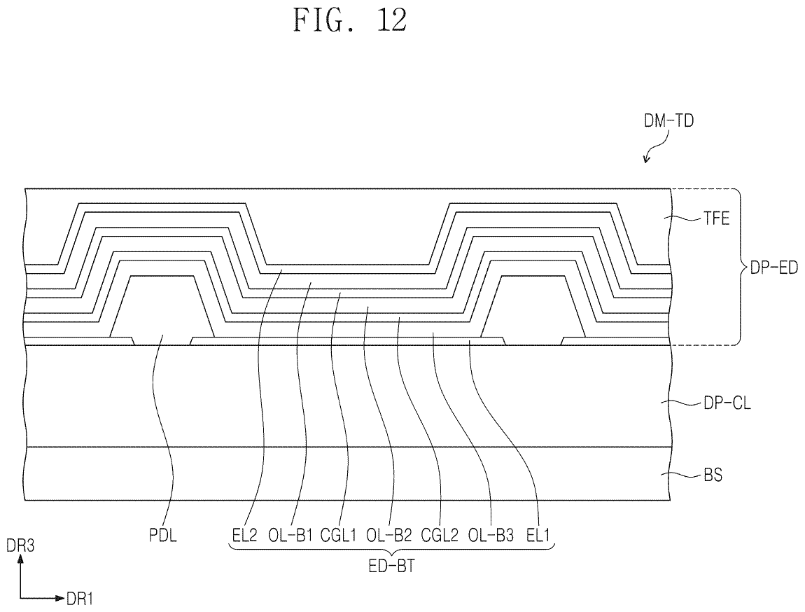

FIG. 11 to FIG. 14 are cross-sectional views of display modules according to embodiments. Hereinafter, in the explanation on the display modules according to embodiments, to be described with reference to FIG. 11 to FIG. 14, the duplicated details with the above-described explanation in FIG. 1 to FIG. 10 will not be explained again, and the different features will be mainly explained.

Referring to FIG. 11, a display module DM-a according to an embodiment may include a display panel DP including a display element layer DP-ED, a light control layer CCL disposed on the display panel DP, and a color filter layer CFL. In an embodiment illustrated in FIG. 11, the display panel DP may include a base layer BS, a circuit layer DP-CL provided on the base layer BS and a display element layer DP-ED, and the display element layer DP-ED may include a light-emitting element ED.

A light-emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. The above-described structures of the light-emitting elements in FIG. 5 to FIG. 10 may be similarly applied to a structure of the light-emitting element ED illustrated in FIG. 11. The light-emitting element ED illustrated in FIG. 11 may include the amine compound according to an embodiment. The light-emitting element ED according to an embodiment includes the amine compound according to an embodiment in the hole transport region HTR and thus may exhibit a low driving voltage and long lifespan characteristics. In addition, the light-emitting element ED according to an embodiment may emit blue fluorescence and exhibit a low driving voltage and long lifespan characteristics.

Referring to FIG. 11, the emission layer EML may be disposed within an opening OH defined in a pixel-defining film PDL. For example, the emission layer EML, which is separated by the pixel-defining film PDL and correspondingly and respectively provided in the emission regions PXA-R, PXA-G, and PXA-B, may emit light of the same wavelength region. In a display module DM-a according to an embodiment, the emission layer EML may emit blue light. Unlike what is illustrated, in an embodiment, the emission layer EML may be provided across all of the emission regions PXA-R, PXA-G, and PXA-G as a common layer.

The light control layer CCL may be disposed on the display panel DP. The light control layer CCL is illustrated to be disposed on the display element layer DP-ED, but an embodiment is not limited thereto, and the light control layer CCL may be disposed under the display element layer DP-ED. The light control layer CCL may include a light converter. The light converter may be quantum dost or a fluorescent substance. The light converter may convert a wavelength of light provided and emit the light. That is, the light control layer CCL may be a layer including quantum dots or a layer including a fluorescent substance.

The light control layer CCL may include a plurality of light control parts CCP1, CCP2 and CCP3. The light control parts CCP1, CCP2 and CCP3 may be spaced apart from each other.

Referring to FIG. 11, a partition pattern BMP may be disposed between the light control parts CCP1, CCP2 and CCP3, spaced apart from each other, but an embodiment is not limited thereto. In FIG. 11, the partition pattern BMP is illustrated not to be overlapped with the light control parts CCP1, CCP2 and CCP3, but at least a portion of an edge of the light control parts CCP1, CCP2 and CCP3 may be overlapped with the partition pattern BMP.

The light control layer CCL may include a first light control part CCP1 including a first quantum dot QD1 converting first color light provided from the light-emitting element ED into second color light, a second light control part CCP2 including a second quantum dot QD2 converting first color light into third color light, and a third light control part CCP3 transmitting first color light.

In an embodiment, the first light control part CCP1 may provide red light which is the second color light, and the second light control part CCP2 may provide green light which is the third color light. The third light control part CCP3 may transmit and provide blue light which is the first color light provided from the light-emitting element ED. For example, the first quantum dot QD1 may be a red quantum dot, and the second quantum dot QD2 may be a green quantum dot. The quantum dots QD1 and QD2 may be applied with the same content as those described previously.

In addition, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include a quantum dot but include the scatterer SP.

The scatterer SP may be an inorganic particle. For example, the scatterer SP may include at least one among TiO₂, ZnO, Al₂O₃, SiO₂, and hollow silica.

The scatterer SP may include any one among TiO₂, ZnO, Al₂O₃, SiO₂, and hollow silica, or may be a mixture of two or more materials selected among TiO₂, ZnO, Al₂O₃, SiO₂, and hollow silica.

The first light control part CCP1, the second light control part CCP2, and the third light control part CCP3 may include base resins BR1. BR2, and BR3 that disperse the quantum dots QD1 and QD2, and the scatterer SP, respectively. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 may be each a medium through which the quantum dots QD1 and QD2 and the scatterer SP are dispersed and may include various resin compositions, which may be generally referred to as a binder.

For example, the base resins BR1, BR2, and BR3 may be each an acrylate-based resin, a urethane-based resin, a silicon-based resin, an epoxy-based resin, etc. The base resins BR1, BR2, and BR3 may be transparent resins. In an embodiment, the first base resin BR1, the second base resin BR2, and the third base resin BR3 may be the same as, or different from each other.

The light control layer CCL may include a barrier layer BFL1. The barrier layer BFL1 may serve to prevent penetration of moisture and/or oxygen (hereinafter, referred to as ‘moisture/oxygen’). The barrier layer BFL1 may be disposed on the light control parts CCP1, CCP2, and CCP3 and prevent the light control parts CCP1, CCP2, and CCP3 from being exposed to moisture/oxygen. The barrier layer BFL1 may cover the light control parts CCP1, CCP2, and CCP3. In addition, a barrier layer BFL2 may also be provided between the light control parts CCP1, CCP2, and CCP3 and a color filter layer CFL.

The barrier layers BFL1 and BFL2 may include at least one inorganic layer. That is, the barrier layers BFL1 and BFL2 may be formed including an inorganic material. For example, the barrier layers BFL1 and BFL2 may include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, a metal thin film having secured light transmittance, etc. The barrier layers BFL1 and BFL2 may further include an organic film. The barrier layers BFL1 and BFL2 may include a single layer or a plurality of layers.

In the display module DM-a according to an embodiment, the color filter layer CFL may be disposed on the light control layer CCL. For example, the color filter layer CFL may be directly disposed on the light control layer CCL. In this case, a second barrier layer BFL2 may be omitted.

The color filter layer CFL may include the filters CF1, CF2, and CF3. The color filter layer CFL may include a first filter CF1 transmitting the second color light, a second filter CF2 transmitting the third color light, and a third filter CF3 transmitting the first color light. For example, the first filter CF1 may be a red filter, the second filter CF2 may be a green filter, and the third filter CF3 may be a blue filter.

The filters CF1, CF2, and CF3 may each include a polymer photosensitive resin, and a pigment or dye. The first filter CF1 may include a red pigment or red dye, the second filter CF2 may include a green pigment or green dye, and the third filter CF3 may include a blue pigment or blue dye.

An embodiment is not limited thereto, and the third filter CF3 may not include a pigment or dye. The third filter CF3 may include a polymer photosensitive resin and may not include a pigment or dye. The third filter CF3 may be transparent. The third filter CF3 may include a transparent photosensitive resin.

In addition, in an embodiment, the first filter CF1 and the second filter CF2 may be yellow filters. The first filter CF1 and the second filter CF2 may not be separated from each other and may be provided as one body.

Although not illustrated, the color filter layer CFL may further include a light-blocking part (not shown). The light-blocking part may be a black matrix. The light-blocking part may include an organic light-blocking material or an inorganic light-blocking material containing a black pigment or black dye. The light-blocking part may prevent a light-leak phenomenon, and may set boundaries between the adjacent filters CF1, CF2, and CF3. In addition, in an embodiment, the light-blocking part may include a blue filter.

The first to third filters CF1, CF2, and CF3 may be respectively and correspondingly disposed in the first emission region PXA-R, the second emission region PXA-G, and the third emission region PXA-B.

A base substrate BL may be disposed on the color filter layer CFL. The base substrate BL may be a member that provides a base surface on which the color filter layer CFL, the light control layer CCL, and the like are disposed. The base substrate BL may be a glass substrate, a metal substrate, a plastic substrate, etc. However, an embodiment is not limited thereto, and the base substrate BL may be an inorganic layer, an organic layer, or a composite material layer. In addition, unlike what is illustrated, the base substrate BL may also be omitted in an embodiment.

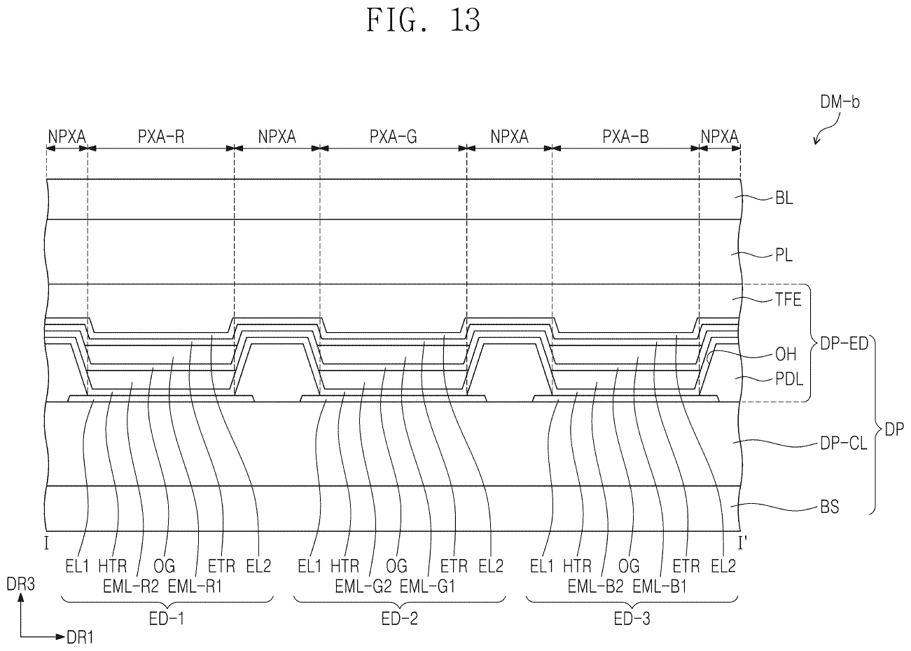

FIG. 12 is a cross-sectional view illustrating a portion of a display module according to an embodiment. In a display module DM-TD according to an embodiment, the light-emitting element ED-BT may include a plurality of emission structures OL-B1, OL-B2, and OL-B3. At least one among the plurality of emission structures OL-B1, OL-B2, and OL-B3 may include the amine compound according to an embodiment. Therefore, the light-emitting element ED-BT may exhibit a low driving voltage and long lifespan characteristics. In addition, the light-emitting element ED-BT according to an embodiment may emit blue light and exhibit a low driving voltage and long lifespan characteristics.

The light-emitting element ED-BT may include a first electrode EL1, a second electrode EL2, facing each other, and the plurality of emission structures OL-B1, OL-B2, and OL-B3, stacked in sequence and provided in a thickness direction between the first electrode EL1 and the second electrode EL2. The emission structures OL-B1, OL-B2, and OL-B3 may each include an emission layer EML (FIG. 5), a hole transport region HTR, and an electron transport region ETR. The emission layer EML (FIG. 5) may be disposed between the hole transport region HTR and the electron transport region ETR. That is, the light-emitting element ED-BT included in the display module DM-TD according to an embodiment may be a light-emitting element having a tandem structure which includes a plurality of emission layers.

In the embodiment illustrated in FIG. 12, light emitted from each of the emission structures OL-B1, OL-B2, and OL-B3 may be blue light. However, an embodiment is not limited thereto, and the emission structures OL-B1, OL-B2, and OL-B3 may emit light in different wavelength regions. For example, the light-emitting element ED-BT, including the plurality of emission structures OL-B1, OL-B2, and OL-B3 that emit light in different wavelength regions, may emit white light.

Charge-generating layers CGL1 and CGL2 may be disposed between the adjacent emission structures OL-B1, OL-B2, and OL-B3. In an embodiment illustrated in FIG. 12, the charge-generating layers CGL1 and CGL2 may include a first charge-generating layer CGL1 disposed between a first emission structure OL-B1 and a second emission structure OL-B2, and a second charge-generating layer CGL2 disposed between the second emission structure OL-B2 and a third emission structure OL-B3. The first and second charge-generating layer CGL1 and CGL2 may each independently include a p-type charge-generating layer and/or a n-type charge-generating layer.

Referring to FIG. 13, a display module DD-b according to an embodiment may include light-emitting elements ED-1, ED-2, and ED-3 in which two emission layers are stacked. At least one among the light-emitting elements ED-1, ED-2, and ED-3 may include the amine compound according to an embodiment in the hole transport region HTR. Therefore, the light-emitting elements ED-1, ED-2, and ED-3 may exhibit a low driving voltage and long lifespan characteristics. In addition, the light-emitting element ED-3 may exhibit a low driving voltage and long lifespan in an emission region of blue light.

The embodiment illustrated in FIG. 13 differs from the display module DM according to the embodiment, illustrated in FIG. 4, in that the first to third light-emitting elements ED-1, ED-2, and ED-3 each include two emission layers stacked in a thickness direction. The two emission layers of each of the first to third light-emitting elements ED-1, ED-2, and ED-3 may emit light in the same wavelength region.

The first light-emitting element ED-1 may include a first red emission layer EML-R1 and a second red emission layer EML-R2. The second light-emitting element ED-2 may include a first green emission layer EML-G1 and a second green emission layer EML-G2. In addition, the third light-emitting element ED-3 may include a first blue emission layer EML-B1 and a second blue emission layer EML-B2. An auxiliary emission part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The auxiliary emission part OG may include a single layer or multiple layers. The auxiliary emission part OG may include a charge-generating layer. More specifically, the auxiliary emission part OG may include an electron transport region, a charge-generating layer, and a hole transport region, stacked in sequence. The auxiliary emission part OG may be provided as a common layer across all of the first to third light-emitting elements ED-1, ED-2, and ED-3. However, an embodiment is not limited thereto, and the auxiliary emission part OG may be provided by being patterned in the opening OH defined by the pixel-defining film PDL.

The first red emission layer EML-R1, the first green emission layer EML-G1, and the first blue emission layer EML-B1 may be disposed between the auxiliary emission part OG and the electron transport region ETR. The second red emission layer EML-R2, the second green emission layer EML-G1, and the second blue emission layer EML-B2 may be disposed between the hole transport region HTR and the auxiliary emission part OG.

That is, the first light-emitting element ED-1 may include the first electrode EL1, the hole transport region HTR, the second red emission layer EML-R2, the auxiliary emission part OG, the first red emission layer EML-R1, the electron transport region ETR, and the second electrode EL2, stacked in sequence. The second light-emitting element ED-2 may include the first electrode EL1, the hole transport region HTR, the second green emission layer EML-G2, the auxiliary emission part OG, the first green emission layer EML-G1, the electron transport region ETR, and the second electrode EL2, stacked in sequence. The third light-emitting element ED-3 may include the first electrode EL1, the hole transport region HTR, the second blue emission layer EML-B2, the auxiliary emission part OG, the first blue emission layer EML-B1, the electron transport region ETR, and the second electrode EL2, stacked in sequence.

An auxiliary optical layer PL may be disposed on the display element layer DP-ED. The auxiliary optical layer PL may include a polarizing layer. The auxiliary optical layer PL may be disposed on the display panel DP and may control reflected light of external light on the display panel DP. Unlike what is illustrated, the auxiliary optical layer PL may also be omitted in the display device according to an embodiment.

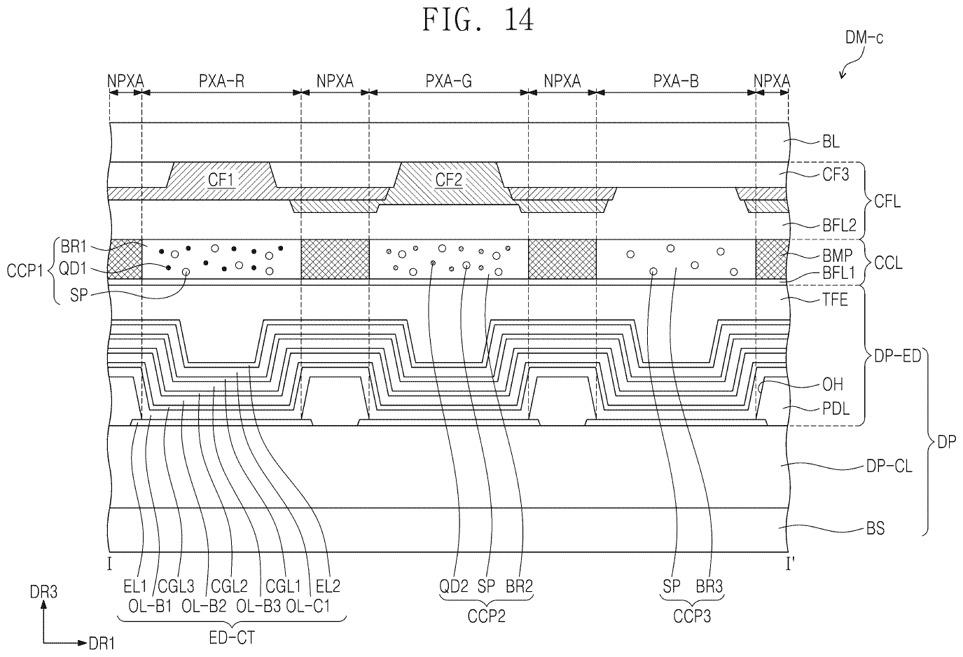

Unlike what is illustrated in FIG. 12 and FIG. 13, FIG. 14 illustrates that a display module DM-c according to an embodiment includes four emission structures OL-B3, OL-B2, OL-B1, and OL-C1. The light-emitting element ED-CT may include a first electrode EL1 and a second electrode EL2, facing each other, and first to fourth emission structures OL-B1, OL-B2, OL-B3, and OL-C1, stacked in sequence in a thickness direction between the first electrode EL1 and the second electrode EL2. At least one among the first to fourth emission structures OL-B1, OL-B2, OL-B3, and OL-C1 may include the amine compound according to an embodiment.

Therefore, the light-emitting element ED-CT may exhibit a low driving voltage and long lifespan characteristics. In addition, the light-emitting element ED-CT according to an embodiment may have a low driving voltage in a blue emission region and exhibit improved lifespan characteristics.

Charge-generating layers CGL1, CGL2, and CGL3 may be disposed between the first to fourth emission structures OL-B1, OL-B2, OL-B3, and OL-C1.

The first to third emission structures OL-B1, OL-B2, and OL-B3 among the four emission structures may emit blue light, and the fourth emission structure OL-C1 may emit green light. However, an embodiment is not limited thereto, and the first to fourth emission structures OL-B1, OL-B2, OL-B3, and OL-C1 may emit light in different wavelength regions from each other. The charge-generating layers CGL3, CGL2, and CGL1 disposed between adjacent emission structures OL-B1, OL-B2, OL-B3, and OL-C1 may include a p-type charge-generating layer and/or a n-type charge-generating layer.

In an embodiment, the electronic apparatus may include a display device that includes a plurality of light-emitting elements, and a control part that controls the display device. In an electronic apparatus according to an embodiment, at least one among the plurality of light-emitting elements may include the amine compound according to an embodiment in the hole transport region. For example, at least one among the plurality of light-emitting elements included in the electronic apparatus according to an embodiment may include the above-described amine compound according to an embodiment in the hole transport layer.

The electronic apparatus according to an embodiment may be activated in response to an electrical signal. The electronic apparatus according to an embodiment may include display devices which include the display module according to an embodiment, described with reference to FIG. 4, and FIG. 11 to FIG. 14. and the like. For example, the electronic apparatus may not only include a large-size electronic apparatus such as a television, a monitor, or an outdoor billboard, but also include a small- and medium-size electronic apparatus such as a personal computer, a laptop computer, a personal digital assistant, a vehicular display device, a game console, a portable electronic apparatus, or a camera.

The electronic apparatus according to an embodiment includes the display module including the amine compound according to an embodiment and thus may have improved reliability and exhibit a low driving voltage and long lifespan characteristics. The electronic apparatus according to an embodiment may have a reduced driving voltage, and an improved display lifespan and exhibit excellent display quality.

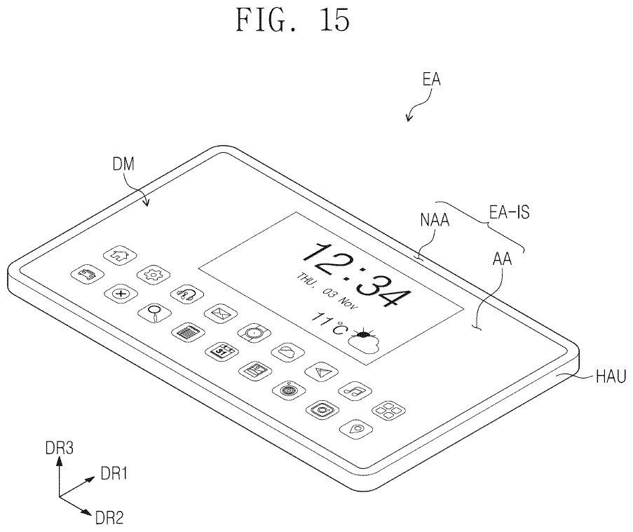

FIG. 15 illustrates a tablet computer as an example of the electronic apparatus EA. The electronic apparatus EA may include the display module DM according to an embodiment. For example, electronic modules, a camera module, a power module, etc. mounted on a main board, may be disposed in a bracket/housing HAU, etc. together with a display module DM to thereby configure the tablet computer.

The electronic apparatus EA according to an embodiment illustrated in FIG. 15 may include the display module according to an embodiment described with reference to FIG. 4, FIG. 11 to FIG. 14, and the like.

In an embodiment, the electronic apparatus EA including the display module DM, which includes a flat display surface, is illustrated, but an embodiment is not limited thereto. The electronic apparatus EA may also include a curved display surface or a three-dimensional display surface. For example, the three-dimensional display surface may include a plurality of display regions indicating different directions, and may also include a bent display surface. The electronic apparatus EA according to this embodiment may be a flexible electronic apparatus. The flexible electronic apparatus may be a foldable electronic apparatus capable of folding.

As illustrated in FIG. 15, a display surface EA-IS includes an active region AA in which an image is displayed, and a bezel region NAA adjacent to the active region AA. The bezel region NAA is a region where an image is not displayed.

FIG. 15 illustrates icon images as examples of an image. The active region AA may be referred to as a display region of the display module DM, and the bezel region NAA may be referred to as a non-display region of the display module DM.

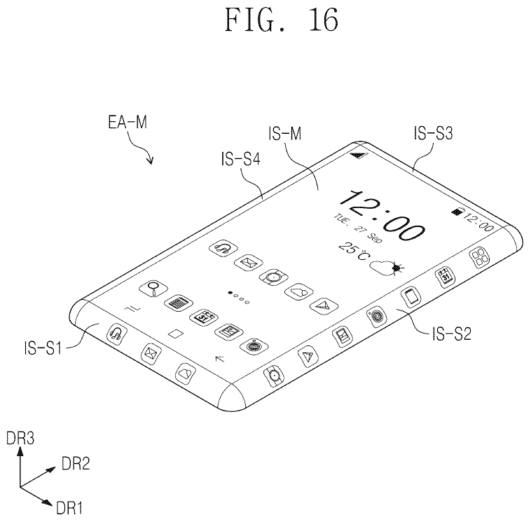

FIG. 16 illustrates a portable device as an example of an electronic apparatus EA-M according to an embodiment. Referring to FIG. 16, the electronic apparatus EA-M according to an embodiment may include a plurality of display surfaces. The electronic apparatus EA-M according to an embodiment may include display surfaces IS-M, IS-S1, IS-S2, IS-S3, and IS-S4 whose main display directions are different from each other.

In an embodiment, the electronic apparatus EA-M may be a three-dimensional display device which includes a top display surface IS-M, and a plurality of side display surfaces IS-S1, IS-S2, IS-S3, and IS-S4. The plurality of side display surfaces IS-S1, IS-S2, IS-S3, and IS-S4 may each be a display surface extended from one side of the top display surface IS-M. In an embodiment, the electronic apparatus EA-M may include a main display surface mainly providing an image in one direction, and a plurality of sub display surfaces providing an image in a direction different from the one direction. In an embodiment of the electronic apparatus EA-M, illustrated in FIG. 16, the main display surface may be the top display surface IS-M, and the sub display surfaces may be the side display surfaces IS-S1, IS-S2, IS-S3, and IS-S4.

Side display surfaces IS-S1, IS-S2, IS-S3, and IS-S4 may have a display surface that is not parallel to the top display surface IS-M. The plurality of sub display surfaces IS-S1, IS-S2, IS-S3, and IS-S4 are each a display region bent and extended from one side of the top display surface IS-M, and for example, the plurality of side surfaces IS-S1, IS-S2, IS-S3, and IS-S4 may be bent display regions.

The electronic apparatus EA-M according to an embodiment, illustrated in FIG. 16, may include the display module according to an embodiment, described with reference to FIG. 4, FIG. 11 to FIG. 14, and the like.

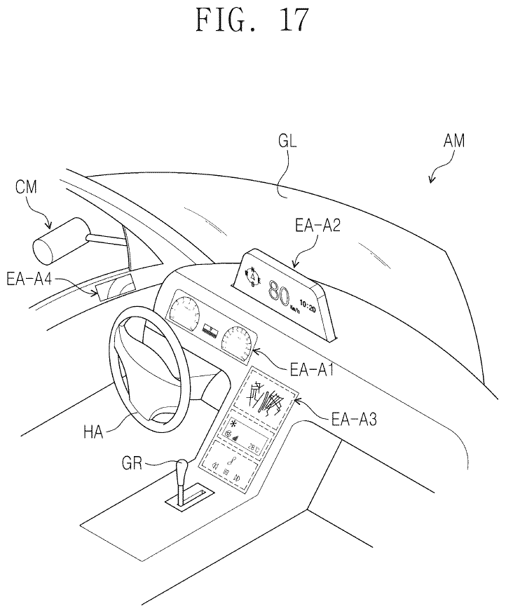

FIG. 17 is a view illustrating a vehicle AM in which first to fourth electronic apparatuses EA-1, EA-2, EA-3, and EA-4 are disposed. At least one among the first to fourth electronic apparatuses EA-1, EA-2, EA-3, and EA-4 may include the display module according to an embodiment, described with reference to FIG. 4, FIG. 11 to FIG. 14.

FIG. 17 illustrates a car as a vehicle AM, but this is an example, and the first to fourth electronic apparatuses EA-1, EA-2, EA-3, and EA-4 may also be disposed in other transportation such as a bicycle, a motorcycle, a train, a ship, and a plane.

At least one among the first to fourth electronic apparatuses EA-1, EA-2, EA-3, and EA-4 may include the light-emitting element ED described with reference to FIG. 5 to FIG. 10. At least one among the first to fourth electronic apparatuses EA-1, EA-2, EA-3, and EA-4 may include the amine compound according to an embodiment. Therefore, the first to fourth electronic apparatuses EA-1, EA-2, EA-3, and EA-4, including the amine compound according to an embodiment may have a reduced driving voltage and an improved display lifespan. In addition, the first to fourth electronic apparatuses EA-1, EA-2, EA-3, and EA-4, including the amine compound according to an embodiment may exhibit excellent display quality and improved reliability characteristics.

Referring to FIG. 17, the vehicle AM may include a handle HA and a gear GR for controlling operation and include a front window GL disposed in front of a driver.

A first electronic apparatus EA-1 may be a digital cluster that displays first information of the vehicle AM. The first information may include a first scale indicating a driving speed, a second scale indicating revolutions per minute (RPM), an image indicating a fuel state, and the like. The first scale and the second scale may be indicated in digital images. In an embodiment illustrated in FIG. 17, the first electronic apparatus EA-1 may be disposed in a first region overlapping the handle HA. However, an embodiment is not limited thereto, and the first electronic apparatus EA-1 may be disposed entirely on a dashboard or disposed separately and respectively in a part facing a driver's seat and, in a part facing a passenger seat.

A second electronic apparatus EA-2 may be disposed in a second region between the driver's seat and the front window GL. For example, the second electronic apparatus EA-2 may be a head-up display (HUD) that displays second information of the vehicle AM. The second electronic apparatus EA-2 may be optically transparent.

The second information may include a digital number indicating the driving speed of the vehicle AM and may further include information such as current time. Unlike what is illustrated, the second information of the second electronic apparatus EA-2 may be displayed by being projected on the front window GL. A display surface of the second electronic apparatus EA-2 may face the driver's seat. In addition, alternatively, the second electronic apparatus EA-2 may provide an image toward the front window GL.

A third electronic apparatus EA-3 may be disposed in a third region adjacent to the gear GR. For example, the electronic apparatus EA-3 may be a center information display (CID) for vehicles, which is disposed between the driver's seat and the passenger seat and displays third information. The passenger seat may be a seat spaced apart from the driver's seat. The gear GR may be disposed between the passenger seat and the driver's seat. The third information may include information on road conditions (for example, navigation information), music or radio play, dynamic image play, temperature inside the vehicle AM, etc.

A fourth electronic apparatus EA-4 may be disposed in a fourth region spaced apart from the handle HA and the gear GR, and adjacent to a side portion of the vehicle AM. For example, the fourth electronic apparatus EA-4 may be a digital side mirror that displays fourth information. The fourth electronic apparatus EA-4 may display external image of the vehicle AM taken from a camera module CM disposed on the outside of the vehicle AM. The fourth information may include an external image of the vehicle AM.

The first to fourth information previously described are examples, and the first to fourth electronic apparatuses EA-1, EA-2, EA-3, and EA-4 may further display information on the inside and outside of the vehicle AM. The first to fourth information may include information different from each other. However, an embodiment is not limited thereto, and some of the first to fourth information may also include the same information to each other.

FIG. 15 to FIG. 17 exemplify an electronic apparatus according to an embodiment or an embodiment including the electronic apparatus, the display module including the light-emitting element which includes the amine compound according to an embodiment may be employed to other electronic apparatuses without departing from the concept.

Hereinafter, with reference to Examples and Comparative Examples, the amine compound according to an embodiment and the light-emitting element according to an embodiment will be described in detail. In addition, Examples in this specification are exemplified only for helping the understanding, and the scope this disclosure is not limited thereto.

### EXAMPLES

### 1. Synthesis of Amine Compound According to Examples

A synthetic method of the amine compounds according to the present embodiment will be explained in detail by exemplifying the synthetic methods of Compounds 1, 2, 4, 12, and 85. In addition, the synthetic methods of the amine compounds explained hereinafter are examples, and a synthetic method of the amine compound according to an embodiment of this disclosure is not limited to the following examples.

### (1) Synthesis of Compound 1

Amine Compound 1 according to one example may be synthesized by, for example, steps of Reaction Scheme 1.

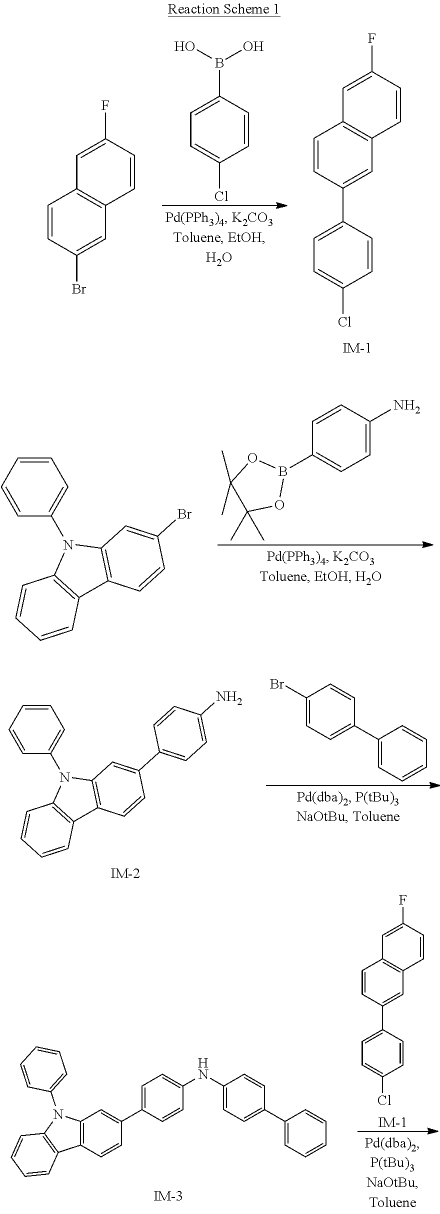

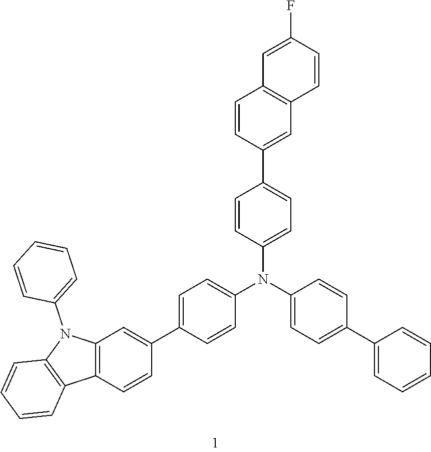 [SMILES: Fc1ccc2cc(-c3ccc(N(c4ccc(-c5ccccc5)cc4)c4ccc(-c5ccc6c7ccccc7n(-c7ccccc7)c6c5)cc4)cc3)ccc2c1]

### Synthesis of Intermediate IM-1

Under an argon atmosphere, 2-bromo-6-fluoronaphthalene (25.00 g, 111.08 mmol), 4-chlorophenylboronic acid (17.37 g, 1.1 equiv., 110.8 mmol), Pd(PPh₃)₄ (6.418 g, 0.05 equiv., 5.55 mmol), K₂CO₃ (30.70 g, 2.0 equiv., 222.16 mmol), toluene (278 mL), EtOH (56 mL), and H₂O (111 mL) were added to a 2,000 mL three-neck flask, one by one, followed by heating, refluxing, and stirring. After cooling to room temperature, water and toluene were added to a reaction solution, and an organic layer was separately taken. The organic layer was washed with a saline solution and then dried over MgSO₄. MgSO₄ was filtered and separated, and the organic layer was concentrated to obtain a crude product. The obtained crude product was purified to obtain Intermediate IM-1 (14.17 g, yield of 50%).

The mass number of Intermediate IM-1, measured by FAB-MS was 256.7.

### Synthesis of Intermediate IM-2

Under an argon atmosphere, 2-bromo-9-phenylcarbazole (40.00 g, 124.14 mmol), 4-(4,4,5,5-tetramethyl-1,3,2-dioxaborolan-2-yl)aniline (27.20 g, 1.0 equiv., 124.14 mmol), Pd(PPh₃)₄(6.207 g, 0.05 equiv., 6.207 mmol), K₂CO₃ (34.31 g, 2.0 equiv., 248.29 mmol), toluene (310 mL), EtOH 62 mL, and H₂O (124 mL) were added to a 1,000 mL three-neck flask, one by one, followed by heating, refluxing, and stirring. After cooling to room temperature, water and toluene were added to a reaction solution, and an organic layer was separately taken. The organic layer was washed with a saline solution and then dried over MgSO₄. MgSO₄ was filtered and separated, and the organic layer was concentrated to obtain a crude product. The obtained crude product was purified to obtain Intermediate IM-2 (29.43 g, yield of 71%).

The mass number of Intermediate IM-2 measured by FAB-MS was 334.42.

### Synthesis of Intermediate IM-3

Under an argon atmosphere, IM-2 (10.20 g, 30.50 mmol), Pd(dba)₂ (0.175 g, 0.01 equiv., 0.31 mmol), NaOtBu (4.40 g, 1.5 equiv., 45.75 mmol), toluene (76 mL), 4-bromobiphenyl (7.11 g, 1.0 equiv., 30.5 mmol), and PtBu₃ (0.61 mL, 0.04 equiv., 1.22 mmol) were added to a 500 mL three-neck flask, one by one, followed by heating, refluxing, and stirring. After cooling to room temperature, water was added to a reaction solution, and an organic layer was separately taken. Toluene was added to an aqueous layer, and the organic layer was additionally extracted. Then, the organic layers were all together washed with a saline solution, and then dried over MgSO₄. MgSO₄ was filtered and separated, and the organic layers were concentrated to obtain a crude product. The obtained crude product was purified to obtain Intermediate IM-3 (12.02 g, yield of 81%).

The mass number of Intermediate IM-3, measured by FAB-MS was 486.62.

### Synthesis of Compound 1

Under an argon atmosphere, IM-3 (6.94 g, 14.27 mmol), Pd(dba)₂ (0.164 g, 0.02 equiv., 0.28 mmol), NaOtBu (2.06 g, 1.5 equiv., 21.4 mmol), toluene (48 mL), IM-1 (3.66 g, 1.0 equiv., 14.27 mmol), and PtBu₃ (0.58 mL, 0.08 equiv., 1.14 mmol) were added to a 200 mL three-neck flask, one by one, followed by heating, refluxing, and stirring. After cooling to room temperature, water was added to a reaction solution, and an organic layer was separately taken. Toluene was added to an aqueous layer, and the organic layer was additionally extracted. Then, the organic layers were collected, then washed with a saline solution, and then dried over MgSO₄.

MgSO₄ was filtered and separated, and the organic layers were concentrated to obtain a crude product. The obtained crude product was purified to obtain Compound 1 (6.25 g, yield of 62%).

The mass number of Compound 1, measured by FAB-MS was 706.86.

### (2) Synthesis of Compound 2

Amine Compound 2 according to one example may be synthesized by, for example, steps of Reaction Scheme 2.

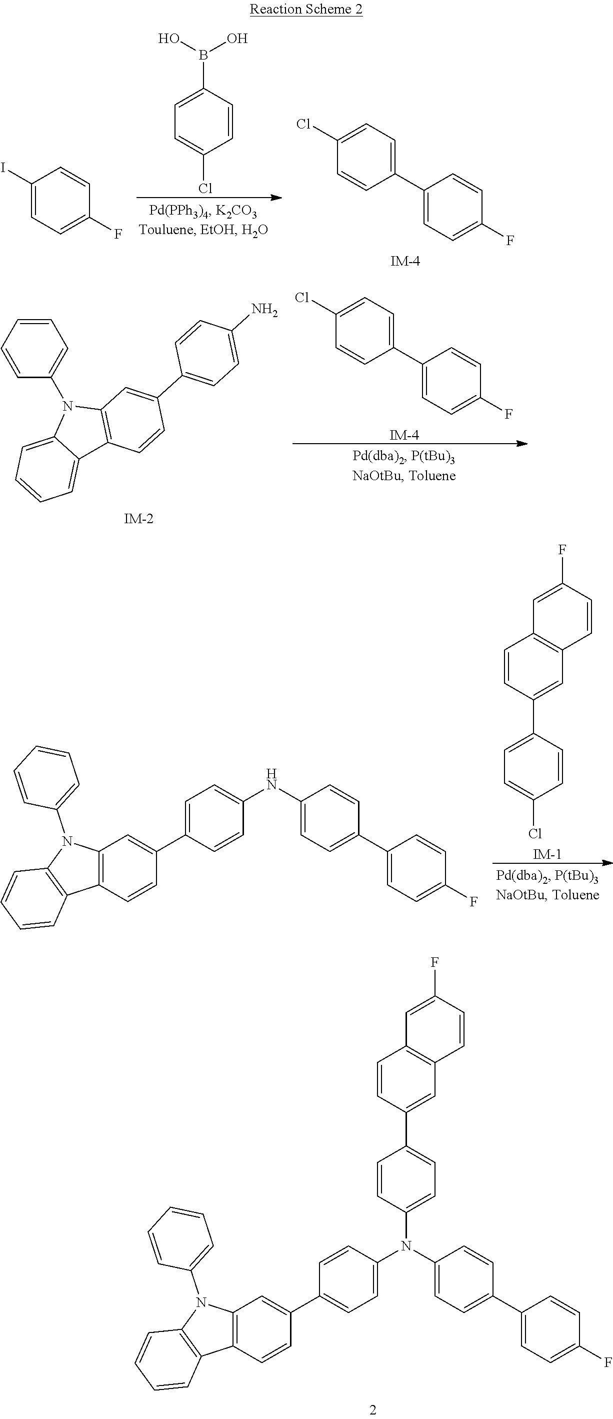

### Synthesis of Intermediate IM-4

Under an argon atmosphere, 1-fluoro-4-iodobenzene (25 g, 112.61 mmol), 4-chlorophenylboronic acid (17.61 g, 1.0 equiv., 112.61 mmol), Pd(PPh₃)₄ (6.507 g, 0.05 equiv., 5.63 mmol), K₂CO₃ (31.13 g, 2.0 equiv., 225.23 mmol), toluene (282 mL), EtOH (56 mL), and H₂O (113 mL) were added to a 1,000 mL three-neck flask, one by one, followed by heating, refluxing, and stirring. After cooling to room temperature, water and toluene were added to a reaction solution, and an organic layer was separately taken. The organic layer was washed with a saline solution and then dried over MgSO₄. MgSO₄ was filtered and separated, and the organic layer was concentrated to obtain a crude product. The obtained crude product was purified to obtain Intermediate IM-4 (20.24 g, yield of 87%).

The mass number of Intermediate IM-4 measured by FAB-MS was 206.64.

### Synthesis of Compound 2

Compound 2 (18.67 g, yield of 52%) was synthesized in the same manner as the synthetic method of Compound 1 except for using IM-4 in place of 4-bromobiphenyl.

The mass number of Compound 2, measured by FAB-MS was 724.85.

### (3) Synthesis of Compound 4

Amine Compound 4 according to one example may be synthesized by, for example, steps of Reaction Scheme 3.

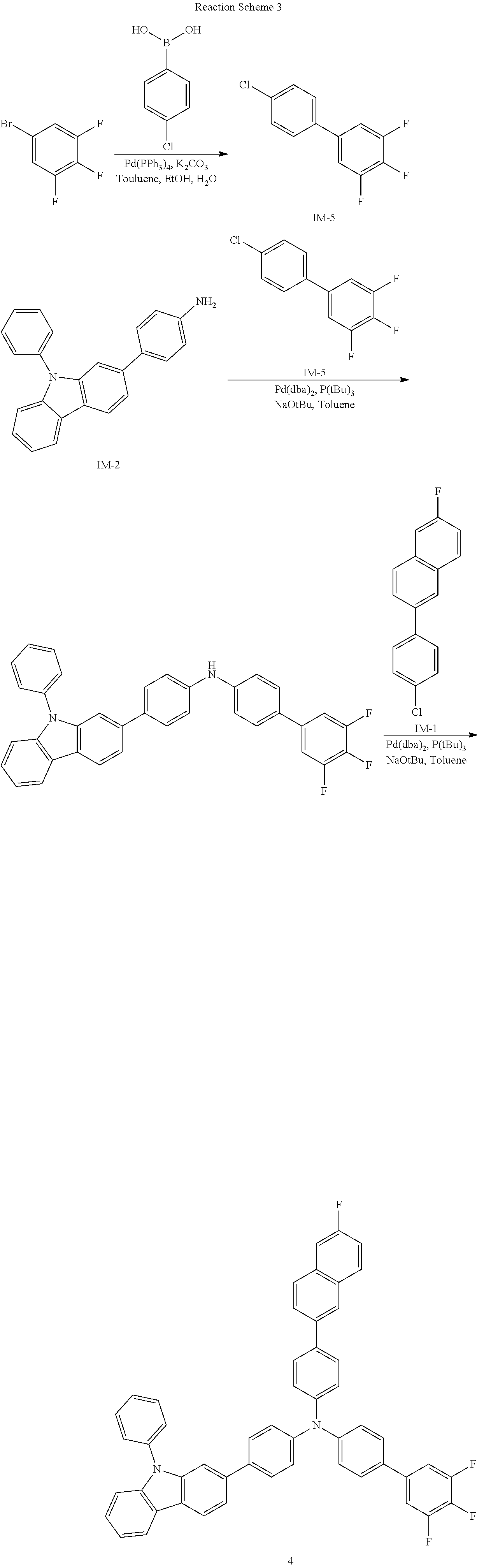

### Synthesis of Intermediate IM-5

Under an argon atmosphere, 1-bromo-3,4,5-trifluorobenzene (17.4 g, 82.47 mmol), 4-chlorophenylboronic acid (12.90 g, 1.0 equiv., 82.47 mmol), Pd(PPh₃)₄(4.77 g, 0.05 equiv., 4.12 mmol), K₂CO₃ (22.8 g, 2.0 equiv., 164.94 mmol), toluene (206 mL), EtOH (41 mL), and H₂O (82 mL) were added to a 500 mL three-neck flask, one by one, followed by heating, refluxing, and stirring. After cooling to room temperature, water and toluene were added to a reaction solution, and an organic layer was separately taken. The organic layer was washed with a saline solution and then dried over MgSO₄. MgSO₄ was filtered and separated, and the organic layer was concentrated to obtain a crude product. The obtained crude product was purified to obtain Intermediate IM-5 (13.61 g, yield of 68%).

The mass number of Intermediate IM-5, measured by FAB-MS was 242.63.

### Synthesis of Compound 4

Compound 4 (16.17 g, yield of 46%) was synthesized in the same manner as the synthetic method of Compound 1 except for using IM-5 in place of 4-bromobiphenyl.

The mass number of Compound 4, measured by FAB-MS was 760.84.

### (4) Synthesis of Compound 12

Amine Compound 12 according to one example may be synthesized by, for example, steps of Reaction Scheme 4.

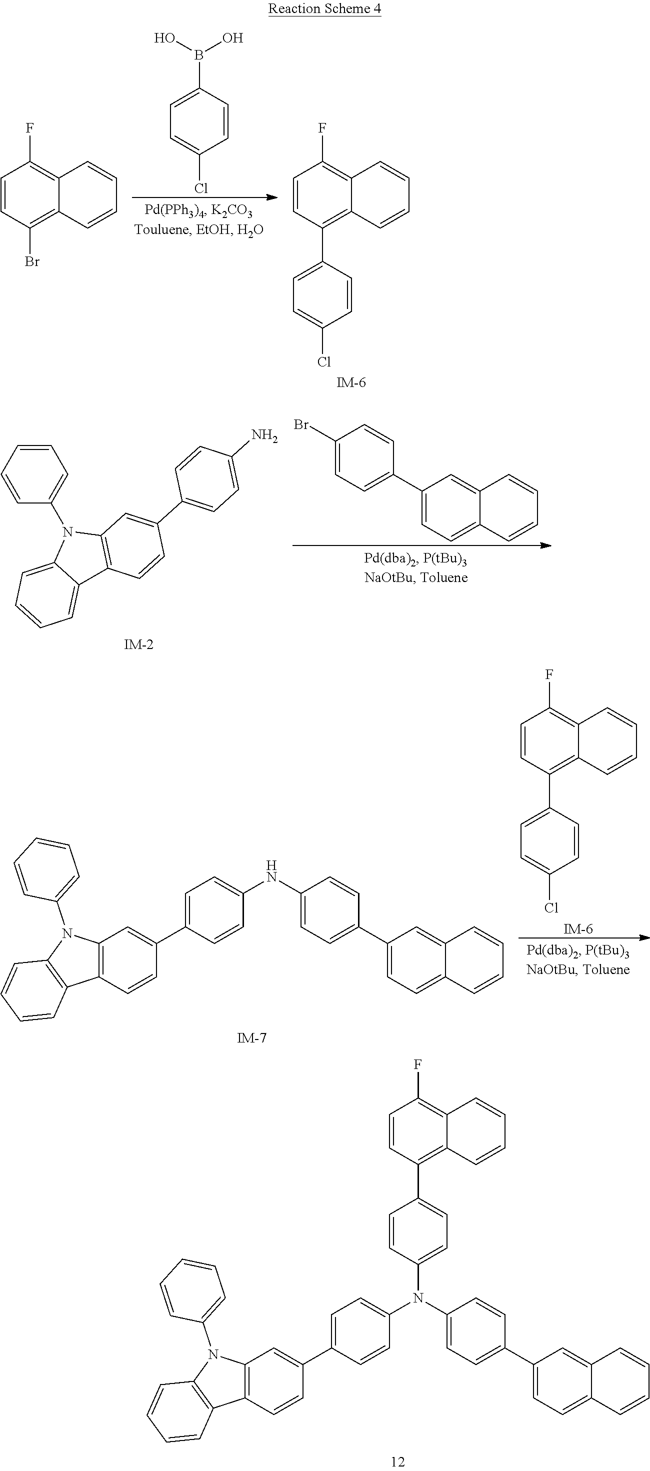

### Synthesis of Intermediate IM-6

Under an argon atmosphere, 1-bromo-4-fluoronaphthalene (25.00 g, 111.08 mmol), 4-chlorophenylboronic acid (17.37 g, 1.1 equiv., 110.8 mmol), Pd(PPh₃)₄ (6.418 g, 0.05 equiv., 5.55 mmol), K₂CO₃ (30.70 g, 2.0 equiv., 222.16 mmol), toluene (278 mL), EtOH (56 mL), and H₂O (111 mL) were added to a 1,000 mL three-neck flask, one by one, followed by heating, refluxing, and stirring. After cooling to room temperature, water and toluene were added to a reaction solution, and an organic layer was separately taken. The organic layer was washed with a saline solution and then dried over MgSO₄. MgSO₄ was filtered and separated, and the organic layer was concentrated to obtain a crude product. The obtained crude product was purified to obtain Intermediate IM-6 (16.82 g, yield of 59%).

The mass number of Intermediate IM-6, measured by FAB-MS was 256.7.

### Synthesis of Intermediate IM-7

Under an argon atmosphere, IM-2 (10.20 g, 30.50 mmol), Pd(dba)₂ (0.175 g, 0.01 equiv., 0.31 mmol), NaOtBu (4.40 g, 1.5 equiv., 45.75 mmol), toluene (76 mL), 2-(4-bromophenyl)naphthalene (8.6 g, 1.0 equiv., 30.5 mmol), and PtBu₃ (0.61 mL, 0.04 equiv., 1.22 mmol) were added to a 500 mL three-neck flask, one by one, followed by heating, refluxing, and stirring. After cooling to room temperature, water was added to a reaction solution, and an organic layer was separately taken. Toluene was added to an aqueous layer, and the organic layer was additionally extracted. Then, the organic layers were collected, washed with a saline solution, and then dried over MgSO₄. MgSO₄ was filtered and separated, and the organic layers were concentrated to obtain a crude product. The obtained crude product was purified to obtain Intermediate IM-7 (11.28 g, yield of 69%).

The mass number of Intermediate IM-7, measured by FAB-MS was 536.23.

### Synthesis of Compound 12

Under an argon atmosphere, IM-7 (7.65 g, 14.27 mmol), Pd(dba)₂ (0.164 g, 0.02 equiv., 0.28 mmol), NaOtBu (2.06 g, 1.5 equiv., 21.4 mmol), toluene (48 mL), IM-6 (3.66 g, 1.0 equiv., 14.27 mmol), and PtBu₃ (0.58 mL, 0.08 equiv., 1.14 mmol) were added to a 200 mL three-neck flask, one by one, followed by heating, refluxing, and stirring. After cooling to room temperature, water was added to a reaction solution, and an organic layer was separately taken. Toluene was added to an aqueous layer, and the organic layer was additionally extracted. Then, the organic layers were collected, washed with a saline solution, and then dried over MgSO₄. MgSO₄ was filtered and separated, and the organic layers were concentrated to obtain a crude product. The obtained crude product was purified to obtain Compound 4 (5.83 g, yield of 54%).

The mass number of Compound 4, measured by FAB-MS was 756.92.

### (5) Synthesis of Compound 85

Amine Compound 85 according to one example may be synthesized by, for example, steps of Reaction Scheme 5.

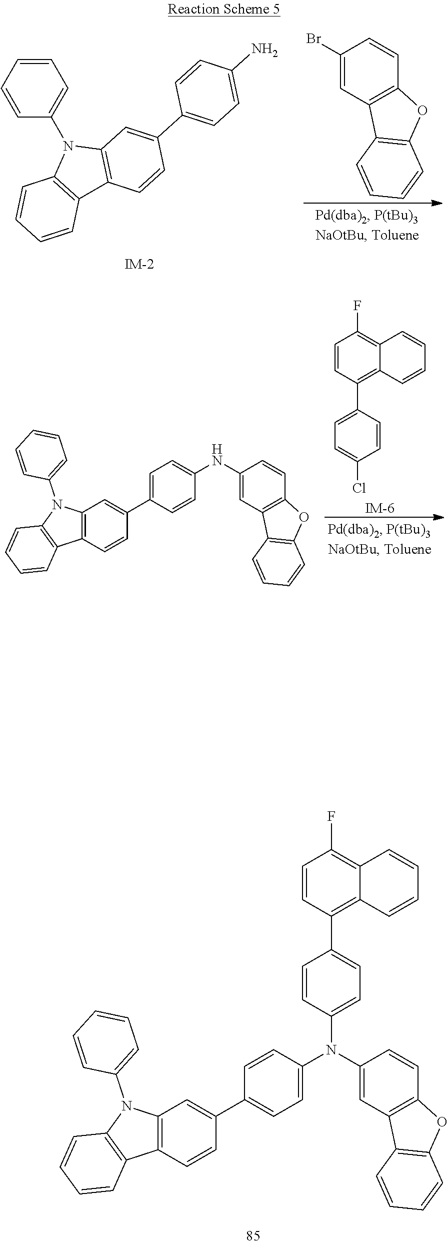

Compound 85 (6.26 g, yield of 58%) was synthesized in the same manner as the synthetic method of Compound 12 except for using 2-bromodibenzofuran in place of 2-(4-bromophenyl)naphthalene.

The mass number of Compound 85, measured by FAB-MS was 720.85.

### (6) Synthesis of Compound 89

Amine Compound 89 according to one example may be synthesized by, for example, steps of Reaction Scheme 6.

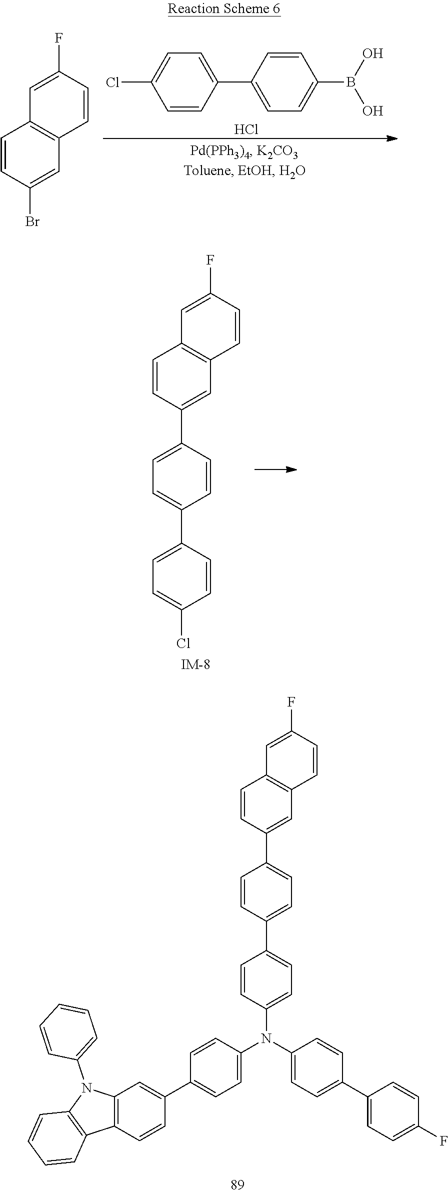

### Synthesis of Intermediate IM-8

Under an argon atmosphere, 2-bromo-6-fluoronaphthalene (25.00 g, 111.08 mmol), 4′-chloro-4-biphenylboronic acid (28.05 g, 1.1 equiv., 122.19 mmol), Pd(PPh₃)₄(6.418 g, 0.05 equiv., 5.55 mmol), K₂CO₃ (30.70 g, 2.0 equiv., 222.16 mmol), toluene (278 mL), EtOH (56 mL), H₂O (111 mL) were added to a 2,000 mL three-neck flask, one by one, followed by heating, refluxing, and stirring. After cooling to room temperature, water and toluene were added to a reaction solution, and an organic layer was separately taken. The organic layer was washed with a saline solution and then dried over MgSO₄. MgSO₄ was filtered and separated, and an organic layer was concentrated to obtain a crude product. The obtained crude product was purified to obtain Intermediate IM-8 (17.80 g, yield of 48%).

The mass number of Intermediate IM-8, measured by FAB-MS was 256.7.

### Synthesis of Compound 89

Compound 89 (4.54 g, yield of 69%) was synthesized in the same manner as the synthetic method of Compound 1 except for using Intermediate IM-8 in place of 4-bromobiphenyl.

The mass number of Compound 89, measured by FAB-MS was 800.3.

### (7) Synthesis of Compound 94

Amine Compound 94 according to one example may be synthesized by, for example, steps of Reaction Scheme 7.

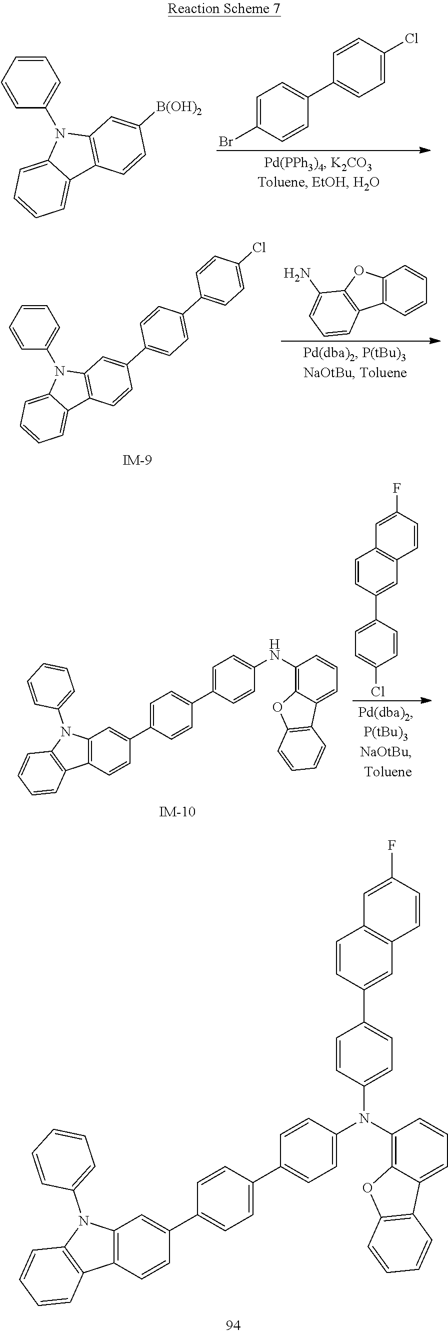

### Synthesis of Intermediate IM-9

Under an argon atmosphere, 9-phenylcarbazole-2-boronic acid (26.83 g, 93.44 mmol), 4-bromo-4′-chloro-1,1′-biphenyl (25 g, 1.0 equiv., 93.44 mmol), Pd(PPh₃)₄(5.40 g, 0.05 equiv., 4.67 mmol), K₂CO₃ (25.83 g, 2.0 equiv., 186.88 mmol), toluene (311 mL), EtOH (47 mL), and H₂O (93 mL) were added to a 1,000 mL three-neck flask, one by one, followed by heating, refluxing, and stirring. After cooling to room temperature, water and toluene were added to a reaction solution, and an organic layer was separately taken. The organic layer was washed with a saline solution and then dried over MgSO₄. MgSO₄ was filtered and separated, and an organic layer was concentrated to obtain a crude product. The obtained crude product was purified to obtain Intermediate IM-9 (47.15 g, yield of 63%).

The mass number of Intermediate IM-9, measured by FAB-MS was 429.13.

### Synthesis of Intermediate IM-10

Under an argon atmosphere, Intermediate IM-9 (39.89 g, 92.79 mmol), Pd(dba)₂ (0.533 g, 0.01 equiv., 0.93 mmol), NaOtBu (13.38 g, 1.5 equiv., 139.18 mmol), toluene (300 mL), dibenzofuran-4-amine (17.00 g, 1.0 equiv., 92.79 mmol) and PtBu₃ (1.86 mL, 0.04 equiv., 3.71 mmol) were added to a 500 mL three-neck flask, one by one, followed by heating, refluxing, and stirring. After cooling to room temperature, water was added to a reaction solution, and an organic layer was separately taken. Toluene was added to an aqueous layer, and the organic layer was additionally extracted. Then, the organic layers were collected, washed with a saline solution, and then dried over MgSO₄. MgSO₄ was filtered and separated, and the organic layers were concentrated to obtain a crude product. The obtained crude product was purified to obtain Intermediate IM-10 (35.32 g, yield of 66%).

The mass number of Intermediate IM-10, measured by FAB-MS was 576.22.

### Synthesis of Compound 94

Under an argon atmosphere, Intermediate IM-10 (5.00 g, 8.67 mmol), Pd(dba)₂ (0.10 g, 0.02 equiv., 0.17 mmol), NaOtBu (1.25 g, 1.5 equiv., 13.01 mmol), toluene (30 mL), Intermediate IM-1 (2.23 g, 1.0 equiv., 8.67 mmol) and PtBu₃ (0.17 mL, 0.04 equiv., 0.35 mmol) were added to a 200 mL three-neck flask, one by one, followed by heating, refluxing, and stirring. After cooling to room temperature, water was added to a reaction solution, and an organic layer was separately taken. Toluene was added to an aqueous layer, and the organic layer was additionally extracted. Then, the organic layers were collected, washed with a saline solution, and then dried over MgSO₄. MgSO₄ was filtered and separated, and the organic layers were concentrated to obtain a crude product. The obtained crude product was purified to obtain Compound 94 (5.17 g, yield of 75%).

The mass number of Compound 94, measured by FAB-MS was 796.29.

### 2. Manufacture and Evaluation of Light-Emitting Element

### (1) Manufacture of Light-Emitting Element

Light-emitting elements, including the amine compound according to Examples, or Comparative Example Compounds in a hole transport layer was manufactured through disclosed methods. Light emitting elements according to Example 1 to Example 7 were manufactured using the amine compounds according to Examples, respectively, as materials of the hole transport layer. Light-emitting elements according to Comparative Example 1 to Comparative Example 3 were manufactured using Comparative Example Compounds C1 to C3, respectively, as materials of the hole transport layer. Comparative Example Compounds used in Comparative Examples 1 to 3 are as follows.

### Comparative Example Compounds

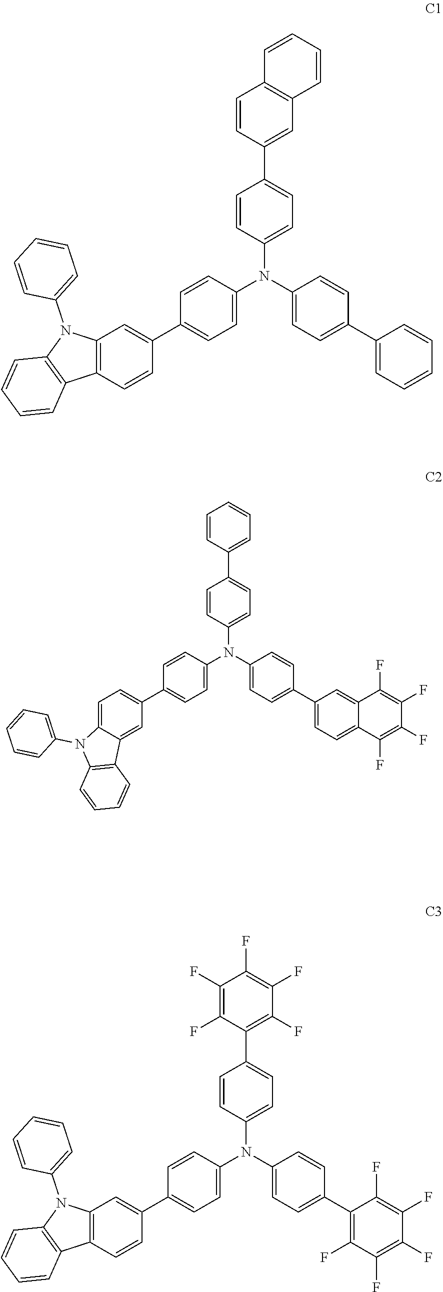 [SMILES: c1ccc(-c2ccc(N(c3ccc(-c4ccc5ccccc5c4)cc3)c3ccc(-c4ccc5c6ccccc6n(-c6ccccc6)c5c4)cc3)cc2)cc1 ; Fc1c(F)c(F)c2cc(-c3ccc(N(c4ccc(-c5ccccc5)cc4)c4ccc(-c5ccc6c(c5)c5ccccc5n6-c5ccccc5)cc4)cc3)ccc2c1F ; Fc1c(F)c(F)c(-c2ccc(N(c3ccc(-c4ccc5c6ccccc6n(-c6ccccc6)c5c4)cc3)c3ccc(-c4c(F)c(F)c(F)c(F)c4F)cc3)cc2)c(F)c1F]

A glass substrate, on which ITO of about 150 nm was patterned, as a first electrode was ultrasonically cleaned with isopropyl alcohol and ultrapure water for about 5 minutes each. After completion of ultrasonic cleaning, the glass substrate was subjected to UV (ultraviolet) irradiation for about 30 minutes and then ozone treatment was carried out. Thereafter, HIL and H-1-1, materials used in manufacture of light-emitting element and shown later, were deposited to a thickness of about 10 nm to form a hole injection layer. HIL and H-1-1 were deposited at a weight ratio of about 2:98. H-1-1 was deposited on the hole injection layer to a thickness of about 120 nm (nanometers) to form a first hole transport layer. Example Compound or Comparative Example Compound was deposited to a thickness of about 10 nm to form a second hole transport layer. The hole injection layer, the first hole transport layer, and the second hole transport layer form the hole transport region.

BD and BH, materials used in manufacture of light-emitting element and shown later, were co-deposited to form an emission layer on the second hole transport layer to a thickness of about 30 nm. BD and BH were co-deposited at a weight ratio of about 2:98. Thereafter, ET1 was deposited on the emission layer to a thickness of about 10 nm to form a first electron transport layer. ET2 was deposited on the first electron transport layer to a thickness of about 20 nm to form a second electron transport layer. LiF was deposited on the second electron transport layer to a thickness of about 1 nm to form an electron injection layer. The first electron transport layer, the second electron transport layer, and the electron injection layer form the electron transport region.

Then, Ag and Mg were deposited to a thickness of about 120 nm to form a second electrode. Ag and Mg were deposited at a weight ratio of about 10:90.

In Examples and Comparative Examples, the hole transport region, the emission layer, the electron transport region, and the second electrode were formed by using a vacuum deposition device.

Compounds used for the manufacture of the light-emitting elements are as follows.

### (Materials Used in Manufacture of Light-Emitting Element)

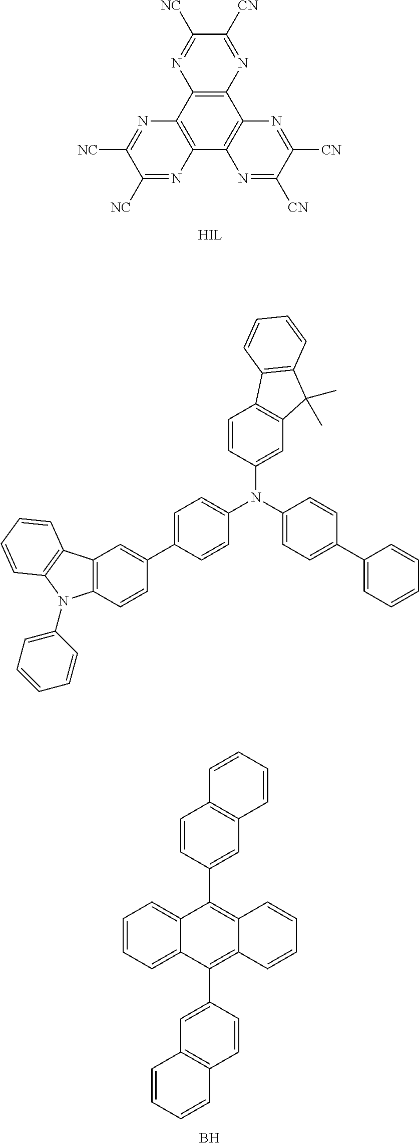

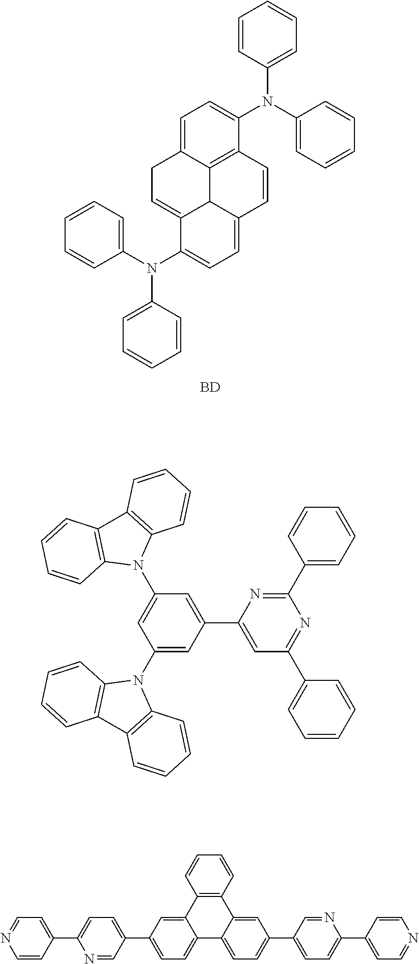

### (2) Evaluation of Light-Emitting Element

The light-emitting elements according to Examples and Comparative Examples were evaluated and the evaluated results are listed in Table 1. Driving voltages and lifespans of the light-emitting elements according to Examples and Comparative Examples were expressed as relative values. The driving voltage was measured by turning the light-emitting element on at 10 mA/cm² (milliampere per centimeter squared). The lifespan was expressed as the relative value of the time taken for luminance to decrease to 50% from 100% of an initial luminance when continuously driven at 10 mA/cm² with respect to the numerical value of Comparative Example 1 of 100%. The driving voltage was relatively expressed as an increase or decrease with respect to that of Comparative Example 1.

TABLE 1

| Examples of element | Materials for hole | Driving | Lifespan |
|---|---|---|---|
| manufacture | transport layer | voltage (V) | (%) |
| Example 1 | Compound 1 | −0.09 | 110 |
| Example 2 | Compound 2 | −0.08 | 119 |
| Example 3 | Compound 4 | −0.11 | 112 |
| Example 4 | Compound 12 | −0.07 | 122 |
| Example 5 | Compound 85 | −0.13 | 105 |
| Example 6 | Compound 89 | −0.09 | 123 |
| Example 7 | Compound 94 | −0.11 | 115 |
| Comparative | Comparative Example | 0.00 | 100 |
| Example 1 | Compound C1 | | |
| Comparative | Comparative Example | +0.05 | 86 |
| Example 2 | Compound C2 | | |
| Comparative | Comparative Example | +0.08 | 89 |
| Example 3 | Compound C3 | | |

Referring to the results in Table 1, the light-emitting elements according to Example 1 to Example 7 exhibit lower driving voltages (V) and higher lifespans than the light-emitting elements according to Comparative Example 1 to Comparative Example 3.

Example Compounds include a 2-carbazole group structure and a structure that includes a naphthyl group substituted with at least one fluorine atom which may result or may be a resulting factor for the high stability observed results recorded in Table 1.

In a carbazole group, reactivity may be lower at a 2-position than at a 3-position.

In Example Compounds, the amine compound that includes a naphthyl group may overlap forming a stack of molecules due to a planar structure of the naphthyl group. The results recorded in Table 1 indicate that in Example Compounds, charges may be easily transferred by hopping, and thus the light-emitting element may exhibit characteristics of the reduced driving voltage when Example Compounds are used in the hole transport region of the light-emitting element.

A fluorine atom has the highest electronegativity among all atoms. Therefore, when a naphthyl group, which is an aromatic compound, is substituted with a fluorine atom, the fluorine atom may serve as an electron-withdrawing group. Therefore, when the naphthyl group is substituted with a fluorine atom, an overall electron density of an aromatic ring decreases, and thus electrophilic substitution reaction may be suppressed. The results recorded in Table 1 indicate that the light-emitting elements according to Examples containing Example Compounds in the hole transport layer exhibit improved lifespan characteristics.

Comparative Example Compound C1 differs from Example Compounds in that a naphthyl group is not substituted with fluorine. Without intent to be bound by theory, it is believed that a molecule of Comparative Example Compound C1 is more susceptible to an influence of an electron than Example Compounds. The light-emitting element according to Comparative Example 1 using Comparative Example Compound C1 exhibit poorer element lifespan characteristics than the light-emitting elements according to Examples.

Comparative Example Compound C2 differs from Example Compounds in that Comparative Example Compound C2 includes a 3-carbazole group structure and an unsubstituted phenyl group instead of the naphthyl group substituted with a fluorine group. The lack of stacking may result in Comparative Example Compound C2 having a lower molecular stability than Example Compounds. The light-emitting element according to Comparative Example 2 using Comparative Example Compound C2 exhibit characteristics that a driving voltage is higher than the light-emitting elements according to Examples, and the element lifespan is lower than the light-emitting elements using Example Compounds.

Comparative Example Compound C3 differs from Example Compounds in that Comparative Example Compound C3 includes a phenyl group substituted with a fluorine atom instead of the naphthyl group substituted with a fluorine atom. Without intent to be bound by theory, it is believed that transfer of charges by hopping is more difficult in Comparative Example Compound C3 than in Example Compounds. Therefore, the light-emitting element according to Comparative Example 3 using Comparative Example Compound C3 exhibit characteristics that the driving voltage is higher than those of the light-emitting elements using Example Compounds and the element lifespan is lower than those of the light-emitting elements using Example Compounds.

The amine compound according to an embodiment may have structural characteristics such that a 2-carbazole group-containing structure and a structure including a naphthyl group substituted with at least one fluorine atom are included, thus allowing the light-emitting element to exhibit excellent stability and hole transporting ability.

The light-emitting element according to an embodiment which includes the amine compound according to an embodiment in the hole transport region may exhibit element characteristics of the reduced driving voltage and the increased lifespan. In addition, the light-emitting element according to an embodiment may emit blue light and includes the amine compound according to an embodiment in the hole transport region, and thus may exhibit the low driving voltage and long lifespan characteristics.

The light-emitting element according to an embodiment may exhibit low driving voltage and long lifespan characteristics by including the amine compound according to an embodiment in the hole transport region.

The amine compound according to an embodiment may be used as a material for achieving improved characteristics of the light-emitting element having a low driving voltage and a long lifespan.

The electronic apparatus according to an embodiment may exhibit excellent display quality and improved reliability characteristics.

In the above, description has been made with reference to embodiments of this disclosure, but those skilled or of ordinary skill in the art may understand that various modifications and changes may be made to the inventive concept insofar as such modifications and changes do not depart from the spirit and technical scope set forth in the claims to be described later.

Therefore, the technical scope of this disclosure is not to be limited to the contents stated in the detailed description of the specification, but should be determined by the claims.

What is claimed is:

1. A light-emitting element comprising:
a first electrode;
a second electrode disposed on the first electrode;
an emission layer disposed between the first electrode and the second electrode; and
a hole transport region disposed between the first electrode and the emission layer, wherein the hole transport region comprises an amine compound according to Formula 1. 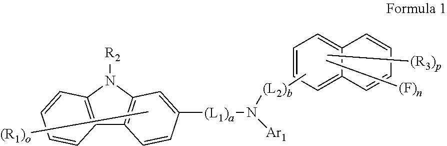
wherein, in Formula 1,
L₁ and L₂ are each independently a substituted or unsubstituted arylene group having 6 to 30 carbon atoms,
Ar₁ is a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms,
each R₁, R₂, and each R₃ are independently a hydrogen atom, a deuterium atom, a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms
a and b are each independently an integer of 0 to 4, provided that:
when a is 0, N of NAr₁ is directly bonded to the 2-position of a carbazole group, and
when b is 0, N of NAr₁ is directly bonded to a naphthyl group,
n is an integer of 1 to 7,
o is an integer of 0 to 7, and
p is an integer of 0 to (7-n).

2. The light-emitting element of claim 1, wherein
the hole transport region comprises at least one of a hole injection layer, a hole transport layer, an electron-blocking layer, and an emission auxiliary layer, and
the amine compound is included in at least one of the hole injection layer, the hole transport layer, the electron-blocking layer, and the emission auxiliary layer.

3. The light-emitting element of claim 1, wherein,
the hole transport region comprises a hole injection layer disposed on the first electrode and a hole transport layer disposed on the hole injection layer, and
the amine compound is included in the hole transport layer.

4. The light-emitting element of claim 1, wherein,
the hole transport region comprises a hole injection layer disposed on the first electrode, a first hole transport layer disposed on the hole injection layer, and a second hole transport layer adjacent to a bottom of the emission layer and disposed on the first hole transport layer, and
the amine compound is included in the second hole transport layer.

5. The light-emitting element of claim 1, wherein the amine compound has a structure according to any one of Formula 2-1 to Formula 2-3: 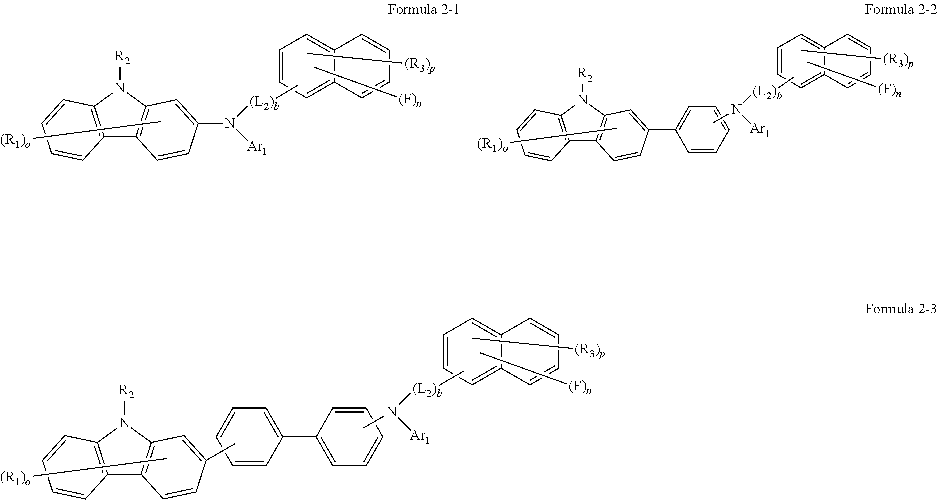
where, in Formula 2-1 to Formula 2-3, L₂, Ar₁, R₁ to R₃, b, n, o, and p are the same as defined in Formula 1.

6. The light-emitting element of claim 1, wherein the amine compound has a structure according to any one of Formula 3-1 to Formula 3-3: 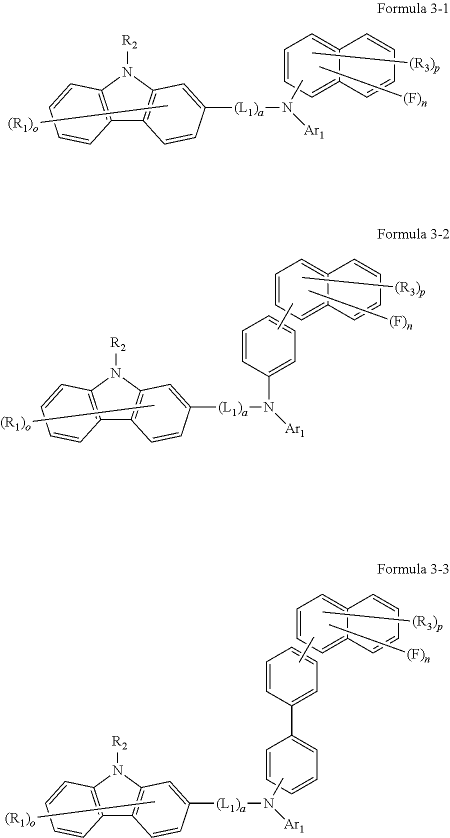
where, in Formula 3-1 to Formula 3-3, L₁, Ar₁, R₁ to R₃, a, n, o, and p are the same as defined in Formula 1.

7. The light-emitting element of claim 1, wherein Ar₁ is a substituted or unsubstituted phenyl group, a substituted or unsubstituted dibenzofuran group, a substituted or unsubstituted dibenzothiophene group, a substituted or unsubstituted naphthyl group, or a substituted or unsubstituted naphthobenzofuran group.

8. The light-emitting element of claim 1, wherein the emission layer includes a compound according to Formula E-1: 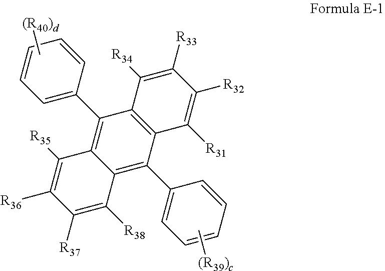
where, in Formula E-1,
c and d are each independently an integer of 0 to 5, and
R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring.

9. The light-emitting element of claim 1, wherein the amine compound has a structure according to any one of the compounds in Compound Group 1: 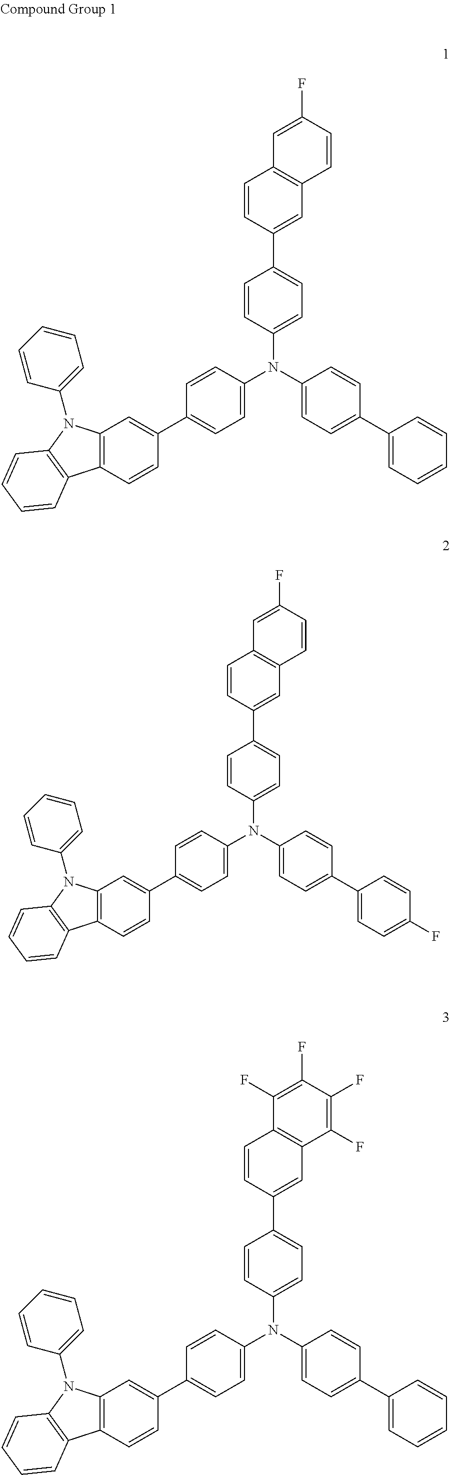 [SMILES: Fc1ccc2cc(-c3ccc(N(c4ccc(-c5ccccc5)cc4)c4ccc(-c5ccc6c7ccccc7n(-c7ccccc7)c6c5)cc4)cc3)ccc2c1 ; Fc1ccc(-c2ccc(N(c3ccc(-c4ccc5cc(F)ccc5c4)cc3)c3ccc(-c4ccc5c6ccccc6n(-c6ccccc6)c5c4)cc3)cc2)cc1 ; Fc1c(F)c(F)c2cc(-c3ccc(N(c4ccc(-c5ccccc5)cc4)c4ccc(-c5ccc6c7ccccc7n(-c7ccccc7)c6c5)cc4)cc3)ccc2c1F] 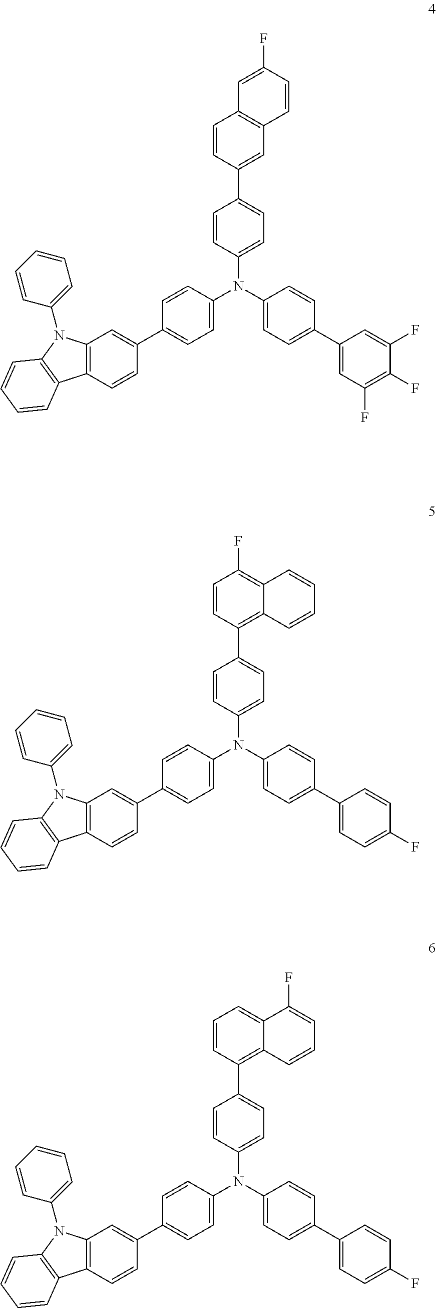 [SMILES: Fc1ccc2cc(-c3ccc(N(c4ccc(-c5cc(F)c(F)c(F)c5)cc4)c4ccc(-c5ccc6c7ccccc7n(-c7ccccc7)c6c5)cc4)cc3)ccc2c1 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)cc1 ; Fc1ccc(-c2ccc(N(c3ccc(-c4ccc5c6ccccc6n(-c6ccccc6)c5c4)cc3)c3ccc(-c4cccc5c(F)cccc45)cc3)cc2)cc1] 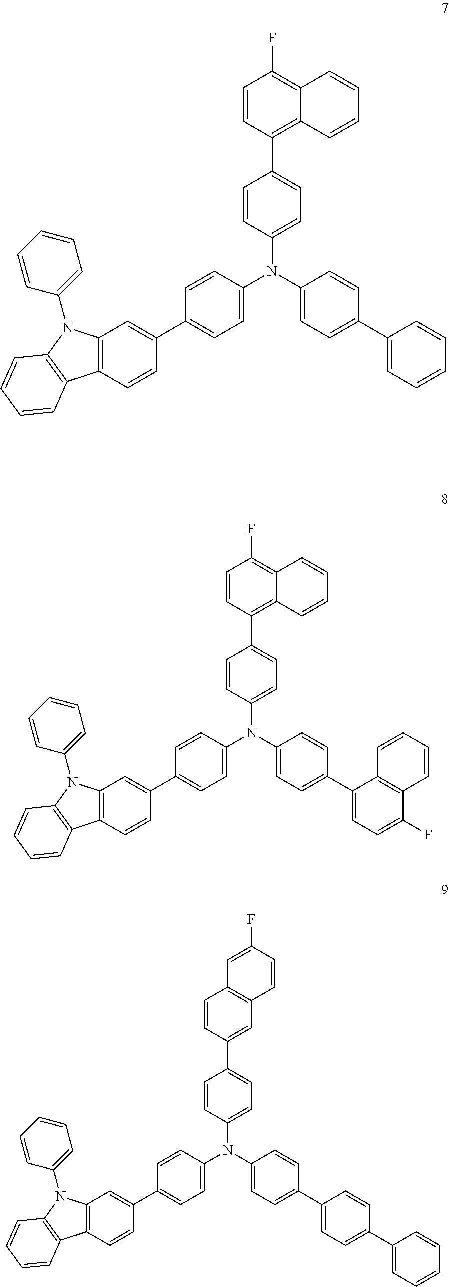 [SMILES: Fc1ccc(-c2ccc(N(c3ccc(-c4ccccc4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)c2ccccc12 ; Fc1ccc2cc(-c3ccc(N(c4ccc(-c5ccc(-c6ccccc6)cc5)cc4)c4ccc(-c5ccc6c7ccccc7n(-c7ccccc7)c6c5)cc4)cc3)ccc2c1] 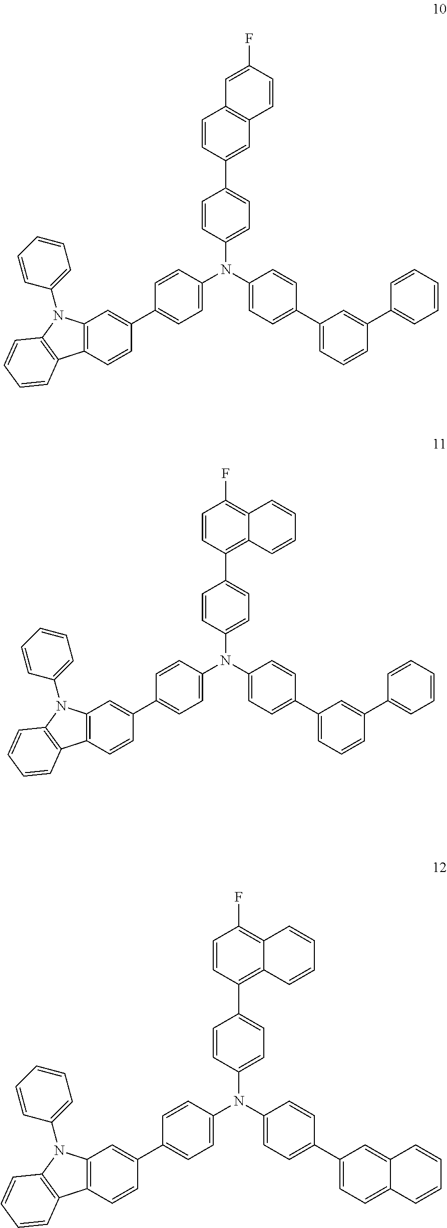 [SMILES: Fc1ccc2cc(-c3ccc(N(c4ccc(-c5cccc(-c6ccccc6)c5)cc4)c4ccc(-c5ccc6c7ccccc7n(-c7ccccc7)c6c5)cc4)cc3)ccc2c1 ; Fc1ccc(-c2ccc(N(c3ccc(-c4cccc(-c5ccccc5)c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5ccccc5c4)cc3)c3ccc(-c4ccc5c6ccccc6n(-c6ccccc6)c5c4)cc3)cc2)c2ccccc12] 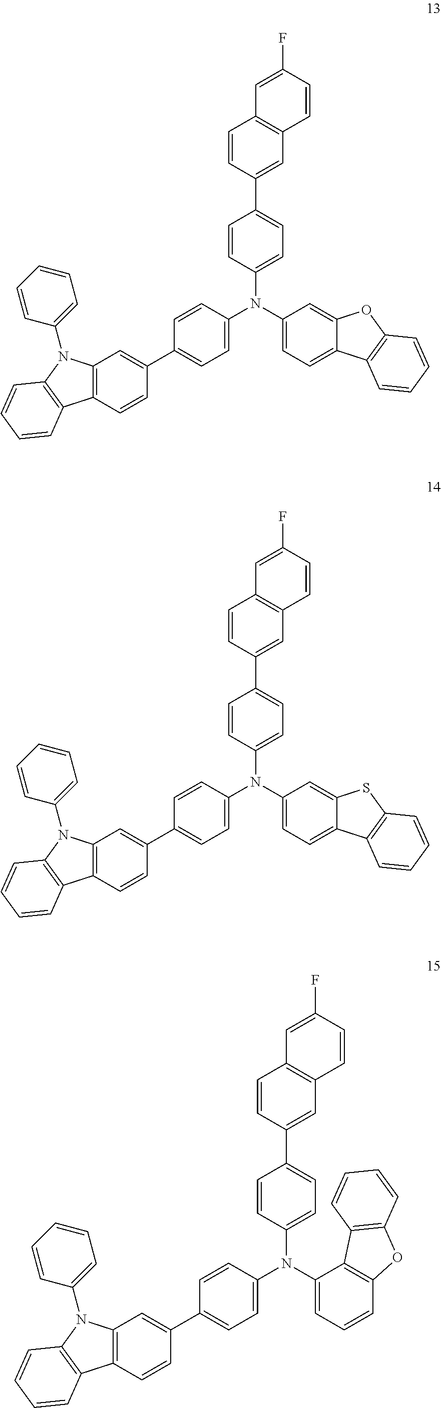 [SMILES: Fc1ccc2cc(-c3ccc(N(c4ccc(-c5ccc6c7ccccc7n(-c7ccccc7)c6c5)cc4)c4ccc5c(c4)oc4ccccc45)cc3)ccc2c1 ; Fc1ccc2cc(-c3ccc(N(c4ccc(-c5ccc6c7ccccc7n(-c7ccccc7)c6c5)cc4)c4ccc5c(c4)sc4ccccc45)cc3)ccc2c1 ; Fc1ccc2cc(-c3ccc(N(c4ccc(-c5ccc6c7ccccc7n(-c7ccccc7)c6c5)cc4)c4cccc5oc6ccccc6c45)cc3)ccc2c1] 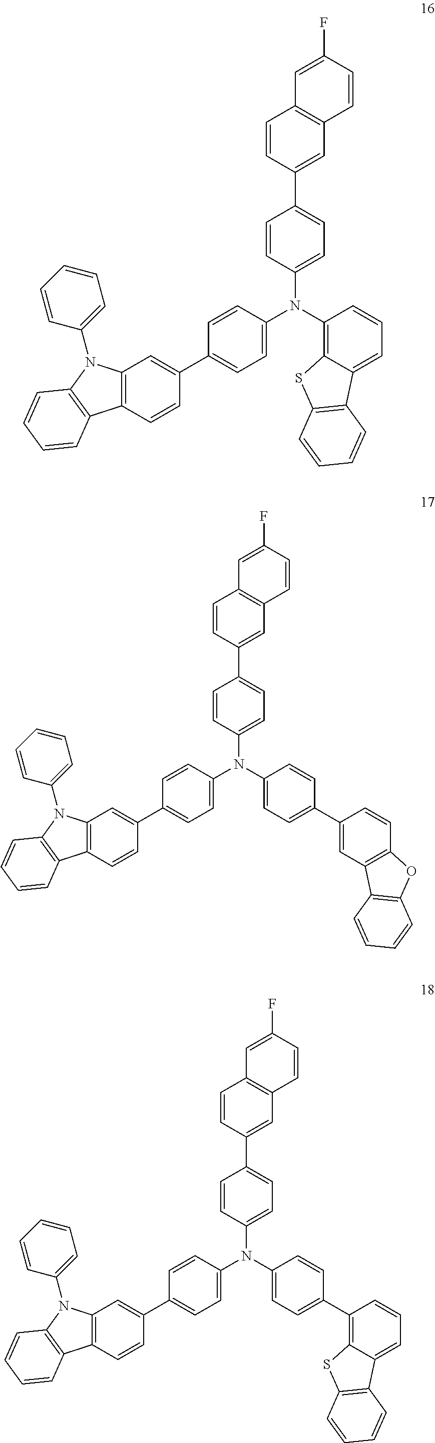 [SMILES: Fc1ccc2cc(-c3ccc(N(c4ccc(-c5ccc6c7ccccc7n(-c7ccccc7)c6c5)cc4)c4cccc5c4sc4ccccc45)cc3)ccc2c1 ; Fc1ccc2cc(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5sc5ccccc56)cc4)cc3)ccc2c1] 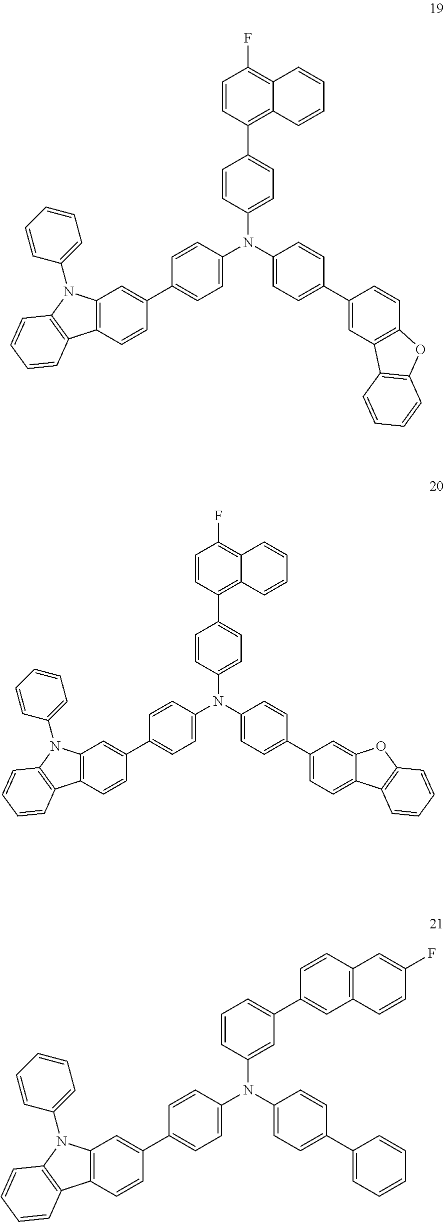 [SMILES: Fc1ccc(-c2ccc(N(c3ccc(-c4ccc5oc6ccccc6c5c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c(c4)oc4ccccc45)cc3)c3ccc(-c4ccc5c6ccccc6n(-c6ccccc6)c5c4)cc3)cc2)c2ccccc12 ; Fc1ccc2cc(-c3cccc(N(c4ccc(-c5ccccc5)cc4)c4ccc(-c5ccc6c7ccccc7n(-c7ccccc7)c6c5)cc4)c3)ccc2c1] 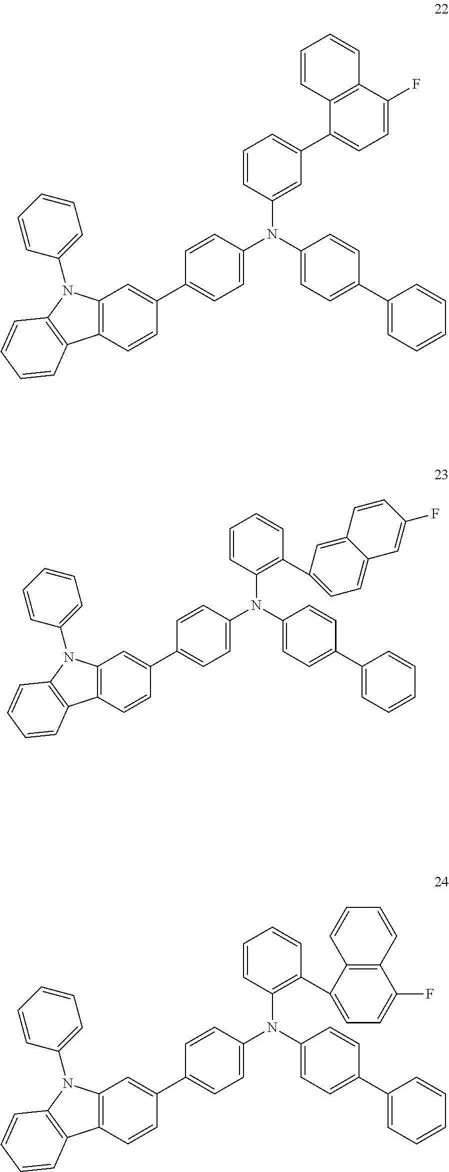 [SMILES: Fc1ccc(-c2cccc(N(c3ccc(-c4ccccc4)cc3)c3ccc(-c4ccc5c6ccccc6n(-c6ccccc6)c5c4)cc3)c2)c2ccccc12 ; Fc1ccc2cc(-c3ccccc3N(c3ccc(-c4ccccc4)cc3)c3ccc(-c4ccc5c6ccccc6n(-c6ccccc6)c5c4)cc3)ccc2c1 ; Fc1ccc(-c2ccccc2N(c2ccc(-c3ccccc3)cc2)c2ccc(-c3ccc4c5ccccc5n(-c5ccccc5)c4c3)cc2)c2ccccc12] 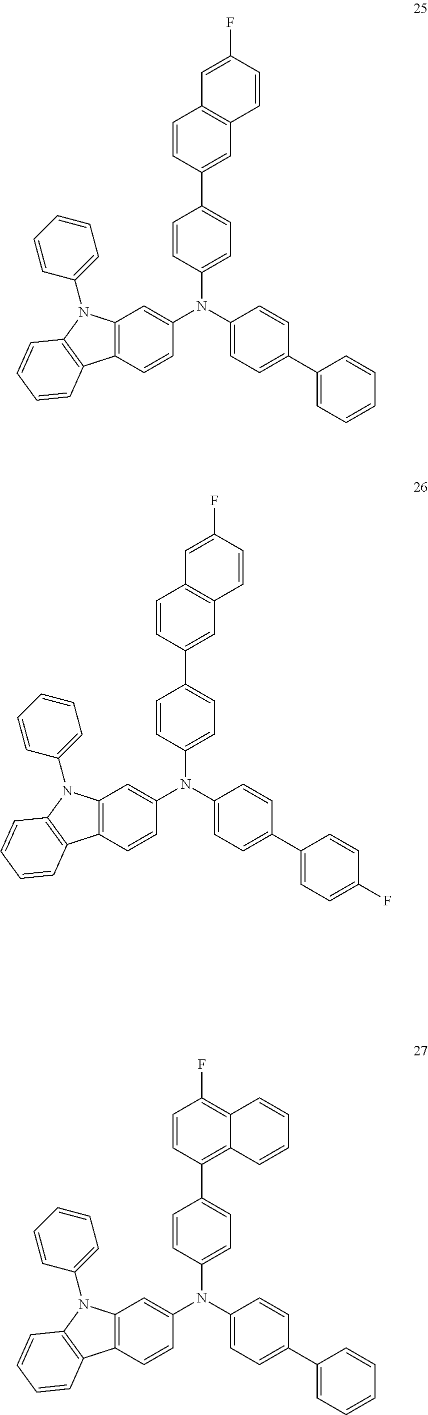 [SMILES: Fc1ccc2cc(-c3ccc(N(c4ccc(-c5ccccc5)cc4)c4ccc5c6ccccc6n(-c6ccccc6)c5c4)cc3)ccc2c1 ; Fc1ccc(-c2ccc(N(c3ccc(-c4ccc5cc(F)ccc5c4)cc3)c3ccc4c5ccccc5n(-c5ccccc5)c4c3)cc2)cc1 ; Fc1ccc(-c2ccc(N(c3ccc(-c4ccccc4)cc3)c3ccc4c5ccccc5n(-c5ccccc5)c4c3)cc2)c2ccccc12] 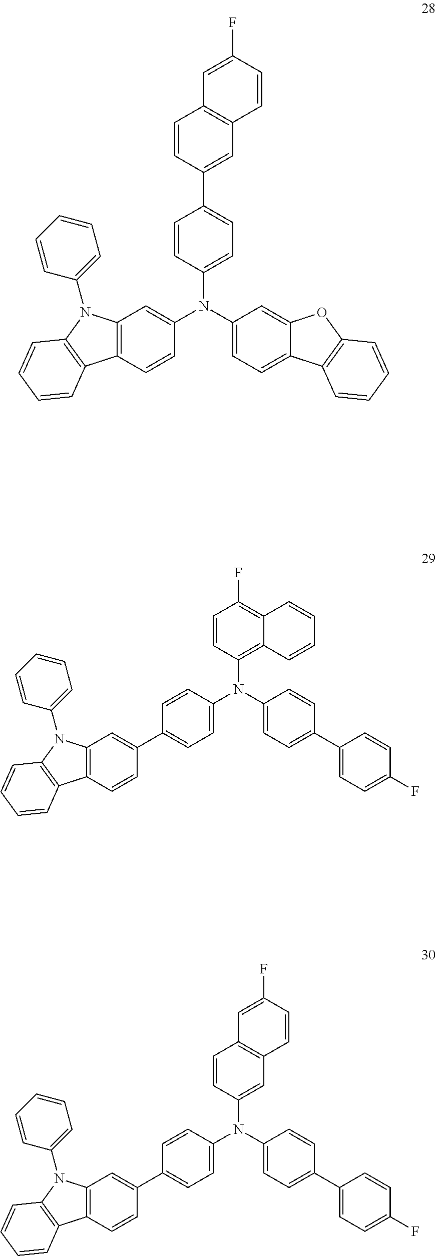 [SMILES: Fc1ccc2cc(-c3ccc(N(c4ccc5c(c4)oc4ccccc45)c4ccc5c6ccccc6n(-c6ccccc6)c5c4)cc3)ccc2c1 ; Fc1ccc(-c2ccc(N(c3ccc(-c4ccc5c6ccccc6n(-c6ccccc6)c5c4)cc3)c3ccc(F)c4ccccc34)cc2)cc1 ; Fc1ccc(-c2ccc(N(c3ccc(-c4ccc5c6ccccc6n(-c6ccccc6)c5c4)cc3)c3ccc4cc(F)ccc4c3)cc2)cc1] 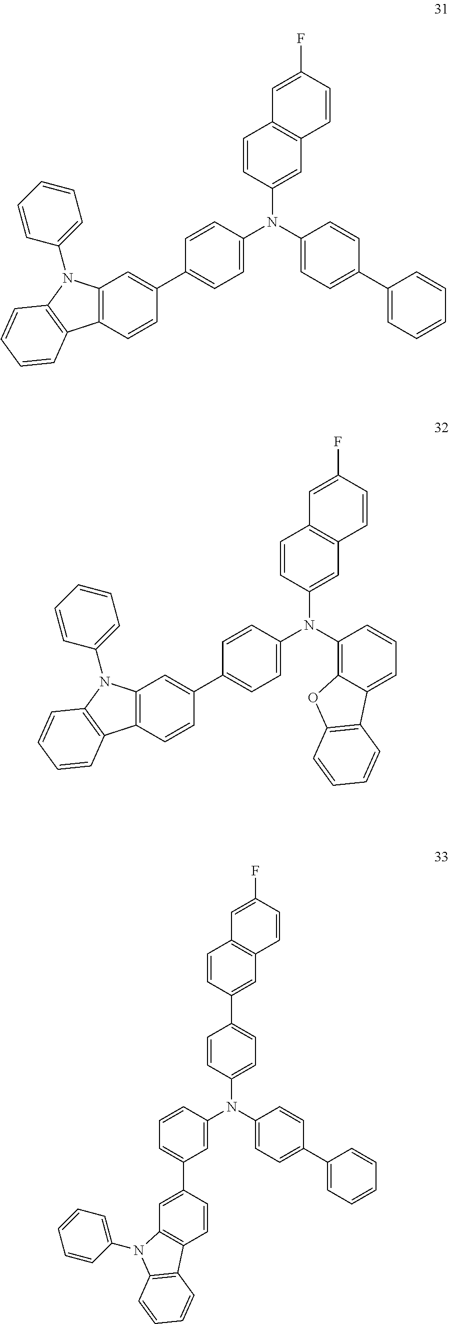 [SMILES: Fc1ccc2cc(N(c3ccc(-c4ccccc4)cc3)c3ccc(-c4ccc5c6ccccc6n(-c6ccccc6)c5c4)cc3)ccc2c1 ; Fc1ccc2cc(N(c3ccc(-c4ccc5c6ccccc6n(-c6ccccc6)c5c4)cc3)c3cccc4c3oc3ccccc34)ccc2c1 ; Fc1ccc2cc(-c3ccc(N(c4ccc(-c5ccccc5)cc4)c4cccc(-c5ccc6c7ccccc7n(-c7ccccc7)c6c5)c4)cc3)ccc2c1] 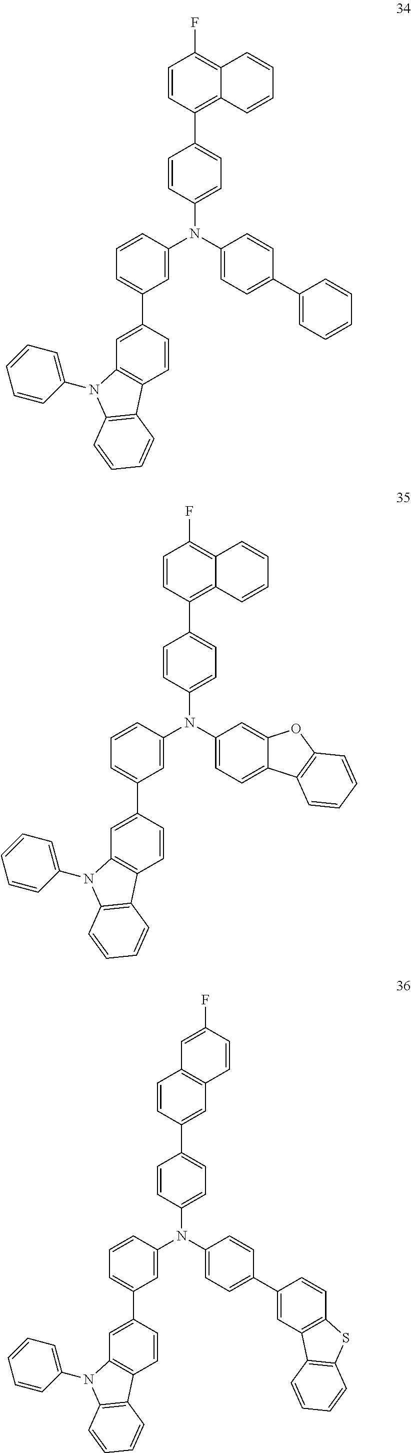 [SMILES: Fc1ccc(-c2ccc(N(c3ccc(-c4ccccc4)cc3)c3cccc(-c4ccc5c6ccccc6n(-c6ccccc6)c5c4)c3)cc2)c2ccccc12 ; Fc1ccc(-c2ccc(N(c3cccc(-c4ccc5c6ccccc6n(-c6ccccc6)c5c4)c3)c3ccc4c(c3)oc3ccccc34)cc2)c2ccccc12 ; Fc1ccc2cc(-c3ccc(N(c4ccc(-c5ccc6sc7ccccc7c6c5)cc4)c4cccc(-c5ccc6c7ccccc7n(-c7ccccc7)c6c5)c4)cc3)ccc2c1] 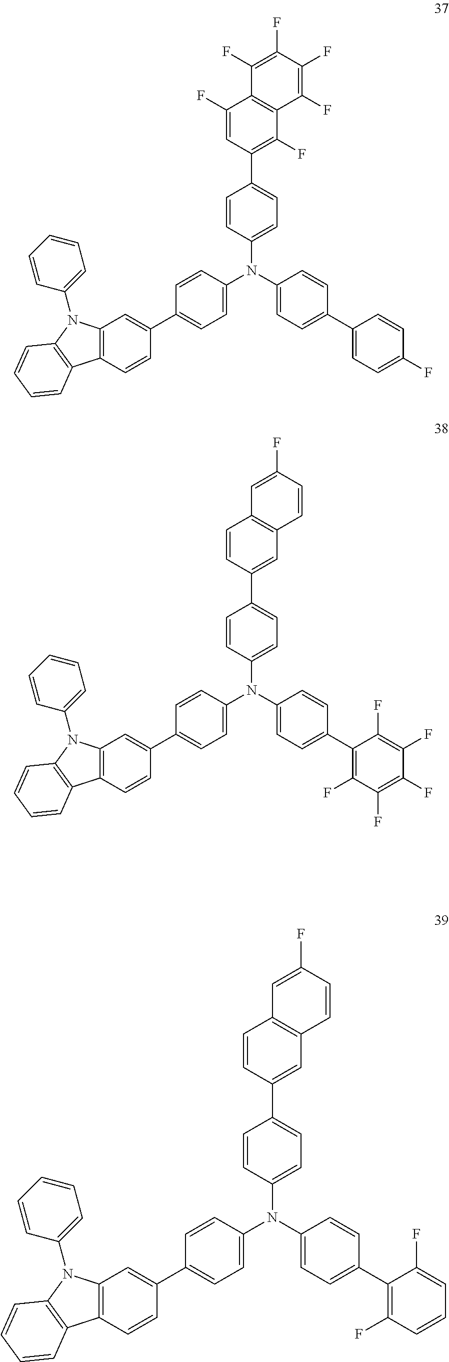 [SMILES: Fc1ccc(-c2ccc(N(c3ccc(-c4ccc5c6ccccc6n(-c6ccccc6)c5c4)cc3)c3ccc(-c4cc(F)c5c(F)c(F)c(F)c(F)c5c4F)cc3)cc2)cc1 ; Fc1ccc2cc(-c3ccc(N(c4ccc(-c5ccc6c7ccccc7n(-c7ccccc7)c6c5)cc4)c4ccc(-c5c(F)c(F)c(F)c(F)c5F)cc4)cc3)ccc2c1 ; Fc1ccc2cc(-c3ccc(N(c4ccc(-c5ccc6c7ccccc7n(-c7ccccc7)c6c5)cc4)c4ccc(-c5c(F)cccc5F)cc4)cc3)ccc2c1] 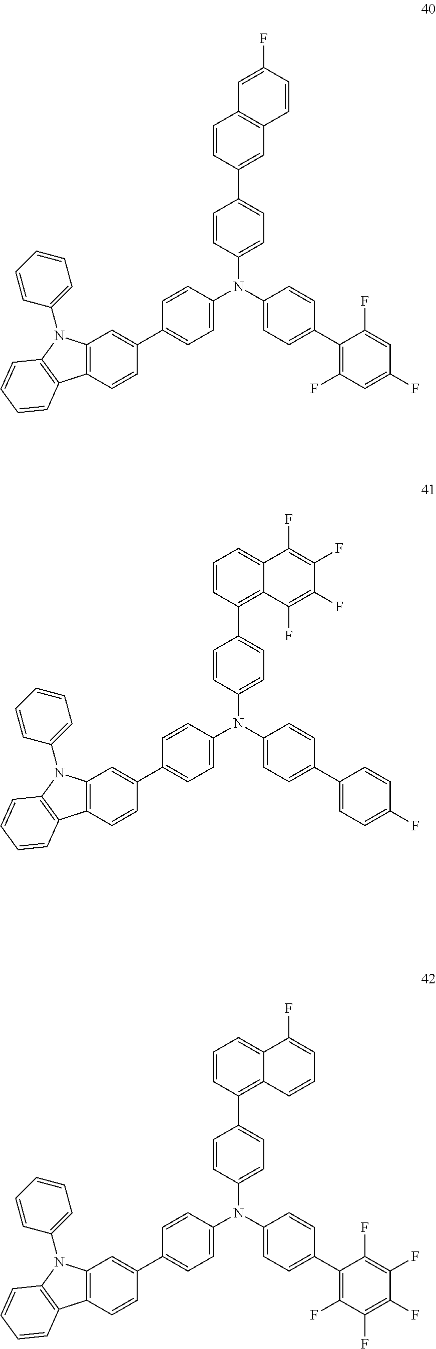 [SMILES: Fc1cc(F)c(-c2ccc(N(c3ccc(-c4ccc5cc(F)ccc5c4)cc3)c3ccc(-c4ccc5c6ccccc6n(-c6ccccc6)c5c4)cc3)cc2)c(F)c1 ; Fc1ccc(-c2ccc(N(c3ccc(-c4ccc5c6ccccc6n(-c6ccccc6)c5c4)cc3)c3ccc(-c4cccc5c(F)c(F)c(F)c(F)c45)cc3)cc2)cc1 ; Fc1c(F)c(F)c(-c2ccc(N(c3ccc(-c4ccc5c6ccccc6n(-c6ccccc6)c5c4)cc3)c3ccc(-c4cccc5c(F)cccc45)cc3)cc2)c(F)c1F] 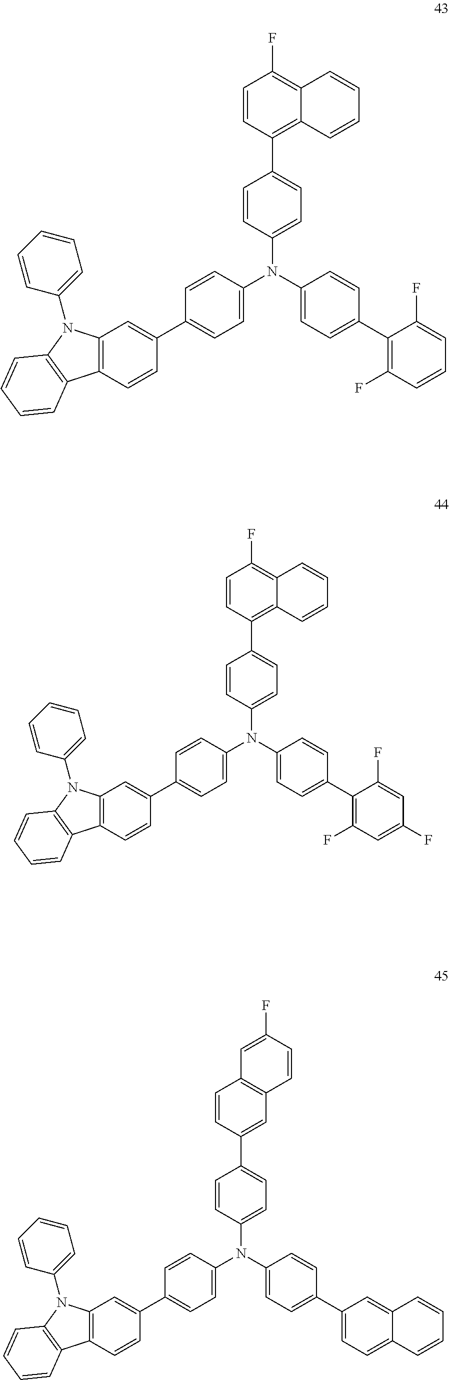 [SMILES: Fc1cccc(F)c1-c1ccc(N(c2ccc(-c3ccc4c5ccccc5n(-c5ccccc5)c4c3)cc2)c2ccc(-c3ccc(F)c4ccccc34)cc2)cc1 ; Fc1cc(F)c(-c2ccc(N(c3ccc(-c4ccc5c6ccccc6n(-c6ccccc6)c5c4)cc3)c3ccc(-c4ccc(F)c5ccccc45)cc3)cc2)c(F)c1 ; Fc1ccc2cc(-c3ccc(N(c4ccc(-c5ccc6ccccc6c5)cc4)c4ccc(-c5ccc6c7ccccc7n(-c7ccccc7)c6c5)cc4)cc3)ccc2c1] 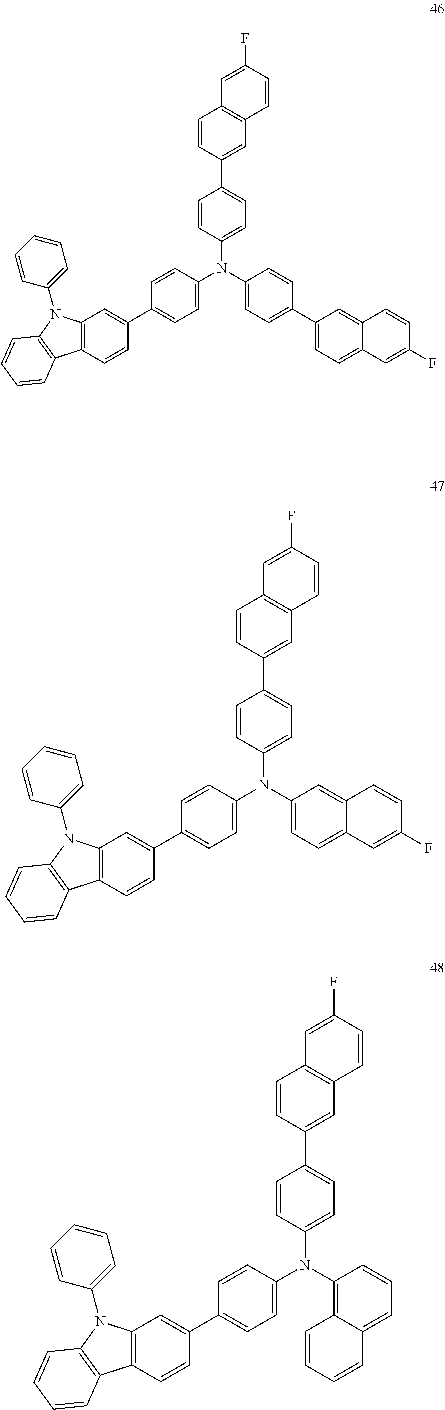 [SMILES: Fc1ccc2cc(-c3ccc(N(c4ccc(-c5ccc6cc(F)ccc6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc5cc(F)ccc5c4)cc3)ccc2c1 ; Fc1ccc2cc(-c3ccc(N(c4ccc(-c5ccc6c7ccccc7n(-c7ccccc7)c6c5)cc4)c4cccc5ccccc45)cc3)ccc2c1] 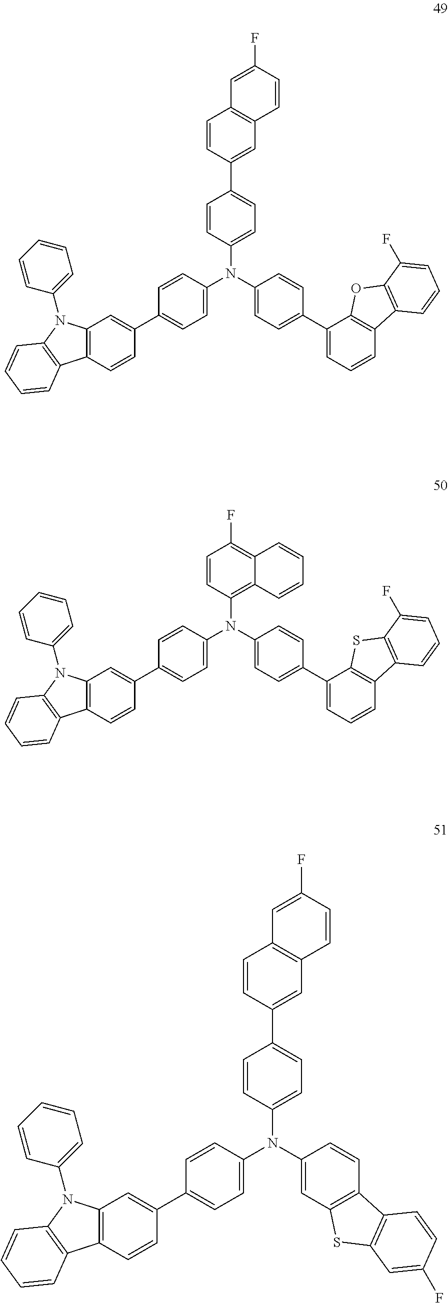 [SMILES: Fc1ccc2cc(-c3ccc(N(c4ccc(-c5ccc6c7ccccc7n(-c7ccccc7)c6c5)cc4)c4ccc(-c5cccc6c5oc5c(F)cccc56)cc4)cc3)ccc2c1 ; Fc1ccc(N(c2ccc(-c3ccc4c5ccccc5n(-c5ccccc5)c4c3)cc2)c2ccc(-c3cccc4c3sc3c(F)cccc34)cc2)c2ccccc12 ; Fc1ccc2cc(-c3ccc(N(c4ccc(-c5ccc6c7ccccc7n(-c7ccccc7)c6c5)cc4)c4ccc5c(c4)sc4cc(F)ccc45)cc3)ccc2c1] 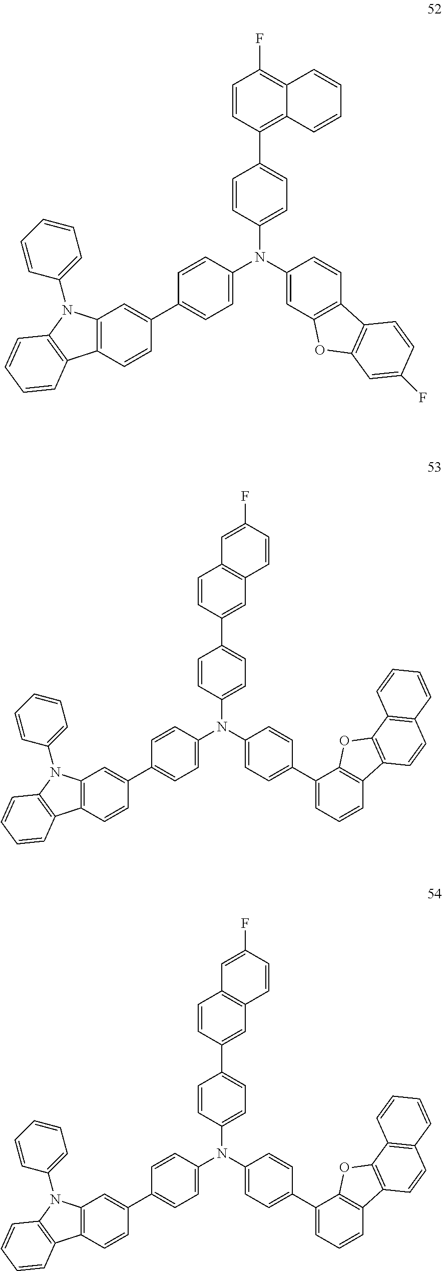 [SMILES: Fc1ccc2c(c1)oc1cc(N(c3ccc(-c4ccc5c6ccccc6n(-c6ccccc6)c5c4)cc3)c3ccc(-c4ccc(F)c5ccccc45)cc3)ccc12 ; Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1] 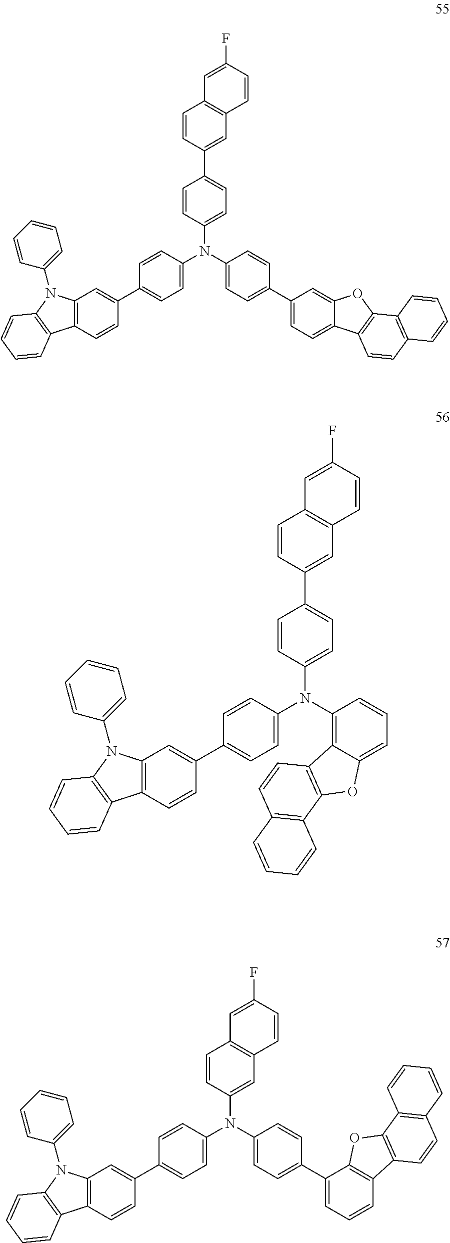 [SMILES: Fc1ccc2cc(-c3ccc(N(c4ccc(-c5ccc6c(c5)oc5c7ccccc7ccc65)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4cccc5oc6c7ccccc7ccc6c45)cc3)ccc2c1 ; Fc1ccc2cc(N(c3ccc(-c4ccc5c6ccccc6n(-c6ccccc6)c5c4)cc3)c3ccc(-c4cccc5c4oc4c6ccccc6ccc54)cc3)ccc2c1] 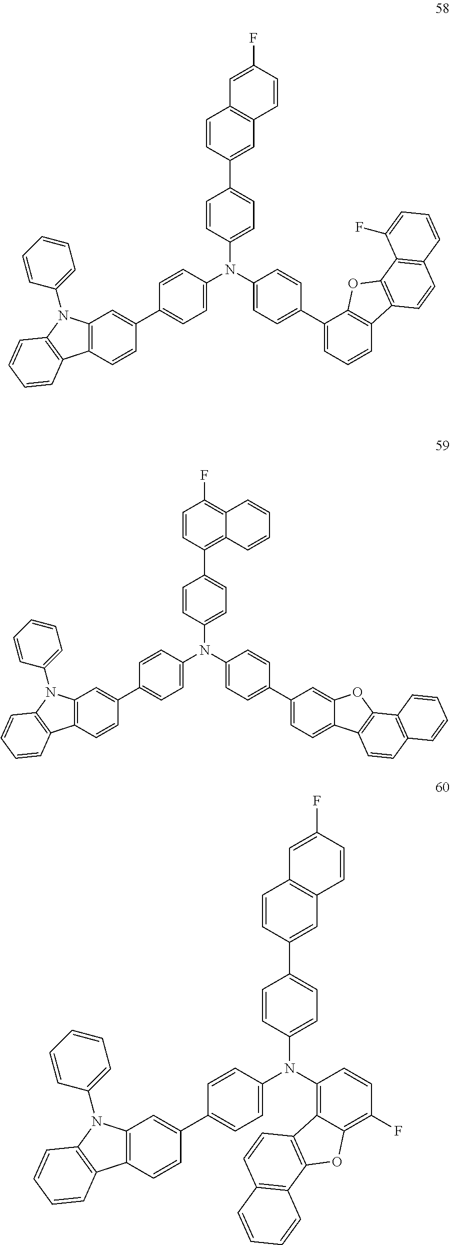 [SMILES: Fc1ccc2cc(-c3ccc(N(c4ccc(-c5ccc6c7ccccc7n(-c7ccccc7)c6c5)cc4)c4ccc(-c5cccc6c5oc5c6ccc6cccc(F)c65)cc4)cc3)ccc2c1 ; Fc1ccc(-c2ccc(N(c3ccc(-c4ccc5c(c4)oc4c6ccccc6ccc54)cc3)c3ccc(-c4ccc5c6ccccc6n(-c6ccccc6)c5c4)cc3)cc2)c2ccccc12 ; Fc1ccc2cc(-c3ccc(N(c4ccc(-c5ccc6c7ccccc7n(-c7ccccc7)c6c5)cc4)c4ccc(F)c5oc6c7ccccc7ccc6c45)cc3)ccc2c1] 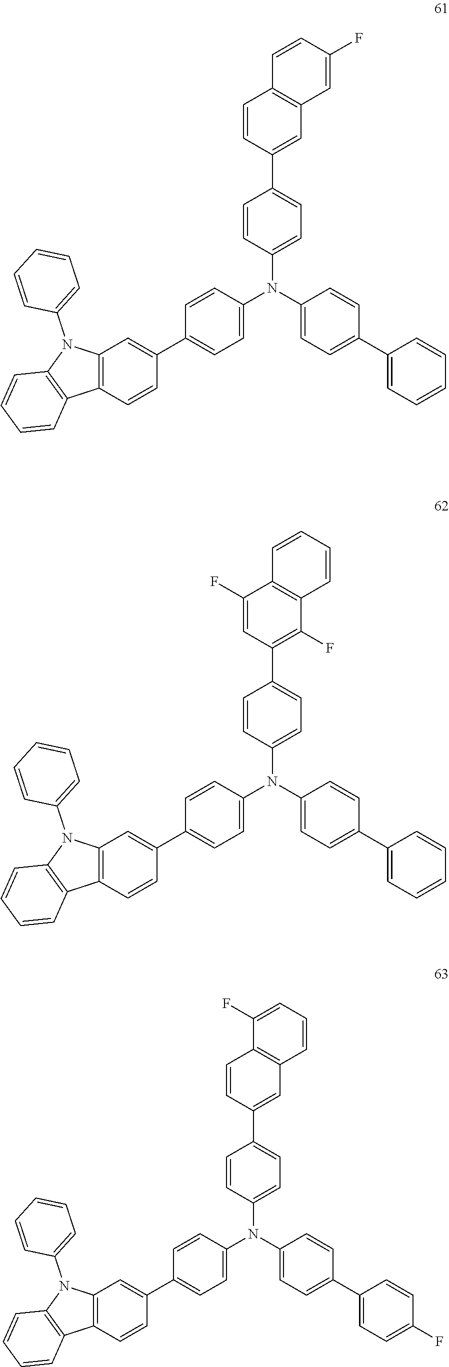 [SMILES: Fc1ccc2ccc(-c3ccc(N(c4ccc(-c5ccccc5)cc4)c4ccc(-c5ccc6c7ccccc7n(-c7ccccc7)c6c5)cc4)cc3)cc2c1 ; Fc1cc(-c2ccc(N(c3ccc(-c4ccccc4)cc3)c3ccc(-c4ccc5c6ccccc6n(-c6ccccc6)c5c4)cc3)cc2)c(F)c2ccccc12 ; Fc1ccc(-c2ccc(N(c3ccc(-c4ccc5c(F)cccc5c4)cc3)c3ccc(-c4ccc5c6ccccc6n(-c6ccccc6)c5c4)cc3)cc2)cc1] 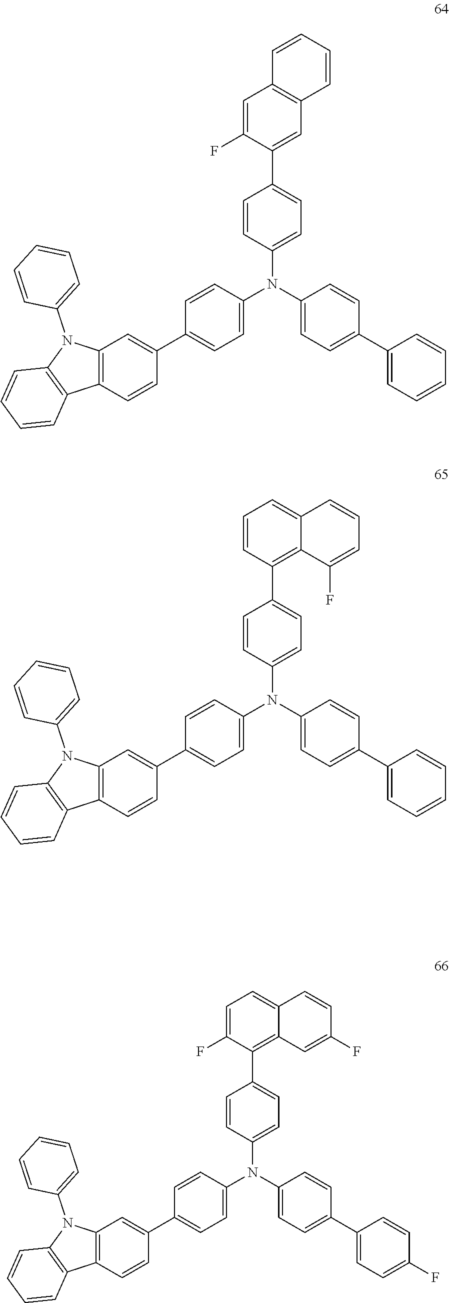 [SMILES: Fc1cc2ccccc2cc1-c1ccc(N(c2ccc(-c3ccccc3)cc2)c2ccc(-c3ccc4c5ccccc5n(-c5ccccc5)c4c3)cc2)cc1 ; Fc1cccc2cccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4c(F)ccc5ccc(F)cc45)cc3)cc2)cc1] 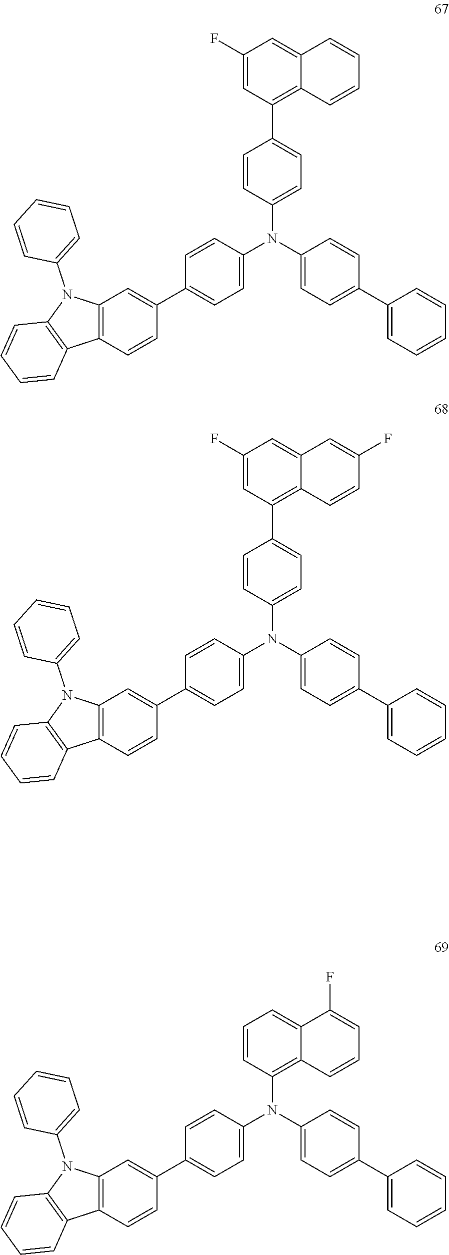 [SMILES: Fc1cc(-c2ccc(N(c3ccc(-c4ccccc4)cc3)c3ccc(-c4ccc5c6ccccc6n(-c6ccccc6)c5c4)cc3)cc2)c2ccccc2c1 ; Fc1ccc2c(-c3ccc(N(c4ccc(-c5ccccc5)cc4)c4ccc(-c5ccc6c7ccccc7n(-c7ccccc7)c6c5)cc4)cc3)cc(F)cc2c1 ; Fc1cccc2c(N(c3ccc(-c4ccccc4)cc3)c3ccc(-c4ccc5c6ccccc6n(-c6ccccc6)c5c4)cc3)cccc12] 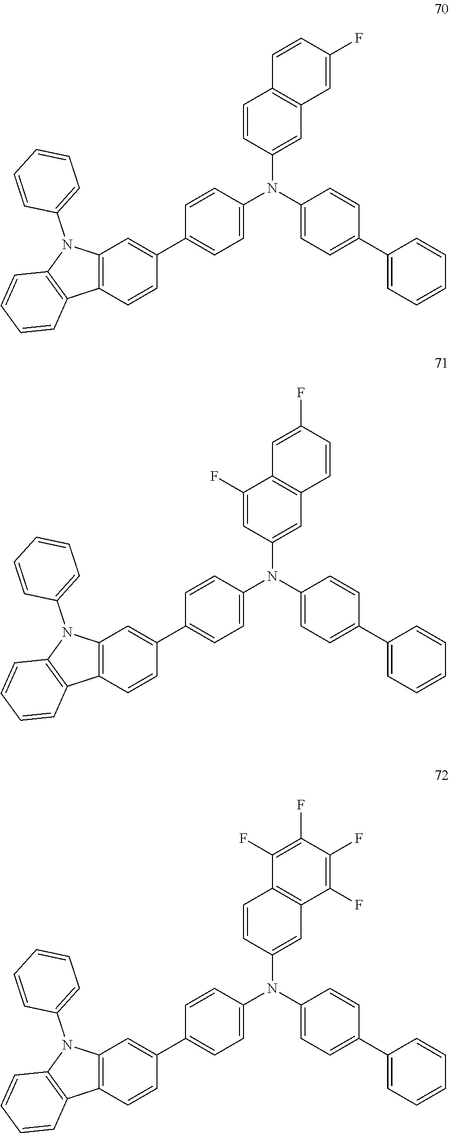 [SMILES: Fc1ccc2ccc(N(c3ccc(-c4ccccc4)cc3)c3ccc(-c4ccc5c6ccccc6n(-c6ccccc6)c5c4)cc3)cc2c1 ; Fc1ccc2cc(N(c3ccc(-c4ccccc4)cc3)c3ccc(-c4ccc5c6ccccc6n(-c6ccccc6)c5c4)cc3)cc(F)c2c1 ; Fc1c(F)c(F)c2cc(N(c3ccc(-c4ccccc4)cc3)c3ccc(-c4ccc5c6ccccc6n(-c6ccccc6)c5c4)cc3)ccc2c1F] 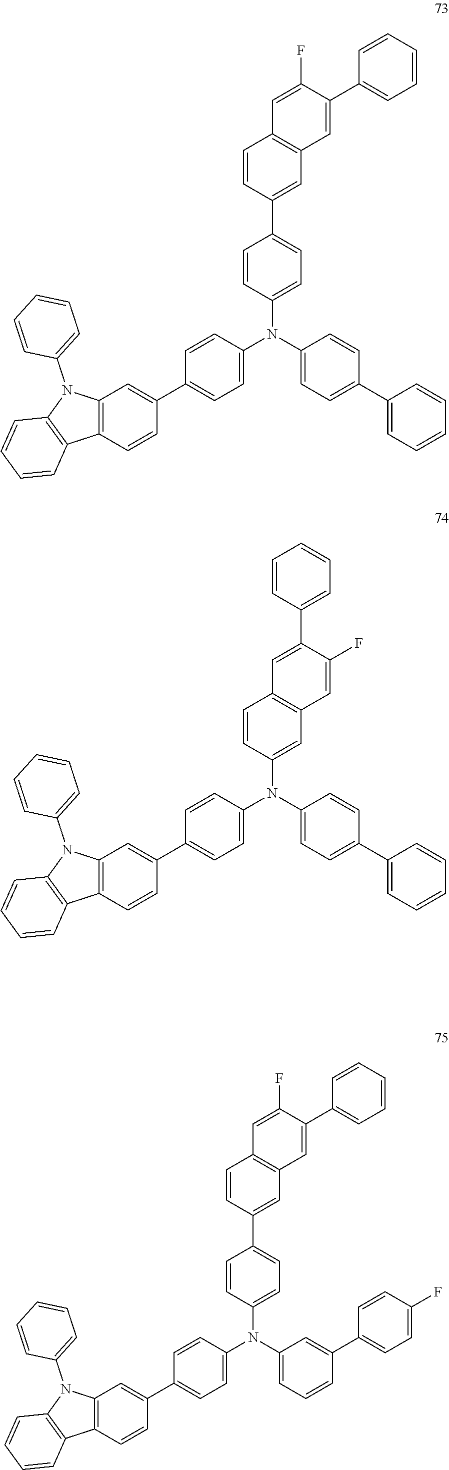 [SMILES: Fc1cc2ccc(-c3ccc(N(c4ccc(-c5ccccc5)cc4)c4ccc(-c5ccc6c7ccccc7n(-c7ccccc7)c6c5)cc4)cc3)cc2cc1-c1ccccc1 ; Fc1cc2cc(N(c3ccc(-c4ccccc4)cc3)c3ccc(-c4ccc5c6ccccc6n(-c6ccccc6)c5c4)cc3)ccc2cc1-c1ccccc1 ; Fc1ccc(-c2cccc(N(c3ccc(-c4ccc5cc(F)c(-c6ccccc6)cc5c4)cc3)c3ccc(-c4ccc5c6ccccc6n(-c6ccccc6)c5c4)cc3)c2)cc1] 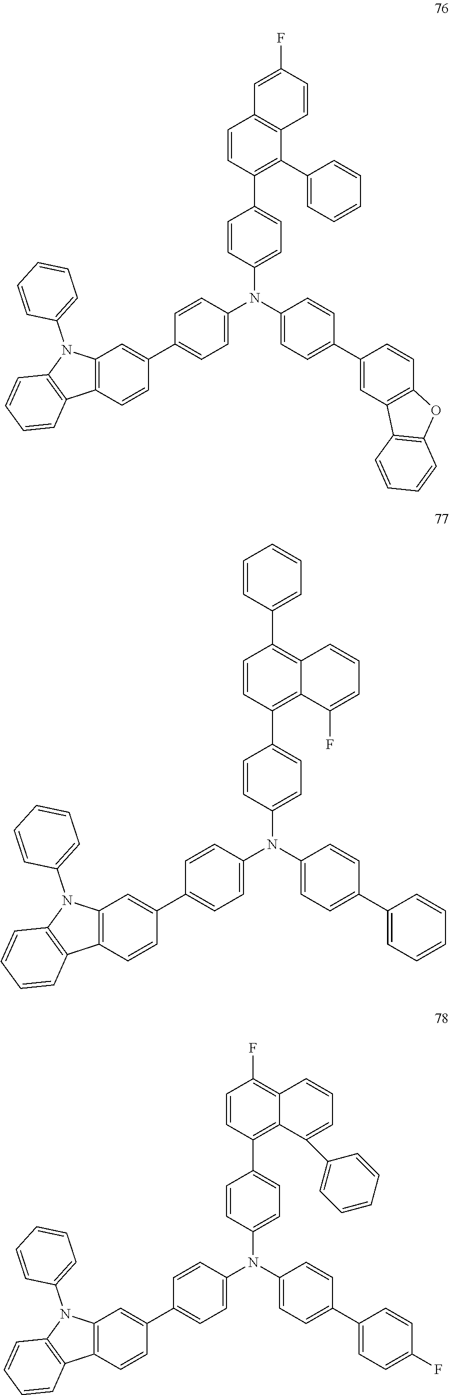 [SMILES: Fc1ccc2c(-c3ccccc3)c(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1cccc2c(-c3ccccc3)ccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4ccc(F)c5cccc(-c6ccccc6)c45)cc3)cc2)cc1] 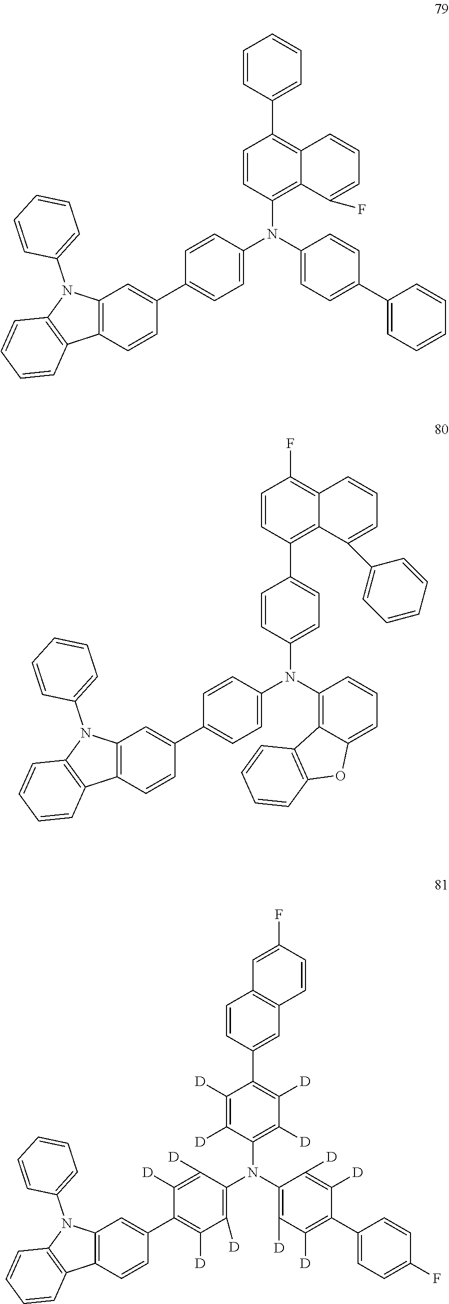 [SMILES: Fc1cccc2c(-c3ccccc3)ccc(N(c3ccc(-c4ccccc4)cc3)c3ccc(-c4ccc5c6ccccc6n(-c6ccccc6)c5c4)cc3)c12 ; Fc1ccc(-c2ccc(N(c3ccc(-c4ccc5c6ccccc6n(-c6ccccc6)c5c4)cc3)c3cccc4oc5ccccc5c34)cc2)c2c(-c3ccccc3)cccc12 ; [2H]c1c([2H])c(N(c2c([2H])c([2H])c(-c3ccc4cc(F)ccc4c3)c([2H])c2[2H])c2c([2H])c([2H])c(-c3ccc4c5ccccc5n(-c5ccccc5)c4c3)c([2H])c2[2H])c([2H])c([2H])c1-c1ccc(F)cc1] 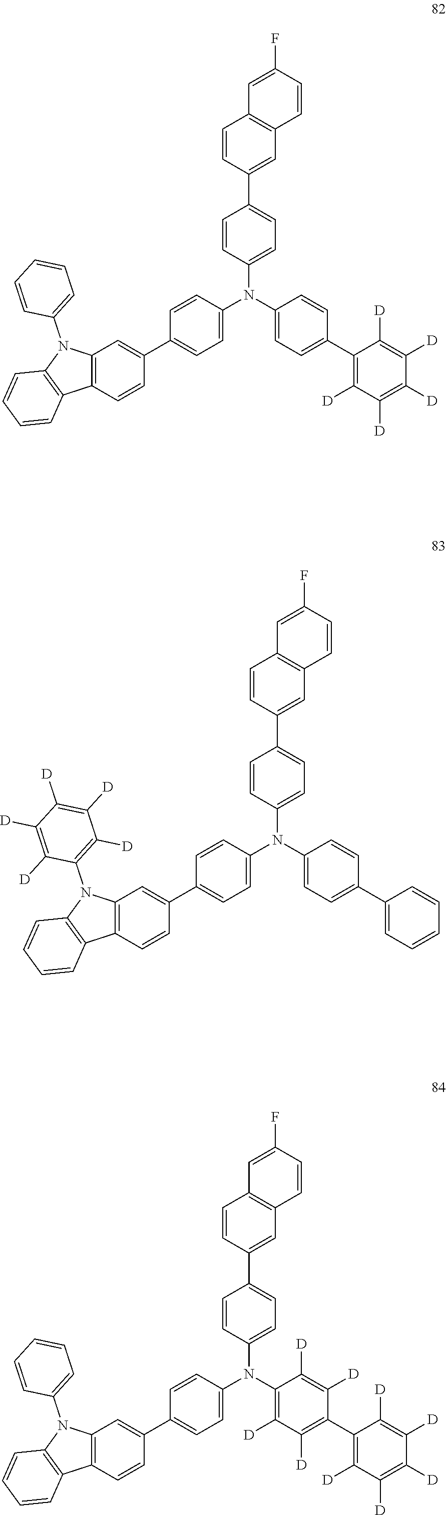 [SMILES: [2H]c1c([2H])c([2H])c(-c2ccc(N(c3ccc(-c4ccc5cc(F)ccc5c4)cc3)c3ccc(-c4ccc5c6ccccc6n(-c6ccccc6)c5c4)cc3)cc2)c([2H])c1[2H] ; [2H]c1c([2H])c([2H])c(-n2c3ccccc3c3ccc(-c4ccc(N(c5ccc(-c6ccccc6)cc5)c5ccc(-c6ccc7cc(F)ccc7c6)cc5)cc4)cc32)c([2H])c1[2H] ; [2H]c1c([2H])c([2H])c(-c2c([2H])c([2H])c(N(c3ccc(-c4ccc5cc(F)ccc5c4)cc3)c3ccc(-c4ccc5c6ccccc6n(-c6ccccc6)c5c4)cc3)c([2H])c2[2H])c([2H])c1[2H]] 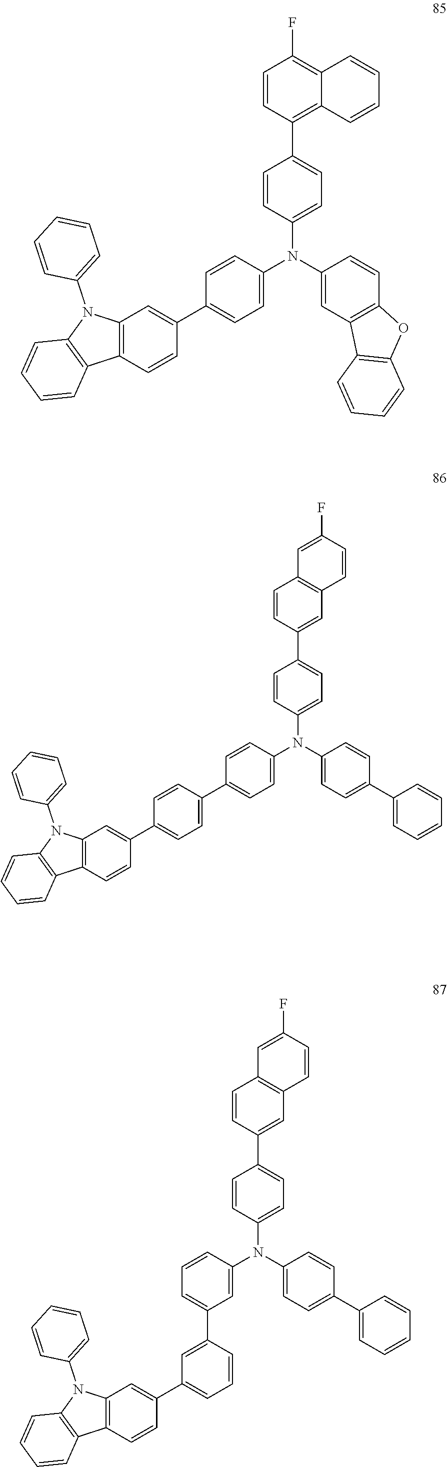 [SMILES: Fc1ccc(-c2ccc(N(c3ccc(-c4ccc5c6ccccc6n(-c6ccccc6)c5c4)cc3)c3ccc4oc5ccccc5c4c3)cc2)c2ccccc12 ; Fc1ccc2cc(-c3ccc(N(c4ccc(-c5ccccc5)cc4)c4ccc(-c5ccc(-c6ccc7c8ccccc8n(-c8ccccc8)c7c6)cc5)cc4)cc3)ccc2c1 ; Fc1ccc2cc(-c3ccc(N(c4ccc(-c5ccccc5)cc4)c4cccc(-c5cccc(-c6ccc7c8ccccc8n(-c8ccccc8)c7c6)c5)c4)cc3)ccc2c1] 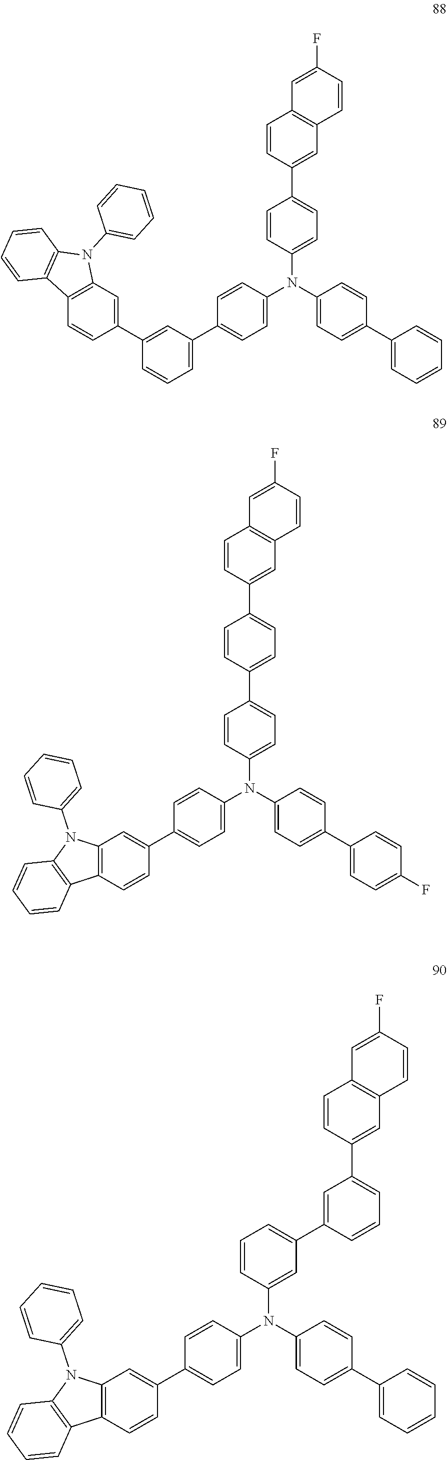 [SMILES: Fc1ccc2cc(-c3ccc(N(c4ccc(-c5ccccc5)cc4)c4ccc(-c5cccc(-c6ccc7c8ccccc8n(-c8ccccc8)c7c6)c5)cc4)cc3)ccc2c1 ; Fc1ccc(-c2ccc(N(c3ccc(-c4ccc(-c5ccc6cc(F)ccc6c5)cc4)cc3)c3ccc(-c4ccc5c6ccccc6n(-c6ccccc6)c5c4)cc3)cc2)cc1 ; Fc1ccc2cc(-c3cccc(-c4cccc(N(c5ccc(-c6ccccc6)cc5)c5ccc(-c6ccc7c8ccccc8n(-c8ccccc8)c7c6)cc5)c4)c3)ccc2c1] 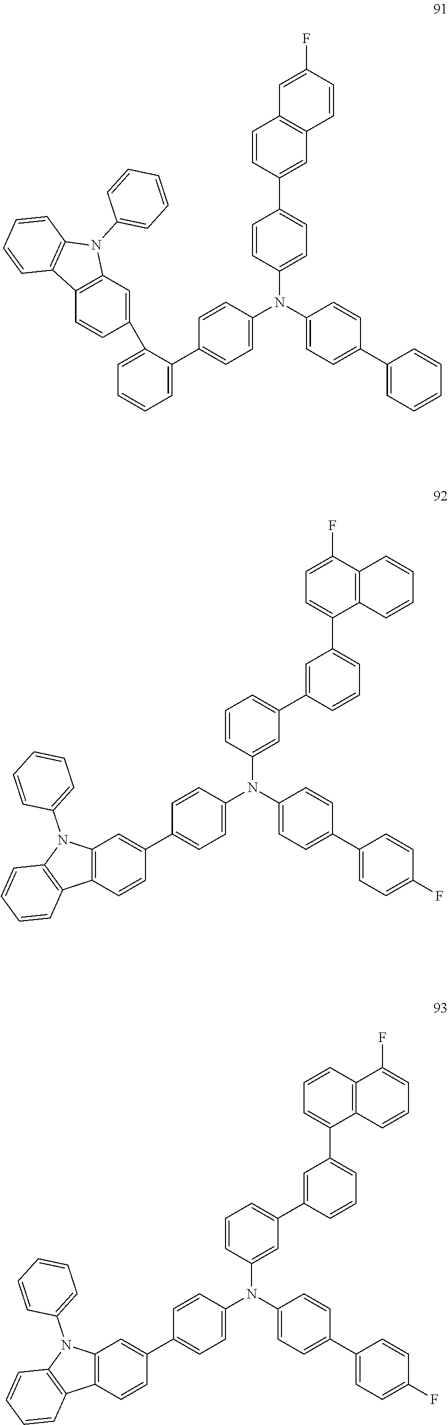 [SMILES: Fc1ccc2cc(-c3ccc(N(c4ccc(-c5ccccc5)cc4)c4ccc(-c5ccccc5-c5ccc6c7ccccc7n(-c7ccccc7)c6c5)cc4)cc3)ccc2c1 ; Fc1ccc(-c2ccc(N(c3ccc(-c4ccc5c6ccccc6n(-c6ccccc6)c5c4)cc3)c3cccc(-c4cccc(-c5ccc(F)c6ccccc56)c4)c3)cc2)cc1 ; Fc1ccc(-c2ccc(N(c3ccc(-c4ccc5c6ccccc6n(-c6ccccc6)c5c4)cc3)c3cccc(-c4cccc(-c5cccc6c(F)cccc56)c4)c3)cc2)cc1] 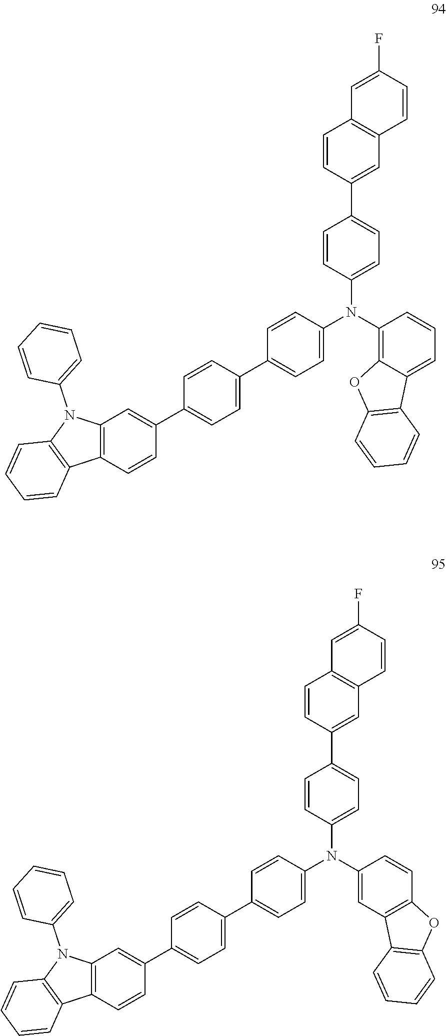 [SMILES: Fc1ccc2cc(-c3ccc(N(c4ccc(-c5ccc(-c6ccc7c8ccccc8n(-c8ccccc8)c7c6)cc5)cc4)c4cccc5c4oc4ccccc45)cc3)ccc2c1 ; Fc1ccc2cc(-c3ccc(N(c4ccc(-c5ccc(-c6ccc7c8ccccc8n(-c8ccccc8)c7c6)cc5)cc4)c4ccc5oc6ccccc6c5c4)cc3)ccc2c1] 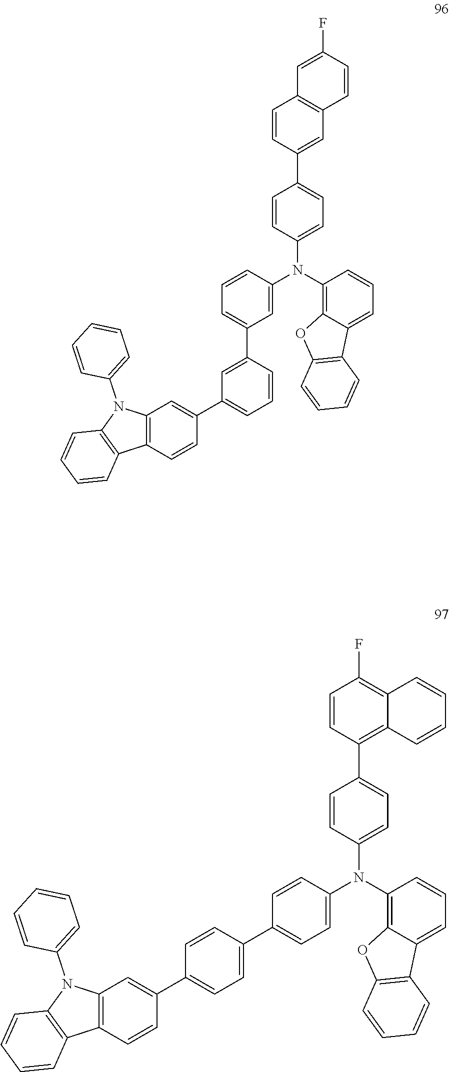 [SMILES: Fc1ccc2cc(-c3ccc(N(c4cccc(-c5cccc(-c6ccc7c8ccccc8n(-c8ccccc8)c7c6)c5)c4)c4cccc5c4oc4ccccc45)cc3)ccc2c1 ; Fc1ccc(-c2ccc(N(c3ccc(-c4ccc(-c5ccc6c7ccccc7n(-c7ccccc7)c6c5)cc4)cc3)c3cccc4c3oc3ccccc34)cc2)c2ccccc12]
where, in Compound Group 1, D is a deuterium atom.

10. An amine compound according to Formula 1: 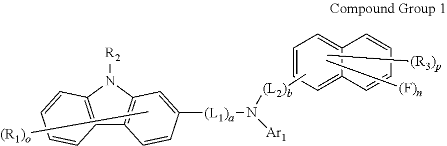
wherein, in Formula 1 above,
L₁ and L₂ are each independently a substituted or unsubstituted arylene group having 6 to 30 carbon atoms,
Ar₁ a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms,
R₁ to R₃ are each independently a hydrogen atom, a deuterium atom, a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms,
a and b are each independently an integer of 0 to 4, provided that;
when a is 0, N of NAr₁ is directly bonded to the 2-position of a carbazole group, and
when b is 0, N of NAr₁ is directly bonded to a naphthyl group,
n is an integer of 1 to 7,
o is an integer of 0 to 7, and
p is an integer of 0 to (7-n).

11. The amine compound of claim 10, wherein Formula 1 above has a structure according to any one of Formula 2-1 to Formula 2-3: 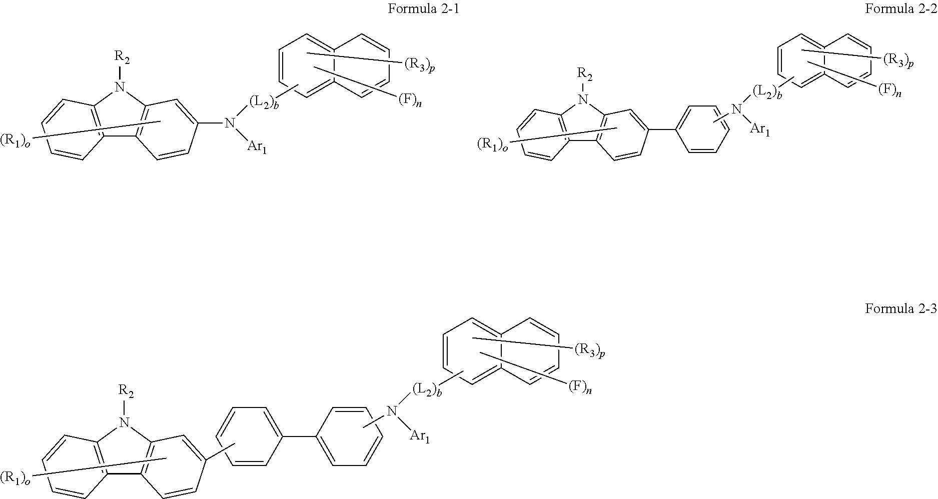
where, in Formula 2-1 to Formula 2-3, L₂, Ar₁, R₁ to R₃, b, n, o, and p are the same as defined in Formula 1.

12. The amine compound of claim 10, wherein Formula 1 has a structure according to any one of Formula 3-1 to Formula 3-3: 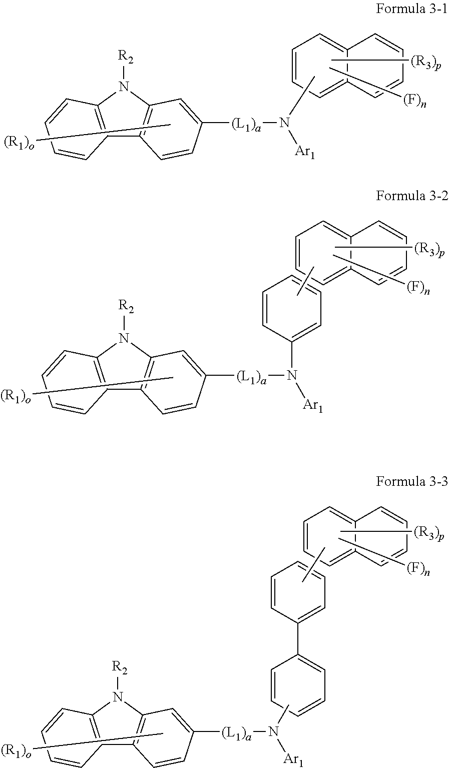
where, in Formula 3-1 to Formula 3-3 above, L₁, Ar₁, R₁ to R₃, a, n, o, and p are the same as defined in Formula 1 above.

13. The amine compound of claim 10, wherein Ar₁ is a substituted or unsubstituted phenyl group, a substituted or unsubstituted dibenzofuran group, a substituted or unsubstituted dibenzothiophene group, a substituted or unsubstituted naphthyl group, or a substituted or unsubstituted naphthobenzofuran group.

14. The amine compound of claim 10, wherein R₂ is a substituted or unsubstituted phenyl group.

15. The amine compound of claim 10, wherein Formula 1 is a monoamine compound each R₁, R₂, and each R₃ does not include an amino group.

16. The amine compound of claim 10, wherein 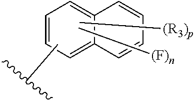
in Formula 1 has a structure according to any one functional group in Functional Group Set A: 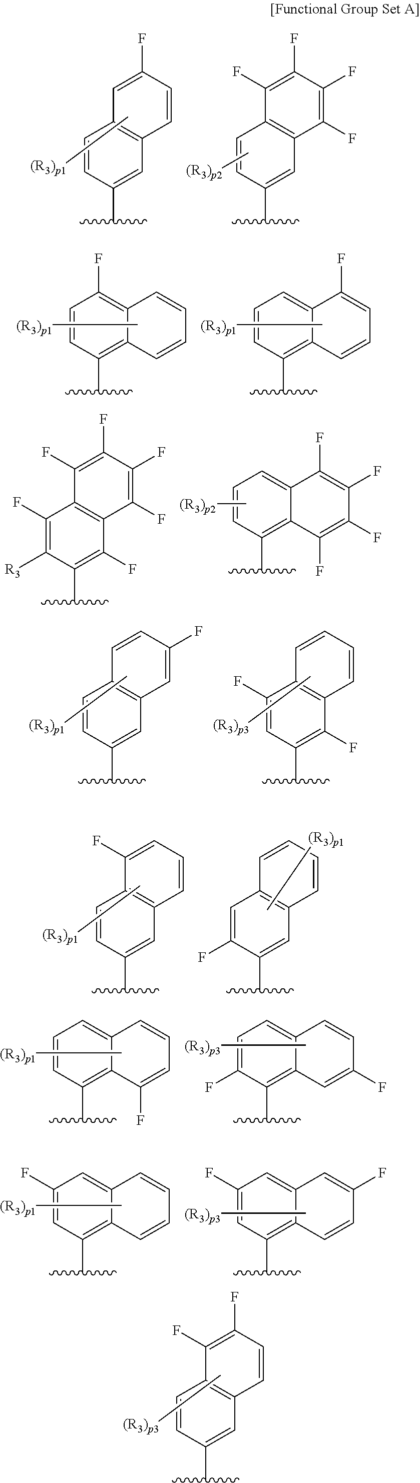
where, in Functional Group Set A,
p1 is an integer of 0 to 6,
p2 is an integer of 0 to 3,
p3 is an integer of 0 to 5, and
R₃ is the same as defined in Formula 1.

17. The amine compound of claim 10 has a structure according to any one of compound in Compound Group 1: 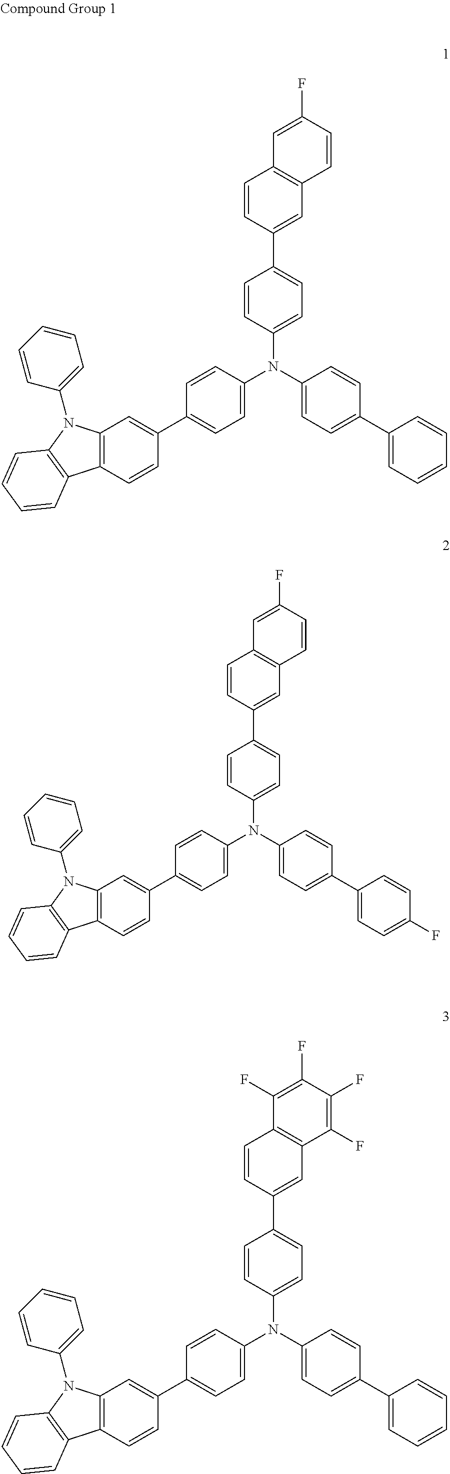 [SMILES: Fc1ccc2cc(-c3ccc(N(c4ccc(-c5ccccc5)cc4)c4ccc(-c5ccc6c7ccccc7n(-c7ccccc7)c6c5)cc4)cc3)ccc2c1 ; Fc1ccc(-c2ccc(N(c3ccc(-c4ccc5cc(F)ccc5c4)cc3)c3ccc(-c4ccc5c6ccccc6n(-c6ccccc6)c5c4)cc3)cc2)cc1 ; Fc1c(F)c(F)c2cc(-c3ccc(N(c4ccc(-c5ccccc5)cc4)c4ccc(-c5ccc6c7ccccc7n(-c7ccccc7)c6c5)cc4)cc3)ccc2c1F] 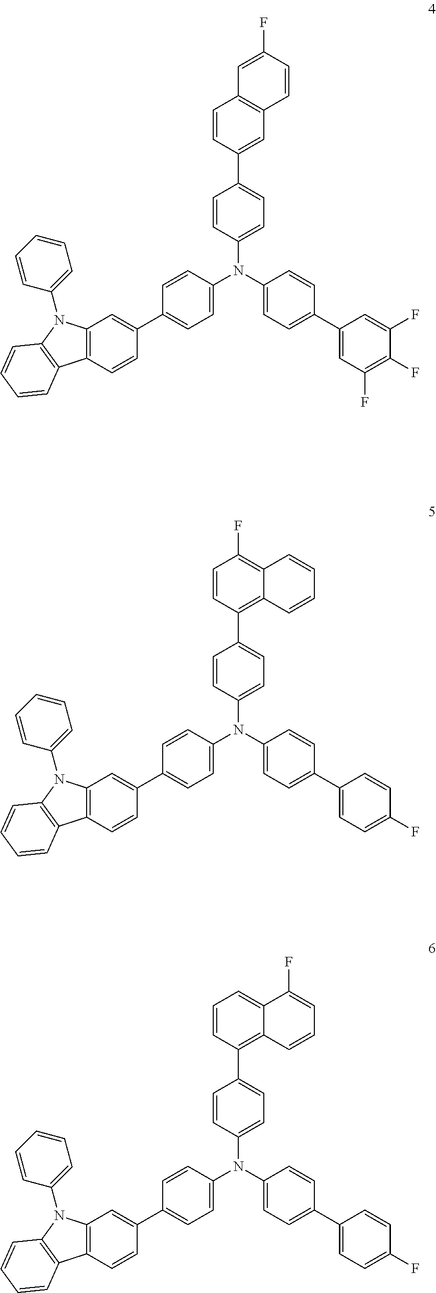 [SMILES: Fc1ccc2cc(-c3ccc(N(c4ccc(-c5cc(F)c(F)c(F)c5)cc4)c4ccc(-c5ccc6c7ccccc7n(-c7ccccc7)c6c5)cc4)cc3)ccc2c1 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)cc1 ; Fc1ccc(-c2ccc(N(c3ccc(-c4ccc5c6ccccc6n(-c6ccccc6)c5c4)cc3)c3ccc(-c4cccc5c(F)cccc45)cc3)cc2)cc1] 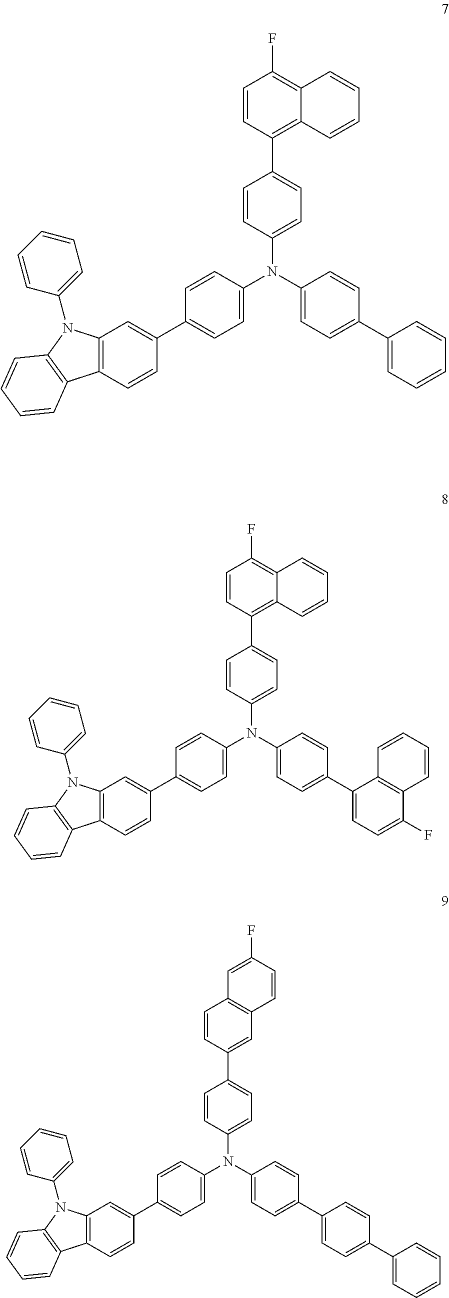 [SMILES: Fc1ccc(-c2ccc(N(c3ccc(-c4ccccc4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c6ccccc6n(-c6ccccc6)c5c4)cc3)c3ccc(-c4ccc(F)c5ccccc45)cc3)cc2)c2ccccc12 ; Fc1ccc2cc(-c3ccc(N(c4ccc(-c5ccc(-c6ccccc6)cc5)cc4)c4ccc(-c5ccc6c7ccccc7n(-c7ccccc7)c6c5)cc4)cc3)ccc2c1] 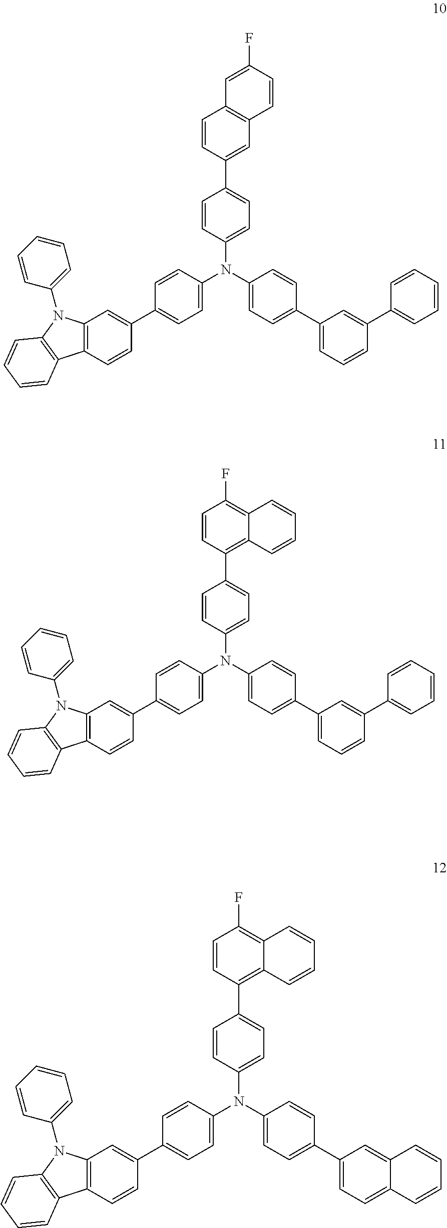 [SMILES: Fc1ccc2cc(-c3ccc(N(c4ccc(-c5cccc(-c6ccccc6)c5)cc4)c4ccc(-c5ccc6c7ccccc7n(-c7ccccc7)c6c5)cc4)cc3)ccc2c1 ; Fc1ccc(-c2ccc(N(c3ccc(-c4cccc(-c5ccccc5)c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5ccccc5c4)cc3)c3ccc(-c4ccc5c6ccccc6n(-c6ccccc6)c5c4)cc3)cc2)c2ccccc12] 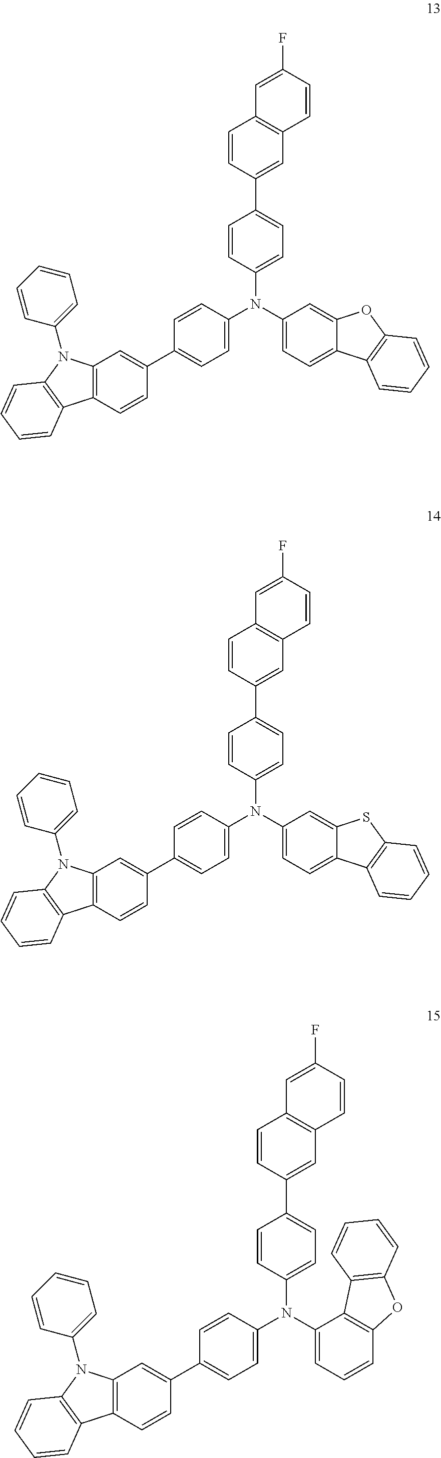 [SMILES: Fc1ccc2cc(-c3ccc(N(c4ccc(-c5ccc6c7ccccc7n(-c7ccccc7)c6c5)cc4)c4ccc5c(c4)oc4ccccc45)cc3)ccc2c1 ; Fc1ccc2cc(-c3ccc(N(c4ccc(-c5ccc6c7ccccc7n(-c7ccccc7)c6c5)cc4)c4ccc5c(c4)sc4ccccc45)cc3)ccc2c1 ; Fc1ccc2cc(-c3ccc(N(c4ccc(-c5ccc6c7ccccc7n(-c7ccccc7)c6c5)cc4)c4cccc5oc6ccccc6c45)cc3)ccc2c1] 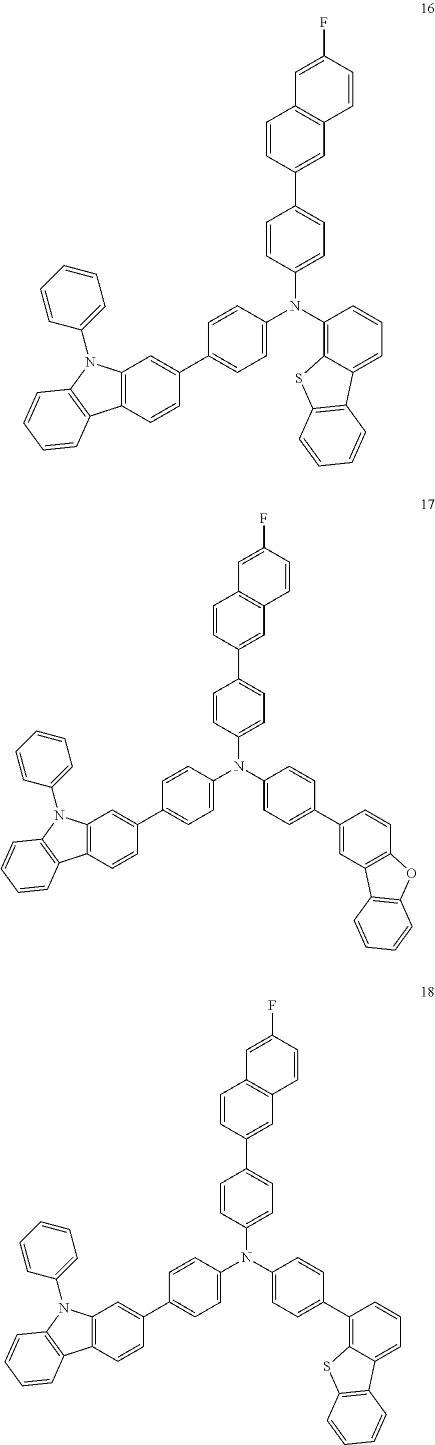 [SMILES: Fc1ccc2cc(-c3ccc(N(c4ccc(-c5ccc6c7ccccc7n(-c7ccccc7)c6c5)cc4)c4cccc5c4sc4ccccc45)cc3)ccc2c1 ; Fc1ccc2cc(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5sc5ccccc56)cc4)cc3)ccc2c1] 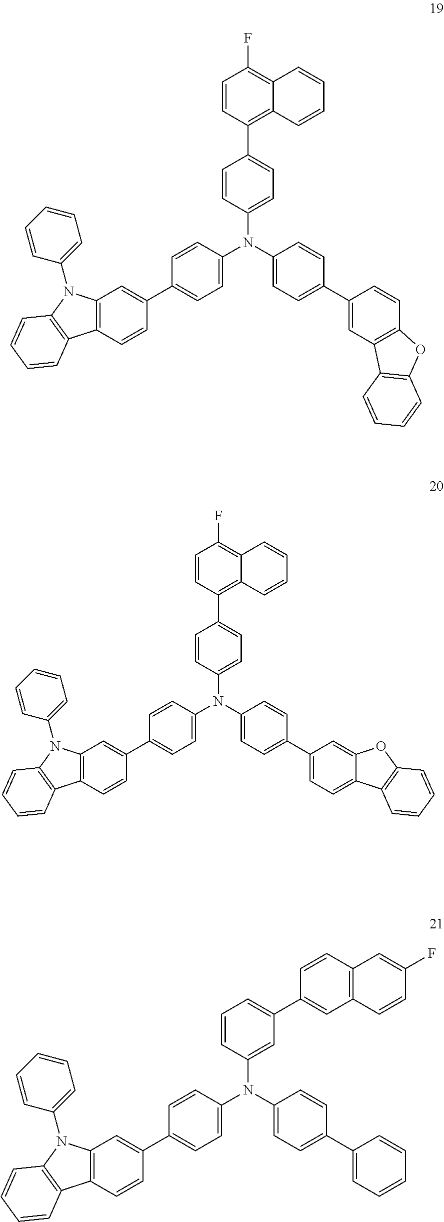 [SMILES: Fc1ccc(-c2ccc(N(c3ccc(-c4ccc5oc6ccccc6c5c4)cc3)c3ccc(-c4ccc5c6ccccc6n(-c6ccccc6)c5c4)cc3)cc2)c2ccccc12 ; Fc1ccc(-c2ccc(N(c3ccc(-c4ccc5c(c4)oc4ccccc45)cc3)c3ccc(-c4ccc5c6ccccc6n(-c6ccccc6)c5c4)cc3)cc2)c2ccccc12 ; Fc1ccc2cc(-c3cccc(N(c4ccc(-c5ccccc5)cc4)c4ccc(-c5ccc6c7ccccc7n(-c7ccccc7)c6c5)cc4)c3)ccc2c1] 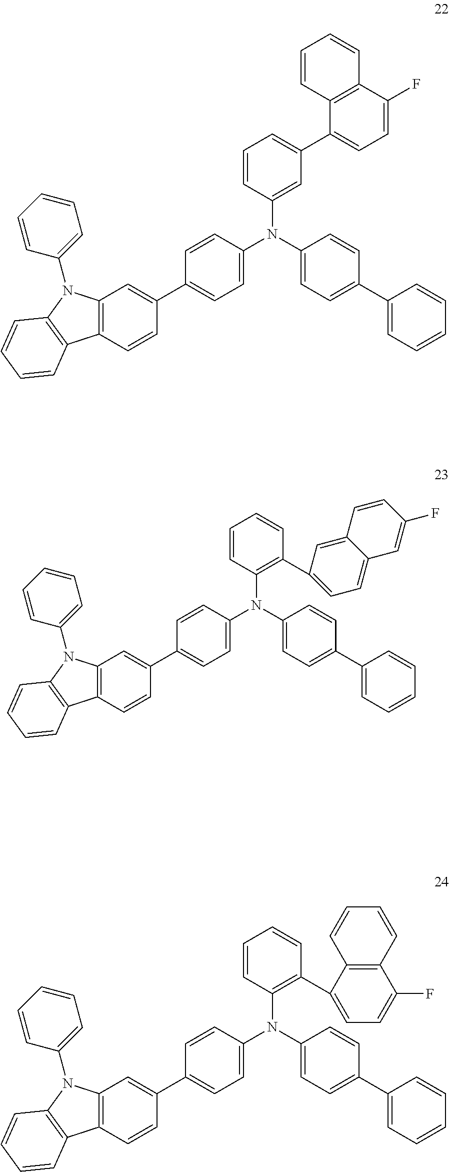 [SMILES: Fc1ccc(-c2cccc(N(c3ccc(-c4ccccc4)cc3)c3ccc(-c4ccc5c6ccccc6n(-c6ccccc6)c5c4)cc3)c2)c2ccccc12 ; Fc1ccc2cc(-c3ccccc3N(c3ccc(-c4ccccc4)cc3)c3ccc(-c4ccc5c6ccccc6n(-c6ccccc6)c5c4)cc3)ccc2c1 ; Fc1ccc(-c2ccccc2N(c2ccc(-c3ccccc3)cc2)c2ccc(-c3ccc4c5ccccc5n(-c5ccccc5)c4c3)cc2)c2ccccc12] 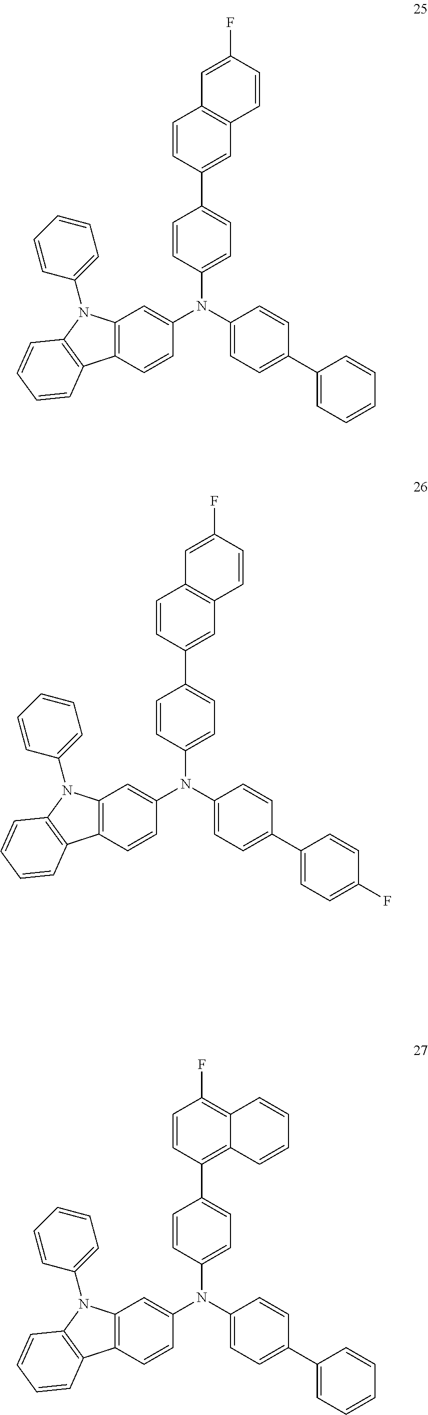 [SMILES: Fc1ccc2cc(-c3ccc(N(c4ccc(-c5ccccc5)cc4)c4ccc5c6ccccc6n(-c6ccccc6)c5c4)cc3)ccc2c1 ; Fc1ccc(-c2ccc(N(c3ccc(-c4ccc5cc(F)ccc5c4)cc3)c3ccc4c5ccccc5n(-c5ccccc5)c4c3)cc2)cc1 ; Fc1ccc(-c2ccc(N(c3ccc(-c4ccccc4)cc3)c3ccc4c5ccccc5n(-c5ccccc5)c4c3)cc2)c2ccccc12] 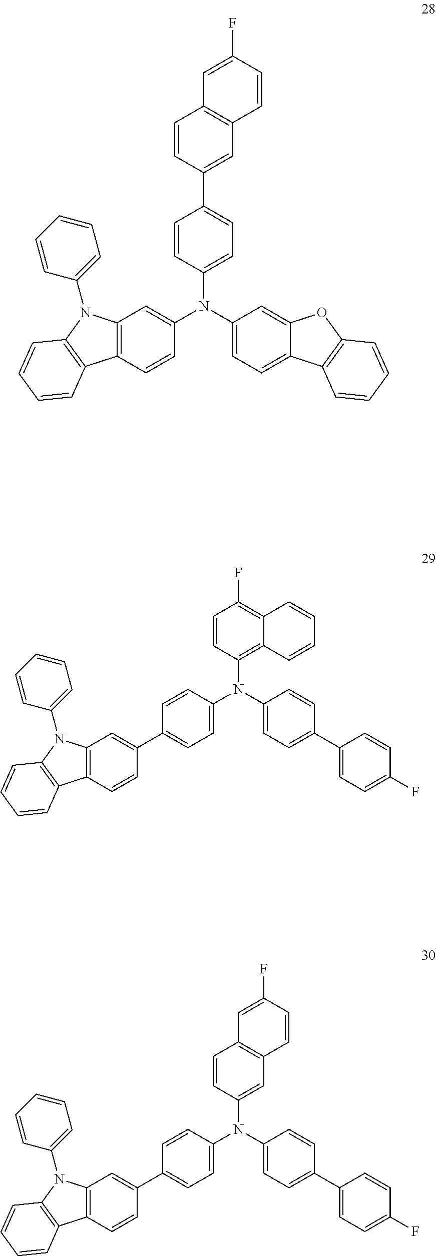 [SMILES: Fc1ccc2cc(-c3ccc(N(c4ccc5c(c4)oc4ccccc45)c4ccc5c6ccccc6n(-c6ccccc6)c5c4)cc3)ccc2c1 ; Fc1ccc(-c2ccc(N(c3ccc(-c4ccc5c6ccccc6n(-c6ccccc6)c5c4)cc3)c3ccc(F)c4ccccc34)cc2)cc1 ; Fc1ccc(-c2ccc(N(c3ccc(-c4ccc5c6ccccc6n(-c6ccccc6)c5c4)cc3)c3ccc4cc(F)ccc4c3)cc2)cc1] 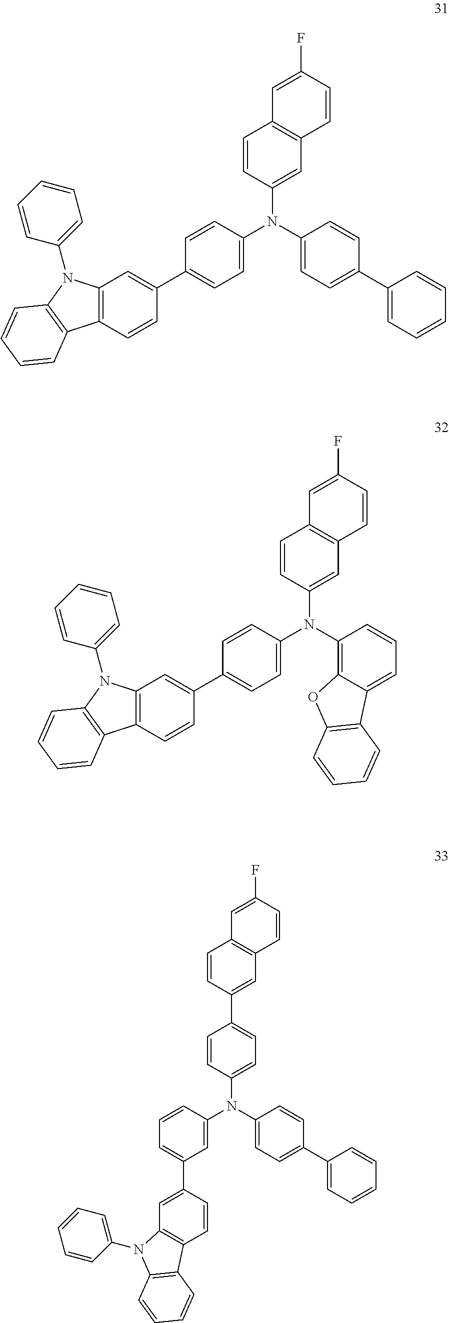 [SMILES: Fc1ccc2cc(N(c3ccc(-c4ccccc4)cc3)c3ccc(-c4ccc5c6ccccc6n(-c6ccccc6)c5c4)cc3)ccc2c1 ; Fc1ccc2cc(N(c3ccc(-c4ccc5c6ccccc6n(-c6ccccc6)c5c4)cc3)c3cccc4c3oc3ccccc34)ccc2c1 ; Fc1ccc2cc(-c3ccc(N(c4ccc(-c5ccccc5)cc4)c4cccc(-c5ccc6c7ccccc7n(-c7ccccc7)c6c5)c4)cc3)ccc2c1] 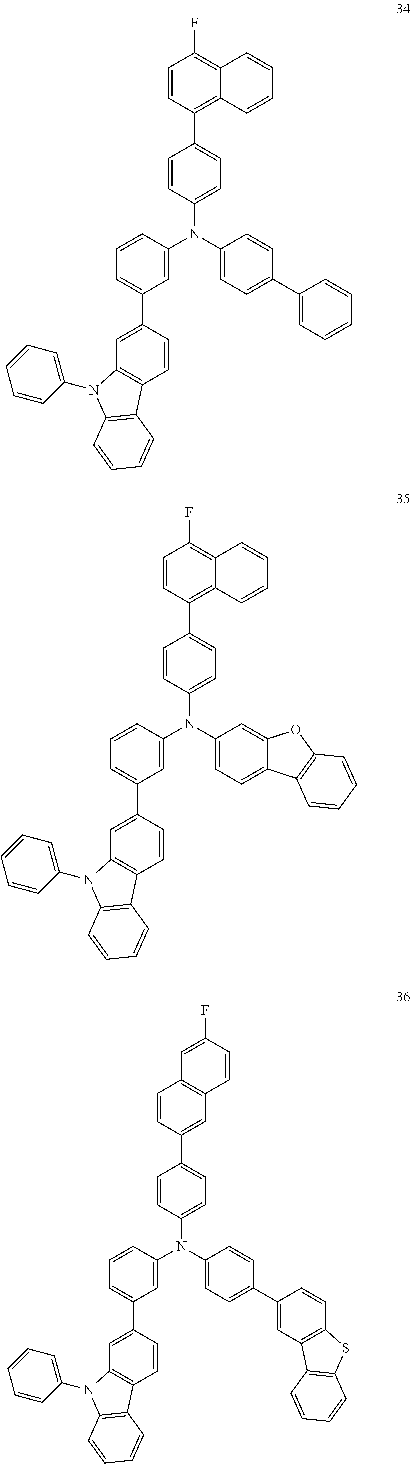 [SMILES: Fc1ccc(-c2ccc(N(c3ccc(-c4ccccc4)cc3)c3cccc(-c4ccc5c6ccccc6n(-c6ccccc6)c5c4)c3)cc2)c2ccccc12 ; Fc1ccc(-c2ccc(N(c3cccc(-c4ccc5c6ccccc6n(-c6ccccc6)c5c4)c3)c3ccc4c(c3)oc3ccccc34)cc2)c2ccccc12 ; Fc1ccc2cc(-c3ccc(N(c4ccc(-c5ccc6sc7ccccc7c6c5)cc4)c4cccc(-c5ccc6c7ccccc7n(-c7ccccc7)c6c5)c4)cc3)ccc2c1] 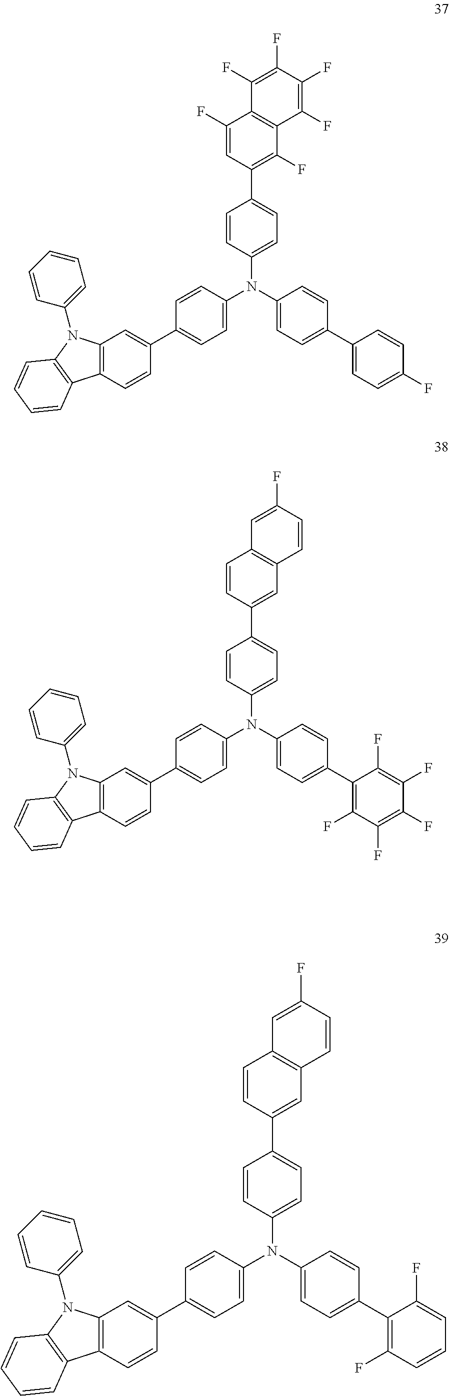 [SMILES: Fc1ccc(-c2ccc(N(c3ccc(-c4ccc5c6ccccc6n(-c6ccccc6)c5c4)cc3)c3ccc(-c4cc(F)c5c(F)c(F)c(F)c(F)c5c4F)cc3)cc2)cc1 ; Fc1ccc2cc(-c3ccc(N(c4ccc(-c5ccc6c7ccccc7n(-c7ccccc7)c6c5)cc4)c4ccc(-c5c(F)c(F)c(F)c(F)c5F)cc4)cc3)ccc2c1 ; Fc1ccc2cc(-c3ccc(N(c4ccc(-c5ccc6c7ccccc7n(-c7ccccc7)c6c5)cc4)c4ccc(-c5c(F)cccc5F)cc4)cc3)ccc2c1] 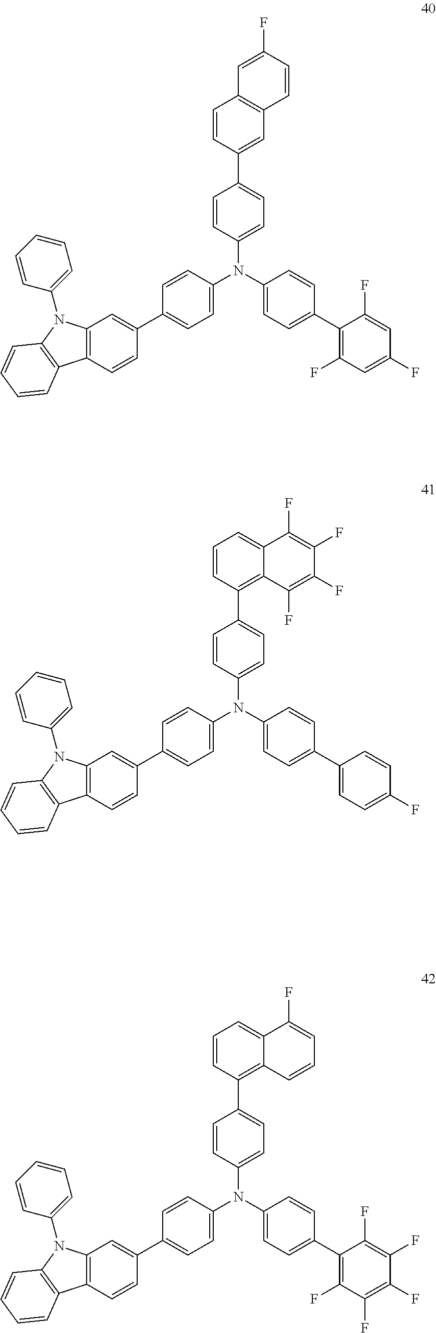 [SMILES: Fc1cc(F)c(-c2ccc(N(c3ccc(-c4ccc5cc(F)ccc5c4)cc3)c3ccc(-c4ccc5c6ccccc6n(-c6ccccc6)c5c4)cc3)cc2)c(F)c1 ; Fc1ccc(-c2ccc(N(c3ccc(-c4ccc5c6ccccc6n(-c6ccccc6)c5c4)cc3)c3ccc(-c4cccc5c(F)c(F)c(F)c(F)c45)cc3)cc2)cc1 ; Fc1c(F)c(F)c(-c2ccc(N(c3ccc(-c4ccc5c6ccccc6n(-c6ccccc6)c5c4)cc3)c3ccc(-c4cccc5c(F)cccc45)cc3)cc2)c(F)c1F] 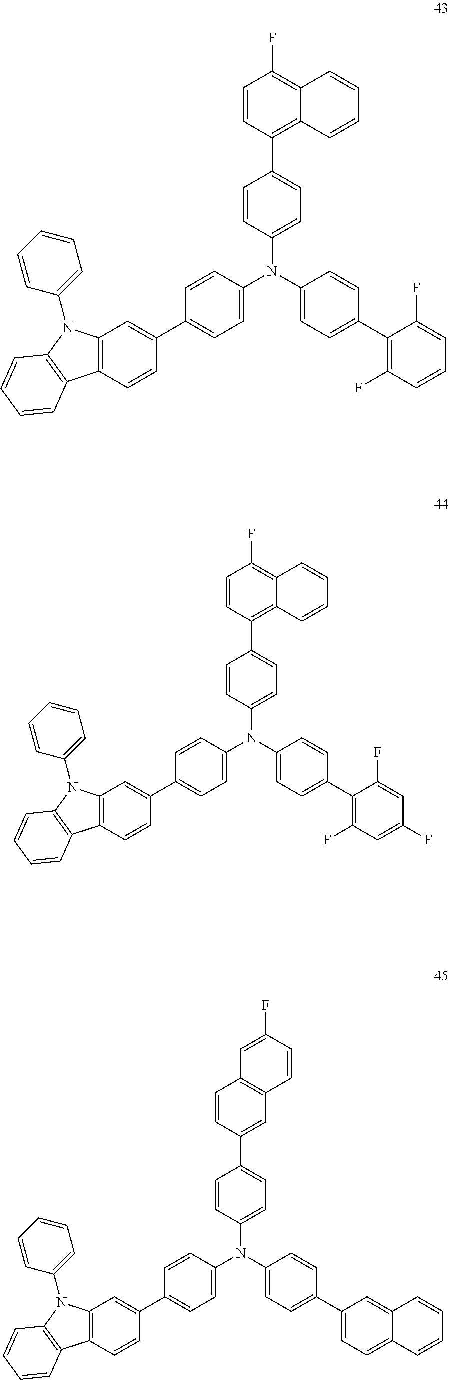 [SMILES: Fc1cccc(F)c1-c1ccc(N(c2ccc(-c3ccc4c5ccccc5n(-c5ccccc5)c4c3)cc2)c2ccc(-c3ccc(F)c4ccccc34)cc2)cc1 ; Fc1cc(F)c(-c2ccc(N(c3ccc(-c4ccc5c6ccccc6n(-c6ccccc6)c5c4)cc3)c3ccc(-c4ccc(F)c5ccccc45)cc3)cc2)c(F)c1 ; Fc1ccc2cc(-c3ccc(N(c4ccc(-c5ccc6ccccc6c5)cc4)c4ccc(-c5ccc6c7ccccc7n(-c7ccccc7)c6c5)cc4)cc3)ccc2c1] 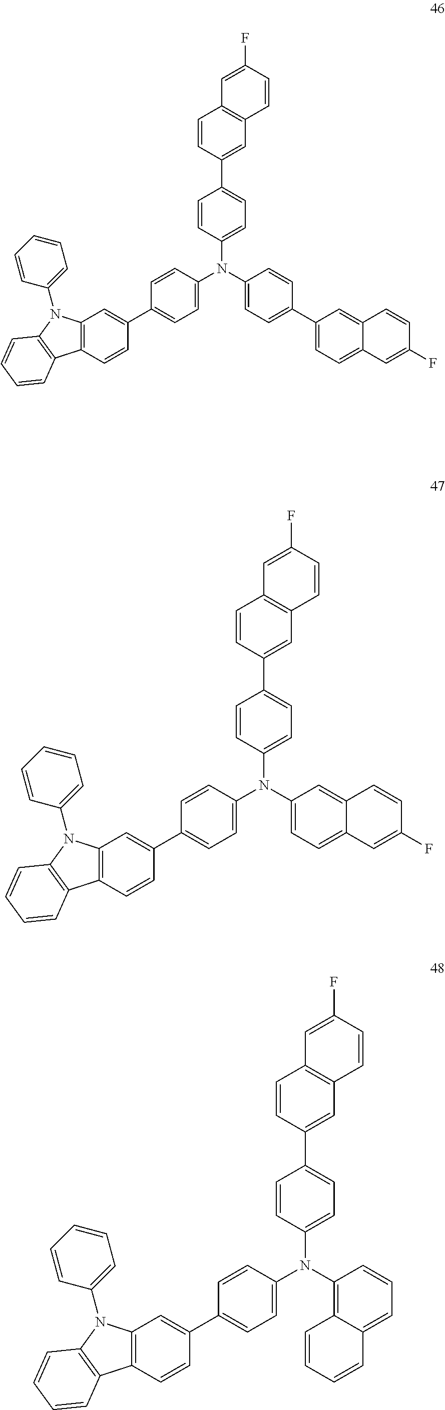 [SMILES: Fc1ccc2cc(-c3ccc(N(c4ccc(-c5ccc6cc(F)ccc6c5)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4ccc5cc(F)ccc5c4)cc3)ccc2c1 ; Fc1ccc2cc(-c3ccc(N(c4ccc(-c5ccc6c7ccccc7n(-c7ccccc7)c6c5)cc4)c4cccc5ccccc45)cc3)ccc2c1] 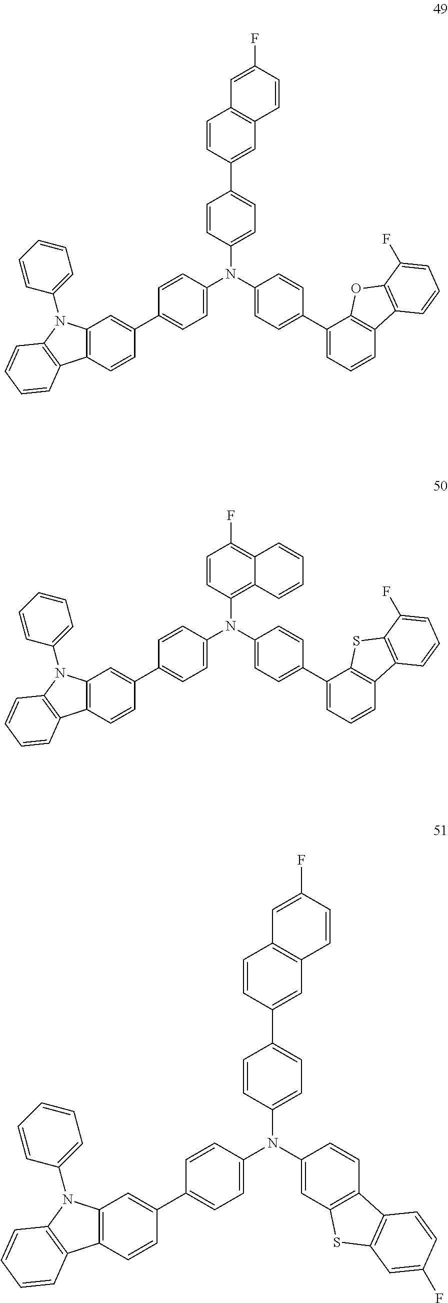 [SMILES: Fc1ccc2cc(-c3ccc(N(c4ccc(-c5ccc6c7ccccc7n(-c7ccccc7)c6c5)cc4)c4ccc(-c5cccc6c5oc5c(F)cccc56)cc4)cc3)ccc2c1 ; Fc1ccc(N(c2ccc(-c3ccc4c5ccccc5n(-c5ccccc5)c4c3)cc2)c2ccc(-c3cccc4c3sc3c(F)cccc34)cc2)c2ccccc12 ; Fc1ccc2cc(-c3ccc(N(c4ccc(-c5ccc6c7ccccc7n(-c7ccccc7)c6c5)cc4)c4ccc5c(c4)sc4cc(F)ccc45)cc3)ccc2c1] 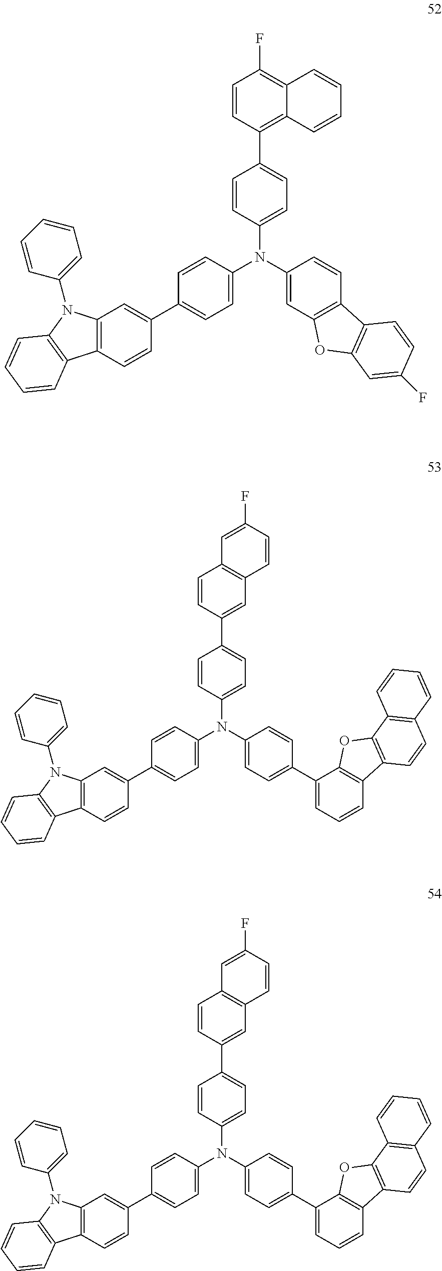 [SMILES: Fc1ccc2c(c1)oc1cc(N(c3ccc(-c4ccc5c6ccccc6n(-c6ccccc6)c5c4)cc3)c3ccc(-c4ccc(F)c5ccccc45)cc3)ccc12 ; Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1 ; Fc1ccc2cc(-c3ccc(N(c4ccc(-c5ccc6c7ccccc7n(-c7ccccc7)c6c5)cc4)c4ccc(-c5cccc6c5oc5c7ccccc7ccc65)cc4)cc3)ccc2c1] 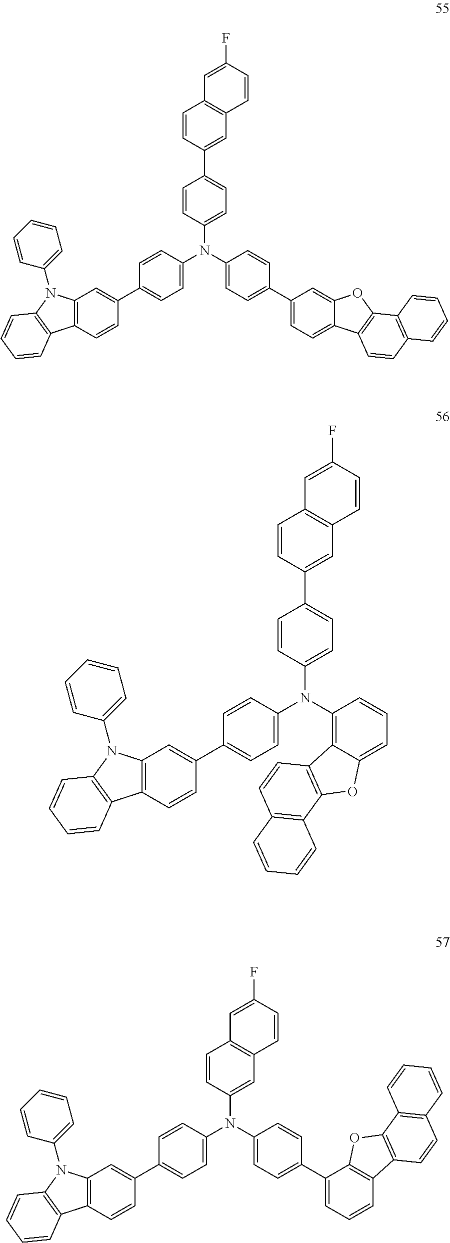 [SMILES: Fc1ccc2cc(-c3ccc(N(c4ccc(-c5ccc6c(c5)oc5c7ccccc7ccc65)cc4)c4ccc(-c5ccc6c7ccccc7n(-c7ccccc7)c6c5)cc4)cc3)ccc2c1 ; Fc1ccc2cc(-c3ccc(N(c4ccc(-c5ccc6c7ccccc7n(-c7ccccc7)c6c5)cc4)c4cccc5oc6c7ccccc7ccc6c45)cc3)ccc2c1 ; Fc1ccc2cc(N(c3ccc(-c4ccc5c6ccccc6n(-c6ccccc6)c5c4)cc3)c3ccc(-c4cccc5c4oc4c6ccccc6ccc54)cc3)ccc2c1] 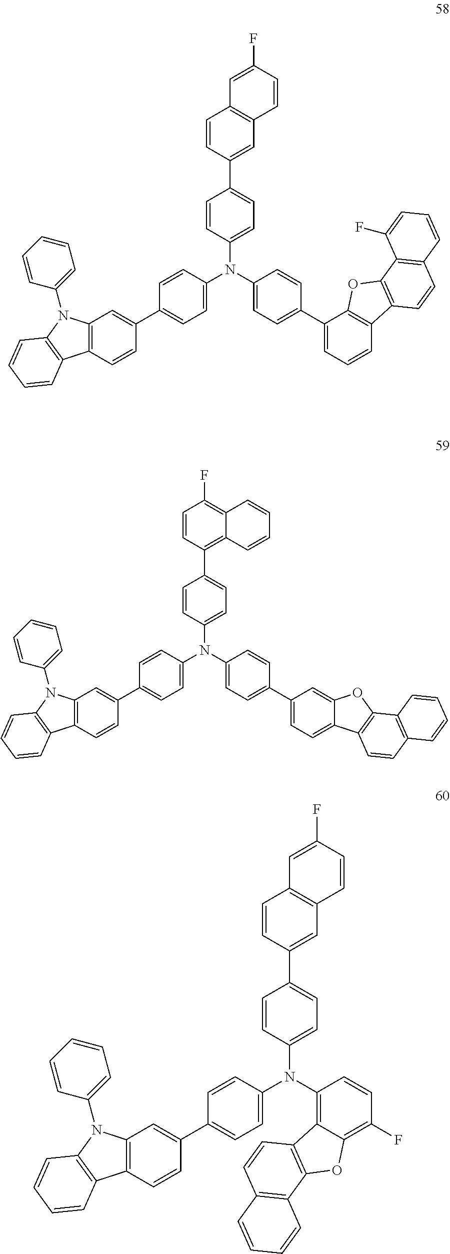 [SMILES: Fc1ccc2cc(-c3ccc(N(c4ccc(-c5ccc6c7ccccc7n(-c7ccccc7)c6c5)cc4)c4ccc(-c5cccc6c5oc5c6ccc6cccc(F)c65)cc4)cc3)ccc2c1 ; Fc1ccc(-c2ccc(N(c3ccc(-c4ccc5c(c4)oc4c6ccccc6ccc54)cc3)c3ccc(-c4ccc5c6ccccc6n(-c6ccccc6)c5c4)cc3)cc2)c2ccccc12 ; Fc1ccc2cc(-c3ccc(N(c4ccc(-c5ccc6c7ccccc7n(-c7ccccc7)c6c5)cc4)c4ccc(F)c5oc6c7ccccc7ccc6c45)cc3)ccc2c1] 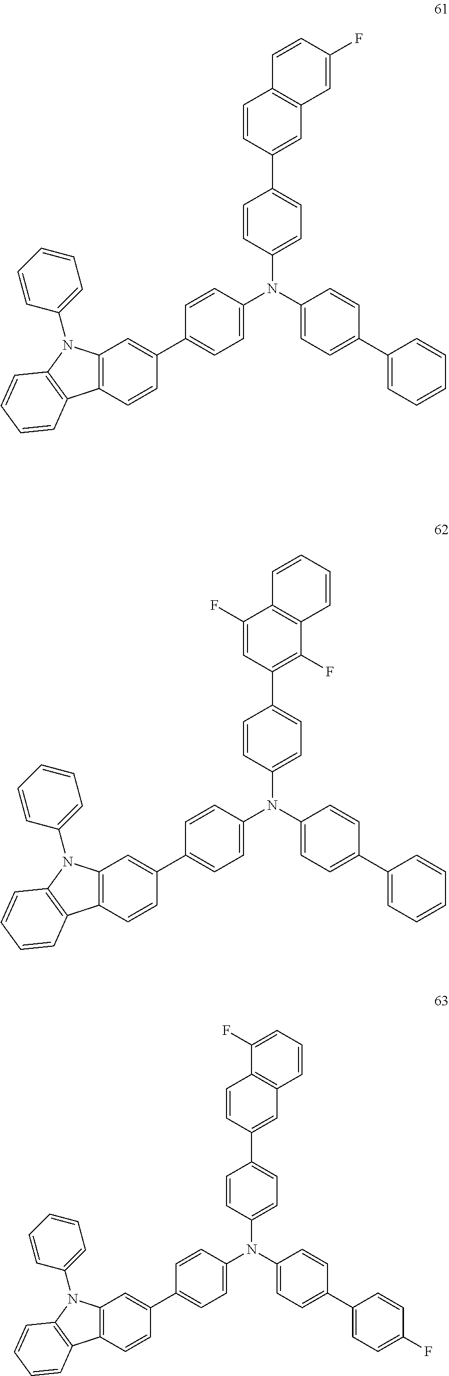 [SMILES: Fc1ccc2ccc(-c3ccc(N(c4ccc(-c5ccccc5)cc4)c4ccc(-c5ccc6c7ccccc7n(-c7ccccc7)c6c5)cc4)cc3)cc2c1 ; Fc1cc(-c2ccc(N(c3ccc(-c4ccccc4)cc3)c3ccc(-c4ccc5c6ccccc6n(-c6ccccc6)c5c4)cc3)cc2)c(F)c2ccccc12 ; Fc1ccc(-c2ccc(N(c3ccc(-c4ccc5c(F)cccc5c4)cc3)c3ccc(-c4ccc5c6ccccc6n(-c6ccccc6)c5c4)cc3)cc2)cc1] 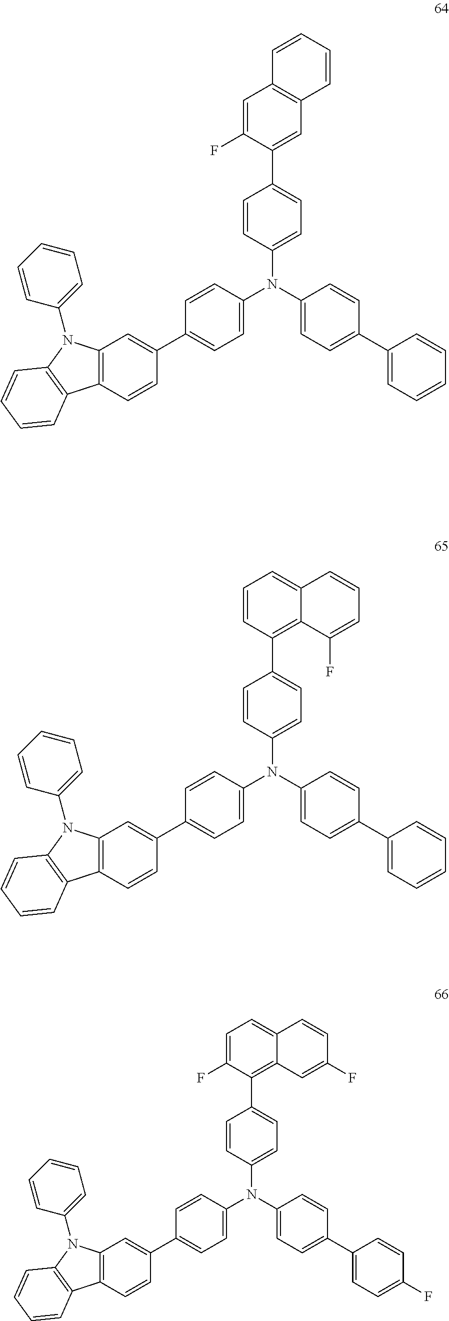 [SMILES: Fc1cc2ccccc2cc1-c1ccc(N(c2ccc(-c3ccccc3)cc2)c2ccc(-c3ccc4c5ccccc5n(-c5ccccc5)c4c3)cc2)cc1 ; Fc1cccc2cccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4c(F)ccc5ccc(F)cc45)cc3)cc2)cc1] 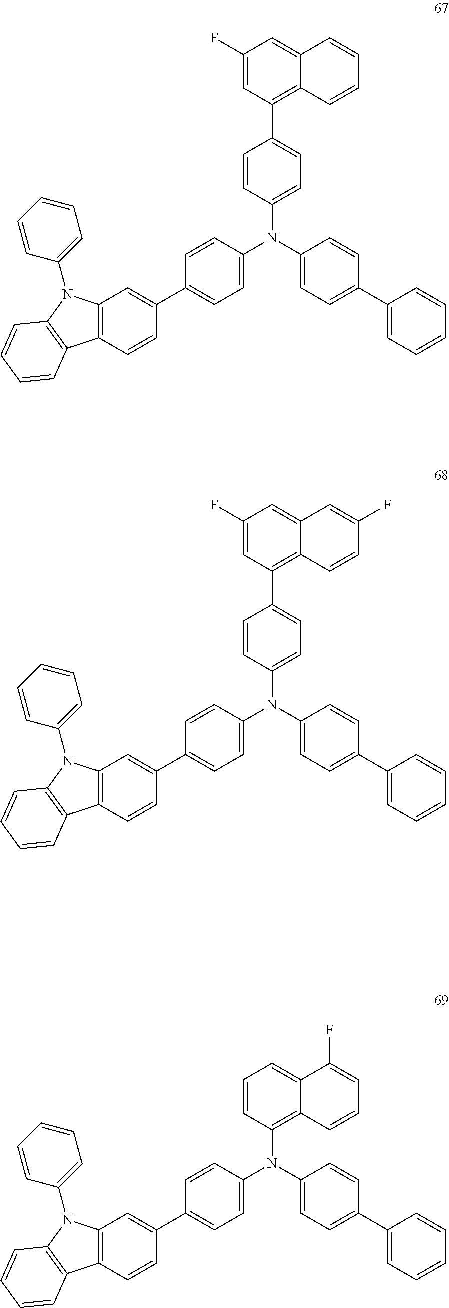 [SMILES: Fc1cc(-c2ccc(N(c3ccc(-c4ccccc4)cc3)c3ccc(-c4ccc5c6ccccc6n(-c6ccccc6)c5c4)cc3)cc2)c2ccccc2c1 ; Fc1ccc2c(-c3ccc(N(c4ccc(-c5ccccc5)cc4)c4ccc(-c5ccc6c7ccccc7n(-c7ccccc7)c6c5)cc4)cc3)cc(F)cc2c1 ; Fc1cccc2c(N(c3ccc(-c4ccccc4)cc3)c3ccc(-c4ccc5c6ccccc6n(-c6ccccc6)c5c4)cc3)cccc12] 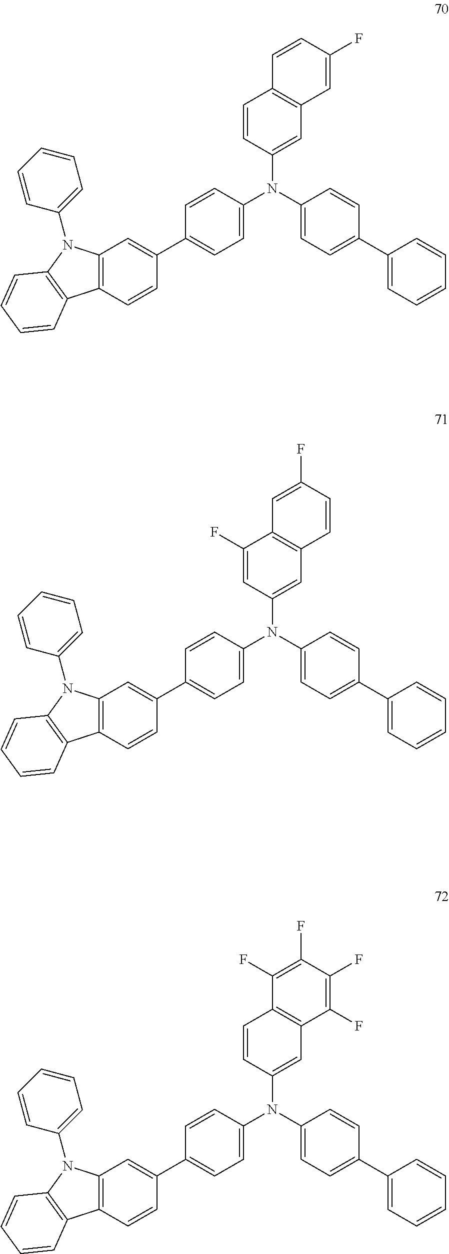 [SMILES: Fc1ccc2ccc(N(c3ccc(-c4ccccc4)cc3)c3ccc(-c4ccc5c6ccccc6n(-c6ccccc6)c5c4)cc3)cc2c1 ; Fc1ccc2cc(N(c3ccc(-c4ccccc4)cc3)c3ccc(-c4ccc5c6ccccc6n(-c6ccccc6)c5c4)cc3)cc(F)c2c1 ; Fc1c(F)c(F)c2cc(N(c3ccc(-c4ccccc4)cc3)c3ccc(-c4ccc5c6ccccc6n(-c6ccccc6)c5c4)cc3)ccc2c1F] 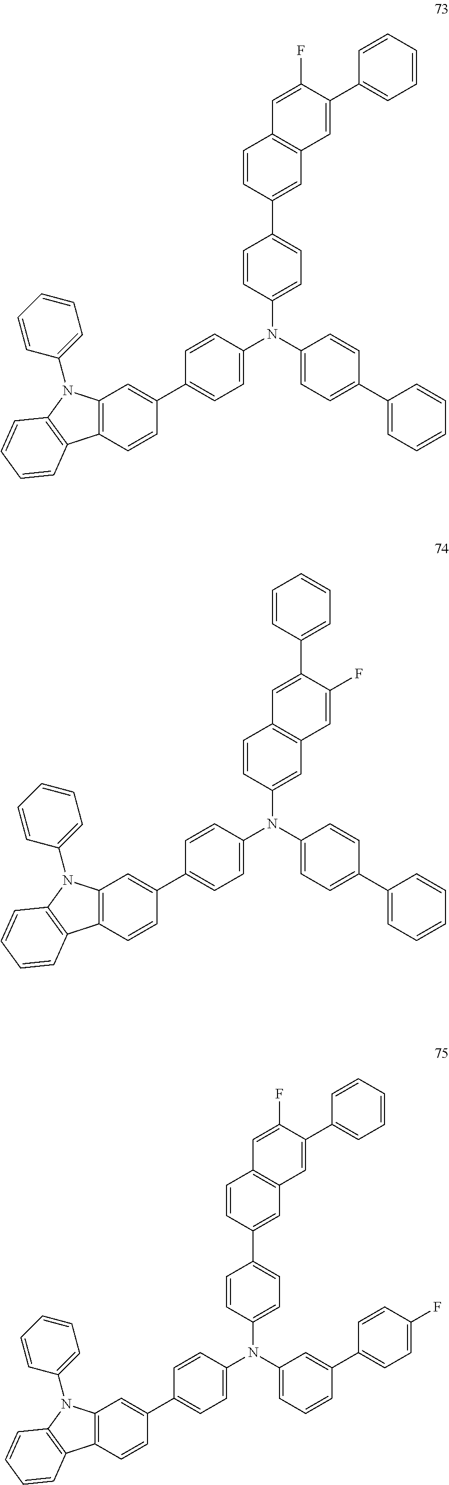 [SMILES: Fc1cc2ccc(-c3ccc(N(c4ccc(-c5ccccc5)cc4)c4ccc(-c5ccc6c7ccccc7n(-c7ccccc7)c6c5)cc4)cc3)cc2cc1-c1ccccc1 ; Fc1cc2cc(N(c3ccc(-c4ccccc4)cc3)c3ccc(-c4ccc5c6ccccc6n(-c6ccccc6)c5c4)cc3)ccc2cc1-c1ccccc1 ; Fc1ccc(-c2cccc(N(c3ccc(-c4ccc5cc(F)c(-c6ccccc6)cc5c4)cc3)c3ccc(-c4ccc5c6ccccc6n(-c6ccccc6)c5c4)cc3)c2)cc1] 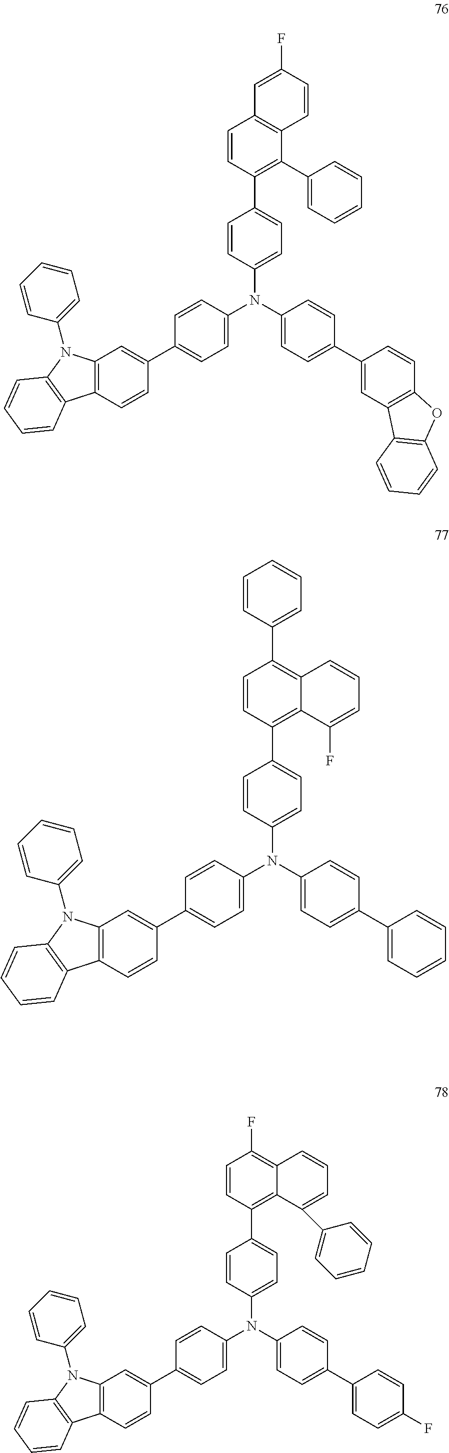 [SMILES: Fc1ccc2c(-c3ccccc3)c(-c3ccc(N(c4ccc(-c5ccc6oc7ccccc7c6c5)cc4)c4ccc(-c5ccc6c7ccccc7n(-c7ccccc7)c6c5)cc4)cc3)ccc2c1 ; Fc1cccc2c(-c3ccccc3)ccc(-c3ccc(N(c4ccc(-c5ccccc5)cc4)c4ccc(-c5ccc6c7ccccc7n(-c7ccccc7)c6c5)cc4)cc3)c12 ; Fc1ccc(-c2ccc(N(c3ccc(-c4ccc5c6ccccc6n(-c6ccccc6)c5c4)cc3)c3ccc(-c4ccc(F)c5cccc(-c6ccccc6)c45)cc3)cc2)cc1] 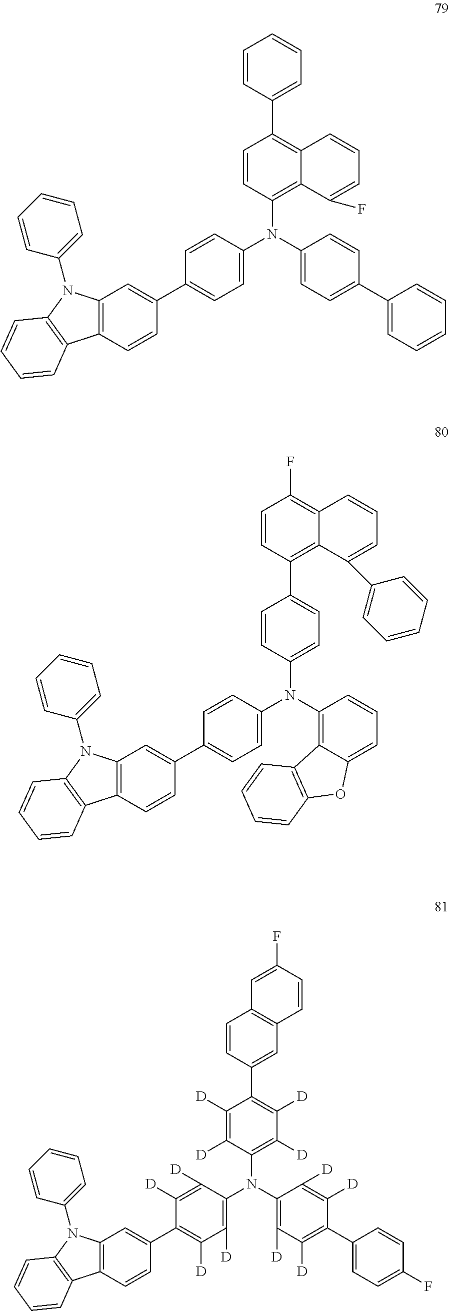 [SMILES: Fc1cccc2c(-c3ccccc3)ccc(N(c3ccc(-c4ccccc4)cc3)c3ccc(-c4ccc5c6ccccc6n(-c6ccccc6)c5c4)cc3)c12 ; Fc1ccc(-c2ccc(N(c3ccc(-c4ccc5c6ccccc6n(-c6ccccc6)c5c4)cc3)c3cccc4oc5ccccc5c34)cc2)c2c(-c3ccccc3)cccc12 ; [2H]c1c([2H])c(N(c2c([2H])c([2H])c(-c3ccc4cc(F)ccc4c3)c([2H])c2[2H])c2c([2H])c([2H])c(-c3ccc4c5ccccc5n(-c5ccccc5)c4c3)c([2H])c2[2H])c([2H])c([2H])c1-c1ccc(F)cc1] 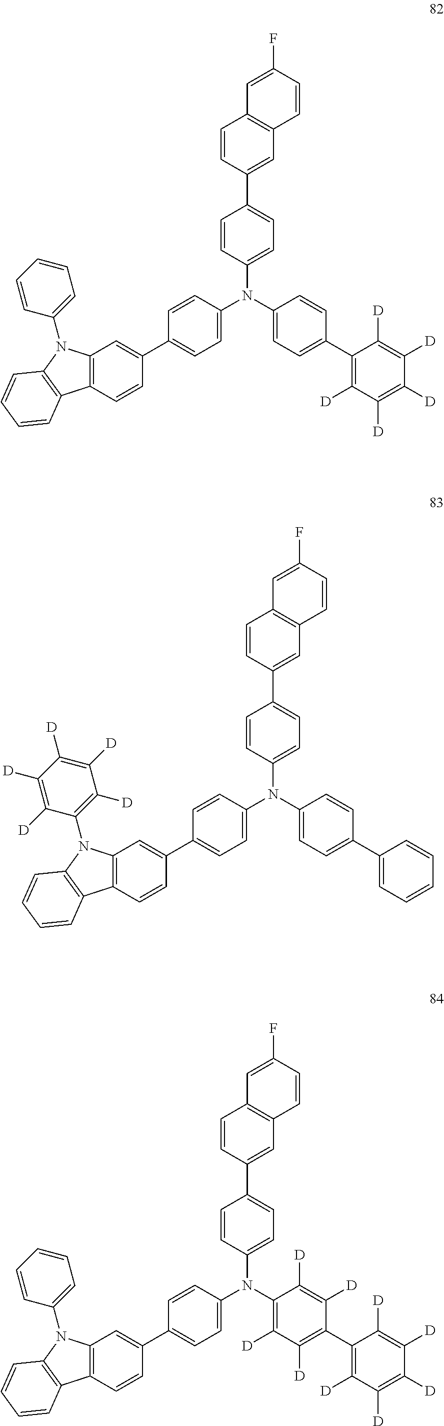 [SMILES: [2H]c1c([2H])c([2H])c(-c2ccc(N(c3ccc(-c4ccc5cc(F)ccc5c4)cc3)c3ccc(-c4ccc5c6ccccc6n(-c6ccccc6)c5c4)cc3)cc2)c([2H])c1[2H] ; [2H]c1c([2H])c([2H])c(-n2c3ccccc3c3ccc(-c4ccc(N(c5ccc(-c6ccccc6)cc5)c5ccc(-c6ccc7cc(F)ccc7c6)cc5)cc4)cc32)c([2H])c1[2H] ; [2H]c1c([2H])c([2H])c(-c2c([2H])c([2H])c(N(c3ccc(-c4ccc5cc(F)ccc5c4)cc3)c3ccc(-c4ccc5c6ccccc6n(-c6ccccc6)c5c4)cc3)c([2H])c2[2H])c([2H])c1[2H]] 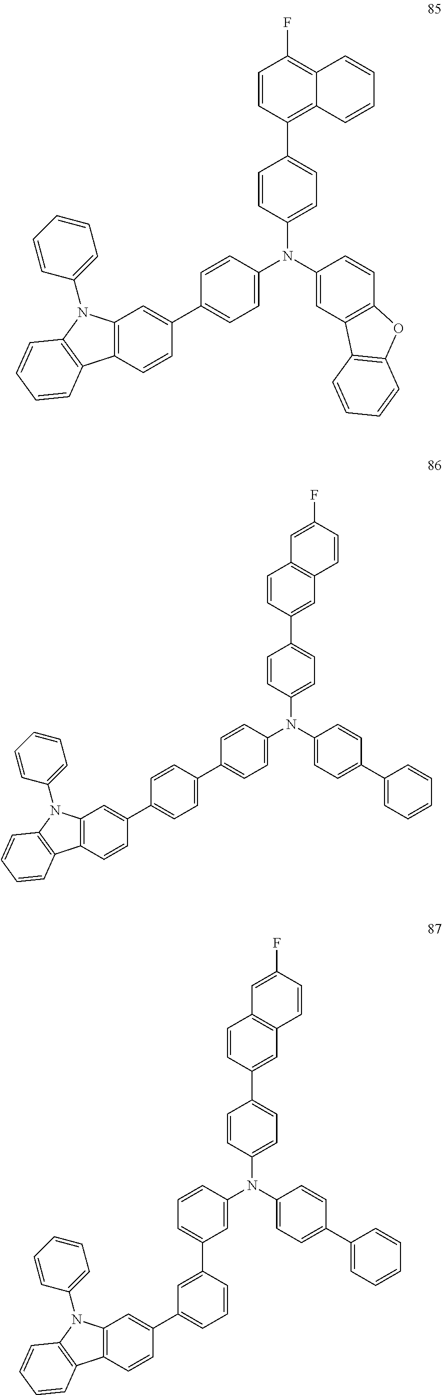 [SMILES: Fc1ccc(-c2ccc(N(c3ccc(-c4ccc5c6ccccc6n(-c6ccccc6)c5c4)cc3)c3ccc4oc5ccccc5c4c3)cc2)c2ccccc12 ; Fc1ccc2cc(-c3ccc(N(c4ccc(-c5ccccc5)cc4)c4ccc(-c5ccc(-c6ccc7c8ccccc8n(-c8ccccc8)c7c6)cc5)cc4)cc3)ccc2c1 ; Fc1ccc2cc(-c3ccc(N(c4ccc(-c5ccccc5)cc4)c4cccc(-c5cccc(-c6ccc7c8ccccc8n(-c8ccccc8)c7c6)c5)c4)cc3)ccc2c1] 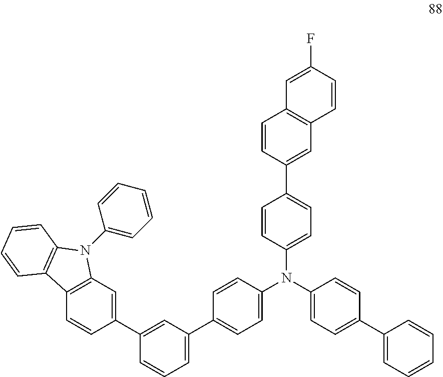 [SMILES: Fc1ccc2cc(-c3ccc(N(c4ccc(-c5ccccc5)cc4)c4ccc(-c5cccc(-c6ccc7c8ccccc8n(-c8ccccc8)c7c6)c5)cc4)cc3)ccc2c1] 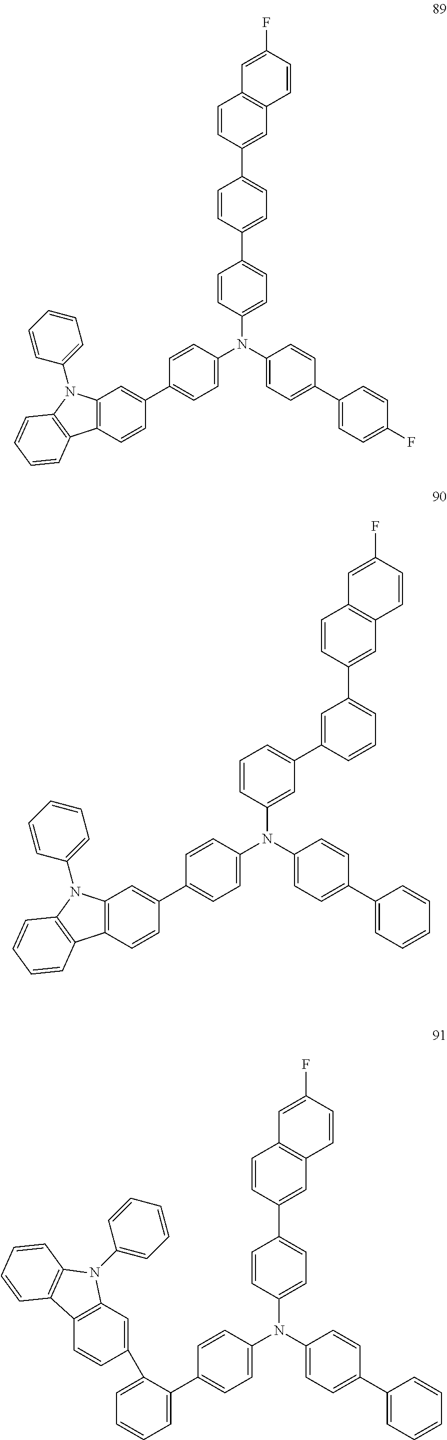 [SMILES: Fc1ccc(-c2ccc(N(c3ccc(-c4ccc(-c5ccc6cc(F)ccc6c5)cc4)cc3)c3ccc(-c4ccc5c6ccccc6n(-c6ccccc6)c5c4)cc3)cc2)cc1 ; Fc1ccc2cc(-c3cccc(-c4cccc(N(c5ccc(-c6ccccc6)cc5)c5ccc(-c6ccc7c8ccccc8n(-c8ccccc8)c7c6)cc5)c4)c3)ccc2c1 ; Fc1ccc2cc(-c3ccc(N(c4ccc(-c5ccccc5)cc4)c4ccc(-c5ccccc5-c5ccc6c7ccccc7n(-c7ccccc7)c6c5)cc4)cc3)ccc2c1] 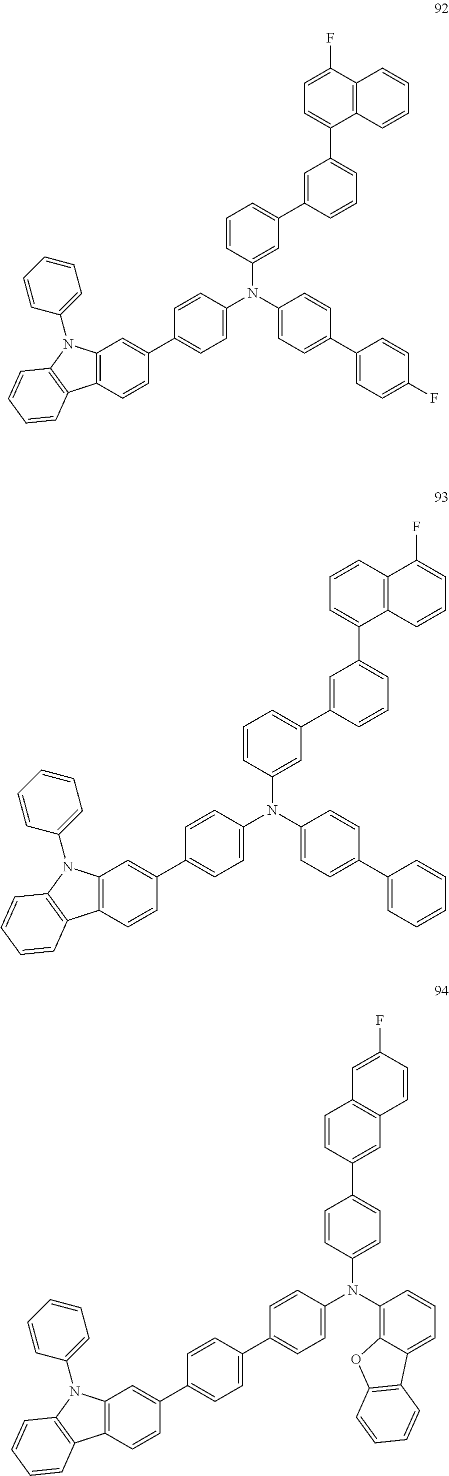 [SMILES: Fc1ccc(-c2ccc(N(c3ccc(-c4ccc5c6ccccc6n(-c6ccccc6)c5c4)cc3)c3cccc(-c4cccc(-c5ccc(F)c6ccccc56)c4)c3)cc2)cc1 ; Fc1cccc2c(-c3cccc(-c4cccc(N(c5ccc(-c6ccccc6)cc5)c5ccc(-c6ccc7c8ccccc8n(-c8ccccc8)c7c6)cc5)c4)c3)cccc12 ; Fc1ccc2cc(-c3ccc(N(c4ccc(-c5ccc(-c6ccc7c8ccccc8n(-c8ccccc8)c7c6)cc5)cc4)c4cccc5c4oc4ccccc45)cc3)ccc2c1] 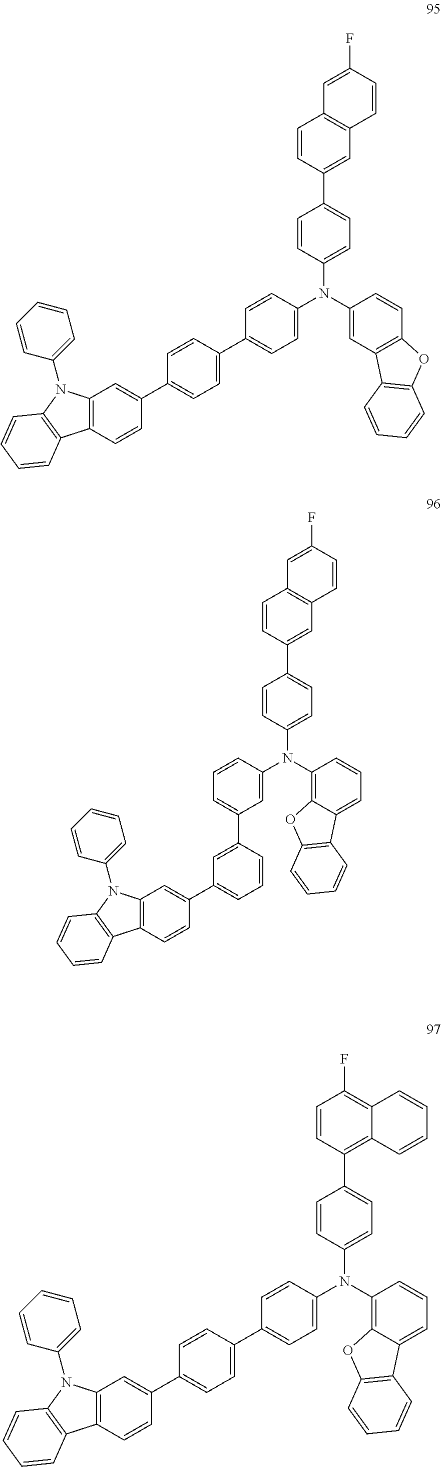 [SMILES: Fc1ccc2cc(-c3ccc(N(c4ccc(-c5ccc(-c6ccc7c8ccccc8n(-c8ccccc8)c7c6)cc5)cc4)c4ccc5oc6ccccc6c5c4)cc3)ccc2c1 ; Fc1ccc2cc(-c3ccc(N(c4cccc(-c5cccc(-c6ccc7c8ccccc8n(-c8ccccc8)c7c6)c5)c4)c4cccc5c4oc4ccccc45)cc3)ccc2c1 ; Fc1ccc(-c2ccc(N(c3ccc(-c4ccc(-c5ccc6c7ccccc7n(-c7ccccc7)c6c5)cc4)cc3)c3cccc4c3oc3ccccc34)cc2)c2ccccc12]
where, in Compound Group 1, D is a deuterium atom.

18. An electronic apparatus comprising a display module including a plurality of light-emitting elements,
wherein at least one among the plurality of light-emitting elements includes a first electrode, a second electrode disposed on the first electrode, an emission layer disposed between the first electrode and the second electrode, and a hole transport region disposed between the first electrode and the emission layer and containing an amine compound according to Formula 1: 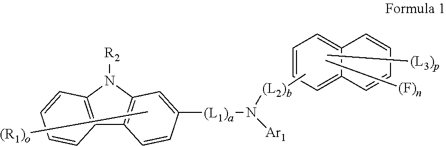
where, in Formula 1,
L₁ and L₂ are each independently a substituted or unsubstituted arylene group having 6 to 30 carbon atoms,
Ar₁ a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms,
each R₁, R₂, and each R₃ are independently a hydrogen atom, a deuterium atom, a substituted or unsubstituted aryl group having 6 to 40 ring-forming carbon atoms, a substituted or unsubstituted heteroaryl group having 5 to 40 ring-forming carbon atoms, or a substituted or unsubstituted alkyl group having 1 to 20 carbon atoms,
a and b are each independently an integer of 0 to 4, provided that;
when a is 0, N of NAr₁ is directly bonded to the 2-position of a carbazole group, and
when b is 0, N of NAr₁ is directly bonded to a naphthyl group,
n is an integer of 1 to 7,
is an integer of 0 to 7, and
p is an integer of 0 to (7-n).

19. The electronic apparatus of claim 18, further comprising a plurality of display devices, controlled independently and each configured to display an image, wherein at least one among the display devices includes the display module.

20. The electronic apparatus of claim 18, further comprising at least one among a processor, a memory, and a power module.
